(19) United States
(12) Patent Application Publication
(10) Pub. No.: US 2024/0412865 A1
(43) Pub. Date: Dec. 12, 2024

(54) BIOMARKERS FOR DIAGNOSING COLORECTAL CANCER OR ADVANCED ADENOMA

(71) Applicant: VENN BIOSCIENCES CORPORATION, South San Francisco, CA (US)

(72) Inventors: Daniel SERIE, South San Francisco, CA (US); Klaus LINDPAINTNER, South San Francisco, CA (US)

(21) Appl. No.: 18/294,950

(22) PCT Filed: Aug. 3, 2022
(86) PCT No.: PCT/US22/74482

§ 371 Date: Feb. 2, 2024

Related U.S. Application Data
(60) Provisional application No. 63/229,185, filed on Aug. 4, 2021.

(51) Int. Cl. G16H 50/20; G01N 33/68; G16B 40/20

(52) U.S. Cl. CPC G16H 50/20; G01N 33/6848; G16B 40/20; G01N 2800/60; G01N 2800/7028

(57) ABSTRACT

Set forth herein are glycopeptide biomarkers useful for diagnosing diseases and conditions, such as colorectal cancer or advanced adenoma. Also set forth herein are methods of generating glycopeptide biomarkers and methods of analyzing glycopeptides using mass spectroscopy. Also set forth herein are methods of analyzing glycopeptides using machine learning algorithms.

### CROSS-REFERENCE TO RELATED APPLICATIONS

This application claims priority to, and the benefit of, U.S. Provisional Patent Application No. 63/229,185, filed Aug. 4, 2021, the entire contents of which are herein incorporated by reference in its entirety for all purposes.

### SEQUENCE LISTING

The contents of the electronic sequence listing (166532000740SEQLIST.xml; Size: 57,343 bytes; and Date of Creation: Jul. 28, 2022) is herein incorporated by reference in its entirety

### FIELD

The instant disclosure is directed to glycoproteomic biomarkers including, but not limited to, glycans, peptides, and glycopeptides, as well as to methods of using these biomarkers with mass spectroscopy and in clinical applications.

### BACKGROUND

Changes in glycosylation have been described in relationship to disease states such as cancer. See, e.g., Dube, D. H.; Bertozzi, C. R. Glycans in Cancer and Inflammation-Potential for Therapeutics and Diagnostics. Nature Rev. Drug Disc. 2005, 4, 477-88, the entire contents of which are herein incorporated by reference in its entirety for all purposes. However, clinically relevant, non-invasive assays for diagnosing cancer, such as colorectal cancer or advanced adenoma, in a patient based on glycosylation changes in a sample from that patient are not yet sufficiently demonstrated.

Conventional clinical assays for diagnosing colorectal cancer or advanced adenoma, for example, include measuring the amount of the protein in a patient's blood by an enzyme-linked immunosorbent assay (ELISA). However, ELISA has limited sensitivity and precision. ELISA, for example, only measures protein at concentrations in the ng/ml range. This narrow measurement range limits the relevance of this assay by failing to measure biomarkers at concentrations substantially above or below this concentration range. Also, the ELISA assay is limited with respect to the types of samples which can be assayed. As a consequence of the lack of more precise and sensitive tests, patients who might otherwise be diagnosed with colorectal cancer or advanced adenoma are not and thereby fail to receive proper follow-up medical attention.

As an alternative, mass spectroscopy (MS) offers sensitive and precise measurement of cancer-specific biomarkers including glycopeptides. See, for example, Ruhaak, L. R., et al., Protein-Specific Differential Glycosylation of Immunoglobulins in Serum of Ovarian Cancer Patients DOI: 10.1021/acs.jproteome.5b01071; J. Proteome Res., 2016, 15, 1002-1010 (2016); also Miyamoto, S., et al., Multiple Reaction Monitoring for the Quantitation of Serum Protein Glycosylation Profiles: Application to Ovarian Cancer, DOI: 10.1021/acs.jproteome. 7b00541, J. Proteome Res. 2018, 17, 222-233 (2017), the entire contents of which are herein incorporated by reference in its entirety for all purposes. However, using MS to diagnose cancer, generally, or colorectal cancer or advanced adenoma specifically, has not been demonstrated to date in a clinically relevant manner.

What is needed are new biomarkers and new methods of using MS to diagnose disease states such as cancer using these biomarkers. Set forth herein in the disclosure below are such biomarkers comprising glycans, peptides, and glycopeptides, as well as fragments thereof, and methods of using the biomarkers with MS to diagnose colorectal cancer or advanced adenoma.

### SUMMARY

In one embodiment, set forth herein is a glycopeptide or peptide consisting of an amino acid sequence selected from SEQ ID NOs: 1-38, and combinations thereof.

In another embodiment, set forth herein is a glycopeptide or peptide consisting essentially of an amino acid sequence selected from SEQ ID NOs: 1-38, and combinations thereof.

In another embodiment, set forth herein is a method for detecting one or more MRM transitions, comprising: obtaining, or having obtained, a biological sample from a patient wherein the biological sample comprises one or more glycoproteins, glycans, or glycoproteins; digesting and/or fragmenting a glycopeptide in the sample; and detecting a multiple-reaction-monitoring (MRM) transition selected from the group consisting of transitions 1-38, described herein.

In another embodiment, set forth herein a method for identifying a classification for a sample, the method comprising: quantifying by mass spectroscopy (MS) one or more glycopeptides in a sample wherein the glycopeptides each, individually in each instance, comprise or consist essentially of or consist of an amino acid sequence selected from the group consisting of SEQ ID NOs: 1-38, together with any associated glycan, for instance as described herein, and combinations thereof; and inputting the quantification into a trained model to generate a output probability; determining if the output probability is above or below a threshold for a classification; and identifying a classification for the sample based on whether the output probability is above or below a threshold for a classification.

In yet another embodiment, set forth herein is a method for classifying a biological sample, comprising: obtaining, or having obtained a biological sample from a patient; digesting and/or fragmenting a glycopeptide in the sample; detecting a MRM transition selected from the group consisting of transitions 1-38; and quantifying the glycopeptides or fragments thereof; inputting the quantification into a trained model to generate a output probability; determining if the output probability is above or below a threshold for a classification; and classifying the biological sample based on whether the output probability is above or below a threshold for a classification.

In another embodiment, set forth herein is a method for treating a patient having colorectal cancer or advanced adenoma; the method comprising: obtaining, or having obtained a biological sample from the patient; digesting and/or fragmenting one or more glycopeptides in the sample; and detecting and quantifying one or more multiple-reaction-monitoring (MRM) transitions selected from the group consisting of transitions 1-38; inputting the quantification into a trained model to generate an output probability; determining if the output probability is above or below a threshold for a classification; and classifying the patient based on whether the output probability is above or below a threshold for a classification, wherein the classification is selected from the group consisting of: (A) a patient in need of resection; (B) a patient in need of a therapeutic agent; (C) a patient in need of an alkylating therapy; (D) a patient in need of a targeted therapeutic agent; (E) a patient in need of an immune therapeutic; (F) a patient in need of immune checkpoint inhibitors; (G) a patient in need of T-cell-related therapies; (H) a patient in need of a cancer vaccine; (I) a patient in need of radiotherapy; (J) a patient in need of a colonoscopy; or (K) a combination thereof; performing, or having performed, a resection if classification A or K is determined; performing, or having performed, a radiotherapy if classification I or K is determined; performing, or having performed, a colonoscopy if classification J or K is determined; or administering a therapeutically effective amount of a therapeutic agent to the patient: wherein the therapeutic agent is selected from a therapeutic agent if classification B or K is determined; or wherein the therapeutic agent is selected from alkylating agent if classification C or K is determined; or wherein the therapeutic agent is selected from targeted therapeutic agent if classification D or K is determined; wherein the therapeutic agent is selected from immune-therapeutic agent if classification E or K is determined; wherein the therapeutic agent is selected from immune checkpoint inhibitor if classification F or K is determined; wherein the therapeutic agent is selected from T-cell-related therapy if classification G or K is determined; and wherein the therapeutic agent is selected from a cancer vaccine if classification H or K is determined.

In another embodiment, set forth herein is a method for training a machine learning algorithm, comprising: providing a first data set of MRM transition signals indicative of a sample comprising a glycopeptide consisting of, or consisting essentially of, an amino acid sequence selected from the group consisting of SEQ ID NOs: 1-38; providing a second data set of MRM transition signals indicative of a control sample; and comparing the first data set with the second data set using a machine learning algorithm. In certain embodiments, LASSO methodology with cross-validation for selection of hyperparameters is used to train the machine learning algorithm.

In another embodiment, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect one or more MRM transitions selected from transitions 1-38 and quantify the glycans, peptides and glycopeptides associated with the MRM transitions; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having colorectal cancer or advanced adenoma based on the diagnostic classification. In some examples, the method includes performing mass spectroscopy of the biological sample using MRM-MS with a QQQ.

In another embodiment, set forth herein is a kit comprising a glycopeptide standard, a buffer, and one or more glycopeptides consisting of, or consisting essentially of, an amino acid sequence selected from the group consisting of SEQ ID NOs: 1-38.

In another embodiment, set forth herein is a glycopeptide consisting of, or consisting essentially of, an amino acid sequence selected from the group consisting of SEQ ID NOs: 1-38.

### BRIEF DESCRIPTIONS OF THE DRAWINGS

FIG. 1 shows a plot of probability of having colorectal cancer using Model 1.

FIG. 2 shows a plot of probability of having an advanced adenoma using Model 2.

FIG. 3A shows an Area Under the Curve (AUC) analysis of Model 1 with respect to the individual markers. FIG. 3B shows an AUC analysis of Model 2 with respect to the individual markers.

### DETAILED DESCRIPTION

The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the inventions herein are not intended to be limited to the embodiments presented, but are to be accorded their widest scope consistent with the principles and novel features disclosed herein.

All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

### I. General

The instant disclosure provides methods and compositions for the profiling, detecting, and/or quantifying of glycans and glycopeptides in a biological sample. In some examples, glycan and glycopeptide panels are described for diagnosing and screening patients having colorectal cancer or advanced adenoma. In some examples, glycan and glycopeptide panels are described for diagnosing and screening patients having cancer.

Certain techniques for analyzing biological samples using mass spectroscopy are known. See, for example, International PCT Patent Application Publication No. WO2019079639A1, filed Oct. 18, 2018 as International Patent Application No. PCT/US2018/56574, and titled IDENTIFICATION AND USE OF BIOLOGICAL PARAMETERS FOR DIAGNOSIS AND TREATMENT MONITORING, the entire contents of which are herein incorporated by reference in its entirety for all purposes. See, also, US Patent Application Publication No. US20190101544A1, filed Aug. 31, 2018 as U.S. patent application Ser. No. 16/120,016, and titled IDENTIFICATION AND USE OF GLYCOPEPTIDES AS BIOMARKERS FOR DIAGNOSIS AND TREATMENT MONITORING, the entire contents of which are herein incorporated by reference in its entirety for all purposes.

### II. Definitions

As used herein, the singular forms “a,” “an” and “the” include plural referents unless the context clearly dictates otherwise.

As used herein, the phrase “biological sample,” refers to a sample derived from, obtained by, generated from, provided from, take from, or removed from an organism; or from fluid or tissue from the organism. Biological samples include, but are not limited to synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, any derivative or fraction of the aforementioned biological samples.

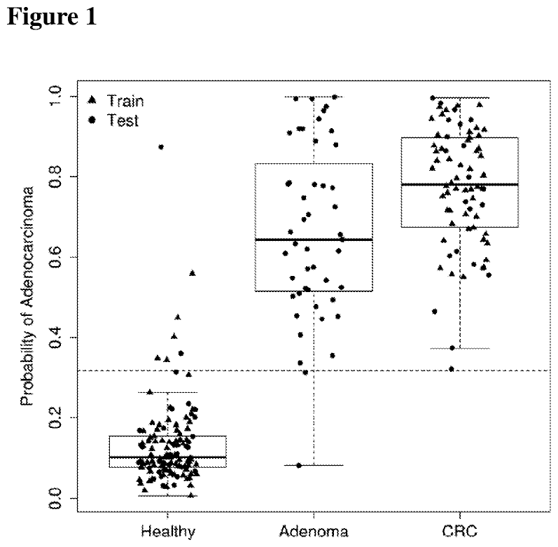

As used herein, the term “glycan” refers to the carbohydrate residue of a glycoconjugate, such as the carbohydrate portion of a glycopeptide, glycoprotein, glycolipid or proteoglycan. Glycan structures are described by a glycan reference code number, and also illustrated in International PCT Patent Application No. PCT/US2020/0162861, filed Jan. 31, 2020, which is herein incorporated by reference in its entirety for all purposes. For example see FIGS. 1 through 14 of PCT Patent Application No. PCT/US2020/0162861, filed Jan. 31, 2020, which are herein incorporated by reference in their entirety for all purposes.

As used herein, the term “glycopeptide,” refers to a peptide having at least one glycan residue bonded thereto. In each embodiment described herein, the glycopeptide may comprise, consist essentially of, or consist of, the amino acid sequence specified by the indicated SEQ ID NO together with one or more glycans, for instance those described herein associated with that SEQ ID NO. For instance, a glycopeptide according to SEQ ID NO:1, as used herein, can refer to a glycopeptide according to the amino acid sequence of SEQ ID NO: 1 and glycan 5411, wherein the glycan is bonded to residue 107. A glycopeptide comprising SEQ ID NO: 1, as used herein, can refer to a glycopeptide comprising the amino acid sequence of SEQ ID NO: 1 and glycan 5411, wherein the glycan is bonded to residue 107. A glycopeptide consisting essentially of SEQ ID NO: 1, as used herein, can refer to a glycopeptide consisting essentially of the amino acid sequence of SEQ ID NO:1 and glycan 5411, wherein the glycan is bonded to residue 107. A glycopeptide consisting of to SEQ ID NO: 1, as used herein, can refer to a glycopeptide consisting of the amino acid sequence of SEQ ID NO: 1 and glycan 5411, wherein the glycan is bonded to residue 107. Similarly usage applies to SEQ ID NOS: 2-38, with the glycans described in sections below.

As used herein, the phrase “glycosylated peptides,” refers to a peptide bonded to a glycan residue.

As used herein, the phrase “glycopeptide fragment” or “glycosylated peptide fragment” refers to a glycosylated peptide (or glycopeptide) having an amino acid sequence that is the same as part (but not all) of the amino acid sequence of the glycosylated protein from which the glycosylated peptide is obtained by digestion, e.g., with one or more protease(s) or by fragmentation, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

As used herein, the phrase “multiple reaction monitoring mass spectrometry (MRM-MS),” refers to a highly sensitive and selective method for the targeted quantification of glycans and peptides in biological samples. Unlike traditional mass spectrometry, MRM-MS is highly selective (targeted), allowing researchers to fine tune an instrument to specifically look for certain peptides fragments of interest. MRM allows for greater sensitivity, specificity, speed and quantitation of peptides fragments of interest, such as a potential biomarker. MRM-MS involves using one or more of a triple quadrupole (QQQ) mass spectrometer and a quadrupole time-of-flight (qTOF) mass spectrometer.

As used herein, the phrase “digesting a glycopeptide,” refers to a biological process that employs enzymes to break specific amino acid peptide bonds. For example, digesting a glycopeptide includes contacting a glycopeptide with an digesting enzyme, e.g., trypsin, to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing.

As used herein, the phrase “fragmenting a glycopeptide,” refers to the ion fragmentation process which occurs in a MRM-MS instrument. Fragmenting may produce various fragments having the same mass but varying with respect to their charge.

As used herein, the term “subject,” refers to a mammal. The non-liming examples of a mammal include a human, non-human primate, mouse, rat, dog, cat, horse, or cow, and the like. Mammals other than humans can be advantageously used as subjects that represent animal models of disease, pre-disease, or a pre-disease condition. A subject can be male or female. A subject can be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition.

As used herein, the term “patient” refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

As used herein, “peptide,” is meant to include glycopeptides unless stated otherwise.

As used herein, the phrase “multiple-reaction-monitoring (MRM) transition,” refers to the mass to charge (m/z) peaks or signals observed when a glycopeptide, or a fragment thereof, is detected by MRM-MS. The MRM transition is detected as the transition of the precursor and product ion.

As used herein, the phrase “detecting a multiple-reaction-monitoring (MRM) transition,” refers to the process in which a mass spectrometer analyzes a sample using tandem mass spectrometer ion fragmentation methods and identifies the mass to charge ratio for ion fragments in a sample. The absolute value of these identified mass to charge ratios are referred to as transitions. In the context of the methods set forth herein, the mass to charge ratio transitions are the values indicative of glycan, peptide or glycopeptide ion fragments. For some glycopeptides set forth herein, there is a single transition peak or signal. For some other glycopeptides set forth herein, there is more than one transition peak or signal. Background information on MRM mass spectrometry can be found in Introduction to Mass Spectrometry: Instrumentation, Applications, and Strategies for Data Interpretation, 4th Edition, J. Throck Watson, O. David Sparkman, ISBN: 978-0-470-51634-8, November 2007, the entire contents of which are here incorporated by reference in its entirety for all purposes.

As used herein, the phrase “detecting a multiple-reaction-monitoring (MRM) transition indicative of a glycopeptide,” refers to a MS process in which a MRM-MS transition is detected and then compare to a calculated mass to charge ratio (m/z) of a glycopeptide, or fragment thereof, in order to identify the glycopeptide. In some examples, herein, a single transition may be indicative of two more glycopeptides, if those glycopeptides have identical MRM-MS fragmentation patterns. A transition peak or signal includes, but is not limited to, those transitions set forth herein were are associated with a glycopeptide consisting essentially of an amino acid sequence selected from SEQ ID NOs: 1-38, and combinations thereof, according to Tables 1-5, e.g., Table 1, Table 2, Table 3, Table 4, Table 5, or a combination thereof. A transition peak or signal includes, but is not limited to, those transitions set forth herein were are associated with a glycopeptide consisting of an amino acid sequence selected from SEQ ID NOs: 1-38, and combinations thereof, according to Tables 1-5, e.g., Table 1, Table 2, Table 3, Table 4, Table 5, or a combination thereof.

As used herein, the term “reference value” refers to a value obtained from a population of individual(s) whose disease state is known. The reference value may be in n-dimensional feature space and may be defined by a maximum-margin hyperplane. A reference value can be determined for any particular population, subpopulation, or group of individuals according to standard methods well known to those of skill in the art.

As used herein, the term “population of individuals” means one or more individuals. In one embodiment, the population of individuals consists of one individual. In one embodiment, the population of individuals comprises multiple individuals. As used herein, the term “multiple” means at least 2 (such as at least 4, 6, 8, 10, 12, 14, 16, 18, 20, 22, 24, 26, 28, or 30) individuals. In one embodiment, the population of individuals comprises at least 10 individuals.

As used herein, the term “treatment” or “treating” means any treatment of a disease or condition in a subject, such as a mammal, including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms. Treating may include administering therapeutic agents to a subject in need thereof.

Glycans are referenced herein using the Symbol Nomenclature for Glycans (SNFG) for illustrating glycans. An explanation of this illustration system is available on the internet at www.ncbi.nlm.nih.gov/glycans/snfg.html, the entire contents of which are herein incorporated by reference in its entirety for all purposes. Symbol Nomenclature for Graphical Representation of Glycans as published in Glycobiology 25:1323-1324, 2015, which is available on the internet at doi.org/10.1093/glycob/cwv091. Additional information showing illustrations of the SNFG system are. Within this system, the term, Hex_i: is interpreted as follows: i indicates the number of green circles (mannose) and the number of yellow circles (galactose). The term, HexNAC_j, uses j to indicate the number of blue squares (GlcNAC's). The term Fuc_d, uses d to indicate the number of red triangles (fucose). The term NeusAC_1, uses 1 to indicate the number of purple diamonds (sialic acid). The glycan reference codes used herein combine these i, j, d, and 1 terms to make a composite 4-5 number glycan reference code, e.g., 5300 or 5320. As an example, glycans 3200 and 3210 in FIG. 1 both include 3 green circles (mannose), 2 blue squares (GlcNAC's), and no purple diamonds (sialic acid) but differ in that glycan 3210 also includes 1 red triangle (fucose). See, for example, FIGS. 1 through 14 of PCT Patent Application No. PCT/US2020/0162861, filed Jan. 31, 2020, which are herein incorporated by reference in their entirety for all purposes.

### III. Biomarkers

Set forth herein are biomarkers. These biomarkers are useful for a variety of applications, including, but not limited to, diagnosing diseases and conditions. For example, certain biomarkers set forth herein, or combinations thereof, are useful for diagnosing colorectal cancer or advanced adenoma cancer. In some other examples, certain biomarkers set forth herein, or combinations thereof, are useful for diagnosing and screening patients having cancer, an autoimmune disease, or fibrosis. In some examples, the biomarkers set forth herein, or combinations thereof, are useful for classifying a patient so that the patient receives the appropriate medical treatment. In some other examples, the biomarkers set forth herein, or combinations thereof, are useful for treating or ameliorating a disease or condition in patient by, for example, identifying a therapeutic agent with which to treat a patient. In some other examples, the biomarkers set forth herein, or combinations thereof, are useful for determining a prognosis of treatment for a patient or a likelihood of success or survivability for a treatment regimen.

In some examples, a sample from a patient is analyzed by MS and the results are used to determine the presence, absolute amount, and/or relative amount of a glycopeptide comprising an amino acid sequence of any of SEQ ID NOs: 1-38 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of an amino acid sequence selected from SEQ ID NOs: 1-38 in the sample. In some examples, a sample from a patient is analyzed by MS and the results are used to determine the presence, absolute amount, and/or relative amount of a glycopeptide consisting essentially of an amino acid sequence selected from SEQ ID NOs: 1-38 in the sample. In some examples, a sample from a patient is analyzed by MS and the results are used to determine the presence, absolute amount, and/or relative amount of a glycopeptide consisting of, or consisting essentially of, an amino acid sequence selected from SEQ ID NOs: 1-38 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide comprising an amino acid sequence of any of SEQ ID NOs: 5, 8-11, 13-14, 16-22, 26-28, 30-31, and 34-38 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of an amino acid sequence selected from SEQ ID NOs: 5, 8-11, 13-14, 16-22, 26-28, 30-31, and 34-38 in the sample. In some examples, a sample from a patient is analyzed by MS and the results are used to determine the presence, absolute amount, and/or relative amount of a glycopeptide consisting essentially of an amino acid sequence selected from SEQ ID NOs: 5, 8-11, 13-14, 16-22, 26-28, 30-31, and 34-38 in the sample. In some examples, a sample from a patient is analyzed by MS and the results are used to determine the presence, absolute amount, and/or relative amount of a glycopeptide consisting of, or consisting essentially of, an amino acid sequence selected from SEQ ID NOs: 5, 8-11, 13-14, 16-22, 26-28, 30-31, and 34-38 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of amino acid sequence SEQ ID NO: 5, in the sample. In some examples, a sample from a patient is analyzed by MS and the results are used to determine the presence, absolute amount, and/or relative amount of a glycopeptide consisting of amino acid sequence SEQ ID NO: 8, in the sample. In some examples, a sample from a patient is analyzed by MS and the results are used to determine the presence, absolute amount, and/or relative amount of a glycopeptide consisting of amino acid sequence SEQ ID NO: 9, in the sample. In some examples, a sample from a patient is analyzed by MS and the results are used to determine the presence, absolute amount, and/or relative amount of a glycopeptide consisting of amino acid sequence SEQ ID NO: 10, in the sample. In some examples, a sample from a patient is analyzed by MS and the results are used to determine the presence, absolute amount, and/or relative amount of a glycopeptide consisting of amino acid sequence SEQ ID NO: 11, in the sample. In some examples, a sample from a patient is analyzed by MS and the results are used to determine the presence, absolute amount, and/or relative amount of a glycopeptide consisting of amino acid sequence SEQ ID NO: 13, in the sample. In some examples, a sample from a patient is analyzed by MS and the results are used to determine the presence, absolute amount, and/or relative amount of a glycopeptide consisting of amino acid sequence SEQ ID NO: 14, in the sample. In some examples, a sample from a patient is analyzed by MS and the results are used to determine the presence, absolute amount, and/or relative amount of a glycopeptide consisting of amino acid sequence SEQ ID NO: 16, in the sample. In some examples, a sample from a patient is analyzed by MS and the results are used to determine the presence, absolute amount, and/or relative amount of a glycopeptide consisting of amino acid sequence SEQ ID NO: 17, in the sample. In some examples, a sample from a patient is analyzed by MS and the results are used to determine the presence, absolute amount, and/or relative amount of a glycopeptide consisting of amino acid sequence SEQ ID NO: 18, in the sample. In some examples, a sample from a patient is analyzed by MS and the results are used to determine the presence, absolute amount, and/or relative amount of a glycopeptide consisting of amino acid sequence SEQ ID NO: 19, in the sample. In some examples, a sample from a patient is analyzed by MS and the results are used to determine the presence, absolute amount, and/or relative amount of a glycopeptide consisting of amino acid sequence SEQ ID NO: 20, in the sample. In some examples, a sample from a patient is analyzed by MS and the results are used to determine the presence, absolute amount, and/or relative amount of a glycopeptide consisting of amino acid sequence SEQ ID NO: 21, in the sample. In some examples, a sample from a patient is analyzed by MS and the results are used to determine the presence, absolute amount, and/or relative amount of a glycopeptide consisting of amino acid sequence SEQ ID NO: 22, in the sample. In some examples, a sample from a patient is analyzed by MS and the results are used to determine the presence, absolute amount, and/or relative amount of a glycopeptide consisting of amino acid sequence SEQ ID NO: 26, in the sample. In some examples, a sample from a patient is analyzed by MS and the results are used to determine the presence, absolute amount, and/or relative amount of a glycopeptide consisting of amino acid sequence SEQ ID NO: 27, in the sample. In some examples, a sample from a patient is analyzed by MS and the results are used to determine the presence, absolute amount, and/or relative amount of a glycopeptide consisting of amino acid sequence SEQ ID NO: 28, in the sample. In some examples, a sample from a patient is analyzed by MS and the results are used to determine the presence, absolute amount, and/or relative amount of a glycopeptide consisting of amino acid sequence SEQ ID NO: 30, in the sample. In some examples, a sample from a patient is analyzed by MS and the results are used to determine the presence, absolute amount, and/or relative amount of a glycopeptide consisting of amino acid sequence SEQ ID NO: 31, in the sample. In some examples, a sample from a patient is analyzed by MS and the results are used to determine the presence, absolute amount, and/or relative amount of a glycopeptide consisting of amino acid sequence SEQ ID NO: 34, in the sample. In some examples, a sample from a patient is analyzed by MS and the results are used to determine the presence, absolute amount, and/or relative amount of a glycopeptide consisting of amino acid sequence SEQ ID NO: 35, in the sample. In some examples, a sample from a patient is analyzed by MS and the results are used to determine the presence, absolute amount, and/or relative amount of a glycopeptide consisting of amino acid sequence SEQ ID NO: 36, in the sample. In some examples, a sample from a patient is analyzed by MS and the results are used to determine the presence, absolute amount, and/or relative amount of a glycopeptide consisting of amino acid sequence SEQ ID NO: 37, in the sample. In some examples, a sample from a patient is analyzed by MS and the results are used to determine the presence, absolute amount, and/or relative amount of a glycopeptide consisting of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting essentially of a sequence SEQ ID NO: 5, in the sample. In some examples, a sample from a patient is analyzed by MS and the results are used to determine the presence, absolute amount, and/or relative amount of a glycopeptide consisting essentially of amino acid sequence SEQ ID NO: 8, in the sample. In some examples, a sample from a patient is analyzed by MS and the results are used to determine the presence, absolute amount, and/or relative amount of a glycopeptide consisting essentially of amino acid sequence SEQ ID NO: 9, in the sample. In some examples, a sample from a patient is analyzed by MS and the results are used to determine the presence, absolute amount, and/or relative amount of a glycopeptide consisting essentially of amino acid sequence SEQ ID NO: 10, in the sample. In some examples, a sample from a patient is analyzed by MS and the results are used to determine the presence, absolute amount, and/or relative amount of a glycopeptide consisting essentially of amino acid sequence SEQ ID NO: 11, in the sample. In some examples, a sample from a patient is analyzed by MS and the results are used to determine the presence, absolute amount, and/or relative amount of a glycopeptide consisting essentially of amino acid sequence SEQ ID NO: 13, in the sample. In some examples, a sample from a patient is analyzed by MS and the results are used to determine the presence, absolute amount, and/or relative amount of a glycopeptide consisting essentially of amino acid sequence SEQ ID NO: 14, in the sample. In some examples, a sample from a patient is analyzed by MS and the results are used to determine the presence, absolute amount, and/or relative amount of a glycopeptide consisting essentially of amino acid sequence SEQ ID NO: 16, in the sample. In some examples, a sample from a patient is analyzed by MS and the results are used to determine the presence, absolute amount, and/or relative amount of a glycopeptide consisting essentially of amino acid sequence SEQ ID NO: 17, in the sample. In some examples, a sample from a patient is analyzed by MS and the results are used to determine the presence, absolute amount, and/or relative amount of a glycopeptide consisting essentially of amino acid sequence SEQ ID NO: 18, in the sample. In some examples, a sample from a patient is analyzed by MS and the results are used to determine the presence, absolute amount, and/or relative amount of a glycopeptide consisting essentially of amino acid sequence SEQ ID NO: 19, in the sample. In some examples, a sample from a patient is analyzed by MS and the results are used to determine the presence, absolute amount, and/or relative amount of a glycopeptide consisting essentially of amino acid sequence SEQ ID NO: 20, in the sample. In some examples, a sample from a patient is analyzed by MS and the results are used to determine the presence, absolute amount, and/or relative amount of a glycopeptide consisting essentially of amino acid sequence SEQ ID NO: 21, in the sample. In some examples, a sample from a patient is analyzed by MS and the results are used to determine the presence, absolute amount, and/or relative amount of a glycopeptide consisting essentially of amino acid sequence SEQ ID NO: 22, in the sample. In some examples, a sample from a patient is analyzed by MS and the results are used to determine the presence, absolute amount, and/or relative amount of a glycopeptide consisting essentially of amino acid sequence SEQ ID NO: 26, in the sample. In some examples, a sample from a patient is analyzed by MS and the results are used to determine the presence, absolute amount, and/or relative amount of a glycopeptide consisting essentially of amino acid sequence SEQ ID NO: 27, in the sample. In some examples, a sample from a patient is analyzed by MS and the results are used to determine the presence, absolute amount, and/or relative amount of a glycopeptide consisting essentially of amino acid sequence SEQ ID NO: 28, in the sample. In some examples, a sample from a patient is analyzed by MS and the results are used to determine the presence, absolute amount, and/or relative amount of a glycopeptide consisting essentially of amino acid sequence SEQ ID NO: 30, in the sample. In some examples, a sample from a patient is analyzed by MS and the results are used to determine the presence, absolute amount, and/or relative amount of a glycopeptide consisting essentially of amino acid sequence SEQ ID NO: 31, in the sample. In some examples, a sample from a patient is analyzed by MS and the results are used to determine the presence, absolute amount, and/or relative amount of a glycopeptide consisting essentially of amino acid sequence SEQ ID NO: 34, in the sample. In some examples, a sample from a patient is analyzed by MS and the results are used to determine the presence, absolute amount, and/or relative amount of a glycopeptide consisting essentially of amino acid sequence SEQ ID NO: 35, in the sample. In some examples, a sample from a patient is analyzed by MS and the results are used to determine the presence, absolute amount, and/or relative amount of a glycopeptide consisting essentially of amino acid sequence SEQ ID NO: 36, in the sample. In some examples, a sample from a patient is analyzed by MS and the results are used to determine the presence, absolute amount, and/or relative amount of a glycopeptide consisting essentially of amino acid sequence SEQ ID NO: 37, in the sample. In some examples, a sample from a patient is analyzed by MS and the results are used to determine the presence, absolute amount, and/or relative amount of a glycopeptide consisting essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of, or consisting essentially of sequence SEQ ID NO: 5, in the sample. In some examples, a sample from a patient is analyzed by MS and the results are used to determine the presence, absolute amount, and/or relative amount of a glycopeptide consisting of, or consisting essentially of amino acid sequence SEQ ID NO: 8, in the sample. In some examples, a sample from a patient is analyzed by MS and the results are used to determine the presence, absolute amount, and/or relative amount of a glycopeptide consisting of, or consisting essentially of amino acid sequence SEQ ID NO: 9, in the sample. In some examples, a sample from a patient is analyzed by MS and the results are used to determine the presence, absolute amount, and/or relative amount of a glycopeptide consisting of, or essentially of amino acid sequence SEQ ID NO: 10, in the sample. In some examples, a sample from a patient is analyzed by MS and the results are used to determine the presence, absolute amount, and/or relative amount of a glycopeptide consisting of, or essentially of amino acid sequence SEQ ID NO: 11, in the sample. In some examples, a sample from a patient is analyzed by MS and the results are used to determine the presence, absolute amount, and/or relative amount of a glycopeptide consisting of, or essentially of amino acid sequence SEQ ID NO: 13, in the sample. In some examples, a sample from a patient is analyzed by MS and the results are used to determine the presence, absolute amount, and/or relative amount of a glycopeptide consisting of, or essentially of amino acid sequence SEQ ID NO: 14, in the sample. In some examples, a sample from a patient is analyzed by MS and the results are used to determine the presence, absolute amount, and/or relative amount of a glycopeptide consisting of, or essentially of amino acid sequence SEQ ID NO: 16, in the sample. In some examples, a sample from a patient is analyzed by MS and the results are used to determine the presence, absolute amount, and/or relative amount of a glycopeptide consisting of, or essentially of amino acid sequence SEQ ID NO: 17, in the sample. In some examples, a sample from a patient is analyzed by MS and the results are used to determine the presence, absolute amount, and/or relative amount of a glycopeptide consisting of, or essentially of amino acid sequence SEQ ID NO: 18, in the sample. In some examples, a sample from a patient is analyzed by MS and the results are used to determine the presence, absolute amount, and/or relative amount of a glycopeptide consisting of, or essentially of amino acid sequence SEQ ID NO: 19, in the sample. In some examples, a sample from a patient is analyzed by MS and the results are used to determine the presence, absolute amount, and/or relative amount of a glycopeptide consisting of, or essentially of amino acid sequence SEQ ID NO: 20, in the sample. In some examples, a sample from a patient is analyzed by MS and the results are used to determine the presence, absolute amount, and/or relative amount of a glycopeptide consisting of, or essentially of amino acid sequence SEQ ID NO: 21, in the sample. In some examples, a sample from a patient is analyzed by MS and the results are used to determine the presence, absolute amount, and/or relative amount of a glycopeptide consisting of, or essentially of amino acid sequence SEQ ID NO: 22, in the sample. In some examples, a sample from a patient is analyzed by MS and the results are used to determine the presence, absolute amount, and/or relative amount of a glycopeptide consisting of, or essentially of amino acid sequence SEQ ID NO: 26, in the sample. In some examples, a sample from a patient is analyzed by MS and the results are used to determine the presence, absolute amount, and/or relative amount of a glycopeptide consisting of, or essentially of amino acid sequence SEQ ID NO: 27, in the sample. In some examples, a sample from a patient is analyzed by MS and the results are used to determine the presence, absolute amount, and/or relative amount of a glycopeptide consisting of, or essentially of amino acid sequence SEQ ID NO: 28, in the sample. In some examples, a sample from a patient is analyzed by MS and the results are used to determine the presence, absolute amount, and/or relative amount of a glycopeptide consisting of, or essentially of amino acid sequence SEQ ID NO: 30, in the sample. In some examples, a sample from a patient is analyzed by MS and the results are used to determine the presence, absolute amount, and/or relative amount of a glycopeptide consisting of, or essentially of amino acid sequence SEQ ID NO: 31, in the sample. In some examples, a sample from a patient is analyzed by MS and the results are used to determine the presence, absolute amount, and/or relative amount of a glycopeptide consisting of, or essentially of amino acid sequence SEQ ID NO: 34, in the sample. In some examples, a sample from a patient is analyzed by MS and the results are used to determine the presence, absolute amount, and/or relative amount of a glycopeptide consisting of, or essentially of amino acid sequence SEQ ID NO: 35, in the sample. In some examples, a sample from a patient is analyzed by MS and the results are used to determine the presence, absolute amount, and/or relative amount of a glycopeptide consisting of, or essentially of amino acid sequence SEQ ID NO: 36, in the sample. In some examples, a sample from a patient is analyzed by MS and the results are used to determine the presence, absolute amount, and/or relative amount of a glycopeptide consisting of, or essentially of amino acid sequence SEQ ID NO: 37, in the sample. In some examples, a sample from a patient is analyzed by MS and the results are used to determine the presence, absolute amount, and/or relative amount of a glycopeptide consisting of, or essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide comprising an amino acid sequence of any of SEQ ID NOs: 3, 7, 9, 28, 29, 32, and 33 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of an amino acid sequence selected from SEQ ID NOs: 3, 7, 9, 28, 29, 32, and 33, in the sample. In some examples, a sample from a patient is analyzed by MS and the results are used to determine the presence, absolute amount, and/or relative amount of a glycopeptide consisting essentially of an amino acid sequence selected from SEQ ID NOs: 3, 7, 9, 28, 29, 32, and 33, in the sample. In some examples, a sample from a patient is analyzed by MS and the results are used to determine the presence, absolute amount, and/or relative amount of a glycopeptide consisting of, or consisting essentially of, an amino acid sequence selected from SEQ ID NOs: 3, 7, 9, 28, 29, 32, and 33, in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide comprising an amino acid sequence of any of SEQ ID NOs: 1-4, 6-7, 12, 15, 23-25, 28, 29, and 32 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of an amino acid sequence selected from SEQ ID NOs: 1-4, 6-7, 12, 15, 23-25, 28, 29, and 32, in the sample. In some examples, a sample from a patient is analyzed by MS and the results are used to determine the presence, absolute amount, and/or relative amount of a glycopeptide consisting essentially of an amino acid sequence selected from SEQ ID NOs: 1-4, 6-7, 12, 15, 23-25, 28, 29, and 32, in the sample. In some examples, a sample from a patient is analyzed by MS and the results are used to determine the presence, absolute amount, and/or relative amount of a glycopeptide consisting of, or consisting essentially of, an amino acid sequence selected from SEQ ID NOs: 1-4, 6-7, 12, 15, 23-25, 28, 29, and 32, in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide comprising at least one amino acid sequence selected from SEQ ID NOs: 1-38 in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting essentially of at least one amino acid sequence selected from SEQ ID NOs: 1-38 in the sample. In some examples, a sample from a patient is analyzed by MS and the results are used to determine the presence, absolute amount, and/or relative amount of a glycopeptide consisting of at least one amino acid sequence selected from SEQ ID NOs: 1-38 in the sample. In some examples, a sample from a patient is analyzed by MS and the results are used to determine the presence, absolute amount, and/or relative amount of a glycopeptide consisting of, or consisting essentially of, at least one amino acid sequence selected from SEQ ID NOs: 1-38 in the sample. In some examples, as described below, the presence, absolute amount, and/or relative amount of a glycopeptide is determined by analyzing the MS results. In some examples, the MS results are analyzed using machine learning.

In some other examples, the methods herein include selecting a patient having a sample analyzed by MS, the results of which are used to determine the presence, absolute amount, and/or relative amount of a glycopeptide comprising of at least one amino acid sequence selected from SEQ ID NOs: 1-38 in the sample. In some examples, as described below, the presence, absolute amount, and/or relative amount of a glycopeptide is determined by analyzing the MS results. In some examples, the MS results are analyzed using machine learning.

In some other examples, the methods herein include selecting a patient having a sample analyzed by MS, the results of which are used to determine the presence, absolute amount, and/or relative amount of a glycopeptide consisting essentially of at least one amino acid sequence selected from SEQ ID NOs: 1-38 in the sample. In some other examples, the methods herein include selecting a patient having a sample analyzed by MS, the results of which are used to determine the presence, absolute amount, and/or relative amount of a glycopeptide consisting of at least one amino acid sequence selected from SEQ ID NOs: 1-38 in the sample. In some examples, the methods herein include selecting a patient having a sample analyzed by MS, the results of which are used to determine the presence, absolute amount, and/or relative amount of a glycopeptide consisting of, or consisting essentially of, at least one amino acid sequence selected from SEQ ID NOs: 1-38 in the sample. In some examples, as described below, the presence, absolute amount, and/or relative amount of a glycopeptide is determined by analyzing the MS results. In some examples, the MS results are analyzed using machine learning.

Set forth herein are biomarkers selected from glycans, peptides, glycopeptides, fragments thereof, and combinations thereof. In some examples, the glycopeptide consists of an amino acid sequence selected from SEQ ID NOs: 1-38. In some examples, the glycopeptide consists essentially of an amino acid sequence selected from SEQ ID NOs: 1-38.

Set forth herein are biomarkers selected from glycans, peptides, glycopeptides, fragments thereof, and combinations thereof. In some examples, the glycopeptide comprises an amino acid sequence selected from SEQ ID NOs: 5, 8-11, 13-14, 16-22, 26-28, 30-31, and 34-38. In some examples, the glycopeptide consists of an amino acid sequence selected from SEQ ID NOs: 5, 8-11, 13-14, 16-22, 26-28, 30-31, and 34-38. In some examples, the glycopeptide consists essentially of an amino acid sequence selected from SEQ ID NOs: 5, 8-11, 13-14, 16-22, 26-28, 30-31, and 34-38.

Set forth herein are biomarkers selected from glycans, peptides, glycopeptides, fragments thereof, and combinations thereof. In some examples, the glycopeptide comprises an amino acid sequence selected from SEQ ID NOs: 3, 7, 9, 28, 29, 32, and 33. In some examples, the glycopeptide consists of an amino acid sequence selected from SEQ ID NOs: 3, 7, 9, 28, 29, 32, and 33. In some examples, the glycopeptide consists essentially of an amino acid sequence selected from SEQ ID NOs: 3, 7, 9, 28, 29, 32, and 33.

Set forth herein are biomarkers selected from glycans, peptides, glycopeptides, fragments thereof, and combinations thereof. In some examples, the glycopeptide comprises an amino acid sequence selected from SEQ ID NOs: 1-4, 6-7, 12, 15, 23-25, 28, 29, and 32. In some examples, the glycopeptide consists of an amino acid sequence selected from SEQ ID NOs: 1-4, 6-7, 12, 15, 23-25, 28, 29, and 32. In some examples, the glycopeptide consists essentially of an amino acid sequence selected from SEQ ID NOs: 1-4, 6-7, 12, 15, 23-25, 28, 29, and 32.

a. O-Glycosylation

In some examples, the glycopeptides set forth herein include O-glycosylated peptides. These peptides include glycopeptides in which a glycan is bonded to the peptide through an oxygen atom of an amino acid. Typically, the amino acid to which the glycan is bonded is threonine (T) or serine(S). In some examples, the amino acid to which the glycan is bonded is threonine (T). In some examples, the amino acid to which the glycan is bonded is serine(S).

In certain examples, the O-glycosylated peptides include those peptides from the group selected from Alpha-1-antitrypsin, Alpha-1B-glycoprotein, Alpha-2-macroglobulin, Alpha-1-antichymotrypsin, Alpha-1-acid glycoprotein 1, Alpha-1-acid glycoprotein 2, Apolipoprotein C-III (APOC3), Apolipoprotein D, Calpain-3, Ceruloplasmin, Haptoglobin, Immunoglobulin heavy chain constant μ, Plasma Kallikrein, Serum paraoxonase/arylesterase 1, Protein unc-13Homolog A, Alpha-2-HS-glycoprotein (FETUA), and combinations thereof.

In certain examples, the O-glycosylated peptide, set forth herein, is an Alpha-1-antitrypsin peptide. In certain examples, the O-glycosylated peptide, set forth herein, is an Alpha-1B-glycoprotein peptide. In certain examples, the O-glycosylated peptide, set forth herein, is an Alpha-2-macroglobulin peptide. In certain examples, the O-glycosylated peptide, set forth herein, is an Alpha-1-antichymotrypsin peptide. In certain examples, the O-glycosylated peptide, set forth herein, is an Alpha-1-acid glycoprotein 1 peptide. In certain examples, the O-glycosylated peptide, set forth herein, is an Alpha-1-acid glycoprotein 2peptide. In certain examples, the O-glycosylated peptide, set forth herein, is an Apolipoprotein C-III (APOC3) peptide. In certain examples, the O-glycosylated peptide, set forth herein, is an Apolipoprotein D peptide. In certain examples, the O-glycosylated peptide, set forth herein, is an Calpain-3 peptide. In certain examples, the O-glycosylated peptide, set forth herein, is an Ceruloplasmin peptide. In certain examples, the O-glycosylated peptide, set forth herein, is an Haptoglobin peptide. In certain examples, the O-glycosylated peptide, set forth herein, is an Immunoglobulin heavy chain constant μ peptide. In certain examples, the O-glycosylated peptide, set forth herein, is an Plasma Kallikrein peptide. In certain examples, the O-glycosylated peptide, set forth herein, is an Serum paraoxonase/arylesterase 1 peptide. In certain examples, the O-glycosylated peptide, set forth herein, is an Protein unc-13Homolog A peptide. In certain examples, the O-glycosylated peptide, set forth herein, is an Alpha-2-HS-glycoprotein (FETUA).

b. N-Glycosylation

In some examples, the glycopeptides set forth herein include N-glycosylated peptides. These peptides include glycopeptides in which a glycan is bonded to the peptide through a nitrogen atom of an amino acid. Typically, the amino acid to which the glycan is bonded is asparagine (N) or arginine (R). In some examples, the amino acid to which the glycan is bonded is asparagine (N). In some examples, the amino acid to which the glycan is bonded is arginine (R).

In certain examples, the N-glycosylated peptides include members selected from the group consisting of In certain examples, the O-glycosylated peptides include those peptides from the group selected from Alpha-1-antitrypsin, Alpha-1B-glycoprotein, Alpha-2-macroglobulin, Alpha-1-antichymotrypsin, Alpha-1-acid glycoprotein 1, Alpha-1-acid glycoprotein 2, Apolipoprotein C-III (APOC3), Apolipoprotein D, Calpain-3, Ceruloplasmin, Haptoglobin, Immunoglobulin heavy chain constant μ, Plasma Kallikrein, Serum paraoxonase/arylesterase 1, Protein unc-13Homolog A, Alpha-2-HS-glycoprotein (FETUA), and combinations thereof.

In certain examples, the N-glycosylated peptide, set forth herein, is an Alpha-1-antitrypsin peptide. In certain examples, the N-glycosylated peptide, set forth herein, is an Alpha-1B-glycoprotein peptide. In certain examples, the N-glycosylated peptide, set forth herein, is an Alpha-2-macroglobulin peptide. In certain examples, the N-glycosylated peptide, set forth herein, is an Alpha-1-antichymotrypsin peptide. In certain examples, the N-glycosylated peptide, set forth herein, is an Alpha-1-acid glycoprotein 1 peptide. In certain examples, the N-glycosylated peptide, set forth herein, is an Alpha-1-acid glycoprotein 2peptide. In certain examples, the N-glycosylated peptide, set forth herein, is an Apolipoprotein C-III (APOC3) peptide. In certain examples, the N-glycosylated peptide, set forth herein, is an Apolipoprotein D peptide. In certain examples, the N-glycosylated peptide, set forth herein, is an Calpain-3 peptide. In certain examples, the N-glycosylated peptide, set forth herein, is an Ceruloplasmin peptide. In certain examples, the N-glycosylated peptide, set forth herein, is an Haptoglobin peptide. In certain examples, the N-glycosylated peptide, set forth herein, is an Immunoglobulin heavy chain constant μ peptide. In certain examples, the N-glycosylated peptide, set forth herein, is an Plasma Kallikrein peptide. In certain examples, the N-glycosylated peptide, set forth herein, is an Serum paraoxonase/arylesterase 1 peptide. In certain examples, the N-glycosylated peptide, set forth herein, is an Protein unc-13Homolog A peptide. In certain examples, the N-glycosylated peptide, set forth herein, is an Alpha-2-HS-glycoprotein (FETUA).

c. Peptides and Glycopeptides

In some examples, set forth herein is a glycopeptide comprising an amino acid sequence selected from the group consisting of SEQ ID NOs: 1-38, and combinations thereof.

In some examples, set forth herein is a glycopeptide consisting of an amino acid sequence selected from the group consisting of SEQ ID NOs: 1-38, and combinations thereof.

In some examples, set forth herein is a glycopeptide consisting essentially of an amino acid sequence selected from the group consisting of SEQ ID NOs: 1-38, and combinations thereof.

In certain examples, the glycopeptide amino acid sequence comprises, consists essentially of, or consists of, an amino acid sequence selected from SEQ ID NO:1. In particular examples, the glycopeptide according to SEQ ID NO:1 further comprises glycan 5411, wherein the glycan(s) is (are) bonded to residue 107. In some examples, the glycopeptide is A1AT-GP001_107_5411, see, e.g., Table 10. Herein A1AT refers to Alpha-1-antitrypsin.

In certain examples, the glycopeptide amino acid sequence comprises, consists essentially, or consists of an amino acid sequence selected from SEQ ID NO:2. In particular examples, the glycopeptide according to SEQ ID NO:2 further comprises glycan 6503, wherein the glycan(s) is (are) bonded to residue 271. In some examples, the glycopeptide is A1AT-GP001_271_6503, see, e.g., Table 10.

In certain examples, the glycopeptide amino acid sequence comprises, consists essentially, or consists of an amino acid sequence selected from SEQ ID NO:3. In particular examples, the glycopeptide according to SEQ ID NO:3 further comprises glycan 5401, wherein the glycan(s) is (are) bonded to residue 271. In some examples, the glycopeptide is A1AT-GP001_271_5401, see, e.g., Table 10.

In certain examples, the glycopeptide amino acid sequence comprises, consists essentially, or consists of an amino acid sequence selected from SEQ ID NO:4. In particular examples, the glycopeptide according to SEQ ID NO:4 further comprises glycan 5402, wherein the glycan(s) is (are) bonded to residue 179. In some examples, the glycopeptide is A1BG-GP002_179_5421/5402, see, e.g., Table 10. Herein A1BG refers to Alpha-1B-glycoprotein. Herein, when two glycans are recited with a forward slash (/) between them, this means, unless specified otherwise explicitly, that the mass spectrometry method is unable to distinguish between these two glycans, e.g., because they share a common mass to charge ratio. Unless specified to the contrary, 5421/5402 means that either glycan 5421 or 5402 is present. The quantification of the amount of glycans 5421/5402 includes a summation of the detected amount of any glycan 5421 as well as the detected amount of any glycan 5402. Herein A1BG refers to Alpha-1B-glycoprotein.

In certain examples, the glycopeptide amino acid sequence comprises, consists essentially, or consists of an amino acid sequence selected from SEQ ID NO:5. In particular examples, the glycopeptide according to SEQ ID NO:5 further comprises glycan 5402, wherein the glycan(s) is (are) bonded to residue 1424. In some examples, the glycopeptide is A2MG-GP004_1424_5402, see, e.g., Table 10. Herein A2MG refers to Alpha-2-macroglobulin.

In certain examples, the glycopeptide amino acid sequence comprises, consists essentially, or consists of an amino acid sequence selected from SEQ ID NO:6. In particular examples, the glycopeptide according to SEQ ID NO:6 further comprises glycan 5412, wherein the glycan(s) is (are) bonded to residue 1424. In some examples, the glycopeptide is A2MG-GP004_1424_5412, see, e.g., Table 10.

In certain examples, the glycopeptide amino acid sequence comprises, consists essentially, or consists of an amino acid sequence selected from SEQ ID NO:7. In particular examples, the glycopeptide according to SEQ ID NO:7 further comprises glycan 5402, wherein the glycan(s) is (are) bonded to residue 55. In some examples, the glycopeptide is A2MG-GP004_55_5402, see, e.g., Table 10.

In certain examples, the glycopeptide amino acid sequence comprises, consists essentially, or consists of an amino acid sequence selected from SEQ ID NO:8. In particular examples, the glycopeptide according to SEQ ID NO:8 further comprises glycan 5401, wherein the glycan(s) is (are) bonded to residue 869. In some examples, the glycopeptide is A2MG-GP004_869_5401, see, e.g., Table 10.

In certain examples, the glycopeptide amino acid sequence comprises, consists essentially, or consists of an amino acid sequence selected from SEQ ID NO:9. In particular examples, the glycopeptide according to SEQ ID NO:9 further comprises glycan 6301, wherein the glycan(s) is (are) bonded to residue 869. In some examples, the glycopeptide is A2MG-GP004_869_6301, see, e.g., Table 10.

In certain examples, the glycopeptide amino acid sequence comprises, consists essentially, or consists of an amino acid sequence selected from SEQ ID NO:10. In particular examples, the glycopeptide according to SEQ ID NO:10 further comprises glycan 7603, wherein the glycan(s) is (are) bonded to residue 271. In some examples, the glycopeptide is AACT-GP005_271_7603, see, e.g., Table 10. Herein AACT refers to Alpha-1-antichymotrypsin.

In certain examples, the glycopeptide amino acid sequence comprises, consists essentially, or consists of an amino acid sequence selected from SEQ ID NO:11. In particular examples, the glycopeptide according to SEQ ID NO:11 further comprises glycan 9804, wherein the glycan(s) is (are) bonded to residue 103. In some examples, the glycopeptide is AGP1-GP007_103_9804, see, e.g., Table 10. Herein AGP refers to Alpha-1-acid glycoprotein 1.

In certain examples, the glycopeptide amino acid sequence comprises, consists essentially, or consists of an amino acid sequence selected from SEQ ID NO:12. In particular examples, the glycopeptide according to SEQ ID NO:12 further comprises glycan 6501, wherein the glycan(s) is (are) bonded to residue 33. In some examples, the glycopeptide is AGP1-GP007_33_6501, see, e.g., Table 10.

In certain examples, the glycopeptide amino acid sequence comprises, consists essentially, or consists of an amino acid sequence selected from SEQ ID NO:13. In particular examples, the glycopeptide according to SEQ ID NO:13 further comprises glycan 6502, wherein the glycan(s) is (are) bonded to residue 93. In some examples, the glycopeptide is AGP1-GP007_93_6502, see, e.g., Table 10.

In certain examples, the glycopeptide amino acid sequence comprises, consists essentially, or consists of an amino acid sequence selected from SEQ ID NO:14. In particular examples, the glycopeptide according to SEQ ID NO:14 further comprises glycan 7611, wherein the glycan(s) is (are) bonded to residue 93. In some examples, the glycopeptide is AGP1-GP007_93_7611, see, e.g., Table 10.

In certain examples, the glycopeptide amino acid sequence comprises, consists essentially, or consists of an amino acid sequence selected from SEQ ID NO:15. In particular examples, the glycopeptide according to SEQ ID NO:15 further comprises glycan 6503, wherein the glycan(s) is (are) bonded to residue 103. In some examples, the glycopeptide is AGP2-GP008_103_6503, see, e.g., Table 10. Herein AGP refers to Alpha-1-acid glycoprotein 2.

In certain examples, the glycopeptide amino acid sequence comprises, consists essentially, or consists of an amino acid sequence selected from SEQ ID NO:16. In particular examples, the glycopeptide according to SEQ ID NO:16 further comprises glycan 1102, wherein the glycan(s) is (are) bonded to residue 74. In some examples, the glycopeptide is APOC3-GP012_74_1102, see, e.g., Table 10. Herein APOC refers to Apolipoprotein C-III.

In certain examples, the glycopeptide amino acid sequence comprises, consists essentially, or consists of an amino acid sequence selected from SEQ ID NO: 17. In particular examples, the glycopeptide according to SEQ ID NO:17 further comprises glycan 5402 or 5421, wherein the glycan(s) is (are) bonded to residue 98. In some examples, the glycopeptide is APOD-GP014_98_5402/5421, see, e.g., Table 10. Herein APOD refers to Apolipoprotein D.

In certain examples, the glycopeptide amino acid sequence comprises, consists essentially, or consists of an amino acid sequence selected from SEQ ID NO:18. In particular examples, the glycopeptide according to SEQ ID NO:18 further comprises glycan 5410, wherein the glycan(s) is (are) bonded to residue 98. In some examples, the glycopeptide is APOD-GP014_98_5410, see, e.g., Table 10.

In certain examples, the glycopeptide amino acid sequence comprises, consists essentially, or consists of an amino acid sequence selected from SEQ ID NO: 19. In particular examples, the glycopeptide according to SEQ ID NO: 19 further comprises glycan 6510, wherein the glycan(s) is (are) bonded to residue 98. In some examples, the glycopeptide is APOD-GP014_98_6510, see, e.g., Table 10.

In certain examples, the glycopeptide amino acid sequence comprises, consists essentially, or consists of an amino acid sequence selected from SEQ ID NO:20. In particular examples, the glycopeptide according to SEQ ID NO:20 further comprises glycan 6530, wherein the glycan(s) is (are) bonded to residue 98. In some examples, the glycopeptide is APOD-GP014_98_6530, see, e.g., Table 10.

In certain examples, the glycopeptide amino acid sequence comprises, consists essentially, or consists of an amino acid sequence selected from SEQ ID NO:21. In particular examples, the glycopeptide according to SEQ ID NO:21 further comprises glycan 9800, wherein the glycan(s) is (are) bonded to residue 98. In some examples, the glycopeptide is APOD-GP014_98_9800, see, e.g., Table 10.

In certain examples, the glycopeptide amino acid sequence comprises, consists essentially, or consists of an amino acid sequence selected from SEQ ID NO:22. In particular examples, the glycopeptide according to SEQ ID NO:22 further comprises glycan 6513, wherein the glycan(s) is (are) bonded to residue 366. In some examples, the glycopeptide is CAN3-GP022_366_6513, see, e.g., Table 10. Herein CAN refers to Calpain-3.

In certain examples, the glycopeptide amino acid sequence comprises, consists essentially, or consists of an amino acid sequence selected from SEQ ID NO:23. In particular examples, the glycopeptide according to SEQ ID NO:23 further comprises glycan 5412, wherein the glycan(s) is (are) bonded to residue 138. In some examples, the glycopeptide is CERU-GP023_138_5412, see, e.g., Table 10. Herein CERU refers to Ceruloplasmin.

In certain examples, the glycopeptide amino acid sequence comprises, consists essentially, or consists of an amino acid sequence selected from SEQ ID NO:24. In particular examples, the glycopeptide according to SEQ ID NO:24 further comprises glycan 5421 or 5402, wherein the glycan(s) is (are) bonded to residue 138. In some examples, the glycopeptide is CERU-GP023_138_5421/5402, see, e.g., Table 10.

In certain examples, the glycopeptide amino acid sequence comprises, consists essentially, or consists of an amino acid sequence selected from SEQ ID NO:25. In particular examples, the glycopeptide according to SEQ ID NO:25 further comprises glycan 5401, wherein the glycan(s) is (are) bonded to residue 176. In some examples, the glycopeptide is FETUA-GP036_176_5401, see, e.g., Table 10. Herein FETUA refers to Alpha-2-HS-glycoprotein.

In certain examples, the glycopeptide amino acid sequence comprises, consists essentially, or consists of an amino acid sequence selected from SEQ ID NO:26. In particular examples, the glycopeptide according to SEQ ID NO:26 further comprises glycan 6513, wherein the glycan(s) is (are) bonded to residue 176. In some examples, the glycopeptide is FETUA-GP036_176_6513, see, e.g., Table 10.

In certain examples, the glycopeptide amino acid sequence comprises, consists essentially, or consists of an amino acid sequence selected from SEQ ID NO:27. In particular examples, the glycopeptide according to SEQ ID NO:27 further comprises glycan 5401, wherein the glycan(s) is (are) bonded to residue 207. In some examples, the glycopeptide is HPT-GP044_207_5401, see, e.g., Table 10. Herein HPT refers to Haptoglobin.

In certain examples, the glycopeptide amino acid sequence comprises, consists essentially, or consists of an amino acid sequence selected from SEQ ID NO:28. In particular examples, the glycopeptide according to SEQ ID NO:28 further comprises glycan 5402 or 5421, wherein the glycan(s) is (are) bonded to residue 241. In some examples, the glycopeptide is HPT-GP044_241_5402/5421, see, e.g., Table 10.

In certain examples, the glycopeptide amino acid sequence comprises, consists essentially, or consists of an amino acid sequence selected from SEQ ID NO:29. In particular examples, the glycopeptide according to SEQ ID NO:29 further comprises glycan 5511, wherein the glycan(s) is (are) bonded to residue 241. In some examples, the glycopeptide is HPT-GP044_241_5511, see, e.g., Table 10.

In certain examples, the glycopeptide amino acid sequence comprises, consists essentially, or consists of an amino acid sequence selected from SEQ ID NO:30. In particular examples, the glycopeptide according to SEQ ID NO:30 further comprises glycan 6511, wherein the glycan(s) is (are) bonded to residue 241. In some examples, the glycopeptide is HPT-GP044_241_6511, see, e.g., Table 10.

In certain examples, the glycopeptide amino acid sequence comprises, consists essentially, or consists of an amino acid sequence selected from SEQ ID NO:31. In particular examples, the glycopeptide according to SEQ ID NO:31 further comprises glycan 7511, wherein the glycan(s) is (are) bonded to residue 241. In some examples, the glycopeptide is HPT-GP044_241_7511, see, e.g., Table 10.

In certain examples, the glycopeptide amino acid sequence comprises, consists essentially, or consists of an amino acid sequence selected from SEQ ID NO:31. In particular examples, the glycopeptide according to SEQ ID NO:31 further comprises glycan 4310, wherein the glycan(s) is (are) bonded to residue 46. In some examples, the glycopeptide is IgM-GP053_46_4310, see, e.g., Table 10. Herein IgM refers to Immunoglobulin heavy chain constant μ.

In certain examples, the glycopeptide amino acid sequence comprises, consists essentially, or consists of an amino acid sequence selected from SEQ ID NO:33. In particular examples, the glycopeptide according to SEQ ID NO:33 further comprises glycan 6503, wherein the glycan(s) is (are) bonded to residue 494. In some examples, the glycopeptide is KLKB1-GP056_494_6503, see, e.g., Table 10. Herein KLKB refers to Plasma Kallikrein.

In certain examples, the glycopeptide amino acid sequence comprises, consists essentially, or consists of an amino acid sequence selected from SEQ ID NO:34. In particular examples, the glycopeptide according to SEQ ID NO:34 further comprises glycan 5420, wherein the glycan(s) is (are) bonded to residue 324. In some examples, the glycopeptide is PON1-GP060_324_5420, see, e.g., Table 10. Herein PON refers to Serum paraoxonase/arylesterase 1.

In certain examples, the glycopeptide amino acid sequence comprises, consists essentially, or consists of an amino acid sequence selected from SEQ ID NO:35. In particular examples, the glycopeptide according to SEQ ID NO:35 further comprises glycan 6501, wherein the glycan(s) is (are) bonded to residue 324. In some examples, the glycopeptide is PON1-GP060_324_6501, see, e.g., Table 10.

In certain examples, the glycopeptide amino acid sequence comprises, consists essentially, or consists of an amino acid sequence selected from SEQ ID NO:36. In particular examples, the glycopeptide according to SEQ ID NO:36 further comprises glycan 6502, wherein the glycan(s) is (are) bonded to residue 324. In some examples, the glycopeptide is PON1-GP060_324_6502, see, e.g., Table 10.

In certain examples, the glycopeptide amino acid sequence comprises, consists essentially, or consists of an amino acid sequence selected from SEQ ID NO:37. In particular examples, the glycopeptide according to SEQ ID NO:37 further comprises glycan 5431, wherein the glycan(s) is (are) bonded to residue 1005. In some examples, the glycopeptide is UN13A-GP066_1005_5431, see, e.g., Table 10. Herein UN13 refers to Protein unc-13Homolog A.

In certain examples, the glycopeptide amino acid sequence comprises, consists essentially, or consists of an amino acid sequence selected from SEQ ID NO:38. In particular examples, the glycopeptide according to SEQ ID NO:38 further comprises glycan 7420, wherein the glycan(s) is (are) bonded to residue 1005. In some examples, the glycopeptide is UN13A-GP066_1005_7420, see, e.g., Table 10.

In some examples, including any of the foregoing, the glycopeptide comprises at least one amino acid sequence selected from SEQ ID NOs: 1-38 or a combination thereof. In some examples, including any of the foregoing, the glycopeptide is a combination of amino acid sequences selected from SEQ ID NOs: 1-38.

In some examples, including any of the foregoing, set forth herein is one or more peptides, in which each peptide, individually in each instance, is a peptide consisting of an amino acid sequence selected from SEQ ID NOs: 5, 8-11, 13-14, 16-22, 26-28, 30-31, and 34-38, and combinations thereof.

In some examples, including any of the foregoing, set forth herein is one or more peptides, in which each peptide, individually in each instance, is a peptide consisting of an amino acid sequence selected from SEQ ID NOs: 3, 7, 9, 28, 29, 32, and 33, and combinations thereof.

In some examples, including any of the foregoing, set forth herein is one or more peptides, in which each peptide, individually in each instance, is a peptide consisting of an amino acid sequence selected from SEQ ID NOs: 1-4, 6-7, 12, 15, 23-25, 28, 29, and 32, and combinations thereof.

In some examples, including any of the foregoing, set forth herein is one or more peptides, in which each peptide, individually in each instance, is a peptide comprising at least one amino acid sequence selected from SEQ ID NOs: 1-38 or combinations thereof. In some examples, including any of the foregoing, set forth herein is one or more peptides, in which each peptide, individually in each instance, is a peptide consisting essentially of at least one amino acid sequence selected from SEQ ID NOs: 1-38, and combinations thereof.

In some examples, including any of the foregoing, set forth herein is one or more peptides, in which each peptide, individually in each instance, is a peptide comprising at least one amino acid sequence selected from SEQ ID NOs: 5, 8-11, 13-14, 16-22, 26-28, 30-31, and 34-38 or combinations thereof. In some examples, including any of the foregoing, set forth herein is one or more peptides, in which each peptide, individually in each instance, is a peptide consisting essentially of an amino acid sequence selected from SEQ ID NOs: 5, 8-11, 13-14, 16-22, 26-28, 30-31, and 34-38, and combinations thereof.

In some examples, including any of the foregoing, set forth herein is one or more peptides, in which each peptide, individually in each instance, is a peptide comprising at least one amino acid sequence selected from SEQ ID NOs: 3, 7, 9, 28, 29, 32, and 33 or combinations thereof. In some examples, including any of the foregoing, set forth herein is one or more peptides, in which each peptide, individually in each instance, is a peptide consisting essentially of an amino acid sequence selected from SEQ ID NOs: 3, 7, 9, 28, 29, 32, and 33, and combinations thereof.

In some examples, including any of the foregoing, set forth herein is one or more peptides, in which each peptide, individually in each instance, is a peptide comprising at least one amino acid sequence selected from SEQ ID NOs: 1-4, 6-7, 12, 15, 23-25, 28, 29, and 32 or combinations thereof. In some examples, including any of the foregoing, set forth herein is one or more peptides, in which each peptide, individually in each instance, is a peptide consisting essentially of an amino acid sequence selected from SEQ ID NOs: 1-4, 6-7, 12, 15, 23-25, 28, 29, and 32, and combinations thereof.

### IV. Methods of Using Biomarkers

### A. Methods for Detecting Glycopeptides

In some embodiments, set forth herein is a method for detecting one or more a multiple-reaction-monitoring (MRM) transition, comprising: obtaining, or having obtained, a biological sample from a patient, wherein the biological sample comprises one or more glycoproteins set forth in Table 9; digesting and/or fragmenting one or more glycoprotein in the sample into one or more glycopeptides; and detecting a multiple-reaction-monitoring (MRM) transition selected from the group consisting of transitions 1-38. In some embodiments, the transitions 1-38 correspond to peptide structure data comprises at least one peptide structure from the biological sample. In some embodiments, the at least one peptide structure comprises one or more glycopeptides structure set forth in Table 10. In some embodiments, the at least one peptide structure comprises one or more glycopeptides comprising the amino acid sequence of any of SEQ ID NOs: 1-38.

In some embodiments, set forth herein is a method for detecting one or more a multiple-reaction-monitoring (MRM) transition, comprising: obtaining, or having obtained, a biological sample from a patient, wherein the biological sample comprises one or more glycopeptides; digesting and/or fragmenting a glycopeptide in the sample; and detecting a multiple-reaction-monitoring (MRM) transition selected from the group consisting of transitions 1-38. These transitions may include, in various examples, any one or more of the transitions in Tables 1-5. These transitions may include, in various examples, any one or more of the transitions in Tables 1-3. These transitions may include, in various examples, any one or more of the transitions in Table 1. These transitions may include, in various examples, any one or more of the transitions in Table 2. These transitions may include, in various examples, any one or more of the transitions in Table 3. These transitions may include, in various examples, any one or more of the transitions in Table 4. These transitions may include, in various examples, any one or more of the transitions in Table 5. These transitions may be indicative of glycopeptides.

In some examples, set forth herein is a method of detecting one or more glycopeptides, wherein each glycopeptide is individually in each instance selected from a glycopeptide consisting of an amino acid sequence selected from the group consisting of SEQ ID NOs: 1-38, and combinations thereof. In some examples, set forth herein is a method of detecting one or more glycopeptides, wherein the one or more glycopeptide is selected from Table 10.

In some examples, set forth herein is a method of detecting one or more glycopeptides, wherein each glycopeptide is individually in each instance selected from a glycopeptide consisting essentially of an amino acid sequence selected from the group consisting of SEQ ID NOs: 1-38, and combinations thereof.

In some examples, set forth herein is a method of detecting one or more glycopeptides, wherein each glycopeptide is individually in each instance selected from a glycopeptide consisting of an amino acid sequence selected from the group consisting of SEQ ID NOs: 5, 8-11, 13-14, 16-22, 26-28, 30-31, and 34-38, and combinations thereof.

In some examples, set forth herein is a method of detecting a glycopeptide, wherein the glycopeptide consists of amino acid sequence SEQ ID NO: 5, in the sample. In some examples, set forth herein is a method of detecting a glycopeptide, wherein the glycopeptide consists of amino acid sequence SEQ ID NO: 8, in the sample. In some examples, set forth herein is a method of detecting a glycopeptide, wherein the glycopeptide consists of amino acid sequence SEQ ID NO: 9, in the sample. In some examples, set forth herein is a method of detecting a glycopeptide, wherein the glycopeptide consists of amino acid sequence SEQ ID NO: 10, in the sample. In some examples, set forth herein is a method of detecting a glycopeptide, wherein the glycopeptide consists of amino acid sequence SEQ ID NO: 11, in the sample. In some examples, set forth herein is a method of detecting a glycopeptide, wherein the glycopeptide consists of amino acid sequence SEQ ID NO: 13, in the sample. In some examples, set forth herein is a method of detecting a glycopeptide, wherein the glycopeptide consists of amino acid sequence SEQ ID NO: 14, in the sample. In some examples, set forth herein is a method of detecting a glycopeptide, wherein the glycopeptide consists of amino acid sequence SEQ ID NO: 16, in the sample. In some examples, set forth herein is a method of detecting a glycopeptide, wherein the glycopeptide consists of amino acid sequence SEQ ID NO: 17, in the sample. In some examples, set forth herein is a method of detecting a glycopeptide, wherein the glycopeptide consists of amino acid sequence SEQ ID NO: 18, in the sample. In some examples, set forth herein is a method of detecting a glycopeptide, wherein the glycopeptide consists of amino acid sequence SEQ ID NO: 19, in the sample. In some examples, set forth herein is a method of detecting a glycopeptide, wherein the glycopeptide consists of amino acid sequence SEQ ID NO: 20, in the sample. In some examples, set forth herein is a method of detecting a glycopeptide, wherein the glycopeptide consists of amino acid sequence SEQ ID NO: 21, in the sample. In some examples, set forth herein is a method of detecting a glycopeptide, wherein the glycopeptide consists of amino acid sequence SEQ ID NO: 22, in the sample. In some examples, set forth herein is a method of detecting a glycopeptide, wherein the glycopeptide consists of amino acid sequence SEQ ID NO: 26, in the sample. In some examples, set forth herein is a method of detecting a glycopeptide, wherein the glycopeptide consists of amino acid sequence SEQ ID NO: 27, in the sample. In some examples, set forth herein is a method of detecting a glycopeptide, wherein the glycopeptide consists of amino acid sequence SEQ ID NO: 28, in the sample. In some examples, set forth herein is a method of detecting a glycopeptide, wherein the glycopeptide consists of amino acid sequence SEQ ID NO: 30, in the sample. In some examples, set forth herein is a method of detecting a glycopeptide, wherein the glycopeptide consists of amino acid sequence SEQ ID NO: 31, in the sample. In some examples, set forth herein is a method of detecting a glycopeptide, wherein the glycopeptide consists of amino acid sequence SEQ ID NO: 34, in the sample. In some examples, set forth herein is a method of detecting a glycopeptide, wherein the glycopeptide consists of amino acid sequence SEQ ID NO: 35, in the sample. In some examples, set forth herein is a method of detecting a glycopeptide, wherein the glycopeptide consists of amino acid sequence SEQ ID NO: 36, in the sample. In some examples, set forth herein is a method of detecting a glycopeptide, wherein the glycopeptide consists of amino acid sequence SEQ ID NO: 37, in the sample. In some examples, set forth herein is a method of detecting a glycopeptide, wherein the glycopeptide consists of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, set forth herein is a method of detecting a glycopeptide, wherein the glycopeptide consists essentially of amino acid sequence SEQ ID NO: 5, in the sample. In some examples, set forth herein is a method of detecting a glycopeptide, wherein the glycopeptide consists essentially of amino acid sequence SEQ ID NO: 8, in the sample. In some examples, set forth herein is a method of detecting a glycopeptide, wherein the glycopeptide consists essentially of amino acid sequence SEQ ID NO: 9, in the sample. In some examples, set forth herein is a method of detecting a glycopeptide, wherein the glycopeptide consists essentially of amino acid sequence SEQ ID NO: 10, in the sample. In some examples, set forth herein is a method of detecting a glycopeptide, wherein the glycopeptide consists essentially of amino acid sequence SEQ ID NO: 11, in the sample. In some examples, set forth herein is a method of detecting a glycopeptide, wherein the glycopeptide consists essentially of amino acid sequence SEQ ID NO: 13, in the sample. In some examples, set forth herein is a method of detecting a glycopeptide, wherein the glycopeptide consists essentially of amino acid sequence SEQ ID NO: 14, in the sample. In some examples, set forth herein is a method of detecting a glycopeptide, wherein the glycopeptide consists essentially of amino acid sequence SEQ ID NO: 16, in the sample. In some examples, set forth herein is a method of detecting a glycopeptide, wherein the glycopeptide consists essentially of amino acid sequence SEQ ID NO: 17, in the sample. In some examples, set forth herein is a method of detecting a glycopeptide, wherein the glycopeptide consists essentially of amino acid sequence SEQ ID NO: 18, in the sample. In some examples, set forth herein is a method of detecting a glycopeptide, wherein the glycopeptide consists essentially of amino acid sequence SEQ ID NO: 19, in the sample. In some examples, set forth herein is a method of detecting a glycopeptide, wherein the glycopeptide consists essentially of amino acid sequence SEQ ID NO: 20, in the sample. In some examples, set forth herein is a method of detecting a glycopeptide, wherein the glycopeptide consists essentially of amino acid sequence SEQ ID NO: 21, in the sample. In some examples, set forth herein is a method of detecting a glycopeptide, wherein the glycopeptide consists essentially of amino acid sequence SEQ ID NO: 22, in the sample. In some examples, set forth herein is a method of detecting a glycopeptide, wherein the glycopeptide consists essentially of amino acid sequence SEQ ID NO: 26, in the sample. In some examples, set forth herein is a method of detecting a glycopeptide, wherein the glycopeptide consists essentially of amino acid sequence SEQ ID NO: 27, in the sample. In some examples, set forth herein is a method of detecting a glycopeptide, wherein the glycopeptide consists essentially of amino acid sequence SEQ ID NO: 28, in the sample. In some examples, set forth herein is a method of detecting a glycopeptide, wherein the glycopeptide consists essentially of amino acid sequence SEQ ID NO: 30, in the sample. In some examples, set forth herein is a method of detecting a glycopeptide, wherein the glycopeptide consists essentially of amino acid sequence SEQ ID NO: 31, in the sample. In some examples, set forth herein is a method of detecting a glycopeptide, wherein the glycopeptide consists essentially of amino acid sequence SEQ ID NO: 34, in the sample. In some examples, set forth herein is a method of detecting a glycopeptide, wherein the glycopeptide consists essentially of amino acid sequence SEQ ID NO: 35, in the sample. In some examples, set forth herein is a method of detecting a glycopeptide, wherein the glycopeptide consists essentially of amino acid sequence SEQ ID NO: 36, in the sample. In some examples, set forth herein is a method of detecting a glycopeptide, wherein the glycopeptide consists essentially of amino acid sequence SEQ ID NO: 37, in the sample. In some examples, set forth herein is a method of detecting a glycopeptide, wherein the glycopeptide consists essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, a sample from a patient is analyzed by MS and the results are used to determine the presence, absolute amount, and/or relative amount of a glycopeptide consisting of, or consisting essentially of sequence SEQ ID NO: 5, in the sample. In some examples, set forth herein is a method of detecting a glycopeptide, wherein the glycopeptide consists of, or essentially of amino acid sequence SEQ ID NO: 8, in the sample. In some examples, set forth herein is a method of detecting a glycopeptide, wherein the glycopeptide consists of, or essentially of amino acid sequence SEQ ID NO: 9, in the sample. In some examples, set forth herein is a method of detecting a glycopeptide, wherein the glycopeptide consists of, or essentially of amino acid sequence SEQ ID NO: 10, in the sample. In some examples, set forth herein is a method of detecting a glycopeptide, wherein the glycopeptide consists of, or essentially of amino acid sequence SEQ ID NO: 11, in the sample. In some examples, set forth herein is a method of detecting a glycopeptide, wherein the glycopeptide consists of, or essentially of amino acid sequence SEQ ID NO: 13, in the sample. In some examples, set forth herein is a method of detecting a glycopeptide, wherein the glycopeptide consists of, or essentially of amino acid sequence SEQ ID NO: 14, in the sample. In some examples, set forth herein is a method of detecting a glycopeptide, wherein the glycopeptide consists of, or essentially of amino acid sequence SEQ ID NO: 16, in the sample. In some examples, set forth herein is a method of detecting a glycopeptide, wherein the glycopeptide consists of, or essentially of amino acid sequence SEQ ID NO: 17, in the sample. In some examples, set forth herein is a method of detecting a glycopeptide, wherein the glycopeptide consists of, or essentially of amino acid sequence SEQ ID NO: 18, in the sample. In some examples, set forth herein is a method of detecting a glycopeptide, wherein the glycopeptide consists of, or essentially of amino acid sequence SEQ ID NO: 19, in the sample. In some examples, set forth herein is a method of detecting a glycopeptide, wherein the glycopeptide consists of, or essentially of amino acid sequence SEQ ID NO: 20, in the sample. In some examples, set forth herein is a method of detecting a glycopeptide, wherein the glycopeptide consists of, or essentially of amino acid sequence SEQ ID NO: 21, in the sample. In some examples, set forth herein is a method of detecting a glycopeptide, wherein the glycopeptide consists of, or essentially of amino acid sequence SEQ ID NO: 22, in the sample. In some examples, set forth herein is a method of detecting a glycopeptide, wherein the glycopeptide consists of, or essentially of amino acid sequence SEQ ID NO: 26, in the sample. In some examples, set forth herein is a method of detecting a glycopeptide, wherein the glycopeptide consists of, or essentially of amino acid sequence SEQ ID NO: 27, in the sample. In some examples, set forth herein is a method of detecting a glycopeptide, wherein the glycopeptide consists of, or essentially of amino acid sequence SEQ ID NO: 28, in the sample. In some examples, set forth herein is a method of detecting a glycopeptide, wherein the glycopeptide consists of, or essentially of amino acid sequence SEQ ID NO: 30, in the sample. In some examples, set forth herein is a method of detecting a glycopeptide, wherein the glycopeptide consists of, or essentially of amino acid sequence SEQ ID NO: 31, in the sample. In some examples, set forth herein is a method of detecting a glycopeptide, wherein the glycopeptide consists of, or essentially of amino acid sequence SEQ ID NO: 34, in the sample. In some examples, set forth herein is a method of detecting a glycopeptide, wherein the glycopeptide consists of, or essentially of amino acid sequence SEQ ID NO: 35, in the sample. In some examples, set forth herein is a method of detecting a glycopeptide, wherein the glycopeptide consists of, or essentially of amino acid sequence SEQ ID NO: 36, in the sample. In some examples, set forth herein is a method of detecting a glycopeptide, wherein the glycopeptide consists of, or essentially of amino acid sequence SEQ ID NO: 37, in the sample. In some examples, set forth herein is a method of detecting a glycopeptide, wherein the glycopeptide consists of, or essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, set forth herein is a method of detecting one or more glycopeptides, wherein each glycopeptide is individually in each instance selected from a glycopeptide consisting of an amino acid sequence selected from the group consisting of SEQ ID NOs: 3, 7, 9, 28, 29, 32, and 33, and combinations thereof.

In some examples, set forth herein is a method of detecting one or more glycopeptides, wherein each glycopeptide is individually in each instance selected from a glycopeptide consisting of an amino acid sequence selected from the group consisting of SEQ ID NOs: 1-4, 6-7, 12, 15, 23-25, 28, 29, and 32, and combinations thereof.

In some examples, set forth herein is a method of detecting one or more glycopeptides, wherein each glycopeptide is individually in each instance selected from a glycopeptide consisting essentially of an amino acid sequence selected from the group consisting of SEQ ID NOs: 5, 8-11, 13-14, 16-22, 26-28, 30-31, and 34-38 and combinations thereof.

In some examples, set forth herein is a method of detecting one or more glycopeptides, wherein each glycopeptide is individually in each instance selected from a glycopeptide consisting essentially of an amino acid sequence selected from the group consisting of SEQ ID NOs: 3, 7, 9, 28, 29, 32, and 33, and combinations thereof.

In some examples, set forth herein is a method of detecting one or more glycopeptides, wherein each glycopeptide is individually in each instance selected from a glycopeptide consisting essentially of an amino acid sequence selected from the group consisting of SEQ ID NOs: 1-4, 6-7, 12, 15, 23-25, 28, 29, and 32, and combinations thereof.

In some examples, set forth herein is a method of detecting one or more glycopeptides. In some examples, set forth herein is a method of detecting one or more glycopeptide fragments. In certain examples, the method includes detecting the glycopeptide group to which the glycopeptide, or fragment thereof, belongs. In some examples, the method includes detecting a glycoprotein set forth in Table 9. In some examples, the method includes detecting a glycoprotein comprising the amino acid sequence of any of SEQ ID NOs: 39-54. In some of these examples, the glycopeptide group is selected from Alpha-1-antitrypsin (A1AT), Alpha-1B-glycoprotein (A1BG), Alpha-2-macroglobulin (A2MG), Alpha-1-antichymotrypsin (AACT), Alpha-1-acid glycoprotein 1 & 2 (AGP12), Alpha-1-acid glycoprotein 1 (AGP1), Alpha-1-acid glycoprotein 2 (AGP2), Apolipoprotein C-III (APOC3), Apolipoprotein D (APOD), Calpain-3 (CAN3), Ceruloplasmin (CERU), Alpha-2-HS glycoprotein (FETUA); Haptoglobin (HPT), Immunoglobulin heavy chain constant μ (IgM), Plasma Kallikrein (KLKB1), Serum paraoxonase/arylesterase 1 (PON1), Protein unc-13HomologA (UN13A), and combinations thereof.

In some of these examples, the glycopeptide group is Alpha-1-antitrypsin (A1AT). In some of these examples, the glycopeptide group is Alpha-1B-glycoprotein (A1BG). In some of these examples, the glycopeptide group is Alpha-2-macroglobulin (A2MG). In some of these examples, the glycopeptide group is Alpha-1-antichymotrypsin (AACT). In some of these examples, the glycopeptide group is Alpha-1-acid glycoprotein 1 & 2 (AGP12). In some of these examples, the glycopeptide group is Alpha-1-acid glycoprotein 1 (AGP1). In some of these examples, the glycopeptide group is Alpha-1-acid glycoprotein 2 (AGP2). In some of these examples, the glycopeptide group is Apolipoprotein C-III (APOC3). In some of these examples, the glycopeptide group is Apolipoprotein D (APOD). In some of these examples, the glycopeptide group is Calpain-3 (CAN3). In some of these examples, the glycopeptide group is Ceruloplasmin (CERU). In some of these examples, the glycopeptide group is Alpha-2-HS glycoprotein (FETUA). In some of these examples, the glycopeptide group is Haptoglobin (HPT). In some of these examples, the glycopeptide group is Immunoglobulin heavy chain constant μ (IgM). In some of these examples, the glycopeptide group is Plasma Kallikrein (KLKB1). In some of these examples, the glycopeptide group is Serum paraoxonase/arylesterase 1 (PON1). In some of these examples, the glycopeptide group is Protein unc-13HomologA (UN13A). In some examples, the glycoprotein group is set forth by one or more of the glycoproteins of Table 9. In some examples, the glycoprotein group comprises the amino acid sequence of any of SEQ ID NOs: 39-54.

In some examples, including any of the foregoing, the method includes detecting a glycopeptide, a glycan on the glycopeptide and the glycosylation site residue where the glycan bonds to the glycopeptide. In certain examples, the method includes detecting a glycan residue. In some examples, the method includes detecting a glycosylation site on a glycopeptide. In some examples, this process is accomplished with mass spectroscopy used in tandem with liquid chromatography.

In some examples, including any of the foregoing, the method includes obtaining, or having obtained a biological sample from a patient. In some examples, the biological sample is synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humour, transudate, or combinations of the foregoing. In certain examples, the biological sample is selected from the group consisting of blood, plasma, saliva, mucus, urine, stool, tissue, sweat, tears, hair, or a combination thereof. In some of these examples, the biological sample is a blood sample. In some of these examples, the biological sample is a plasma sample. In some of these examples, the biological sample is a saliva sample. In some of these examples, the biological sample is a mucus sample. In some of these examples, the biological sample is a urine sample. In some of these examples, the biological sample is a stool sample. In some of these examples, the biological sample is a sweat sample. In some of these examples, the biological sample is a tear sample. In some of these examples, the biological sample is a hair sample.

In some examples, including any of the foregoing, the method also includes digesting and/or fragmenting a glycopeptide in the sample. In certain examples, the method includes digesting a glycopeptide in the sample. In certain examples, the method includes fragmenting a glycopeptide in the sample. In some examples, the digested or fragmented glycopeptide is analyzed using mass spectroscopy. In some examples, the glycopeptide is digested or fragmented in the solution phase using digestive enzymes. In some examples, the glycopeptide is digested or fragmented in the gaseous phase inside a mass spectrometer, or the instrumentation associated with a mass spectrometer. In some examples, the mass spectroscopy results are analyzed using machine learning algorithms. In some examples, the mass spectroscopy results are the quantification of the glycopeptides, glycans, peptides, and fragments thereof. In some examples, this quantification is used as an input in a trained model to generate an output probability. The output probability is a probability of being within a given category or classification, e.g., the classification of having colorectal cancer or advanced adenoma or the classification of not having colorectal cancer or advanced adenoma. In some other examples, the output probability is a probability of being within a given category or classification, e.g., the classification of having cancer or the classification of not having cancer. In some other examples, the output probability is a probability of being within a given category or classification, e.g., the classification of having an autoimmune disease or the classification of not having an autoimmune disease. In some other examples, the output probability is a probability of being within a given category or classification, e.g., the classification of having fibrosis or the classification of not having an fibrosis.

In some examples, including any of the foregoing, the method includes introducing the sample, or a portion thereof, into a mass spectrometer.

In some examples, including any of the foregoing, the method includes fragmenting a glycopeptide in the sample after introducing the sample, or a portion thereof, into the mass spectrometer.

In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay is used in combination with mass spectroscopy.

In some examples, including any of the foregoing, the method includes digesting a glycopeptide in the sample occurs before introducing the sample, or a portion thereof, into the mass spectrometer.

In some examples, including any of the foregoing, the method includes fragmenting a glycopeptide in the sample to provide a glycopeptide ion, a peptide ion, a glycan ion, a glycan adduction, or a glycan fragment ion.

In some examples, including any of the foregoing, the method includes digesting and/or fragmenting a glycopeptide in the sample to provide a glycopeptide consisting of an amino acid sequence selected from the group consisting of SEQ ID NOs: 1-38, and combinations thereof. In some examples, the methods provides a glycopeptide consisting of an amino acid sequence selected from the group consisting of SEQ ID NOs: 5, 8-11, 13-14, 16-22, 26-28, 30-31, and 34-38, and combinations thereof. In some examples, the method provides a glycopeptide consisting of an amino acid sequence selected from the group consisting of SEQ ID NOs: 3, 7, 9, 28, 29, 32, and 33, and combinations thereof. In some examples, the method provides a glycopeptide consisting of an amino acid sequence selected from the group consisting of SEQ ID NOs: 1-4, 6-7, 12, 15, 23-25, 28, 29, and 32, and combinations thereof.

In some examples, including any of the foregoing, the method includes digesting and/or fragmenting a glycopeptide in the sample to provide a glycopeptide consisting essentially of an amino acid sequence selected from the group consisting of SEQ ID NOs: 1-38, and combinations thereof. In some examples, the methods provides a glycopeptide consisting essentially of an amino acid sequence selected from the group consisting of SEQ ID NOs: 5, 8-11, 13-14, 16-22, 26-28, 30-31, and 34-38, and combinations thereof. In some examples, the method provides a glycopeptide consisting essentially of an amino acid sequence selected from the group consisting of SEQ ID NOs: 3, 7, 9, 28, 29, 32, and 33, and combinations thereof. In some examples, the methods provides a glycopeptide consisting essentially of an amino acid sequence selected from the group consisting of SEQ ID NOs: 1-4, 6-7, 12, 15, 23-25, 28, 29, and 32, and combinations thereof.

In some examples, including any of the foregoing, the method includes digesting a glycopeptide in the sample to provide a glycopeptide consisting of an amino acid sequence selected from the group consisting of SEQ ID NOs: 1-38, and combinations thereof. In some examples, the methods provides a glycopeptide consisting of an amino acid sequence selected from the group consisting of SEQ ID NOs: 5, 8-11, 13-14, 16-22, 26-28, 30-31, and 34-38, and combinations thereof. In some examples, the method provides a glycopeptide consisting of an amino acid sequence selected from the group consisting of SEQ ID NOs: 3, 7, 9, 28, 29, 32, and 33, and combinations thereof. In some examples, the method provides a glycopeptide consisting of an amino acid sequence selected from the group consisting of SEQ ID NOs: 1-4, 6-7, 12, 15, 23-25, 28, 29, and 32, and combinations thereof.

In some examples, including any of the foregoing, the method includes digesting a glycopeptide in the sample to provide a glycopeptide consisting essentially of an amino acid sequence selected from the group consisting of SEQ ID NOs: 1-38, and combinations thereof. In some examples, the methods provides a glycopeptide consisting essentially of an amino acid sequence selected from the group consisting of SEQ ID NOs: 5, 8-11, 13-14, 16-22, 26-28, 30-31, and 34-38, and combinations thereof. In some examples, the method provides a glycopeptide consisting essentially of an amino acid sequence selected from the group consisting of SEQ ID NOs: 3, 7, 9, 28, 29, 32, and 33, and combinations thereof. In some examples, the methods provides a glycopeptide consisting essentially of an amino acid sequence selected from the group consisting of SEQ ID NOs: 1-4, 6-7, 12, 15, 23-25, 28, 29, and 32, and combinations thereof.

In some examples, including any of the foregoing, the method includes fragmenting a glycopeptide in the sample to provide a glycopeptide consisting of an amino acid sequence selected from the group consisting of SEQ ID NOs: 1-38, and combinations thereof. In some examples, the methods provides a glycopeptide consisting of an amino acid sequence selected from the group consisting of SEQ ID NOs: 5, 8-11, 13-14, 16-22, 26-28, 30-31, and 34-38, and combinations thereof. In some examples, the method provides a glycopeptide consisting of an amino acid sequence selected from the group consisting of SEQ ID NOs: 3, 7, 9, 28, 29, 32, and 33, and combinations thereof. In some examples, the method provides a glycopeptide consisting of an amino acid sequence selected from the group consisting of SEQ ID NOs: 1-4, 6-7, 12, 15, 23-25, 28, 29, and 32, and combinations thereof.

In some examples, including any of the foregoing, the method includes fragmenting a glycopeptide in the sample to provide a glycopeptide consisting essentially of an amino acid sequence selected from the group consisting of SEQ ID NOs: 1-38, and combinations thereof. In some examples, the methods provides a glycopeptide consisting essentially of an amino acid sequence selected from the group consisting of SEQ ID NOs: 5, 8-11, 13-14, 16-22, 26-28, 30-31, 3-38, and combinations thereof. In some examples, the method provides a glycopeptide consisting essentially of an amino acid sequence selected from the group consisting of SEQ ID NOs: 3, 7, 9, 28, 29, 32, and 33, and combinations thereof. In some examples, the methods provides a glycopeptide consisting essentially of an amino acid sequence selected from the group consisting of SEQ ID NOs: 1-4, 6-7, 12, 15, 23-25, 28, 29, and 32, and combinations thereof.

In some examples, including any of the foregoing, the method includes detecting a multiple-reaction-monitoring (MRM) transition selected from the group consisting of transitions 1-38. In some examples, the method includes detecting a MRM transition indicative of a glycopeptide or glycan residue, wherein the glycopeptide consists essentially of an amino acid sequence selected from the group consisting of SEQ ID NOs: 1-38 and combinations thereof. In some examples, the method includes detecting a MRM transition indicative of a glycopeptide consisting essentially of an amino acid sequence selected from the group consisting of SEQ ID NOs: 1-38 and combinations thereof. In some examples, the method includes detecting more than one MRM transition selected from a combination of members from the group consisting of transitions 1-38. In some examples, the method includes detecting more than one MRM transition indicative of a combination of glycopeptides having amino acid sequences selected from SEQ ID NOs: 1-38.

In some examples, the method includes detecting a MRM transition indicative of a glycopeptide or glycan residue, wherein the glycopeptide consists essentially of an amino acid sequence selected from the group consisting of SEQ ID NOs: 5, 8-11, 13-14, 16-22, 26-28, 30-31, and 34-38, and combinations thereof. In some examples, the method includes detecting a MRM transition indicative of a glycopeptide or glycan residue, wherein the glycopeptide consists essentially of an amino acid sequence selected from the group consisting of SEQ ID NOs: 3, 7, 9, 28, 29, 32, and 33, and combinations thereof. In some examples, the method includes detecting a MRM transition indicative of a glycopeptide or glycan residue, wherein the glycopeptide consists essentially of an amino acid sequence selected from the group consisting of SEQ ID NOs: 1-4, 6-7, 12, 15, 23-25, 28, 29, and 32, and combinations thereof.

In some examples, the method includes detecting a MRM transition indicative of a glycopeptide consisting essentially of an amino acid sequence selected from the group consisting of SEQ ID NOs: 5, 8-11, 13-14, 16-22, 26-28, 30-31, and 34-38, and combinations thereof. In some examples, the method includes detecting a MRM transition indicative of a glycopeptide consisting essentially of an amino acid sequence selected from the group consisting of SEQ ID NOs: 3, 7, 9, 28, 29, 32, and 33, and combinations. In some examples, the method includes detecting a MRM transition indicative of a glycopeptide consisting essentially of an amino acid sequence selected from the group consisting of SEQ ID NOs: 1-4, 6-7, 12, 15, 23-25, 28, 29, and 32, and combinations.

In some examples, the method includes detecting more than one MRM transition indicative of a combination of glycopeptides having amino acid sequences selected from SEQ ID NOs: 5, 8-11, 13-14, 16-22, 26-28, 30-31, and 34-38. In some examples, the method includes detecting more than one MRM transition indicative of a combination of glycopeptides having amino acid sequences selected from SEQ ID NOs: 3, 7, 9, 28, 29, 32, and 33. In some examples, the method includes detecting more than one MRM transition indicative of a combination of glycopeptides having amino acid sequences selected from SEQ ID NOs: 1-4, 6-7, 12, 15, 23-25, 28, 29, and 32.

In some examples, including any of the foregoing, the method includes performing mass spectroscopy on the biological sample using multiple-reaction-monitoring mass spectroscopy (MRM-MS).

In some examples, including any of the foregoing, the method includes digesting a glycopeptide in the sample to provide a glycopeptide consisting essentially of an amino acid sequence selected from the group consisting of SEQ ID NOs: 1-38, and combinations thereof. In certain examples, the biological sample is combined with chemical reagents. In certain examples, the biological sample is combined with enzymes. In some examples, the enzymes are lipases. In some examples, the enzymes are proteases. In some examples, the enzymes are serine proteases. In some of these examples, the enzyme is selected from the group consisting of trypsin, chymotrypsin, thrombin, elastase, and subtilisin. In some of these examples, the enzyme is trypsin. In some examples, the method includes contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, including any of the foregoing, the method includes detecting a multiple-reaction-monitoring (MRM) transition selected from the group consisting of transitions 1-38. In some examples, the method includes detecting a MRM transition indicative of a glycopeptide or glycan residue, wherein the glycopeptide consisting of, or consisting essentially of, an amino acid sequence selected from the group consisting of SEQ ID NOs: 1-38 and combinations thereof. In some examples, the method includes detecting a MRM transition indicative of a glycopeptide or glycan residue, wherein the glycopeptide consists essentially of an amino acid sequence selected from the group consisting of SEQ ID NOs: 1-38 and combinations thereof. In some examples, the method includes detecting a MRM transition indicative of a glycopeptide consisting essentially of an amino acid sequence selected from the group consisting of SEQ ID NOs: 1-38 and combinations thereof. In some examples, the method includes detecting more than one MRM transition selected from a combination of members from the group consisting of transitions 1-38. In some examples, the method includes detecting more than one MRM transition indicative of a combination of glycopeptides having amino acid sequences selected from SEQ ID NOs: 1-38.

In some examples, the method includes detecting a MRM transition indicative of a glycopeptide or glycan residue, wherein the glycopeptide consisting of, or consisting essentially of, an amino acid sequence selected from the group consisting of SEQ ID NOs: 5, 8-11, 13-14, 16-22, 26-28, 30-31, and 34-38, and combinations thereof. In some examples, the method includes detecting a MRM transition indicative of a glycopeptide or glycan residue, wherein the glycopeptide consisting of, or consisting essentially of, an amino acid sequence selected from the group consisting of SEQ ID NOs: 3, 7, 9, 28, 29, 32, and 33, and combinations thereof. In some examples, the method includes detecting a MRM transition indicative of a glycopeptide or glycan residue, wherein the glycopeptide consisting of, or consisting essentially of, an amino acid sequence selected from the group consisting of SEQ ID NOs: 1-4, 6-7, 12, 15, 23-25, 28, 29, and 32, and combinations thereof.

In some examples, the method includes detecting a MRM transition indicative of a glycopeptide or glycan residue, wherein the glycopeptide consists essentially of an amino acid sequence selected from the group consisting of SEQ ID NOs: 5, 8-11, 13-14, 16-22, 26-28, 30-31, and 34-38, and combinations thereof. In some examples, the method includes detecting a MRM transition indicative of a glycopeptide or glycan residue, wherein the glycopeptide consists essentially of an amino acid sequence selected from the group consisting of SEQ ID NOs: 3, 7, 9, 28, 29, 32, and 33, and combinations thereof. of. In some examples, the method includes detecting a MRM transition indicative of a glycopeptide or glycan residue, wherein the glycopeptide consists essentially of an amino acid sequence selected from the group consisting of SEQ ID NOs: 1-4, 6-7, 12, 15, 23-25, 28, 29, 32 and combinations thereof.

In some examples, the method includes detecting a MRM transition indicative of a glycopeptide consisting essentially of an amino acid sequence selected from the group consisting of SEQ ID NOs: 5, 8-11, 13-14, 16-22, 26-28, 30-31, and 34-38, and combinations thereof. In some examples, the method includes detecting a MRM transition indicative of a glycopeptide consisting essentially of an amino acid sequence selected from the group consisting of SEQ ID NOs: 3, 7, 9, 28, 29, 32, and 33, and combinations thereof. In some examples, the method includes detecting a MRM transition indicative of a glycopeptide consisting essentially of an amino acid sequence selected from the group consisting of SEQ ID NOs: 1-4, 6-7, 12, 15, 23-25, 28, 29, and 32, and combinations thereof.

In some examples, the method includes detecting more than one MRM transition selected from a combination of members from the group consisting of transitions 5, 8-11, 13-14, 16-22, 26-28, 30-31, and 34-38. In some examples, the method includes detecting more than one MRM transition selected from a combination of members from the group consisting of transitions 3, 7, 9, 28, 29, 32, and 33. In some examples, the method includes detecting more than one MRM transition selected from a combination of members from the group consisting of transitions 1-4, 6-7, 12, 15, 23-25, 28, 29, and 32.

In some examples, the method includes detecting more than one MRM transition indicative of a combination of glycopeptides having amino acid sequences selected from SEQ ID NOs: 5, 8-11, 13-14, 16-22, 26-28, 30-31, and 34-38. In some examples, the method includes detecting more than one MRM transition indicative of a combination of glycopeptides having amino acid sequences selected from SEQ ID NOs: 3, 7, 9, 28, 29, 32, and 33. In some examples, the method includes detecting more than one MRM transition indicative of a combination of glycopeptides having amino acid sequences selected from SEQ ID NOs: 1-4, 6-7, 12, 15, 23-25, 28, 29, 32.

In some examples, including any of the foregoing, the method includes performing mass spectroscopy on the biological sample using multiple-reaction-monitoring mass spectroscopy (MRM-MS).

In some examples, including any of the foregoing, the method includes digesting a glycopeptide in the sample to provide a glycopeptide consisting of an amino acid sequence selected from the group consisting of SEQ ID NOs: 1-38, and combinations thereof. In certain examples, the biological sample is contacted with one or more chemical reagents. In certain examples, the biological sample is contacted with one or more enzymes. In some examples, the enzymes are lipases. In some examples, the enzymes are proteases. In some examples, the enzymes are serine proteases. In some of these examples, the enzyme is selected from the group consisting of trypsin, chymotrypsin, thrombin, elastase, and subtilisin. In some of these examples, the enzyme is trypsin. In some examples, the method includes contacting at least two proteases with a glycopeptide in a sample. In some examples, the at least two proteases are selected from the group consisting of serine protease, threonine protease, cysteine protease, aspartate protease. In some examples, the at least two proteases are selected from the group consisting of trypsin, chymotrypsin, endoproteinase, Asp-N, Arg-C, Glu-C, Lys-C, pepsin, thermolysin, elastase, papain, proteinase K, subtilisin, clostripain, and carboxypeptidase protease, glutamic acid protease, metalloprotease, and asparagine peptide lyase.

In some examples, including any of the foregoing, the MRM transition is selected from the transitions, or any combinations thereof, in any one of Tables 1, 2 or 3.

In some examples, including any of the foregoing, the method includes conducting tandem liquid chromatography-mass spectroscopy on the biological sample.

In some examples, including any of the foregoing, the method includes multiple-reaction-monitoring mass spectroscopy (MRM-MS) mass spectroscopy on the biological sample.

In some examples, including any of the foregoing, the method includes detecting a MRM transition using a triple quadrupole (QQQ) and/or a quadrupole time-of-flight (qTOF) mass spectrometer. In certain examples, the method includes detecting a MRM transition using a QQQ mass spectrometer. In certain other examples, the method includes detecting using a qTOF mass spectrometer. In some examples, a suitable instrument for use with the instant methods is an Agilent 6495B Triple Quadrupole LC/MS, which can be found at www.agilent.com/en/products/mass-spectrometry/lc-ms-instruments/triple-quadrupole-lc-ms/6495b-triple-quadrupole-lc-ms. In certain other examples, the method includes detecting using a QQQ mass spectrometer. In some examples, a suitable instrument for use with the instant methods is an Agilent 6545 LC/Q-TOF, which can be found at https://www.agilent.com/en/products/liquid-chromatography-mass-spectrometry-lc-ms/lc-ms-instruments/quadrupole-time-of-flight-lc-ms/6545-q-tof-lc-ms.

In some examples, including any of the foregoing, the method includes detecting more than one MRM transition using a QQQ and/or qTOF mass spectrometer. In certain examples, the method includes detecting more than one MRM transition using a QQQ mass spectrometer. In certain examples, the method includes detecting more than one MRM transition using a qTOF mass spectrometer. In certain examples, the method includes detecting more than one MRM transition using a QQQ mass spectrometer.

In some examples, including any of the foregoing, the methods herein include quantifying one or more glycomic parameters of the one or more biological samples comprises employing a coupled chromatography procedure. In some examples, these glycomic parameters include the identification of a glycopeptide group, identification of glycans on the glycopeptide, identification of a glycosylation site, identification of part of an amino acid sequence which the glycopeptide includes. In some examples, the coupled chromatography procedure comprises: performing or effectuating a liquid chromatography-mass spectrometry (LC-MS) operation. In some examples, the coupled chromatography procedure comprises: performing or effectuating a multiple reaction monitoring mass spectrometry (MRM-MS) operation. In some examples, the methods herein include a coupled chromatography procedure which comprises: performing or effectuating a liquid chromatography-mass spectrometry (LC-MS) operation; and effectuating a multiple reaction monitoring mass spectrometry (MRM-MS) operation. In some examples, the methods include training a machine learning algorithm using one or more glycomic parameters of the one or more biological samples obtained by one or more of a triple quadrupole (QQQ) mass spectrometry operation and/or a quadrupole time-of-flight (qTOF) mass spectrometry operation. In some examples, the methods include training a machine learning algorithm using one or more glycomic parameters of the one or more biological samples obtained a triple quadrupole (QQQ) mass spectrometry operation. In some examples, the methods include training a machine learning algorithm using one or more glycomic parameters of the one or more biological samples obtained by a quadrupole time-of-flight (qTOF) mass spectrometry operation. In some examples, the methods include quantifying one or more glycomic parameters of the one or more biological samples comprises employing one or more of a triple quadrupole (QQQ) mass spectrometry operation and a quadrupole time-of-flight (qTOF) mass spectrometry operation. In some examples, machine learning algorithms are used to quantify these glycomic parameters. In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay (e.g., ELISA) is used in combination with mass spectroscopy.

In some examples, including any of the foregoing, the glycopeptide or combination thereof consists of an amino acid sequence selected from the group consisting of SEQ ID NOs: 1-38 and combinations thereof.

In some examples, including any of the foregoing, the glycopeptide or combination thereof consists essentially of an amino acid sequence selected from the group consisting of SEQ ID NOs: 1-38 and combinations thereof.

In some examples, including any of the foregoing, the method includes digesting and/or fragmenting a glycopeptide in the sample to provide a glycopeptide consisting of an amino acid sequence selected from the group consisting of SEQ ID NOs: 1-38 and combinations thereof.

In some examples, including any of the foregoing, the method includes digesting and/or fragmenting a glycopeptide in the sample to provide a glycopeptide consisting essentially of an amino acid sequence selected from the group consisting of SEQ ID NOs: 1-38 and combinations thereof.

In some examples, including any of the foregoing, the glycopeptide or combination thereof consists of an amino acid sequence selected from the group consisting of SEQ ID NOs: 5, 8-11, 13-14, 16-22, 26-28, 30-31, and 34-38, and combinations thereof. In some examples, including any of the foregoing, the glycopeptide or combination thereof consists of an amino acid sequence selected from the group consisting of SEQ ID NOs: 3, 7, 9, 28, 29, 32, and 33, and combinations thereof. In some examples, including any of the foregoing, the glycopeptide or combination thereof consists of an amino acid sequence selected from the group consisting of SEQ ID NOs: 1-4, 6-7, 12, 15, 23-25, 28, 29, and 32, and combinations thereof.

In some examples, including any of the foregoing, the glycopeptide or combination thereof consists essentially of an amino acid sequence selected from the group consisting of SEQ ID NOs: 5, 8-11, 13-14, 16-22, 26-28, 30-31, and 34-38, and combinations thereof. In some examples, including any of the foregoing, the glycopeptide or combination thereof consists essentially of an amino acid sequence selected from the group consisting of SEQ ID NOs: 3, 7, 9, 28, 29, 32, and 33, and combinations thereof. In some examples, including any of the foregoing, the glycopeptide or combination thereof consists essentially of an amino acid sequence selected from the group consisting of SEQ ID NOs: 1-4, 6-7, 12, 15, 23-25, 28, 29, and 32, and combinations thereof.

In some examples, including any of the foregoing, the method includes digesting and/or fragmenting a glycopeptide in the sample to provide a glycopeptide consisting of an amino acid sequence selected from the group consisting of SEQ ID NOs: 5, 8-11, 13-14, 16-22, 26-28, 30-31, and 34-38, and combinations thereof. In some examples, including any of the foregoing, the method includes digesting and/or fragmenting a glycopeptide in the sample to provide a glycopeptide consisting of an amino acid sequence selected from the group consisting of SEQ ID NOs: 3, 7, 9, 28, 29, 32, and 33, and combinations thereof. In some examples, including any of the foregoing, the method includes digesting and/or fragmenting a glycopeptide in the sample to provide a glycopeptide consisting of an amino acid sequence selected from the group consisting of SEQ ID NOs: 1-4, 6-7, 12, 15, 23-25, 28, 29, and 32, and combinations thereof.

In some examples, including any of the foregoing, the method includes digesting and/or fragmenting a glycopeptide in the sample to provide a glycopeptide consisting essentially of an amino acid sequence selected from the group consisting of SEQ ID NOs: 5, 8-11, 13-14, 16-22, 26-28, 30-31, and 34-38, and combinations thereof. In some examples, including any of the foregoing, the method includes digesting and/or fragmenting a glycopeptide in the sample to provide a glycopeptide consisting essentially of an amino acid sequence selected from the group consisting of SEQ ID NOs: 3, 7, 9, 28, 29, 32, and 33, and combinations thereof. In some examples, including any of the foregoing, the method includes digesting and/or fragmenting a glycopeptide in the sample to provide a glycopeptide consisting essentially of an amino acid sequence selected from the group consisting of SEQ ID NOs: 1-4, 6-7, 12, 15, 23-25, 28, 29, and 32, and combinations thereof.

In some examples, including any of the foregoing, the method includes detecting one or more MRM transitions indicative of glycans selected from the group consisting of glycan 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof. Herein, these glycans are illustrated in FIGS. 1-14.

In some examples, including any of the foregoing, the method includes quantifying a glycan.

In some examples, including any of the foregoing, the method includes quantifying a first glycan and quantifying a second glycan; and further comprising comparing the quantification of the first glycan with the quantification of the second glycan.

In some examples, including any of the foregoing, the method includes associating the detected glycan with a peptide residue site, whence the glycan was bonded.

In some examples, including any of the foregoing, the method includes generating a glycosylation profile of the sample.

In some examples, including any of the foregoing, the method includes spatially profiling glycans on a tissue section associated with the sample. In some examples, including any of the foregoing, the method includes spatially profiling glycopeptides on a tissue section associated with the sample. In some examples, the method includes matrix-assisted laser desorption ionization time-of-flight mass spectrometry (MALDI-TOF) mass spectroscopy in combination with the methods herein.

In some examples, including any of the foregoing, the method includes quantifying relative abundance of a glycan and/or a peptide.

In some examples, including any of the foregoing, the method includes normalizing the amount of a glycopeptide by quantifying a glycopeptide consisting essentially of an amino acid sequence selected from the group consisting of SEQ ID NOs: 1-38, and combinations thereof and comparing that quantification to the amount of another chemical species. In some examples, the method includes normalizing the amount of a peptide by quantifying a glycopeptide consisting of an amino acid sequence selected from the group consisting of SEQ ID NOs: 1-38, and combinations thereof, and comparing that quantification to the amount of another glycopeptide consisting of an amino acid sequence selected from the group consisting of SEQ ID NOs: 1-38. In some examples, the method includes normalizing the amount of a peptide by quantifying a glycopeptide consisting essentially of an amino acid sequence selected from the group consisting of SEQ ID NOs: 1-38, and combinations thereof, and comparing that quantification to the amount of another glycopeptide consisting essentially of an amino acid sequence selected from the group consisting of SEQ ID NOs: 1-38.

### B. Methods for Classifying Samples Comprising Glycopeptides

In another embodiment, set forth herein a method for identifying a classification for a sample, the method comprising: quantifying by mass spectroscopy (MS) one or more glycopeptides in a sample wherein the glycopeptides each, individually in each instance, comprise an amino acid sequence or any of SEQ ID NOs: 1-38, together with any associated glycan of Table 10 and as described herein, and combinations thereof; and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, comprise or consist essentially of or consist of an amino acid sequence selected from the group consisting of, or consisting essentially of, SEQ ID NOs: 1-38, together with any associated glycan, for instance as described herein, and combinations thereof; and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, comprise an amino acid sequence or any of SEQ ID NOs: 5, 8-11, 13-14, 16-22, 26-28, 30-31, and 34-38, together with any associated glycan of Table 10 and as described herein, and combinations thereof; and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, consist essentially of an amino acid sequence selected from the group consisting of, or consisting essentially of, SEQ ID NOs: 5, 8-11, 13-14, 16-22, 26-28, 30-31, and 34-38, together with any associated glycan, for instance as described herein, and combinations thereof; and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, comprise an amino acid sequence or any of SEQ ID NOs: 3, 7, 9, 28, 29, 32, and 33, together with any associated glycan of Table 10 and as described herein, and combinations thereof; and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, consist essentially of an amino acid sequence selected from the group consisting of, or consisting essentially of, SEQ ID NOs: 3, 7, 9, 28, 29, 32, and 33, together with any associated glycan, for instance as described herein, and combinations thereof; and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, comprise an amino acid sequence or any of SEQ ID NOs: 1-4, 6-7, 12, 15, 23-25, 28, 29, and 32, together with any associated glycan of Table 10 and as described herein, and combinations thereof, and inputting the quantification into a trained model to generate a output probability; determining if the output probability is above or below a threshold for a classification; and identifying a classification for the sample based on whether the output probability is above or below a threshold for a classification.

In another embodiment, set forth herein a method for identifying a classification for a sample, the method comprising: quantifying by mass spectroscopy (MS) one or more glycopeptides in a sample wherein the glycopeptides each, individually in each instance, consist essentially of an amino acid sequence selected from the group consisting of, or consisting essentially of, SEQ ID NOs: 1-4, 6-7, 12, 15, 23-25, 28, 29, and 32, together with any associated glycan, for instance as described herein, and combinations thereof; and inputting the quantification into a trained model to generate a output probability; determining if the output probability is above or below a threshold for a classification; and identifying a classification for the sample based on whether the output probability is above or below a threshold for a classification.

In some examples, set forth herein is a method for classifying glycopeptides, comprising: obtaining, or having obtained a biological sample from a patient; digesting and/or fragmenting a glycopeptide in the sample; detecting a multiple-reaction-monitoring (MRM) transition selected from the group consisting of transitions 1-38; and classifying the glycopeptides based on the MRM transitions detected. In some examples, a machine learning algorithm is used to train a model using the analyzed the MRM transitions as inputs. In some examples, a machine learning algorithm is trained using the MRM transitions as a training data set. In some examples, the methods herein include identifying glycopeptides, peptides, and glycans based on their mass spectroscopy relative abundance. In some examples, a machine learning algorithm or algorithms select and/or identify peaks in a mass spectroscopy spectrum.

In some examples, set forth herein is a method for classifying glycopeptides, comprising: obtaining, or having obtained a biological sample from an individual; digesting and/or fragmenting a glycopeptide in the sample; detecting a multiple-reaction-monitoring (MRM) transition selected from the group consisting of transitions 1-38; and classifying the glycopeptides based on the MRM transitions detected. In some examples, a machine learning algorithm is used to train a model using the analyzed the MRM transitions as inputs. In some examples, a machine learning algorithm is trained using the MRM transitions as a training data set. In some examples, the methods herein include identifying glycopeptides, peptides, and glycans based on their mass spectroscopy relative abundance. In some examples, a machine learning algorithm or algorithms select and/or identify peaks in a mass spectroscopy spectrum.

In some examples, set forth herein is a method of training a machine learning algorithm using MRM transitions as an input data set. In some examples, set forth herein is a method for identifying a classification for a sample, the method comprising quantifying by mass spectroscopy (MS) a glycopeptide in a sample wherein the glycopeptide consisting of, or consisting essentially of, an amino acid sequence selected from the group consisting of SEQ ID NOs: 5, 8-11, 13-14, 16-22, 26-28, 30-31, and 34-38, together with any associated glycan, for instance as described herein, and combinations thereof; and identifying a classification based on the quantification. In some examples, the quantifying includes determining the presence or absence of a glycopeptide, or combination of glycopeptides, in a sample. In some examples, the quantifying includes determining the relative abundance of a glycopeptide, or combination of glycopeptides, in a sample. In some examples, a trained model is used to generate an output probability based on inputting the quantification of the detected polypeptides or the MRM transitions.

In some examples, set forth herein is a method of training a machine learning algorithm using MRM transitions as an input data set. In some examples, set forth herein is a method for identifying a classification for a sample, the method comprising quantifying by mass spectroscopy (MS) a glycopeptide in a sample wherein the glycopeptide consisting of, or consisting essentially of, an amino acid sequence selected from the group consisting of SEQ ID NOs: 3, 7, 9, 28, 29, 32, and 33, together with any associated glycan, for instance as described herein, and combinations thereof; and identifying a classification based on the quantification. In some examples, the quantifying includes determining the presence or absence of a glycopeptide, or combination of glycopeptides, in a sample. In some examples, the quantifying includes determining the relative abundance of a glycopeptide, or combination of glycopeptides, in a sample. In some examples, a trained model is used to generate an output probability based on inputting the quantification of the detected polypeptides or the MRM transitions.

In some examples, set forth herein is a method of training a machine learning algorithm using MRM transitions as an input data set. In some examples, set forth herein is a method for identifying a classification for a sample, the method comprising quantifying by mass spectroscopy (MS) a glycopeptide in a sample wherein the glycopeptide consisting of, or consisting essentially of, an amino acid sequence selected from the group consisting of SEQ ID NOs: 1-4, 6-7, 12, 15, 23-25, 28, 29, and 32, together with any associated glycan, for instance as described herein, and combinations thereof; and identifying a classification based on the quantification. In some examples, the quantifying includes determining the presence or absence of a glycopeptide, or combination of glycopeptides, in a sample. In some examples, the quantifying includes determining the relative abundance of a glycopeptide, or combination of glycopeptides, in a sample. In some examples, a trained model is used to generate an output probability based on inputting the quantification of the detected polypeptides or the MRM transitions.

In some examples, set forth herein is a method of training a machine learning algorithm using MRM transitions as an input data set. In some examples, set forth herein is a method for identifying a classification for a sample, the method comprising quantifying by mass spectroscopy (MS) a glycopeptide in a sample wherein the glycopeptide consisting of, or consisting essentially of, an amino acid sequence selected from the group consisting of SEQ ID NOs: 1-38, together with any associated glycan, for instance as described herein, and combinations thereof; and identifying a classification based on the quantification. In some examples, the quantifying includes determining the presence or absence of a glycopeptide, or combination of glycopeptides, in a sample. In some examples, the quantifying includes determining the relative abundance of a glycopeptide, or combination of glycopeptides, in a sample. In some examples, a trained model is used to generate an output probability based on inputting the quantification of the detected polypeptides or the MRM transitions.

In some examples, including any of the foregoing, the sample is a biological sample from a patient having a disease or condition.

In some examples, including any of the foregoing, the patient has colorectal cancer or advanced adenoma.

In some examples, including any of the foregoing, the patient has cancer.

In some examples, including any of the foregoing, the patient has fibrosis.

In some examples, including any of the foregoing, the patient has an autoimmune disease.

In some examples, including any of the foregoing, the disease or condition is colorectal cancer or advanced adenoma.

In some examples, including any of the foregoing, the MS is MRM-MS with a QQQ and/or qTOF mass spectrometer.

In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay is used in combination with mass spectroscopy.

In some examples, including any of the foregoing, the machine learning algorithm is selected from the group consisting of a deep learning algorithm, a neural network algorithm, an artificial neural network algorithm, a supervised machine learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, a regularized regression algorithm, or a combination thereof. In certain examples, the machine learning algorithm is LASSO regression. In certain examples, the machine learning algorithm is combined discriminant analysis.

In some examples, including any of the foregoing, the method includes classifying a sample as within, or embraced by, a disease classification or a disease severity classification.

In some examples, including any of the foregoing, the method includes quantifying by MS the glycopeptide in a sample at a first time point; quantifying by MS the glycopeptide in a sample at a second time point; and comparing the quantification at the first time point with the quantification at the second time point.

In some examples, including any of the foregoing, the method includes quantifying by MS a different glycopeptide in a sample at a third time point; quantifying by MS the different glycopeptide in a sample at a fourth time point; and comparing the quantification at the fourth time point with the quantification at the third time point.

In some examples, including any of the foregoing, the method includes monitoring the health status of a patient.

In some examples, including any of the foregoing, monitoring the health status of a patient includes monitoring the onset and progression of disease in a patient with risk factors such as genetic mutations, as well as detecting cancer recurrence. In some embodiments, the patient has one or more risk factors or clinical indicators of colorectal cancer (CRC). In some embodiments, the subject has one or more risk factors associated with CRC. In some embodiments, the risk factor for CRC is selected from the group consisting of age, irritable bowel disease, type 2 diabetes, a family history of CRC, a genetic syndrome (e.g., Lynch syndrome), obesity, smoking, alcohol consumption, dietary choices, and limited physical activity. In some embodiments, the clinical indicator of CRC is selected from the group consisting of changes in bowel habits, bloody stool, diarrhea, constipation, persistent abdominal pain, persistent abdominal cramps, and unexplained weight loss. In some embodiments, the individual is determined have a healthy state, wherein a healthy state comprises the absence of CRC or AA. In some embodiments, the method further comprises generating a report that includes a diagnosis based on the corresponding state detected for the subject.

In some examples, including any of the foregoing, the method includes quantifying by MS a glycopeptide consisting of an amino acid sequence selected from the group consisting of SEQ ID NOs: 1-38.

In some examples, including any of the foregoing, the method includes quantifying by MS a glycopeptide consisting essentially of an amino acid sequence selected from the group consisting of SEQ ID NOs: 1-38 together with any associated glycan, for instance as described herein.

In some examples, including any of the foregoing, the method includes quantifying by MS one or more glycans selected from the group consisting of glycan 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof.

In some examples, including any of the foregoing, the method includes diagnosing a patient with a disease or condition based on the quantification.

In some examples, including any of the foregoing, the method includes diagnosing the patient as having colorectal cancer or advanced adenoma based on the quantification.

In some examples, including any of the foregoing, the method includes treating the patient with a therapeutically effective amount of a therapeutic agent selected from the group consisting of a chemotherapeutic, an immunotherapy, a hormone therapy, a targeted therapy, a neoadjuvant therapy, surgery, and combinations thereof.

### C. Methods of Treatment

In some examples, set forth herein is a method for treating a patient having a disease or condition, comprising measuring by mass spectroscopy a glycopeptide in a sample from the patient. In some examples, the patient is a human. In certain examples, the patient is a female. In certain other examples, the patient is a female with colorectal cancer or advanced adenoma. In certain examples, the patient is a female with colorectal cancer or advanced adenoma at Stage 1. In certain examples, the patient is a female with colorectal cancer or advanced adenoma at Stage 2. In certain examples, the patient is a female with colorectal cancer or advanced adenoma at Stage 3. In certain examples, the patient is a female with colorectal cancer or advanced adenoma at Stage 4. In some examples, the female has an age equal or between 10-20 years. In some examples, the female has an age equal or between 20-30 years. In some examples, the female has an age equal or between 30-40 years. In some examples, the female has an age equal or between 40-50 years. In some examples, the female has an age equal or between 50-60 years. In some examples, the female has an age equal or between 60-70 years. In some examples, the female has an age equal or between 70-80 years. In some examples, the female has an age equal or between 80-90 years. In some examples, the female has an age equal or between 90-100 years.

In some examples, set forth herein is a method for treating a patient having a disease or condition, comprising measuring by mass spectroscopy a glycopeptide in a sample from the patient. In some examples, the patient is a human. In certain examples, the patient is a male. In certain other examples, the patient is a male with colorectal cancer or advanced adenoma. In certain examples, the patient is a male with colorectal cancer or advanced adenoma at Stage 1. In certain examples, the patient is a male with colorectal cancer or advanced adenoma at Stage 2. In certain examples, the patient is a male with colorectal cancer or advanced adenoma at Stage 3. In certain examples, the patient is a male with colorectal cancer or advanced adenoma at Stage 4. In some examples, the male has an age equal or between 10-20 years. In some examples, the male has an age equal or between 20-30 years. In some examples, the male has an age equal or between 30-40 years. In some examples, the male has an age equal or between 40-50 years. In some examples, the male has an age equal or between 50-60 years. In some examples, the male has an age equal or between 60-70 years. In some examples, the male has an age equal or between 70-80 years. In some examples, the male has an age equal or between 80-90 years. In some examples, the male has an age equal or between 90-100 years.

In another embodiment, set forth herein is a method for treating a patient having colorectal cancer or advanced adenoma; the method comprising: obtaining, or having obtained a biological sample from the patient; digesting and/or fragmenting one or more glycopeptides in the sample; and detecting and quantifying one or more multiple-reaction-monitoring (MRM) transitions selected from the group consisting of transitions 1-38; inputting the quantification into a trained model to generate an output probability; determining if the output probability is above or below a threshold for a classification; and classifying the patient based on whether the output probability is above or below a threshold for a classification, wherein the classification is selected from the group consisting of: (A) a patient in need of resection; (B) a patient in need of a therapeutic agent; (C) a patient in need of an alkylating therapy; (D) a patient in need of a targeted therapeutic agent; (E) a patient in need of an immune therapeutic; (F) a patient in need of immune checkpoint inhibitors; (G) a patient in need of T-cell-related therapies; (H) a patient in need of a cancer vaccine; (I) a patient in need of radiotherapy; (J) a patient in need of a colonoscopy; or (K) a combination thereof; performing, or having performed, a resection if classification A or K is determined; performing, or having performed, a radiotherapy if classification I or K is determined; performing, or having performed, a colonoscopy if classification J or K is determined; or administering a therapeutically effective amount of a therapeutic agent to the patient: wherein the therapeutic agent is selected from a therapeutic agent if classification B or K is determined; or wherein the therapeutic agent is selected from alkylating agent if classification C or K is determined; or wherein the therapeutic agent is selected from targeted therapeutic agent if classification D or K is determined; wherein the therapeutic agent is selected from immune-therapeutic agent if classification E or K is determined; wherein the therapeutic agent is selected from immune checkpoint inhibitor if classification F or K is determined; wherein the therapeutic agent is selected from T-cell-related therapy if classification G or K is determined; and wherein the therapeutic agent is selected from a cancer vaccine if classification H or K is determined.

In another embodiment, set forth herein is a method for treating a patient having colorectal cancer or advanced adenoma; the method comprising: selecting a patient having a biological sample comprising one or more glycopeptides; wherein the one or more glycopeptides in the sample were digested and/or fragmented; and wherein the one or more glycopeptides in the sample were detected and quantified using one or more multiple-reaction-monitoring (MRM) transitions selected from the group consisting of transitions 1-38; wherein the quantification was input into a trained model to generate an output probability; and wherein an output probability was determined to be above or below a threshold for a classification; and classifying the patient based on whether the output probability is above or below a threshold for a classification, wherein the classification is selected from the group consisting of: (A) a patient in need of resection; (B) a patient in need of a therapeutic agent; (C) a patient in need of an alkylating therapy; (D) a patient in need of a targeted therapeutic agent; (E) a patient in need of an immune therapeutic; (F) a patient in need of immune checkpoint inhibitors; (G) a patient in need of T-cell-related therapies; (H) a patient in need of a cancer vaccine; (I) a patient in need of radiotherapy; (J) a patient in need of a colonoscopy; or (K) a combination thereof; performing, or having performed, a resection if classification A or K is determined; performing, or having performed, a radiotherapy if classification I or K is determined; performing, or having performed, a colonoscopy if classification J or K is determined; or administering a therapeutically effective amount of a therapeutic agent to the patient: wherein the therapeutic agent is selected from a therapeutic agent if classification B or K is determined; or wherein the therapeutic agent is selected from alkylating agent if classification C or K is determined; or wherein the therapeutic agent is selected from targeted therapeutic agent if classification D or K is determined; wherein the therapeutic agent is selected from immune-therapeutic agent if classification E or K is determined; wherein the therapeutic agent is selected from immune checkpoint inhibitor if classification F or K is determined; wherein the therapeutic agent is selected from T-cell-related therapy if classification G or K is determined; and wherein the therapeutic agent is selected from a cancer vaccine if classification H or K is determined.

In some examples, MRM transitions are quantified and this quantification is used as an input in a trained model to generate an output probability. The output probability is a probability of being within a given category or classification, e.g., the classification of having colorectal cancer or advanced adenoma or the classification of not having colorectal cancer or advanced adenoma. In some other examples, the output probability is a probability of being within a given category or classification, e.g., the classification of having cancer or the classification of not having cancer. In some other examples, the output probability is a probability of being within a given category or classification, e.g., the classification of having an autoimmune disease or the classification of not having an autoimmune disease. In some other examples, the output probability is a probability of being within a given category or classification, e.g., the classification of having fibrosis or the classification of not having an fibrosis. In some examples, the methods comprise treating a patient after inputting quantified MRM transitions into a trained model to generate an output probability, and treating the patient in accordance with the output probability.

In some examples, the machine learning is used to identify MS peaks associated with MRM transitions. In some examples, the MRM transitions are analyzed using machine learning. In some examples, the MRM transitions are analyzed with a trained machine learning algorithm. In some of these examples, the trained machine learning algorithm was trained using MRM transitions observed by analyzing samples from patients known to have colorectal cancer or advanced adenoma.

In some examples, the trained model is used to treat a patient having colorectal cancer or advanced adenoma. In some examples, the trained model is used to identify MS peaks associated with MRM transitions to treat a patient. In some examples, the trained model is used to identify machine a MRM transitions to treat a patient. In some examples, the trained model quantifies the amount of glycopeptides associated with an MRM transition(s) and generates an output probability that is used to treat a patient. In some of these examples, the trained model uses MRM transitions observed by analyzing samples from patients known to have colorectal cancer or advanced adenoma to treat a patient.

In some examples, one or more risk factors or clinical indicators of colorectal cancer (CRC) are considered in diagnosing and treating a patient. In some embodiments, the patient being diagnosed has one or more risk factors associated with CRC. In some embodiments, the patient being treated has one or more risk factors associated with CRC. In some embodiments, the risk factor for CRC comprises one or more of age, irritable bowel disease, type 2 diabetes, a family history of CRC, a genetic syndrome (e.g., Lynch syndrome), obesity, smoking, alcohol consumption, dietary choices, limited physical activity and combinations thereof. In some embodiments, the patient being diagnosed has one or more clinical indicator associated with CRC. In some embodiments, the patient being treated has one or more clinical indicator associated with CRC. In some embodiments, the clinical indicator of CRC comprises one or more of changes in bowel habits, bloody stool, diarrhea, constipation, persistent abdominal pain, persistent abdominal cramps, and unexplained weight loss. In some embodiments, the individual is determined have a healthy state, wherein a healthy state comprises the absence of CRC or AA. In some embodiments, the method further comprises generating a report that includes a diagnosis based on the corresponding state detected for the subject.

In some examples, after diagnosing the patient as having colorectal cancer, the patient is treated with surgery. In some examples, after diagnosing the patient as having colorectal cancer, the patient is treated with a resection. In some embodiments, the surgery to treat colorectal cancer (CRC) comprises the removal of one or more parts of the colon. In some embodiments, the therapy comprises a polypectomy, a local excision, a transanal excision (TAE), lymph node removal, a transanal endoscopic microsurgery (TEM), a low anterior resection (LAR), a proctectomy with colo-anal anastomosis, an abdominoperineal resection (APR), a pelvic exenteration, or a diverting colostomy. In some embodiments, the surgery may comprise cryosurgery.

In some examples, after diagnosing the patient as having colorectal cancer, the patient is treated with a therapeutically effective amount of an antimetabolite such as Leucovorin, Fluorouracil (5FOU), Capecitabine, and Trifluridine/Tipiricil. In some embodiments, the chemotherapeutic therapy to treat colorectal cancer (CRC) comprises 5-fluorouracil, capecitabine, oxaliplatin, irinotecan, trifluridine and tipiracil, or a combination thereof. 5-fluorouracil can be dosed to a human subject with a range of about 0.4 g/m² per day to about 3 g/m² per day. Capecitabine can be dosed to a human subject at about 1250 mg/m² BID×2 weeks, followed by 1-week rest period, given as 3-week cycles. Oxaliplatin can be dosed to a human subject with a range of about 85 g/m² per day to about 600 mg/m² per day. Irinotecan can be dosed to a human subject with a range of about 125 mg/m² per day to about 350 mg/m² per day. Trifluridine/tipiracil can be dosed to a human subject with a range of about 35 mg/m² PO BID to about a not to exceed 80 mg. It should be noted that m² can refer to the approximate surface area of the human subject, PO can mean per oral or by mouth, and BID can refer bis in die or twice a day.

In some examples, after diagnosing the patient as having colorectal cancer, the patient is treated with a therapeutically effective amount of a topoisomerase inhibitor such as Irinotecan.

In some examples, patients are treated with a therapeutically effective amount of an alkylating agent. In certain examples the alkylating agent comprises drugs such as oxaliplatin and eloxatin.

In some examples, patients are treated with a therapeutically effective amount of a targeted therapeutic agent. In certain examples, the targeted therapeutic agent is a drug that targets blood vessel that targets vascular endothelial growth factor (VEGF) such as Bevacizumab (Avastin), Ramucirumab (Cyramza), and Ziv-aflibercept (Zaltrap). In certain examples, the targeted therapeutic agent is a epidermal growth factor receptor (EGFR) such as Cetuximab (Erbitux), or Panitumumab (Vectibix). In certain examples, the targeted therapeutic agent is a kinase inhibitor such as Regorafenib (Stivarga). In some embodiments, the targeted therapeutic agent is selected based on patient-specific changes in tumor cell gene expression including but not limited to changes in VEGF, EGFR, BRAF, and MEK genes. In some embodiments, the targeted therapeutic agent is an inhibitor of an oncogene. In some embodiments, the targeted therapeutic agent is an inhibitor of one or more of VEGF, EGFR, BRAF, and MEK. In some embodiments, the targeted therapeutic agent comprises aflibercept, cetuximab, panitumumab, encorafenib, and combinations thereof. In some embodiments the targeted therapeutic agent comprises an angiogenesis inhibitor. In some embodiments, the angiogenesis inhibitor comprises one of bevacizumab (Avastin, BEV) and ramucirumab (Cyramza, RAM). In some embodiments, the therapy for CRC comprises a combination of one or more targeted therapeutic agents.

In some examples, patients are treated with a therapeutically effective amount of an immune-therapeutic. In certain examples, the immune-therapeutic is selected from the group consisting of immune checkpoint inhibitors. In certain examples, the checkpoint inhibitors are selected from the group consisting of PD-1-, PD-L1-, CTLA-4-inhibitors, and combinations thereof. In some embodiments, immunotherapy is an the antibody. In some embodiments, the antibody is directed towards an immune system checkpoint protein including but not limited to PD-1, PD-L1, and CTLA-4. In some embodiments, the antibody targeting PD-1 comprises nivolumab (Opdivo), pembrolizumab (Keytruda), and cemiplimab (Libtayo). In some embodiments, the antibody targeting PD-L1 comprises atezolizumab (Tecentriq), durvalumab (Imfinzi), and avelumab (Bavencio). In some embodiments, the antibody targeting CTLA-4 comprises ipilimumab (Yervoy). In some embodiments, the therapy for CRC comprises a combination of one or more antibody that targets PD-1, PD-L1, and CTLA-4.

In some examples, patients are treated with a therapeutically effective amount of T-cell-related therapies. In certain examples, the T-cell-related therapies are selected from the group consisting of CAR-T-approaches, TCR-approaches, and combinations thereof.

In some examples, patients are treated with a therapeutically effective amount of a cancer vaccine.

In some examples, patients are treated with a therapeutically effective amount of radiotherapy. In certain examples, the radiotherapy is selected from the group consisting of external beam-radiotherapy and internal-radiotherapy, chemoradiation, brachytherapy, and combinations thereof. In some embodiments, the radiotherapy is a radiation procedure comprising the use of high-energy rays or particles to treat colorectal cancer (CRC). In some embodiments, the radiation procedure comprises external beam radiation therapy (EBRT) and internal radiation therapy (also referred to as brachytherapy). In some embodiments, the EBRT comprises one or more of stereotactic ablative radiotherapy (SABR), three-dimensional conformal radiation therapy (3D-CRT), intensity modulated radiation therapy (IMRT), stereotactic body radiation therapy (SBRT) stereotactic radiosurgery (SRS) or a combination thereof. In some embodiments, the brachytherapy comprises the placement of radioactive material in or adjacent to the tumor in the colon (e.g., rectal cavity).

In some examples, the patient is treated with a therapeutic agent selected from targeted therapy. In some examples, the methods herein include administering a therapeutically effective amount of a 5-Fluorouracil (5-FU); Capecitabine (Xeloda), Irinotecan (Camptosar), Oxaliplatin (Eloxatin), Trifluridine, and tipiracil (Lonsurf).

In some examples, the therapeutic agent is administered at 150 mg, 250 mg, 300 mg, 350 mg, and 600 mg doses. In some examples, the therapeutic agent is administered twice daily.

Chemotherapeutic agents include, but are not limited to, platinum-based drug such as carboplatin (Paraplatin) or cisplatin with a taxane such as paclitaxel (Taxol) or docetaxel (Taxotere). Paraplatin may be administered at 10 mg/mL injectable concentrations (in vials of 50, 150, 450, and 600 mg). For advanced carcinomas a single agent dose of 360 mg/m² IV for 4 weeks may be administered. Paraplatin may be administered in combination=as 300 mg/m² IV (plus cyclophosphamide 600 mg/m² IV) q4Weeks. Taxol may be administered at 175 mg/m² IV over 3 hours q3Weeks (follow with cisplatin). Taxol may be administered at 135 mg/m² IV over 24 hours q3Weeks (follow with cisplatin). Taxol may be administered at 135-175 mg/m² IV over 3 hours q3Weeks.

Targeted therapeutic agents include, but are not limited to, PARP inhibitors.

In some examples, including any of the foregoing, the method includes conducting multiple-reaction-monitoring mass spectroscopy (MRM-MS) on the biological sample and/or or a control sample.

In some examples, including any of the foregoing, the mass spectroscopy is performed using multiple reaction monitoring (MRM) mode. In some examples, the mass spectroscopy is performed using QTOF MS in data-dependent acquisition. In some examples, the mass spectroscopy is performed using or MS-only mode. In some examples, an immunoassay (e.g., ELISA) is used in combination with mass spectroscopy.

In some examples, including any of the foregoing, the method includes quantifying one or more glycopeptides comprising the amino acid sequence of SEQ ID NOs: 1-38 and combinations thereof.

In some examples, including any of the foregoing, the method includes quantifying one or more glycopeptides consisting of an amino acid sequence selected from the group consisting of SEQ ID NOs: 1-38 and combinations thereof.

In some examples, including any of the foregoing, the method includes quantifying one or more glycopeptides consisting essentially of an amino acid sequence selected from the group consisting of SEQ ID NOs: 1-38 and combinations thereof.

In some examples, including any of the foregoing, the method includes quantifying one or more glycopeptides comprising the amino acid sequence of SEQ ID NOs: 5, 8-11, 13-14, 16-22, 26-28, 30-31, and 34-38 and combinations thereof. In some examples, including any of the foregoing, the method includes quantifying one or more glycopeptides comprising the amino acid sequence of SEQ ID NOs: 3, 7, 9, 28, 29, 32, and 33 and combinations thereof. In some examples, including any of the foregoing, the method includes quantifying one or more glycopeptides comprising the amino acid sequence of SEQ ID NOs: 1-4, 6-7, 12, 15, 23-25, 28, 29, and 32 and combinations thereof.

In some examples, including any of the foregoing, the method includes quantifying one or more glycopeptides consisting of an amino acid sequence selected from the group consisting of SEQ ID NOs: 5, 8-11, 13-14, 16-22, 26-28, 30-31, and 34-38, and combinations thereof. In some examples, including any of the foregoing, the method includes quantifying one or more glycopeptides consisting of an amino acid sequence selected from the group consisting of SEQ ID NOs: 3, 7, 9, 28, 29, 32, and 33, and combinations thereof. In some examples, including any of the foregoing, the method includes quantifying one or more glycopeptides consisting of an amino acid sequence selected from the group consisting of SEQ ID NOs: 1-4, 6-7, 12, 15, 23-25, 28, 29, and 32, and combinations thereof.

In some examples, including any of the foregoing, the method includes quantifying one or more glycopeptides consisting essentially of an amino acid sequence selected from the group consisting of SEQ ID NOs: 5, 8-11, 13-14, 16-22, 26-28, 30-31, and 34-38, and combinations thereof. In some examples, including any of the foregoing, the method includes quantifying one or more glycopeptides consisting essentially of an amino acid sequence selected from the group consisting of SEQ ID NOs: 3, 7, 9, 28, 29, 32, and 33, and combinations thereof. In some examples, including any of the foregoing, the method includes quantifying one or more glycopeptides consisting essentially of an amino acid sequence selected from the group consisting of SEQ ID NOs: 1-4, 6-7, 12, 15, 23-25, 28, 29, and 32, and combinations thereof.

In some examples, including any of the foregoing, the method includes detecting a multiple-reaction-monitoring (MRM) transition selected from the group consisting of transitions 1-38 using a QQQ and/or a qTOF mass spectrometer. In some embodiments, including any of the foregoing, the method includes detecting one or more peptide structures from Table 10, using a QQQ and/or a qTOF mass spectrometer. In some embodiments, including any of the foregoing, the method includes detecting one or more peptide structures comprising the amino acid sequence of SEQ ID NOs: 1-38, using a QQQ and/or a qTOF mass spectrometer.

In some examples, including any of the foregoing, the method includes training a machine learning algorithm to identify a classification based on the quantifying step.

In some examples, including any of the foregoing, the method includes using a machine learning algorithm to identify a classification based on the quantifying step.

In some examples, including any of the foregoing, the machine learning algorithm is selected from the group consisting of a deep learning algorithm, a neural network algorithm, an artificial neural network algorithm, a supervised machine learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, a regularized regression algorithm, or a combination thereof.

### D. Methods for Diagnosing Patients

In some examples, set forth herein is a method for diagnosing a patient having a disease or condition, comprising measuring by mass spectroscopy a glycopeptide in a sample from the patient.

In another embodiment, set forth herein is a method for diagnosing a patient having colorectal cancer or advanced adenoma; the method comprising: obtaining, or having obtained, a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect and quantify one or more MRM transitions selected from transitions 1-38; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having colorectal cancer or advanced adenoma based on the diagnostic classification.

In another embodiment, set forth herein is a method for diagnosing a patient having colorectal cancer or advanced adenoma; the method comprising: inputting the quantification of detected glycopeptides or MRM transitions into a trained model to generate an output probability, determining if the output probability is above or below a threshold for a classification; and identifying a diagnostic classification for the patient based on whether the output probability is above or below a threshold for a classification; and diagnosing the patient as having colorectal cancer or advanced adenoma based on the diagnostic classification. In some examples, the method includes obtaining, or having obtained a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect and quantify one or more MRM transitions selected from transitions 1-38.

In some examples, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-38; or to detect one or more MRM transitions selected from transitions 1-38; analyzing the detected glycopeptides or the MRM transitions using a trained model or training a model to identify a diagnostic classification; and diagnosing the patient as having colorectal cancer or advanced adenoma based on the diagnostic classification. In some examples, the method includes obtaining, or having obtained a biological sample from the patient; and performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-38; or to detect one or more MRM transitions selected from transitions 1-38.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-38; or to detect one or more MRM transitions selected from transitions 1-38; analyzing the detected glycopeptides or the MRM transitions using a trained model to identify a diagnostic classification; and diagnosing, monitoring, or classifying the individual as having an aging classification based on the diagnostic classification.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-38; or to detect one or more MRM transitions selected from transitions 1-38; training a model using the detected glycopeptides or the MRM transitions to identify a diagnostic classification; and diagnosing, monitoring, or classifying the individual as having an aging classification based on the diagnostic classification.

In some examples, set forth herein is a method for diagnosing, monitoring, or classifying aging in an individual; the method comprising: performing mass spectroscopy of a biological sample using MRM-MS with a QQQ and/or qTOF spectrometer to detect one or more glycopeptides consisting or, or consisting essentially of, an amino acid sequence selected from the group consisting of SEQ ID NOs: 1-38; or to detect one or more MRM transitions selected from transitions 1-38; analyzing the detected glycopeptides or the MRM transitions using a trained model to identify a diagnostic classification; and diagnosing, monitoring, or classifying an individual as having an aging classification based on the diagnostic classification.

In another embodiment, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 5, 8-11, 13-14, 16-22, 26-28, 30-31, and 34-38; inputting the quantification of the detected glycopeptides or the MRM transitions into a trained model or training a model to generate an output probability, determining if the output probability is above or below a threshold for a classification; and identifying a diagnostic classification for the patient based on whether the output probability is above or below a threshold for a classification; and diagnosing the patient as having colorectal cancer or advanced adenoma based on the diagnostic classification.

In another embodiment, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 3, 7, 9, 28, 29, 32, and 33; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having colorectal cancer or advanced adenoma based on the diagnostic classification.

In another embodiment, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 3, 7, 9, 28, 29, 32, and 33; and training a model using the quantification of the detected glycopeptides or the MRM transitions to generate an output probability.

In another embodiment, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect and quantify one or more glycopeptides consisting essentially of an amino acid sequence selected from the group consisting of SEQ ID NOs: 1-4, 6-7, 12, 15, 23-25, 28, 29, and 32; inputting the quantification of the detected glycopeptides or the MRM transitions into a trained model to generate an output probability, determining if the output probability is above or below a threshold for a classification; and identifying a diagnostic classification for the patient based on whether the output probability is above or below a threshold for a classification; and diagnosing the patient as having colorectal cancer or advanced adenoma based on the diagnostic classification.

In some examples, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 5, 8-11, 13-14, 16-22, 26-28, 30-31, and 34-38; analyzing the detected glycopeptides or the MRM transitions using a trained model to generate a diagnostic classification; and diagnosing the patient as having colorectal cancer or advanced adenoma based on the diagnostic classification. In some examples, the one or more glycopeptides consisting or, or consisting essentially of, an amino acid sequence selected from the group consisting of SEQ ID NOs: 5, 8-11, 13-14, 16-22, 26-28, 30-31, and 34-38 are used to train a model to generate a diagnostic classification.

In some examples, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 3, 7, 9, 28, 29, 32, and 33; analyzing the detected glycopeptides or the MRM transitions using a trained model to identify a diagnostic classification; and diagnosing the patient as having colorectal cancer or advanced adenoma based on the diagnostic classification. In some examples, the one or more glycopeptides consisting or, or consisting essentially of, an amino acid sequence selected from the group consisting of SEQ ID NOs: 3, 7, 9, 28, 29, 32, and 33 are used to train a model to identify a diagnostic classification.

In some examples, set forth herein is a method for diagnosing a patient having colorectal cancer or advanced adenoma; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-4, 6-7, 12, 15, 23-25, 28, 29, and 32; analyzing the detected glycopeptides or the MRM transitions using a trained model to identify a diagnostic classification; and diagnosing the patient as having colorectal cancer or advanced adenoma based on the diagnostic classification. In some examples, the one or more glycopeptides consisting or, or consisting essentially of, an amino acid sequence selected from the group consisting of SEQ ID NOs: 1-4, 6-7, 12, 15, 23-25, 28, 29, and 32 are used to train a model to identify a diagnostic classification.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 5, 8-11, 13-14, 16-22, 26-28, 30-31, and 34-38; analyzing the detected glycopeptides or the MRM transitions to using a trained model to identify a diagnostic classification; and diagnosing, monitoring, or classifying the individual as having an aging classification based on the diagnostic classification. In some examples, the one or more glycopeptides consisting or, or consisting essentially of, an amino acid sequence selected from the group consisting of SEQ ID NOs: 5, 8-11, 13-14, 16-22, 26-28, 30-31, and 34-38 are used to train a model to identify a diagnostic classification.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 3, 7, 9, 28, 29, 32, and 33; analyzing the detected glycopeptides or the MRM transitions using a trained model to identify a diagnostic classification; and diagnosing, monitoring, or classifying the individual as having an aging classification based on the diagnostic classification. In some examples, the one or more glycopeptides consisting or, or consisting essentially of, an amino acid sequence selected from the group consisting of SEQ ID NOs: 3, 7, 9, 28, 29, 32, and 33 are used to train model to identify a diagnostic classification.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-4, 6-7, 12, 15, 23-25, 28, 29, and 32; and analyzing the detected glycopeptides or the MRM transitions using a trained model to identify a diagnostic classification; and diagnosing, monitoring, or classifying the individual as having an aging classification based on the diagnostic classification. In some examples, the one or more glycopeptides consisting or, or consisting essentially of, an amino acid sequence selected from the group consisting of SEQ ID NOs: 1-4, 6-7, 12, 15, 23-25, 28, 29, and 32 are used to train a model to identify a diagnostic classification.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-4, 6-7, 12, 15, 23-25, 28, 29, and 32; analyzing the detected glycopeptides or the MRM transitions using a trained model to identify a diagnostic classification; and diagnosing, monitoring, or classifying the individual as having an aging classification based on the diagnostic classification. In some examples, the one or more glycopeptides consisting or, or consisting essentially of, an amino acid sequence selected from the group consisting of SEQ ID NOs: 1-4, 6-7, 12, 15, 23-25, 28, 29, and 32 are used to train model to identify a diagnostic classification.

In some examples, set forth herein is a method for diagnosing, monitoring, or classifying aging in an individual; the method comprising: obtaining, or having obtained a biological sample from the patient; performing mass spectroscopy of the biological sample using MRM-MS with a QQQ and/or qTOF spectrometer to detect one or more glycopeptides consisting or, or consisting essentially of, an amino acid sequence selected from the group consisting of SEQ ID NOs: 1-4, 6-7, 12, 15, 23-25, 28, 29, and 32; training a model to identify a diagnostic classification. In some other steps, the methods may include diagnosing, monitoring, or classifying the individual as having an aging classification based on the diagnostic classification. In some examples, the one or more glycopeptides consisting or, or consisting essentially of, an amino acid sequence selected from the group consisting of SEQ ID NOs: 1-4, 6-7, 12, 15, 23-25, 28, 29, and 32 are used to train a model to identify a diagnostic classification.

### E. Diseases and Conditions

Set forth herein are biomarkers for diagnosing a variety of diseases and conditions.

In some examples, the diseases and conditions include cancer. In some examples, the diseases and conditions are not limited to cancer.

In some examples, the diseases and conditions include colorectal cancer or advanced adenoma. In some examples, the diseases and conditions are not limited to colorectal cancer or advanced adenoma.

In some embodiments, colorectal cancer (CRC) is cancer of the lower gastrointestinal tract, for example, as the colon, the rectum and/or the appendix. In some embodiments, the CRC can develop from a colon polyp. In some embodiments, the colon polyp grows on the lining of the large intestine or rectum. In some embodiments, the colon polyp is benign. In some embodiments, the colon polyp is malignant. In some embodiments, the colon polyp progresses to colorectal adenoma if it is not diagnosed and/or treated. In some embodiments, the colon polyp progresses to advanced colorectal adenomas if it is not diagnosed and/or treated. In some embodiments, the colon polyp progresses to CRC if it is not diagnosed and/or treated. Without timely diagnosis and/or treatment, an individual having CRC has a significantly lower survival rates.

In some embodiments, provided herein is a method for classifying an individual as having CRC or not having CRC. In some embodiments, provided herein is a method for classifying an individual as having advanced adenoma (AA) or not having AA. In some embodiments, provided herein is a method for diagnosing an individual as having CRC or not having CRC. In some embodiments, provided herein is a method for diagnosing an individual as having advanced adenoma (AA) or not having AA. In some embodiments, provided herein is a method for treating an individual having CRC. In some embodiments, provided herein is a method for treating an individual having advanced adenoma (AA). In some embodiments, the method for treating an individual having CRC or AA comprising selecting a particular therapy and/or administrating the particular therapy. In any of the embodiments described herein, the method comprises inputting quantification data identified from peptide structure data for a set of peptides and/or glycopeptides into one or more machine-learning model trained to identify a disease indicator. In some embodiments, the method comprises classifying the sample as having CRC or AA or not having CRC or AA based upon the disease indicator. In some embodiments, the therapy is selected based upon presence and/or amount of at least one peptide structures from Table 10. In some embodiments, the therapy is selected based upon presence and/or amount of at least one glycopeptides comprising the amino acid sequence of SEQ ID NOs: 1-38. In some embodiments, the therapy is selected based upon presence and/or amount of at least one glycopeptides comprising the amino acid sequence of SEQ ID NOs: 5, 8-11, 13-14, 16-22, 26-28, 30-31, and 34-38. In some embodiments, the therapy is selected based upon presence and/or amount of at least one glycopeptides comprising the amino acid sequence of SEQ ID NOs: 3, 7, 9, 28, 29, 32, and 33. In some embodiments, the therapy is selected based upon presence and/or amount of at least one glycopeptides comprising the amino acid sequence of SEQ ID NOs: 1-4, 6-7, 12, 15, 23-25, 28, 29, 32.

Provided herein is a method of diagnosis and treatment for an individual. Further provided herein is a method of diagnosis and treatment for an individual with one or more risk factors associated with colorectal cancer (CRC) or advanced adenoma (AA). In some embodiments, the method comprises measuring the amount/presence or absence of one or more peptides structures from Table 10 in an individual with one or more risk factors associated with CRC or AA. In some embodiments, the method involves diagnosing an individual based upon presence and/or amount of one or more peptide structures from Table 10. In some embodiments, the method involves diagnosing an individual based upon presence and/or amount of one or more glycopeptides from Table 10. In some embodiments, the diagnosis is based upon the presence and/or amount of one or more peptides and/or glycopeptides comprising the amino acid sequence of SEQ ID NOs: 1-38 set forth in Table 10. In some embodiments, the diagnosis is based upon the presence and/or amount of one or more glycopeptides comprising the amino acid sequence of SEQ ID NOs: 1-38 set forth in Table 10. In some embodiments, the diagnosis is based upon the presence and/or amount of one or more glycopeptides consisting of the amino acid sequence of SEQ ID NOs: 1-38 along with the associated glycan set forth in Table 10. In some embodiments, the individual diagnosed with CRC or AA is administered one or more CRC or AA therapies described herein, based on the disease indicator determined by the diagnosis. In some embodiments, the individual is administered one or more CRC or AA therapies described herein, based on the disease indicator determined by the diagnosis. In some embodiments, the individual confirmed to have CRC or AA is treated based on the disease indicator determined by the diagnosis.

In some embodiments, the individual is diagnosed, wherein one or more peptide structures from Table 10 are detected and are distinct from a healthy control sample. In some embodiments, the individual is diagnosed, wherein one or more peptide structures comprising the amino acid sequence of SEQ ID NOs: 1-38 are detected and are distinct from a healthy control sample. In some embodiments, the individual is diagnosed, wherein one or more glycopeptides comprising the amino acid sequence of SEQ ID NOs: 1-38 are detected and are distinct from a healthy control sample. In some embodiments, the amount of at least one peptide structure is none, or below a detection limit. In some embodiments, the amount of at least one glycopeptide structure is none, or below a detection limit. In some embodiments, the amount of at least one peptide structure from Table 10 is none, or below a detection limit. In some embodiments, the amount of at least one peptide structure comprising the amino acid sequence of SEQ ID NOs: 1-38 set forth in Table 10 is none, or below a detection limit. In some embodiments, the amount of at least one peptide structure is significantly lower than a control sample from a healthy individual. In some embodiments, the amount of at least one glycopeptide structure is significantly lower than a control sample from a healthy individual. In some embodiments, the amount of at least one peptide structure from Table 10 is significantly lower than a control sample from a healthy individual. In some embodiments, the amount of at least one peptide structure comprising the amino acid sequence of SEQ ID NOs: 1-38 set forth in Table 10 is significantly lower than a control sample from a healthy individual. In some embodiments, the amount of at least one peptide structure is significantly higher than a control sample from a healthy individual. In some embodiments, the amount of at least one glycopeptide structure is significantly higher than a control sample from a healthy individual. In some embodiments, the amount of at least one peptide structure from Table 10 is significantly higher than a control sample from a healthy individual. In some embodiments, the amount of at least one peptide structure comprising the amino acid sequence of SEQ ID NOs: 1-38 set forth in Table 10 is significantly higher than a control sample from a healthy individual. In some embodiments, the individual is diagnosed and treated according to the presence and/or amount of one or more peptide structures from Table 10. In some embodiments, the individual is diagnosed and treated according to the presence and/or amount of one or more peptide structures comprising the amino acid sequence of SEQ ID NOs: 1-38 along with the associated glycan set forth in Table 10.

In some embodiments, the individual has CRC or AA. In some embodiments, the individual has stage 0, stage I, stage II, stage III, or stage IV CRC. In some embodiments, the individual has early-stage CRC. In some embodiments, the individual has late-stage CRC or advanced CRC. In some embodiments, the individual has CRC that has not spread from the site of origination. In some embodiments, the individual has CRC that has spread locally to the surrounding tissue. In some embodiments, the individual has CRC that has spread beyond the original tumor and/or the local tumor environment. In some embodiments, the individual has CRC that has spread to one or more organs beyond the lungs. In some embodiments, the individual has metastatic CRC. In some embodiments, the individual has CRC and has relapsed and/or progressed. In some embodiments, the method comprises classifying a biological sample with respect to a plurality of states associated with CRC based upon one or more peptide structures provided in Table 10. In some embodiments, the method comprises classifying a biological sample with respect to a plurality of states associated with CRC or AA based upon one or more glycopeptides provided in Table 10. In some embodiments, the method comprises inputting quantification data identified from peptide structure data for a set of peptides and/or glycopeptides into one or more machine-learning model trained to identify a disease indicator. In some embodiments, the method comprises classifying the sample as having CRC or AA or not having CRC or AA based upon the disease indicator. In some embodiments, the peptide structure data comprises one or more peptide structure provided in Table 10. In some embodiments, the presence, absence, and/or amount of one or more peptides and/or glycopeptide is determined by MRM-MS. In some embodiments, the method comprises selecting a particular therapy described herein based upon the presence, amount, and/or relative amount of one or more biomarkers comprising the peptide structures provided in Table 10. In some embodiments, the method comprises selecting a particular therapy described herein based upon the presence, amount, and/or relative amount of one or more biomarkers comprising the glycopeptides provided in Table 10. In some embodiments, the method comprises administering a particular therapy described herein based upon the presence, amount, and/or relative amount of one or more biomarkers comprising the peptide structures provided in Table 10. In some embodiments, the method comprises administering a particular therapy described herein based upon the presence, amount, and/or relative amount of one or more biomarkers comprising the glycopeptides provided in Table 10. In some embodiments, the method further comprises selecting a particular therapy described herein based upon the disease indicator and/or classification. In some embodiments, the method further comprises administering a particular therapy described herein based upon the disease indicator and/or classification.

In some embodiments, the individual has had prior lines of therapy for treating CRC or AA. In some embodiments, the individual has had at least 1, at least 2, or at least 3 prior lines of therapy for treating CRC or AA. In some embodiments, the individual has had no more than 1, no more than 2, or no more than 3 prior lines of therapy for treating CRC or AA. In some embodiments, the individual has not had prior therapy for treating CRC or AA.

In some embodiments, the individual has altered gene expression relevant for colorectal cancer (CRC) treatment. In some embodiments, the individual has altered oncogene expression. In some embodiments, the individual has altered tumor cell gene expression. In some embodiments, the altered gene expression comprises altered gene expression of one or more of VEGF, EGFR, BRAF, and MEK. In some embodiments, the altered gene expression comprises altered gene expression of one or more immune system checkpoint proteins PD-1, PD-L1, and CTLA-4. In some embodiments, the individual having altered gene expression relevant for CRC treatment may benefit from a therapy comprising one or more antibody that targets PD-1, PD-L1, and CTLA-4, or a combination thereof.

In some embodiments, the individual is at risk of developing colorectal cancer (CRC) or advanced adenoma (AA). In some embodiments, the risk of CRC or AA is determined based upon presence and/or amount of at least one peptide structures from Table 10. In some embodiments, the risk of CRC is determined based upon the presence and/or amount of one or more peptides comprising the amino acid sequence of SEQ ID NOs: 1-38. In some embodiments, the individual is positive for one or more risk factor that increases the chances of developing CRC. In some embodiments, the one or more risk factor is selected from a group consisting of age, irritable bowel disease, type 2 diabetes, a family history of CRC, a genetic syndrome (e.g., Lynch syndrome), obesity, smoking, tobacco use, alcohol consumption, dietary choices, and limited physical activity. In some embodiments, the individual has at least 1, at least 2, at least 3, at least 4, at least 5, or at least 6 risk factors for CRC.

In some embodiments, the individual is positive for one or more risk factor that increases the chances of developing colorectal cancer (CRC) or advanced adenoma (AA). In some embodiments, the one or more risk factor comprises the age of the individual. In some embodiments, the individual is at least 30, at least 35, at least 40, at least 45, at least 50, at least 55, at least 60, at least 65, at least 70, at least 75, at least 80, at least 85, or at least 90 years old. In some embodiments, the individual is at least 30 years old. In some embodiments, the individual is at least 40 years old. In some embodiments, the individual is at least 50 years old. In some embodiments, the individual is at least 60 years old.

In some embodiments, the individual at risk of developing colorectal cancer (CRC) or advanced adenoma (AA) is overweight or obese. In some embodiments, the individual at risk of developing CRC has a body mass index (BMI)≥30 kg/m. In some embodiments, the individual at risk of developing CRC has a BMI≥35 kg/m. In some embodiments, the individual at risk of developing CRC has a BMI≥40 kg/m. In some embodiments, the individual is considered extremely obese.

In some embodiments, the individual at risk of developing colorectal cancer (CRC) or advanced adenoma (AA) has a genetic syndrome. In some embodiments, the genetic syndrome comprises familial adenomatous polyposis (FAP) or hereditary non-polyposis colorectal cancer (Lynch syndrome).

In some embodiments, the individual at risk of developing colorectal cancer (CRC) or advanced adenoma (AA) consumes foods that may increase the risk of CRC or AA. In some embodiments, the individual consumes an abundance of red or processed meat. In some embodiments, the individual at risk of developing CRC or AA does not consume foods that may decrease the risk of CRC or AA. In some embodiments, the individual consumes a limited amount of vegetables and fiber.

In some embodiments, the individual at risk of developing colorectal cancer (CRC) or advanced adenoma (AA) is a smoker or consumer of tobacco products. In some embodiments, the individual smokes cigarettes, cigars, pipes, and other tobacco-based products. In some embodiments, the individual is a smoker. In some embodiments, the individual uses tobacco-containing products.

In some embodiments, the individual is positive for one or more clinical indicators of colorectal cancer (CRC) described herein. In some embodiments, the one or more clinical indicators of CRC comprise changes in bowel habits, bloody stool, diarrhea, constipation, persistent abdominal pain, persistent abdominal cramps, and unexplained weight loss. In some embodiments, the individual has at least 1, at least 2, at least 3, at least 4, at least 5, or at least 6 clinical indicators of CRC. In some embodiments, the individual has any combination of clinical indicators of CRC described herein.

In some examples, the condition is aging. In some examples, the “patient” described herein is equivalently described as an “individual.” For example, in some methods herein, set forth are biomarkers for monitoring or diagnosing aging or aging conditions in an individual. In some of these examples, the individual is not necessarily a patient who has a medical condition in need of therapy. In some examples, the individual is a male. In some examples, the individual is a female. In some examples, the individual is a male mammal. In some examples, the individual is a female mammal. In some examples, the individual is a male human. In some examples, the individual is a female human.

In some examples, the individual is 1 year old. In some examples, the individual is 2 years old. In some examples, the individual is 3 years old. In some examples, the individual is 4 years old. In some examples, the individual is 5 years old. In some examples, the individual is 6 years old. In some examples, the individual is 7 years old. In some examples, the individual is 8 years old. In some examples, the individual is 9 years old. In some examples, the individual is 10 years old. In some examples, the individual is 11 years old. In some examples, the individual is 12 years old. In some examples, the individual is 13 years old. In some examples, the individual is 14 years old. In some examples, the individual is 15 years old. In some examples, the individual is 16 years old. In some examples, the individual is 17 years old. In some examples, the individual is 18 years old. In some examples, the individual is 19 years old. In some examples, the individual is 20 years old. In some examples, the individual is 21 years old. In some examples, the individual is 22 years old. In some examples, the individual is 23 years old. In some examples, the individual is 24 years old. In some examples, the individual is 25 years old. In some examples, the individual is 26 years old. In some examples, the individual is 27 years old. In some examples, the individual is 28 years old. In some examples, the individual is 29 years old. In some examples, the individual is 30 years old. In some examples, the individual is 31 years old. In some examples, the individual is 32 years old. In some examples, the individual is 33 years old. In some examples, the individual is 34 years old. In some examples, the individual is 35 years old. In some examples, the individual is 36 years old. In some examples, the individual is 37 years old. In some examples, the individual is 38 years old. In some examples, the individual is 39 years old. In some examples, the individual is 40 years old. In some examples, the individual is 41 years old. In some examples, the individual is 42 years old. In some examples, the individual is 43 years old. In some examples, the individual is 44 years old. In some examples, the individual is 45 years old. In some examples, the individual is 46 years old. In some examples, the individual is 47 years old. In some examples, the individual is 48 years old. In some examples, the individual is 49 years old. In some examples, the individual is 50 years old. In some examples, the individual is 51 years old. In some examples, the individual is 52 years old. In some examples, the individual is 53 years old. In some examples, the individual is 54 years old. In some examples, the individual is 55 years old. In some examples, the individual is 56 years old. In some examples, the individual is 57 years old. In some examples, the individual is 58 years old. In some examples, the individual is 59 years old. In some examples, the individual is 60 years old. In some examples, the individual is 61 years old. In some examples, the individual is 62 years old. In some examples, the individual is 63 years old. In some examples, the individual is 64 years old. In some examples, the individual is 65 years old. In some examples, the individual is 66 years old. In some examples, the individual is 67 years old. In some examples, the individual is 68 years old. In some examples, the individual is 69 years old. In some examples, the individual is 70 years old. In some examples, the individual is 71 years old. In some examples, the individual is 72 years old. In some examples, the individual is 73 years old. In some examples, the individual is 74 years old. In some examples, the individual is 75 years old. In some examples, the individual is 76 years old. In some examples, the individual is 77 years old. In some examples, the individual is 78 years old. In some examples, the individual is 79 years old. In some examples, the individual is 80 years old. In some examples, the individual is 81 years old. In some examples, the individual is 82 years old. In some examples, the individual is 83 years old. In some examples, the individual is 84 years old. In some examples, the individual is 85 years old. In some examples, the individual is 86 years old. In some examples, the individual is 87 years old. In some examples, the individual is 88 years old. In some examples, the individual is 89 years old. In some examples, the individual is 90 years old. In some examples, the individual is 91 years old. In some examples, the individual is 92 years old. In some examples, the individual is 93 years old. In some examples, the individual is 94 years old. In some examples, the individual is 95 years old. In some examples, the individual is 96 years old. In some examples, the individual is 97 years old. In some examples, the individual is 98 years old. In some examples, the individual is 99 years old. In some examples, the individual is 100 years old. In some examples, the individual is more than 100 years old.

### V. Machine Learning

In some examples, including any of the foregoing, the methods herein include quantifying one or more glycopeptides comprising one or more peptide structure from Table 10 using mass spectroscopy (MS) and/or liquid chromatography (LC). In some examples, including any of the foregoing, the methods herein include quantifying one or more glycopeptides comprising an amino acid sequence selected from the amino acid sequence of any one of SEQ ID NOs: 1-38 using MS and/or LC. In some examples, the methods includes quantifying one or more glycopeptides comprising an amino acid sequence selected from the amino acid sequence of any one of SEQ ID NOs: 5, 8-11, 13-14, 16-22, 26-28, 30-31, and 34-38, and combinations thereof using MS and/or LC. In some examples, the methods includes quantifying one or more glycopeptides comprising an amino acid sequence selected from the amino acid sequence of any one of SEQ ID NOs: 3, 7, 9, 28, 29, 32, and 33, and combinations thereof using MS and/or LC. In some examples, the methods includes quantifying one or more glycopeptides comprising an amino acid sequence selected from the amino acid sequence of any one of SEQ ID NOs: 1-4, 6-7, 12, 15, 23-25, 28, 29, and 32, and combinations thereof using MS and/or LC.

In some examples, including any of the foregoing, the methods herein include quantifying one or more glycopeptides consisting essentially of an amino acid sequence selected from the group consisting of SEQ ID NOs: 1-38 using mass spectroscopy and/or liquid chromatography. In some examples, the methods includes quantifying one or more glycopeptides consisting essentially of an amino acid sequence selected from the group consisting of SEQ ID NOs: 5, 8-11, 13-14, 16-22, 26-28, 30-31, and 34-38, and combinations thereof using mass spectroscopy and/or liquid chromatography. In some examples, the methods includes quantifying one or more glycopeptides consisting essentially of an amino acid sequence selected from the group consisting of SEQ ID NOs: 3, 7, 9, 28, 29, 32, and 33, and combinations thereof using mass spectroscopy and/or liquid chromatography. In some examples, the methods includes quantifying one or more glycopeptides consisting essentially of an amino acid sequence selected from the group consisting of SEQ ID NOs: 1-4, 6-7, 12, 15, 23-25, 28, 29, and 32, and combinations thereof using mass spectroscopy and/or liquid chromatography.

In some examples, the quantification results are used as inputs in a trained model. In some examples, the quantification results are classified or categorized with a diagnostic algorithm based on the absolute amount, relative amount, and/or type of each glycan or glycopeptide quantified in the test sample, wherein the diagnostic algorithm is trained on corresponding values for each marker obtained from a population of individuals having known diseases or conditions. In some examples, the disease or condition is colorectal cancer or advanced adenoma.

In some examples, including any of the foregoing, the methods herein include quantifying one or more glycopeptides comprising one or more peptide structure from Table 10 using mass spectroscopy (MS) and/or liquid chromatography (LC). In some examples, including any of the foregoing, the methods herein include quantifying one or more glycopeptides comprising an amino acid sequence selected from the amino acid sequence of any one of SEQ ID NOs: 1-38 using MS and/or LC.

In some examples, including any of the foregoing, set forth herein is a method for training a machine learning algorithm, comprising: providing a first data set of MRM transition signals indicative of a sample comprising one or more glycopeptide comprising an amino acid sequence of any one of SEQ ID NOs: 1-38; providing a second data set of MRM transition signals indicative of a control sample; and comparing the first data set with the second data set using a machine learning algorithm. In some examples, the methods include providing a first data set of MRM transition signals indicative of a sample comprising one or more glycopeptide comprising an amino acid sequence of any one of SEQ ID NOs: 5, 8-11, 13-14, 16-22, 26-28, 30-31, and 34-38 and combinations thereof. In some examples, the methods include providing a first data set of MRM transition signals indicative of a sample comprising one or more glycopeptide comprising an amino acid sequence of any one of SEQ ID NOs: 3, 7, 9, 28, 29, 32, and 33 and combinations thereof. In some examples, the methods include providing a first data set of MRM transition signals indicative of a sample comprising one or more glycopeptide comprising an amino acid sequence of any one of SEQ ID NOs: 1-4, 6-7, 12, 15, 23-25, 28, 29, and 32, and combinations thereof.

In some examples, including any of the foregoing, set forth herein is a method for training a machine learning algorithm, comprising: providing a first data set of MRM transition signals indicative of a sample comprising a glycopeptide consisting essentially of an amino acid sequence selected from the group consisting of SEQ ID NOs: 1-38; providing a second data set of MRM transition signals indicative of a control sample; and comparing the first data set with the second data set using a machine learning algorithm. In some examples, the methods include providing a first data set of MRM transition signals indicative of a sample comprising a glycopeptide consisting essentially of an amino acid sequence selected from the group consisting of SEQ ID NOs: 5, 8-11, 13-14, 16-22, 26-28, 30-31, and 34-38 and combinations thereof. In some examples, the methods include providing a first data set of MRM transition signals indicative of a sample comprising a glycopeptide consisting essentially of an amino acid sequence selected from the group consisting of SEQ ID NOs: 3, 7, 9, 28, 29, 32, and 33 and combinations thereof. In some examples, the methods include providing a first data set of MRM transition signals indicative of a sample comprising a glycopeptide consisting essentially of an amino acid sequence selected from the group consisting of SEQ ID NOs: 1-4, 6-7, 12, 15, 23-25, 28, 29, and 32, and combinations thereof.

In some examples, including any of the foregoing, the method herein include using a sample comprising a glycopeptide consisting of an amino acid sequence selected from the group consisting of SEQ ID NOs: 1-38 is a sample from a patient having colorectal cancer or advanced adenoma.

In some examples, including any of the foregoing, the method herein include using a sample comprising a glycopeptide consisting essentially of an amino acid sequence selected from the group consisting of SEQ ID NOs: 1-38 is a sample from a patient having colorectal cancer or advanced adenoma.

In some examples, including any of the foregoing, the method herein include using a sample comprising one or more glycopeptide comprising an amino acid sequence of any one of SEQ ID NOs: 5, 8-11, 13-14, 16-22, 26-28, 30-31, and 34-38, and combinations thereof is a sample from a patient having colorectal cancer or advanced adenoma. In some examples, including any of the foregoing, the method herein include using a sample comprising one or more glycopeptide comprising an amino acid sequence of any one of SEQ ID NOs: 3, 7, 9, 28, 29, 32, and 33, and combinations thereof is a sample from a patient having colorectal cancer or advanced adenoma. In some examples, including any of the foregoing, the method herein include using a sample comprising one or more glycopeptide comprising an amino acid sequence of any one of SEQ ID NOs: 1-4, 6-7, 12, 15, 23-25, 28, 29, and 32, and combinations thereof is a sample from a patient having colorectal cancer or advanced adenoma.

In some examples, including any of the foregoing, the method herein include using a sample comprising a glycopeptide consisting of an amino acid sequence selected from the group consisting of SEQ ID NOs: 5, 8-11, 13-14, 16-22, 26-28, 30-31, and 34-38, and combinations thereof is a sample from a patient having colorectal cancer or advanced adenoma. In some examples, including any of the foregoing, the method herein include using a sample comprising a glycopeptide consisting of an amino acid sequence selected from the group consisting of SEQ ID NOs: 3, 7, 9, 28, 29, 32, and 33, and combinations thereof is a sample from a patient having colorectal cancer or advanced adenoma. In some examples, including any of the foregoing, the method herein include using a sample comprising a glycopeptide consisting of an amino acid sequence selected from the group consisting of SEQ ID NOs: 1-4, 6-7, 12, 15, 23-25, 28, 29, and 32, and combinations thereof is a sample from a patient having colorectal cancer or advanced adenoma.

In some examples, including any of the foregoing, the method herein include using a sample comprising a glycopeptide consisting essentially of an amino acid sequence selected from the group consisting of SEQ ID NOs: 5, 8-11, 13-14, 16-22, 26-28, 30-31, and 34-38, and combinations thereof is a sample from a patient having colorectal cancer or advanced adenoma. In some examples, including any of the foregoing, the method herein include using a sample comprising a glycopeptide consisting essentially of an amino acid sequence selected from the group consisting of SEQ ID NOs: 3, 7, 9, 28, 29, 32, and 33, and combinations thereof is a sample from a patient having colorectal cancer or advanced adenoma. In some examples, including any of the foregoing, the method herein include using a sample comprising a glycopeptide consisting essentially of an amino acid sequence selected from the group consisting of SEQ ID NOs: 1-4, 6-7, 12, 15, 23-25, 28, 29, and 32, and combinations thereof is a sample from a patient having colorectal cancer or advanced adenoma.

In some examples, including any of the foregoing, the method herein include using a control sample, wherein the control sample is a sample from a patient not having colorectal cancer or advanced adenoma.

In some examples, including any of the foregoing, the method herein include using a sample comprising a glycopeptide consisting essentially of an amino acid sequence selected from the group consisting of SEQ ID NOs: 1-38, which is a pooled sample from one or more patients having colorectal cancer or advanced adenoma.

In some examples, including any of the foregoing, the method herein include using a sample comprising one or more glycopeptide comprising an amino acid sequence of any one of SEQ ID NOs: 5, 8-11, 13-14, 16-22, 26-28, 30-31, and 34-38, and combinations thereof, which is a pooled sample from one or more patients having colorectal cancer or advanced adenoma. In some examples, including any of the foregoing, the method herein include using a sample comprising one or more glycopeptide comprising an amino acid sequence of any one of SEQ ID NOs: 3, 7, 9, 28, 29, 32, and 33, and combinations thereof, which is a pooled sample from one or more patients having colorectal cancer or advanced adenoma. In some examples, including any of the foregoing, the method herein include using a sample comprising one or more glycopeptide comprising an amino acid sequence of any one of SEQ ID NOs: 1-4, 6-7, 12, 15, 23-25, 28, 29, and 32, and combinations thereof, which is a pooled sample from one or more patients having colorectal cancer or advanced adenoma.

In some examples, including any of the foregoing, the method herein include using a sample comprising a glycopeptide consisting essentially of an amino acid sequence selected from the group consisting of SEQ ID NOs: 5, 8-11, 13-14, 16-22, 26-28, 30-31, and 34-38, and combinations thereof, which is a pooled sample from one or more patients having colorectal cancer or advanced adenoma. In some examples, including any of the foregoing, the method herein include using a sample comprising a glycopeptide consisting essentially of an amino acid sequence selected from the group consisting of SEQ ID NOs: 3, 7, 9, 28, 29, 32, and 33, and combinations thereof, which is a pooled sample from one or more patients having colorectal cancer or advanced adenoma. In some examples, including any of the foregoing, the method herein include using a sample comprising a glycopeptide consisting essentially of an amino acid sequence selected from the group consisting of SEQ ID NOs: 1-4, 6-7, 12, 15, 23-25, 28, 29, and 32, and combinations thereof, which is a pooled sample from one or more patients having colorectal cancer or advanced adenoma.

In some examples, including any of the foregoing, the method herein include using a control sample, which is a pooled sample from one or more patients not having colorectal cancer or advanced adenoma.

In some examples, including any of the foregoing, the methods include generating machine learning models trained using mass spectrometry data (e.g., MRM-MS transition signals) from patients having a disease or condition and patients not having a disease or condition. In some examples, the disease or condition is colorectal cancer or advanced adenoma. In some examples, the methods include optimizing the machine learning models by cross-validation with known standards or other samples. In some examples, the methods include qualifying the performance using the mass spectrometry data to form panels of glycans and glycopeptides with individual sensitivities and specificities. In certain examples, the methods include determining a confidence percent in relation to a diagnosis. In some examples, one to ten glycopeptides consisting essentially of an amino acid sequence selected from the group consisting of SEQ ID NOs: 1-38 may be useful for diagnosing a patient with colorectal cancer or advanced adenoma with a certain confidence percent. In some examples, ten to fifty glycopeptides consisting essentially of an amino acid sequence selected from the group consisting of SEQ ID NOs: 1-38 may be useful for diagnosing a patient with colorectal cancer or advanced adenoma with a higher confidence percent.

In some examples, including any of the foregoing, the methods include performing MRM-MS and/or LC-MS on a biological sample. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NOs: 1-38. In some examples, the methods include comparing, by the computing device, the mass spectra data with the theoretical mass spectra data to generate comparison data indicative of a similarity of each of the plurality of mass spectra to each of the plurality of theoretical target mass spectra associated with a corresponding glycopeptide of the plurality of glycopeptides.

In some examples, including any of the foregoing, the methods include generating machine learning models trained using mass spectrometry data (e.g., MRM-MS transition signals) from patients having a disease or condition and patients not having a disease or condition. In some examples, the disease or condition is colorectal cancer or advanced adenoma. In some examples, the methods include optimizing the machine learning models by cross-validation with known standards or other samples. In some examples, the methods include qualifying the performance using the mass spectrometry data to form panels of glycans and glycopeptides with individual sensitivities and specificities. In certain examples, the methods include determining a confidence percent in relation to a diagnosis.

In some examples, at least one glycopeptides comprising an amino acid sequence of any one of SEQ ID NOs: 5, 8-11, 13-14, 16-22, 26-28, 30-31, and 34-38, may be useful for diagnosing a patient with colorectal cancer or advanced adenoma with a certain confidence percent. In some examples, at least one glycopeptide comprising an amino acid sequence of any one of SEQ ID NOs: 3, 7, 9, 28, 29, 32, and 33, may be useful for diagnosing a patient with colorectal cancer or advanced adenoma with a certain confidence percent. In some examples, at least one glycopeptides comprising an amino acid sequence of any one of SEQ ID NOs: 1-4, 6-7, 12, 15, 23-25, 28, 29, and 32, may be useful for diagnosing a patient with colorectal cancer or advanced adenoma with a certain confidence percent.

In some examples, at least one glycopeptides consisting essentially of an amino acid sequence selected from the group consisting of SEQ ID NOs: 5, 8-11, 13-14, 16-22, 26-28, 30-31, and 34-38, may be useful for diagnosing a patient with colorectal cancer or advanced adenoma with a certain confidence percent. In some examples, at least one glycopeptide consisting essentially of an amino acid sequence selected from the group consisting of SEQ ID NOs: 3, 7, 9, 28, 29, 32, and 33, may be useful for diagnosing a patient with colorectal cancer or advanced adenoma with a certain confidence percent. In some examples, at least one glycopeptides consisting essentially of an amino acid sequence selected from the group consisting of SEQ ID NOs: 1-4, 6-7, 12, 15, 23-25, 28, 29, and 32, may be useful for diagnosing a patient with colorectal cancer or advanced adenoma with a certain confidence percent.

In some examples, at least one glycopeptides comprising an amino acid sequence of any one of SEQ ID NOs: 5, 8-11, 13-14, 16-22, 26-28, 30-31, and 34-38, may be useful for diagnosing a patient with colorectal cancer or advanced adenoma with a higher confidence percent. In some examples, at least one glycopeptide comprising an amino acid sequence of any one of SEQ ID NOs: 3, 7, 9, 28, 29, 32, and 33, may be useful for diagnosing a patient with colorectal cancer or advanced adenoma with a higher confidence percent. In some examples, at least one glycopeptides comprising an amino acid sequence of any one of SEQ ID NOs: 1-4, 6-7, 12, 15, 23-25, 28, 29, and 32, may be useful for diagnosing a patient with colorectal cancer or advanced adenoma with a higher confidence percent.

In some examples, at least one glycopeptides consisting essentially of an amino acid sequence selected from the group consisting of SEQ ID NOs: 5, 8-11, 13-14, 16-22, 26-28, 30-31, and 34-38 may be useful for diagnosing a patient with colorectal cancer or advanced adenoma with a higher confidence percent. In some examples, at least one glycopeptides consisting essentially of an amino acid sequence selected from the group consisting of SEQ ID NOs: 3, 7, 9, 28, 29, 32, and 33, may be useful for diagnosing a patient with colorectal cancer or advanced adenoma with a higher confidence percent. In some examples, at least one glycopeptides consisting essentially of an amino acid sequence selected from the group consisting of SEQ ID NOs: 1-4, 6-7, 12, 15, 23-25, 28, 29, and 32, may be useful for diagnosing a patient with colorectal cancer or advanced adenoma with a higher confidence percent.

In some examples, including any of the foregoing, the methods include performing MRM-MS and/or LC-MS on a biological sample. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NOs: 5, 8-11, 13-14, 16-22, 26-28, 30-31, and 34-38. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NOs: 3, 7, 9, 28, 29, 32, and 33. In some examples, the methods include constructing, by a computing device, theoretical mass spectra data representing a plurality of mass spectra, wherein each of the plurality of mass spectra corresponds to one or more glycopeptides consisting essentially of an amino acid sequence selected from the group consisting of SEQ ID NOs: 1-4, 6-7, 12, 15, 23-25, 28, 29, and 32.

In some examples, the methods include comparing, by the computing device, the mass spectra data with the theoretical mass spectra data to generate comparison data indicative of a similarity of each of the plurality of mass spectra to each of the plurality of theoretical target mass spectra associated with a corresponding glycopeptide of the plurality of glycopeptides.

In some examples, machine learning algorithms are used to determine, by the computing device and based on the MRM-MS data, a distribution of a plurality of characteristic ions in the plurality of mass spectra; and determining, by the computing device and based on the distribution, whether one or more of the plurality of characteristic ions is a glycopeptide ion.

In some examples, the methods herein include training a diagnostic algorithm. Herein, training the diagnostic algorithm may refer to supervised learning of a diagnostic algorithm on the basis of values for one or more glycopeptides consisting of, or consisting essentially of, an amino acid sequence selected from the group consisting of SEQ ID NOs: 1-38. Training the diagnostic algorithm may refer to variable selection in a statistical model on the basis of values for one or more glycopeptides consisting essentially of an amino acid sequence selected from the group consisting of SEQ ID NOs: 1-38. Training a diagnostic algorithm may for example include determining a weighting vector in feature space for each category, or determining a function or function parameters.

In some examples, the methods herein include training a diagnostic algorithm. Herein, training the diagnostic algorithm may refer to supervised learning of a diagnostic algorithm on the basis of values for one or more glycopeptides consisting of, or consisting essentially of, an amino acid sequence selected from the group consisting of SEQ ID NOs: 5, 8-11, 13-14, 16-22, 26-28, 30-31, and 34-38, and combinations thereof. Herein, training the diagnostic algorithm may refer to supervised learning of a diagnostic algorithm on the basis of values for one or more glycopeptides consisting of, or consisting essentially of, an amino acid sequence selected from the group consisting of SEQ ID NOs: 3, 7, 9, 28, 29, 32, and 33, and combinations thereof. Herein, training the diagnostic algorithm may refer to supervised learning of a diagnostic algorithm on the basis of values for one or more glycopeptides consisting of, or consisting essentially of, an amino acid sequence selected from the group consisting of SEQ ID NOs: 1-4, 6-7, 12, 15, 23-25, 28, 29, and 32, and combinations thereof.

Training the diagnostic algorithm may refer to variable selection in a statistical model on the basis of values for one or more glycopeptides consisting essentially of an amino acid sequence selected from the group consisting of SEQ ID NOs: 5, 8-11, 13-14, 16-22, 26-28, 30-31, and 34-38, and combinations thereof. Training the diagnostic algorithm may refer to variable selection in a statistical model on the basis of values for one or more glycopeptides consisting essentially of an amino acid sequence selected from the group consisting of SEQ ID NOs: 3, 7, 9, 28, 29, 32, and 33, and combinations thereof. Training the diagnostic algorithm may refer to variable selection in a statistical model on the basis of values for one or more glycopeptides consisting essentially of an amino acid sequence selected from the group consisting of SEQ ID NOs: 1-4, 6-7, 12, 15, 23-25, 28, 29, and 32, and combinations thereof.

Training a diagnostic algorithm may for example include determining a weighting vector in feature space for each category, or determining a function or function parameters.

In some examples, including any of the foregoing, the machine learning algorithm is selected from the group consisting of a deep learning algorithm, a neural network algorithm, an artificial neural network algorithm, a supervised machine learning algorithm, a linear discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, a regularized regression algorithm, or a combination thereof. In certain examples, the machine learning algorithm is lasso regression.

In certain examples, the machine learning algorithm is LASSO, Ridge Regression, Random Forests, K-nearest Neighbors (KNN), Deep Neural Networks (DNN), and Principal Components Analysis (PCA). In certain examples, DNN's are used to process mass spec data into analysis-ready forms. In some examples, DNN's are used for peak picking from a mass spectra. In some examples, PCA is useful in feature detection. In some examples, the machine learning is combined discriminant analysis.

In some examples, LASSO is used to provide feature selection.

In some examples, machine learning algorithms are used to quantify peptides from each protein that are representative of the protein abundance. In some examples, this quantification includes quantifying proteins for which glycosylation is not measured.

In some examples, glycopeptide sequences are identified by fragmentation in the mass spectrometer and database search using Byonic software.

In some examples, the methods herein include unsupervised learning to detect features of MRMS-MS data that represent known biological quantities, such as protein function or glycan motifs. In certain examples, these features are used as input for classifying by machine. In some examples, the classification is performed using LASSO, Ridge Regression, or Random Forest nature.

In some examples, the methods herein include mapping input data (e.g., MRM transition peaks) to a value (e.g., a scale based on 0-100) before processing the value in an algorithm. For example, after a MRM transition is identified and the peak characterized, the methods herein include assessing the MS scans in an m/z and retention time window around the peak for a given patient. In some examples, the resulting chromatogram is integrated by a machine learning algorithm that determines the peak start and stop points, and calculates the area bounded by those points and the intensity (height). The resulting integrated value is the abundance, which then feeds into machine learning and statistical analyses training and data sets.

In some examples, machine learning output, in one instance, is used as machine learning input in another instance. For example, in addition to the PCA being used for a classification process, the DNN data processing feeds into PCA and other analyses. This results in at least three levels of algorithmic processing. Other hierarchical structures are contemplated within the scope of the instant disclosure.

In some examples, including any of the foregoing, the methods include comparing the amount of each glycan or glycopeptide quantified in the sample to corresponding reference values for each glycan or glycopeptide in a diagnostic algorithm. In some examples, the method includes a comparative process by which the amount of a glycan or glycopeptide quantified in the sample is compared to a reference value for the same glycan or glycopeptide using a diagnostic algorithm. The comparative process may be part of a classification by a diagnostic algorithm. The comparative process may occur at an abstract level, e.g., in n-dimensional feature space or in a higher dimensional space.

In some examples, the methods herein include classifying a patient's sample based on the amount of each glycan or glycopeptide quantified in the sample with a diagnostic algorithm. In some examples, the methods include using statistical or machine learning classification processes by which the amount of a glycan or glycopeptide quantified in the test sample is used to determine a category of health with a diagnostic algorithm. In some examples, the diagnostic algorithm is a statistical or machine learning classification algorithm.

In some examples, including any of the foregoing, classification by a diagnostic algorithm may include scoring likelihood of a panel of glycan or glycopeptide values belonging to each possible category, and determining the highest-scoring category. Classification by a diagnostic algorithm may include comparing a panel of marker values to previous observations by means of a distance function. Examples of diagnostic algorithms suitable for classification include random forests, support vector machines, logistic regression (e.g. multiclass or multinomial logistic regression, and/or algorithms adapted for sparse logistic regression), or regularized regression. A wide variety of other diagnostic algorithms that are suitable for classification may be used, as known to a person skilled in the art.

In some examples, the methods herein include supervised learning of a diagnostic algorithm on the basis of values for each glycan or glycopeptide obtained from a population of individuals having a disease or condition (e.g., colorectal cancer or advanced adenoma). In some examples, the methods include variable selection in a statistical model on the basis of values for each glycan or glycopeptide obtained from a population of individuals having colorectal cancer or advanced adenoma. Training a diagnostic algorithm may for example include determining a weighting vector in feature space for each category, or determining a function or function parameters.

In one embodiment, the reference value is the amount of a glycan or glycopeptide in a sample or samples derived from one individual. Alternatively, the reference value may be derived by pooling data obtained from multiple individuals, and calculating an average (for example, mean or median) amount for a glycan or glycopeptide. Thus, the reference value may reflect the average amount of a glycan or glycopeptide in multiple individuals. Said amounts may be expressed in absolute or relative terms, in the same manner as described herein.

In some examples, the reference value may be derived from the same sample as the sample that is being tested, thus allowing for an appropriate comparison between the two. For example, if the sample is derived from urine, the reference value is also derived from urine. In some examples, if the sample is a blood sample (e.g., a plasma or a serum sample), then the reference value will also be a blood sample (e.g., a plasma sample or a serum sample, as appropriate). When comparing between the sample and the reference value, the way in which the amounts are expressed is matched between the sample and the reference value. Thus, an absolute amount can be compared with an absolute amount, and a relative amount can be compared with a relative amount. Similarly, the way in which the amounts are expressed for classification with the diagnostic algorithm is matched to the way in which the amounts are expressed for training the diagnostic algorithm.

When the amounts of the glycan or glycopeptide are determined, the method may comprise comparing the amount of each glycan or glycopeptide to its corresponding reference value. When the cumulative amount of one, some or all the glycan or glycopeptides are determined, the method may comprise comparing the cumulative amount to a corresponding reference value. When the amounts of the glycan or glycopeptides are combined with each other in a formula to form an index value, the index value can be compared to a corresponding reference index value derived in the same manner.

The reference values may be obtained either within (i.e., constituting a step of) or external to the (i.e., not constituting a step of) methods described herein. In some examples, the methods include a step of establishing a reference value for the quantity of the markers. In other examples, the reference values are obtained externally to the method described herein and accessed during the comparison step of the invention.

In some examples, including any of the foregoing, training of a diagnostic algorithm may be obtained either within (i.e., constituting a step of) or external to (i.e., not constituting a step of) the methods set forth herein. In some examples, the methods include a step of training of a diagnostic algorithm. In some examples, the diagnostic algorithm is trained externally to the method herein and accessed during the classification step of the invention. The reference value may be determined by quantifying the amount of a glycan or glycopeptide in a sample obtained from a population of healthy individual(s). The diagnostic algorithm may be trained by quantifying the amount of a glycan or glycopeptide in a sample obtained from a population of healthy individual(s). As used herein, the term “healthy individual” refers to an individual or group of individuals who are in a healthy state, e.g., patients who have not shown any symptoms of the disease, have not been diagnosed with the disease and/or are not likely to develop the disease. Preferably said healthy individual(s) is not on medication affecting the disease and has not been diagnosed with any other disease. The one or more healthy individuals may have a similar sex, age and body mass index (BMI) as compared with the test individual. The reference value may be determined by quantifying the amount of a glycan or glycopeptide in a sample obtained from a population of individual(s) suffering from the disease. The diagnostic algorithm may be trained by quantifying the amount of a marker in a sample obtained from a population of individual(s) suffering from the disease. More preferably such individual(s) may have similar sex, age and body mass index (BMI) as compared with the test individual. The reference value may be obtained from a population of individuals suffering from colorectal cancer or advanced adenoma. The diagnostic algorithm may be trained by quantifying the amount of a glycan or glycopeptide in a sample obtained from a population of individuals suffering from colorectal cancer or advanced adenoma. Once the characteristic glycan or glycopeptide profile of colorectal cancer or advanced adenoma is determined, the profile of markers from a biological sample obtained from an individual may be compared to this reference profile to determine whether the test subject also has colorectal cancer or advanced adenoma. Once the diagnostic algorithm is trained to classify colorectal cancer or advanced adenoma, the profile of markers from a biological sample obtained from an individual may be classified by the trained diagnostic algorithm to determine whether the test subject is also at that particular stage of colorectal cancer or advanced adenoma.

### VI. Compositions and Kits

Provided herein are compositions comprising one or more peptide structures from Table 10. Provided herein are compositions comprising one or more glycopeptides from Table 10. In some embodiments, provided herein is a composition comprising two or more peptide structures from Table 10. In some embodiments, provided herein is a composition comprising three or more peptide structures from Table 10. In some embodiments, provided herein is a composition comprising four or more peptide structures from Table 10. In some embodiments, provided herein is a composition comprising five or more peptide structures from Table 10. In some embodiments, provided herein is a composition comprising 10 or more peptide structures from Table 10. In some embodiments, provided herein is a composition comprising 15 or more peptide structures from Table 10. In some embodiments, provided herein is a composition comprising 20 or more peptide structures from Table 10. In some embodiments, provided herein is a composition comprising 25 or more peptide structures from Table 10. In some embodiments, provided herein is a composition comprising 30 or more peptide structures from Table 10. In some embodiments, provided herein is a composition comprising 35 or more peptide structures from Table 10. In some embodiments, the composition is from a biological sample. In some embodiments, the composition comprises one or more purified peptide structures. In some embodiments, the composition comprises one or more purified glycopeptides. In some embodiments, the composition comprises enzymatically digested peptide and/or glycopeptide fragments, such as those in Table 10. In some embodiments, the composition comprises enzymatically digested glycopeptide fragments, such as those in Table 10. In some embodiments, the composition comprises at least one, at least two, at least three, at least four, at least five, at least 10, at least 15, at least 20, at least 25, at least 30, or at least 35 peptides and/or glycopeptides comprising a sequence set forth in SEQ ID NOs: 1-38 along with the associated glycan set forth in Table 10.

In some embodiments, provided herein is a composition comprising at least one peptide and/or glycopeptide comprising a sequence set forth in SEQ ID NOs: 1-38 along with the associated glycan set forth in Table 10. In some embodiments, provided herein is a composition comprising at least two peptides and/or glycopeptides comprising a sequence set forth in SEQ ID NOs: 1-38 along with the associated glycan set forth in Table 10. In some embodiments, provided herein is a composition comprising at least three peptides and/or glycopeptides comprising a sequence set forth in SEQ ID NOs: 1-38 along with the associated glycan set forth in Table 10. In some embodiments, provided herein is a composition comprising at least four peptides and/or glycopeptides comprising a sequence set forth in SEQ ID NOs: 1-38 along with the associated glycan set forth in Table 10. In some embodiments, provided herein is a composition comprising at least five peptides and/or glycopeptides comprising a sequence set forth in SEQ ID NOs: 1-38 along with the associated glycan set forth in Table 10. In some embodiments, provided herein is a composition comprising at least 10 peptides and/or glycopeptides comprising a sequence set forth in SEQ ID NOs: 1-38 along with the associated glycan set forth in Table 10. In some embodiments, provided herein is a composition comprising at least 15 peptides and/or glycopeptides comprising a sequence set forth in SEQ ID NOs: 1-38 along with the associated glycan set forth in Table 10. In some embodiments, provided herein is a composition comprising 20 peptides and/or glycopeptides comprising sequences set forth in SEQ ID NOs: 1-38 along with the associated glycan set forth in Table 10. In some embodiments, provided herein is a composition comprising 25 peptides and/or glycopeptides comprising sequences set forth in SEQ ID NOs: 1-38 along with the associated glycan set forth in Table 10. In some embodiments, provided herein is a composition comprising 30 peptides and/or glycopeptides comprising sequences set forth in SEQ ID NOs: 1-38 along with the associated glycan set forth in Table 10. In some embodiments, provided herein is a composition comprising 35 peptides and/or glycopeptides comprising sequences set forth in SEQ ID NOs: 1-38 along with the associated glycan set forth in Table 10.

In some embodiments, provided herein are peptides and/or glycopeptides set forth in Table 10. In some embodiments, provided herein are glycopeptides set forth in Table 10. In some embodiments, provided herein are peptides and/or glycopeptides comprising a sequence set forth in SEQ ID NOs: 1-38 along with the associated glycan set forth in Table 10.

Also provided herein, in some examples, including any of the foregoing, is a kit comprising one or more glycopeptide standard(s), a buffer, and one or more peptides comprising the sequence set forth in SEQ ID NOs: 1-38.

In some examples, including any of the foregoing, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptide standard(s), a buffer, and one or more peptides comprising the sequence set forth in SEQ ID NOs: 1-38. In some examples, the kit comprises one or more glycopeptide standard(s), a buffer, and one or more peptides comprising the sequence set forth in SEQ ID NOs: 5, 8-11, 13-14, 16-22, 26-28, 30-31, and 34-38, and combinations thereof. In some examples, the kit comprises one or more glycopeptide standard(s), a buffer, and one or more peptides comprising the sequence set forth in SEQ ID NOs: 3, 7, 9, 28, 29, 32, and 33, and combinations thereof. In some examples, the kit comprises one or more glycopeptide standard(s), a buffer, and one or more peptides comprising the sequence set forth in SEQ ID NOs: 1-4, 6-7, 12, 15, 23-25, 28, 29, and 32, and combinations thereof.

In some examples, including any of the foregoing, set forth herein is a kit comprising a glycopeptide standard, a buffer, and one or more glycopeptides consisting of an amino acid sequence selected from the group consisting of SEQ ID NOs: 1-38.

In some examples, including any of the foregoing, set forth herein is a kit comprising a glycopeptide standard, a buffer, and one or more glycopeptides consisting essentially of an amino acid sequence selected from the group consisting of SEQ ID NOs: 1-38.

In some examples, including any of the foregoing, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptides consisting of an amino acid sequence selected from the group consisting of SEQ ID NOs: 1-38. In some examples, the kit comprises one or more glycopeptides consisting essentially of an amino acid sequence selected from the group consisting of SEQ ID NOs: 1-38.

In some examples, including any of the foregoing, set forth herein is a kit comprising a glycopeptide standard, a buffer, and one or more glycopeptides consisting of an amino acid sequence selected from the group consisting of SEQ ID NOs: 5, 8-11, 13-14, 16-22, 26-28, 30-31, and 34-38. In some examples, including any of the foregoing, set forth herein is a kit comprising a glycopeptide standard, a buffer, and one or more glycopeptides consisting of an amino acid sequence selected from the group consisting of SEQ ID NOs: 3, 7, 9, 28, 29, 32, and 33. In some examples, including any of the foregoing, set forth herein is a kit comprising a glycopeptide standard, a buffer, and one or more glycopeptides consisting of an amino acid sequence selected from the group consisting of SEQ ID NOs: 1-4, 6-7, 12, 15, 23-25, 28, 29, and 32.

In some examples, including any of the foregoing, set forth herein is a kit comprising a glycopeptide standard, a buffer, and one or more glycopeptides consisting essentially of an amino acid sequence selected from the group consisting of SEQ ID NOs: 5, 8-11, 13-14, 16-22, 26-28, 30-31, and 34-38. In some examples, including any of the foregoing, set forth herein is a kit comprising a glycopeptide standard, a buffer, and one or more glycopeptides consisting essentially of an amino acid sequence selected from the group consisting of SEQ ID NOs: 3, 7, 9, 28, 29, 32, and 33. In some examples, including any of the foregoing, set forth herein is a kit comprising a glycopeptide standard, a buffer, and one or more glycopeptides consisting essentially of an amino acid sequence selected from the group consisting of SEQ ID NOs: 1-4, 6-7, 12, 15, 23-25, 28, 29, and 32.

In some examples, including any of the foregoing, set forth herein is a kit for diagnosing or monitoring cancer in an individual wherein the glycan or glycopeptide profile of a sample from said individual is determined and the measured profile is compared with a profile of a normal patient or a profile of a patient with a family history of cancer. In some examples, the kit comprises one or more glycopeptides consisting of an amino acid sequence selected from the group consisting of SEQ ID NOs: 5, 8-11, 13-14, 16-22, 26-28, 30-31, and 34-38. In some examples, the kit comprises one or more glycopeptides consisting of an amino acid sequence selected from the group consisting of SEQ ID NOs: 3, 7, 9, 28, 29, 32, and 33. In some examples, the kit comprises one or more glycopeptides consisting of an amino acid sequence selected from the group consisting of SEQ ID NOs: 1-4, 6-7, 12, 15, 23-25, 28, 29, and 32.

In some examples, the kit comprises one or more glycopeptides consisting essentially of an amino acid sequence selected from the group consisting of SEQ ID NOs: 5, 8-11, 13-14, 16-22, 26-28, 30-31, and 34-38. In some examples, the kit comprises one or more glycopeptides consisting essentially of an amino acid sequence selected from the group consisting of SEQ ID NOs: 3, 7, 9, 28, 29, 32, and 33. In some examples, the kit comprises one or more glycopeptides consisting essentially of an amino acid sequence selected from the group consisting of SEQ ID NOs: 1-4, 6-7, 12, 15, 23-25, 28, 29, and 32.

In some examples, the kit comprises a glycopeptide of amino acid sequence SEQ ID NO: 5, in the sample. In some examples, the kit comprises a glycopeptide of amino acid sequence SEQ ID NO: 8, in the sample. In some examples, the kit comprises a glycopeptide of amino acid sequence SEQ ID NO: 9, in the sample. In some examples, the kit comprises a glycopeptide of amino acid sequence SEQ ID NO: 10, in the sample. In some examples, the kit comprises a glycopeptide of amino acid sequence SEQ ID NO: 11, in the sample. In some examples, the kit comprises a glycopeptide of amino acid sequence SEQ ID NO: 13, in the sample. In some examples, the kit comprises a glycopeptide of amino acid sequence SEQ ID NO: 14, in the sample. In some examples, the kit comprises a glycopeptide of amino acid sequence SEQ ID NO: 16, in the sample. In some examples, the kit comprises a glycopeptide of amino acid sequence SEQ ID NO: 17, in the sample. In some examples, the kit comprises a glycopeptide of amino acid sequence SEQ ID NO: 18, in the sample. In some examples, the kit comprises a glycopeptide of amino acid sequence SEQ ID NO: 19, in the sample. In some examples, the kit comprises a glycopeptide of amino acid sequence SEQ ID NO: 20, in the sample. In some examples, the kit comprises a glycopeptide of amino acid sequence SEQ ID NO: 21, in the sample. In some examples, the kit comprises a glycopeptide of amino acid sequence SEQ ID NO: 22, in the sample. In some examples, the kit comprises a glycopeptide of amino acid sequence SEQ ID NO: 26, in the sample. In some examples, the kit comprises a glycopeptide of amino acid sequence SEQ ID NO: 27, in the sample. In some examples, the kit comprises a glycopeptide of amino acid sequence SEQ ID NO: 28, in the sample. In some examples, the kit comprises a glycopeptide of amino acid sequence SEQ ID NO: 30, in the sample. In some examples, the kit comprises a glycopeptide of amino acid sequence SEQ ID NO: 31, in the sample. In some examples, the kit comprises a glycopeptide of amino acid sequence SEQ ID NO: 34, in the sample. In some examples, the kit comprises a glycopeptide of amino acid sequence SEQ ID NO: 35, in the sample. In some examples, the kit comprises a glycopeptide of amino acid sequence SEQ ID NO: 36, in the sample. In some examples, the kit comprises a glycopeptide of amino acid sequence SEQ ID NO: 37, in the sample. In some examples, the kit comprises a glycopeptide of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, the kit comprises a glycopeptide essentially of a sequence SEQ ID NO: 5, in the sample. In some examples, the kit comprises a glycopeptide essentially of amino acid sequence SEQ ID NO: 8, in the sample. In some examples, the kit comprises a glycopeptide essentially of amino acid sequence SEQ ID NO: 9, in the sample. In some examples, the kit comprises a glycopeptide essentially of amino acid sequence SEQ ID NO: 10, in the sample. In some examples, the kit comprises a glycopeptide essentially of amino acid sequence SEQ ID NO: 11, in the sample. In some examples, the kit comprises a glycopeptide essentially of amino acid sequence SEQ ID NO: 13, in the sample. In some examples, the kit comprises a glycopeptide essentially of amino acid sequence SEQ ID NO: 14, in the sample. In some examples, the kit comprises a glycopeptide essentially of amino acid sequence SEQ ID NO: 16, in the sample. In some examples, the kit comprises a glycopeptide essentially of amino acid sequence SEQ ID NO: 17, in the sample. In some examples, the kit comprises a glycopeptide essentially of amino acid sequence SEQ ID NO: 18, in the sample. In some examples, the kit comprises a glycopeptide essentially of amino acid sequence SEQ ID NO: 19, in the sample. In some examples, the kit comprises a glycopeptide essentially of amino acid sequence SEQ ID NO: 20, in the sample. In some examples, the kit comprises a glycopeptide essentially of amino acid sequence SEQ ID NO: 21, in the sample. In some examples, the kit comprises a glycopeptide essentially of amino acid sequence SEQ ID NO: 22, in the sample. In some examples, the kit comprises a glycopeptide essentially of amino acid sequence SEQ ID NO: 26, in the sample. In some examples, the kit comprises a glycopeptide essentially of amino acid sequence SEQ ID NO: 27, in the sample. In some examples, the kit comprises a glycopeptide essentially of amino acid sequence SEQ ID NO: 28, in the sample. In some examples, the kit comprises a glycopeptide essentially of amino acid sequence SEQ ID NO: 30, in the sample. In some examples, the kit comprises a glycopeptide essentially of amino acid sequence SEQ ID NO: 31, in the sample. In some examples, the kit comprises a glycopeptide essentially of amino acid sequence SEQ ID NO: 34, in the sample. In some examples, the kit comprises a glycopeptide essentially of amino acid sequence SEQ ID NO: 35, in the sample. In some examples, the kit comprises a glycopeptide essentially of amino acid sequence SEQ ID NO: 36, in the sample. In some examples, the kit comprises a glycopeptide essentially of amino acid sequence SEQ ID NO: 37, in the sample. In some examples, the kit comprises a glycopeptide essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, the kit comprises a glycopeptide of, or consisting essentially of sequence SEQ ID NO: 5, in the sample. In some examples, the kit comprises a glycopeptide of, or consisting essentially of amino acid sequence SEQ ID NO: 8, in the sample. In some examples, the kit comprises a glycopeptide of, or consisting essentially of amino acid sequence SEQ ID NO: 9, in the sample. In some examples, the kit comprises a glycopeptide of, or essentially of amino acid sequence SEQ ID NO: 10, in the sample. In some examples, the kit comprises a glycopeptide of, or essentially of amino acid sequence SEQ ID NO: 11, in the sample. In some examples, the kit comprises a glycopeptide of, or essentially of amino acid sequence SEQ ID NO: 13, in the sample. In some examples, the kit comprises a glycopeptide of, or essentially of amino acid sequence SEQ ID NO: 14, in the sample. In some examples, the kit comprises a glycopeptide of, or essentially of amino acid sequence SEQ ID NO: 16, in the sample. In some examples, the kit comprises a glycopeptide of, or essentially of amino acid sequence SEQ ID NO: 17, in the sample. In some examples, the kit comprises a glycopeptide of, or essentially of amino acid sequence SEQ ID NO: 18, in the sample. In some examples, the kit comprises a glycopeptide of, or essentially of amino acid sequence SEQ ID NO: 19, in the sample. In some examples, the kit comprises a glycopeptide of, or essentially of amino acid sequence SEQ ID NO: 20, in the sample. In some examples, the kit comprises a glycopeptide of, or essentially of amino acid sequence SEQ ID NO: 21, in the sample. In some examples, the kit comprises a glycopeptide of, or essentially of amino acid sequence SEQ ID NO: 22, in the sample. In some examples, the kit comprises a glycopeptide of, or essentially of amino acid sequence SEQ ID NO: 26, in the sample. In some examples, the kit comprises a glycopeptide of, or essentially of amino acid sequence SEQ ID NO: 27, in the sample. In some examples, the kit comprises a glycopeptide of, or essentially of amino acid sequence SEQ ID NO: 28, in the sample. In some examples, the kit comprises a glycopeptide of, or essentially of amino acid sequence SEQ ID NO: 30, in the sample. In some examples, the kit comprises a glycopeptide of, or essentially of amino acid sequence SEQ ID NO: 31, in the sample. In some examples, the kit comprises a glycopeptide of, or essentially of amino acid sequence SEQ ID NO: 34, in the sample. In some examples, the kit comprises a glycopeptide of, or essentially of amino acid sequence SEQ ID NO: 35, in the sample. In some examples, the kit comprises a glycopeptide of, or essentially of amino acid sequence SEQ ID NO: 36, in the sample. In some examples, the kit comprises a glycopeptide of, or essentially of amino acid sequence SEQ ID NO: 37, in the sample. In some examples, the kit comprises a glycopeptide of, or essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, including any of the foregoing, set forth herein is a kit comprising the reagents for quantification of the oxidized, nitrated, and/or glycated free adducts derived from glycopeptides.

### VII. Clinical Assays

In some examples, including any of the foregoing, the biomarkers, methods, and/or kits may be used in a clinical setting for diagnosing patients. In some of these examples, the analysis of samples includes the use of internal standards. These standards may include one or more glycopeptides consisting of an amino acid sequence selected from the group consisting of SEQ ID NOs: 1-38. These standards may include one or more glycopeptides consisting essentially of an amino acid sequence selected from the group consisting of SEQ ID NOs: 1-38.

In a clinical setting, samples may be prepared (e.g., by digestion) to include one or more glycopeptides consisting of an amino acid sequence selected from the group consisting of SEQ ID NOs: 1-38.

In a clinical setting, samples may be prepared (e.g., by digestion) to include one or more glycopeptides consisting essentially of an amino acid sequence selected from the group consisting of SEQ ID NOs: 1-38.

In some examples, the amount of a glycan or glycopeptide may be assessed by comparing the amount of one or more glycopeptides consisting of an amino acid sequence selected from the group consisting of SEQ ID NOs: 1-38 to the concentration of another biomarker.

In some examples, the amount of a glycan or glycopeptide may be assessed by comparing the amount of one or more glycopeptides consisting essentially of an amino acid sequence selected from the group consisting of SEQ ID NOs: 1-38 to the concentration of another biomarker.

In some examples, the amount of a glycan or glycopeptide may be assessed by comparing the amount of one or more glycopeptides consisting of an amino acid sequence selected from the group consisting of SEQ ID NOs: 1-38 to the amount of one or more glycopeptides consisting of an amino acid sequence selected from the group consisting of SEQ ID NOs: 1-38.

In some examples, the amount of a glycan or glycopeptide may be assessed by comparing the amount of one or more glycopeptides consisting essentially of an amino acid sequence selected from the group consisting of SEQ ID NOs: 1-38 to the amount of one or more glycopeptides consisting essentially of an amino acid sequence selected from the group consisting of SEQ ID NOs: 1-38.

In some examples, including any of the foregoing, the kit may include software for computing the normalization of a glycopeptide MRM transition signal.

In some examples, including any of the foregoing, the kit may include software for quantifying the amount of a glycopeptide consisting of, or consisting essentially of, an amino acid sequence selected from the group consisting of SEQ ID NOs: 1-38.

In some examples, including any of the foregoing, the kit may include software for quantifying the relative amount of a glycopeptide consisting of, or consisting essentially of, an amino acid sequence selected from the group consisting of SEQ ID NOs: 1-38.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

In some examples, including any of the foregoing, a trained model is stored on a server which is accessed by a clinician performing a method, set forth herein. In some examples, the clinician inputs the quantification of the glycopeptide or glycopeptides consisting of, or consisting essentially of, an amino acid sequence selected from the group consisting of SEQ ID NOs: 1-38 from a patient's sample into a trained model which are stored on a server. In some examples, the server is accessed by the internet, wireless communication, or other digital or telecommunication methods.

In some examples, including any of the foregoing, MRM transition signals 1-38 are stored on a server which is accessed by a clinician performing a method, set forth herein. In some examples, the clinician compares the MRM transition signals from a patient's sample to the MRM transition signals 1-38 which are stored on a server. In some examples, the server is accessed by the internet, wireless communication, or other digital or telecommunication methods.

In some examples, including any of the foregoing, machine learning algorithm, which has been trained using the MRM transition signals 1-38, described herein, is stored on a server which is accessed by a clinician performing a method, set forth herein. In some examples, the machine learning algorithm, accessed remotely on a server, analyzes the MRM transition signals from a patient's sample. In some examples, the server is accessed by the internet, wireless communication, or other digital or telecommunication methods.

In some examples, including any of the foregoing, the biomarkers, methods, and/or kits may be used in a clinical setting for diagnosing patients. In some of these examples, the analysis of samples includes the use of internal standards. These standards may include one or more glycopeptides consisting of an amino acid sequence selected from the group consisting of SEQ ID NOs: 5, 8-11, 13-14, 16-22, 26-28, 30-31, and 34-38. These standards may include one or more glycopeptides consisting of an amino acid sequence selected from the group consisting of SEQ ID NOs: 3, 7, 9, 28, 29, 32, and 33. These standards may include one or more glycopeptides consisting of an amino acid sequence selected from the group consisting of SEQ ID NOs: 1-4, 6-7, 12, 15, 23-25, 28, 29, 32.

In some examples, the standard comprises a glycopeptide of amino acid sequence SEQ ID NO: 5, in the sample. In some examples, the standard comprises a glycopeptide of amino acid sequence SEQ ID NO: 8, in the sample. In some examples, the standard comprises a glycopeptide of amino acid sequence SEQ ID NO: 9, in the sample. In some examples, the standard comprises a glycopeptide of amino acid sequence SEQ ID NO: 10, in the sample. In some examples, the standard comprises a glycopeptide of amino acid sequence SEQ ID NO: 11, in the sample. In some examples, the standard comprises a glycopeptide of amino acid sequence SEQ ID NO: 13, in the sample. In some examples, the standard comprises a glycopeptide of amino acid sequence SEQ ID NO: 14, in the sample. In some examples, the standard comprises a glycopeptide of amino acid sequence SEQ ID NO: 16, in the sample. In some examples, the standard comprises a glycopeptide of amino acid sequence SEQ ID NO: 17, in the sample. In some examples, the standard comprises a glycopeptide of amino acid sequence SEQ ID NO: 18, in the sample. In some examples, the standard comprises a glycopeptide of amino acid sequence SEQ ID NO: 19, in the sample. In some examples, the standard comprises a glycopeptide of amino acid sequence SEQ ID NO: 20, in the sample. In some examples, the standard comprises a glycopeptide of amino acid sequence SEQ ID NO: 21, in the sample. In some examples, the standard comprises a glycopeptide of amino acid sequence SEQ ID NO: 22, in the sample. In some examples, the standard comprises a glycopeptide of amino acid sequence SEQ ID NO: 26, in the sample. In some examples, the standard comprises a glycopeptide of amino acid sequence SEQ ID NO: 27, in the sample. In some examples, the standard comprises a glycopeptide of amino acid sequence SEQ ID NO: 28, in the sample. In some examples, the standard comprises a glycopeptide of amino acid sequence SEQ ID NO: 30, in the sample. In some examples, the standard comprises a glycopeptide of amino acid sequence SEQ ID NO: 31, in the sample. In some examples, the standard comprises a glycopeptide of amino acid sequence SEQ ID NO: 34, in the sample. In some examples, the standard comprises a glycopeptide of amino acid sequence SEQ ID NO: 35, in the sample. In some examples, the standard comprises a glycopeptide of amino acid sequence SEQ ID NO: 36, in the sample. In some examples, the standard comprises a glycopeptide of amino acid sequence SEQ ID NO: 37, in the sample. In some examples, the standard comprises a glycopeptide of amino acid sequence SEQ ID NO: 38, in the sample. In particular embodiments, each glycopeptide comprises or is bonded to a glycan, for instance as described herein. In some examples, as described below, the presence, absolute amount, and/or relative amount of a glycopeptide is determined by analyzing the MS results. In some examples, the MS results are analyzed using machine learning.

In some examples, the standard comprises a glycopeptide essentially of a sequence SEQ ID NO: 5, in the sample. In some examples, the standard comprises a glycopeptide essentially of amino acid sequence SEQ ID NO: 8, in the sample. In some examples, the standard comprises a glycopeptide essentially of amino acid sequence SEQ ID NO: 9, in the sample. In some examples, the standard comprises a glycopeptide essentially of amino acid sequence SEQ ID NO: 10, in the sample. In some examples, the standard comprises a glycopeptide essentially of amino acid sequence SEQ ID NO: 11, in the sample. In some examples, the standard comprises a glycopeptide essentially of amino acid sequence SEQ ID NO: 13, in the sample. In some examples, the standard comprises a glycopeptide essentially of amino acid sequence SEQ ID NO: 14, in the sample. In some examples, the standard comprises a glycopeptide essentially of amino acid sequence SEQ ID NO: 16, in the sample. In some examples, the standard comprises a glycopeptide essentially of amino acid sequence SEQ ID NO: 17, in the sample. In some examples, the standard comprises a glycopeptide essentially of amino acid sequence SEQ ID NO: 18, in the sample. In some examples, the standard comprises a glycopeptide essentially of amino acid sequence SEQ ID NO: 19, in the sample. In some examples, the standard comprises a glycopeptide essentially of amino acid sequence SEQ ID NO: 20, in the sample. In some examples, the standard comprises a glycopeptide essentially of amino acid sequence SEQ ID NO: 21, in the sample. In some examples, the standard comprises a glycopeptide essentially of amino acid sequence SEQ ID NO: 22, in the sample. In some examples, the standard comprises a glycopeptide essentially of amino acid sequence SEQ ID NO: 26, in the sample. In some examples, the standard comprises a glycopeptide essentially of amino acid sequence SEQ ID NO: 27, in the sample. In some examples, the standard comprises a glycopeptide essentially of amino acid sequence SEQ ID NO: 28, in the sample. In some examples, the standard comprises a glycopeptide essentially of amino acid sequence SEQ ID NO: 30, in the sample. In some examples, the standard comprises a glycopeptide essentially of amino acid sequence SEQ ID NO: 31, in the sample. In some examples, the standard comprises a glycopeptide essentially of amino acid sequence SEQ ID NO: 34, in the sample. In some examples, the standard comprises a glycopeptide essentially of amino acid sequence SEQ ID NO: 35, in the sample. In some examples, the standard comprises a glycopeptide essentially of amino acid sequence SEQ ID NO: 36, in the sample. In some examples, the standard comprises a glycopeptide essentially of amino acid sequence SEQ ID NO: 37, in the sample. In particular embodiments, each glycopeptide comprises or is bonded to a glycan, for instance as described herein. In some examples, the standard comprises a glycopeptide essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In some examples, the standard comprises a glycopeptide of, or consisting essentially of sequence SEQ ID NO: 5, in the sample. In some examples, the standard comprises a glycopeptide of, or consisting essentially of amino acid sequence SEQ ID NO: 8, in the sample. In some examples, the standard comprises a glycopeptide of, or consisting essentially of amino acid sequence SEQ ID NO: 9, in the sample. In some examples, the standard comprises a glycopeptide of, or essentially of amino acid sequence SEQ ID NO: 10, in the sample. In some examples, the standard comprises a glycopeptide of, or essentially of amino acid sequence SEQ ID NO: 11, in the sample. In some examples, the standard comprises a glycopeptide of, or essentially of amino acid sequence SEQ ID NO: 13, in the sample. In some examples, the standard comprises a glycopeptide of, or essentially of amino acid sequence SEQ ID NO: 14, in the sample. In some examples, the standard comprises a glycopeptide of, or essentially of amino acid sequence SEQ ID NO: 16, in the sample. In some examples, the standard comprises a glycopeptide of, or essentially of amino acid sequence SEQ ID NO: 17, in the sample. In some examples, the standard comprises a glycopeptide of, or essentially of amino acid sequence SEQ ID NO: 18, in the sample. In some examples, the standard comprises a glycopeptide of, or essentially of amino acid sequence SEQ ID NO: 19, in the sample. In some examples, the standard comprises a glycopeptide of, or essentially of amino acid sequence SEQ ID NO: 20, in the sample. In some examples, the standard comprises a glycopeptide of, or essentially of amino acid sequence SEQ ID NO: 21, in the sample. In some examples, the standard comprises a glycopeptide of, or essentially of amino acid sequence SEQ ID NO: 22, in the sample. In some examples, the standard comprises a glycopeptide of, or essentially of amino acid sequence SEQ ID NO: 26, in the sample. In some examples, the standard comprises a glycopeptide of, or essentially of amino acid sequence SEQ ID NO: 27, in the sample. In some examples, the standard comprises a glycopeptide of, or essentially of amino acid sequence SEQ ID NO: 28, in the sample. In some examples, the standard comprises a glycopeptide of, or essentially of amino acid sequence SEQ ID NO: 30, in the sample. In some examples, the standard comprises a glycopeptide of, or essentially of amino acid sequence SEQ ID NO: 31, in the sample. In some examples, the standard comprises a glycopeptide of, or essentially of amino acid sequence SEQ ID NO: 34, in the sample. In some examples, the standard comprises a glycopeptide of, or essentially of amino acid sequence SEQ ID NO: 35, in the sample. In some examples, the standard comprises a glycopeptide of, or essentially of amino acid sequence SEQ ID NO: 36, in the sample. In some examples, the standard comprises a glycopeptide of, or essentially of amino acid sequence SEQ ID NO: 37, in the sample. In some examples, the standard comprises a glycopeptide of, or essentially of amino acid sequence SEQ ID NO: 38, in the sample. In particular embodiments, each glycopeptide comprises or is bonded to a glycan, for instance as described herein. In some examples, as described below, the presence, absolute amount, and/or relative amount of a glycopeptide is determined by analyzing the MS results. In some examples, the MS results are analyzed using machine learning.

These standards may include one or more glycopeptides consisting essentially of an amino acid sequence selected from the group consisting of SEQ ID NOs: 5, 8-11, 13-14, 16-22, 26-28, 30-31, and 34-38. These standards may include one or more glycopeptides consisting essentially of an amino acid sequence selected from the group consisting of SEQ ID NOs: 3, 7, 9, 28, 29, 32, and 33. These standards may include one or more glycopeptides consisting essentially of an amino acid sequence selected from the group consisting of SEQ ID NOs: 1-4, 6-7, 12, 15, 23-25, 28, 29, 32.

In a clinical setting, samples may be prepared (e.g., by digestion) to include one or more glycopeptides consisting of an amino acid sequence selected from the group consisting of SEQ ID NOs: 5, 8-11, 13-14, 16-22, 26-28, 30-31, and 34-38. In a clinical setting, samples may be prepared (e.g., by digestion) to include one or more glycopeptides consisting of an amino acid sequence selected from the group consisting of SEQ ID NOs: 3, 7, 9, 28, 29, 32, and 33. In a clinical setting, samples may be prepared (e.g., by digestion) to include one or more glycopeptides consisting of an amino acid sequence selected from the group consisting of SEQ ID NOs: 1-4, 6-7, 12, 15, 23-25, 28, 29, 32.

In a clinical setting, samples may be prepared (e.g., by digestion) to include a glycopeptide consisting of amino acid sequence SEQ ID NO: 5, in the sample. In a clinical setting, samples may be prepared (e.g., by digestion) to include a glycopeptide consisting of amino acid sequence SEQ ID NO: 8, in the sample. In a clinical setting, samples may be prepared (e.g., by digestion) to include a glycopeptide consisting of amino acid sequence SEQ ID NO: 9, in the sample. In a clinical setting, samples may be prepared (e.g., by digestion) to include a glycopeptide consisting of amino acid sequence SEQ ID NO: 10, in the sample. In a clinical setting, samples may be prepared (e.g., by digestion) to include a glycopeptide consisting of amino acid sequence SEQ ID NO: 11, in the sample. In a clinical setting, samples may be prepared (e.g., by digestion) to include a glycopeptide consisting of amino acid sequence SEQ ID NO: 13, in the sample. In a clinical setting, samples may be prepared (e.g., by digestion) to include a glycopeptide consisting of amino acid sequence SEQ ID NO: 14, in the sample. In a clinical setting, samples may be prepared (e.g., by digestion) to include a glycopeptide consisting of amino acid sequence SEQ ID NO: 16, in the sample. In a clinical setting, samples may be prepared (e.g., by digestion) to include a glycopeptide consisting of amino acid sequence SEQ ID NO: 17, in the sample. In a clinical setting, samples may be prepared (e.g., by digestion) to include a glycopeptide consisting of amino acid sequence SEQ ID NO: 18, in the sample. In a clinical setting, samples may be prepared (e.g., by digestion) to include a glycopeptide consisting of amino acid sequence SEQ ID NO: 19, in the sample. In a clinical setting, samples may be prepared (e.g., by digestion) to include a glycopeptide consisting of amino acid sequence SEQ ID NO: 20, in the sample. In a clinical setting, samples may be prepared (e.g., by digestion) to include a glycopeptide consisting of amino acid sequence SEQ ID NO: 21, in the sample. In a clinical setting, samples may be prepared (e.g., by digestion) to include a glycopeptide consisting of amino acid sequence SEQ ID NO: 22, in the sample. In a clinical setting, samples may be prepared (e.g., by digestion) to include a glycopeptide consisting of amino acid sequence SEQ ID NO: 26, in the sample. In a clinical setting, samples may be prepared (e.g., by digestion) to include a glycopeptide consisting of amino acid sequence SEQ ID NO: 27, in the sample. In a clinical setting, samples may be prepared (e.g., by digestion) to include a glycopeptide consisting of amino acid sequence SEQ ID NO: 28, in the sample. In a clinical setting, samples may be prepared (e.g., by digestion) to include a glycopeptide consisting of amino acid sequence SEQ ID NO: 30, in the sample. In a clinical setting, samples may be prepared (e.g., by digestion) to include a glycopeptide consisting of amino acid sequence SEQ ID NO: 31, in the sample. In a clinical setting, samples may be prepared (e.g., by digestion) to include a glycopeptide consisting of amino acid sequence SEQ ID NO: 34, in the sample. In a clinical setting, samples may be prepared (e.g., by digestion) to include a glycopeptide consisting of amino acid sequence SEQ ID NO: 35, in the sample. In a clinical setting, samples may be prepared (e.g., by digestion) to include a glycopeptide consisting of amino acid sequence SEQ ID NO: 36, in the sample. In a clinical setting, samples may be prepared (e.g., by digestion) to include a glycopeptide consisting of amino acid sequence SEQ ID NO: 37, in the sample. In a clinical setting, samples may be prepared (e.g., by digestion) to include a glycopeptide consisting of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In a clinical setting, samples may be prepared (e.g., by digestion) to include a glycopeptide consisting essentially of a sequence SEQ ID NO: 5, in the sample. In a clinical setting, samples may be prepared (e.g., by digestion) to include a glycopeptide consisting essentially of amino acid sequence SEQ ID NO: 8, in the sample. In a clinical setting, samples may be prepared (e.g., by digestion) to include a glycopeptide consisting essentially of amino acid sequence SEQ ID NO: 9, in the sample. In a clinical setting, samples may be prepared (e.g., by digestion) to include a glycopeptide consisting essentially of amino acid sequence SEQ ID NO: 10, in the sample. In a clinical setting, samples may be prepared (e.g., by digestion) to include a glycopeptide consisting essentially of amino acid sequence SEQ ID NO: 11, in the sample. In a clinical setting, samples may be prepared (e.g., by digestion) to include a glycopeptide consisting essentially of amino acid sequence SEQ ID NO: 13, in the sample. In a clinical setting, samples may be prepared (e.g., by digestion) to include a glycopeptide consisting essentially of amino acid sequence SEQ ID NO: 14, in the sample. In a clinical setting, samples may be prepared (e.g., by digestion) to include a glycopeptide consisting essentially of amino acid sequence SEQ ID NO: 16, in the sample. In a clinical setting, samples may be prepared (e.g., by digestion) to include a glycopeptide consisting essentially of amino acid sequence SEQ ID NO: 17, in the sample. In a clinical setting, samples may be prepared (e.g., by digestion) to include a glycopeptide consisting essentially of amino acid sequence SEQ ID NO: 18, in the sample. In a clinical setting, samples may be prepared (e.g., by digestion) to include a glycopeptide consisting essentially of amino acid sequence SEQ ID NO: 19, in the sample. In a clinical setting, samples may be prepared (e.g., by digestion) to include a glycopeptide consisting essentially of amino acid sequence SEQ ID NO: 20, in the sample. In a clinical setting, samples may be prepared (e.g., by digestion) to include a glycopeptide consisting essentially of amino acid sequence SEQ ID NO: 21, in the sample. In a clinical setting, samples may be prepared (e.g., by digestion) to include a glycopeptide consisting essentially of amino acid sequence SEQ ID NO: 22, in the sample. In a clinical setting, samples may be prepared (e.g., by digestion) to include a glycopeptide consisting essentially of amino acid sequence SEQ ID NO: 26, in the sample. In a clinical setting, samples may be prepared (e.g., by digestion) to include a glycopeptide consisting essentially of amino acid sequence SEQ ID NO: 27, in the sample. In a clinical setting, samples may be prepared (e.g., by digestion) to include a glycopeptide consisting essentially of amino acid sequence SEQ ID NO: 28, in the sample. In a clinical setting, samples may be prepared (e.g., by digestion) to include a glycopeptide consisting essentially of amino acid sequence SEQ ID NO: 30, in the sample. In a clinical setting, samples may be prepared (e.g., by digestion) to include a glycopeptide consisting essentially of amino acid sequence SEQ ID NO: 31, in the sample. In a clinical setting, samples may be prepared (e.g., by digestion) to include a glycopeptide consisting essentially of amino acid sequence SEQ ID NO: 34, in the sample. In a clinical setting, samples may be prepared (e.g., by digestion) to include a glycopeptide consisting essentially of amino acid sequence SEQ ID NO: 35, in the sample. In a clinical setting, samples may be prepared (e.g., by digestion) to include a glycopeptide consisting essentially of amino acid sequence SEQ ID NO: 36, in the sample. In a clinical setting, samples may be prepared (e.g., by digestion) to include a glycopeptide consisting essentially of amino acid sequence SEQ ID NO: 37, in the sample. In a clinical setting, samples may be prepared (e.g., by digestion) to include a glycopeptide consisting essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In a clinical setting, samples may be prepared (e.g., by digestion) to include a glycopeptide consisting of, or consisting essentially of sequence SEQ ID NO: 5, in the sample. In a clinical setting, samples may be prepared (e.g., by digestion) to include a glycopeptide consisting of, or consisting essentially of amino acid sequence SEQ ID NO: 8, in the sample. In a clinical setting, samples may be prepared (e.g., by digestion) to include a glycopeptide consisting of, or consisting essentially of amino acid sequence SEQ ID NO: 9, in the sample. In a clinical setting, samples may be prepared (e.g., by digestion) to include a glycopeptide consisting of, or essentially of amino acid sequence SEQ ID NO: 10, in the sample. In a clinical setting, samples may be prepared (e.g., by digestion) to include a glycopeptide consisting of, or essentially of amino acid sequence SEQ ID NO: 11, in the sample. In a clinical setting, samples may be prepared (e.g., by digestion) to include a glycopeptide consisting of, or essentially of amino acid sequence SEQ ID NO: 13, in the sample. In a clinical setting, samples may be prepared (e.g., by digestion) to include a glycopeptide consisting of, or essentially of amino acid sequence SEQ ID NO: 14, in the sample. In a clinical setting, samples may be prepared (e.g., by digestion) to include a glycopeptide consisting of, or essentially of amino acid sequence SEQ ID NO: 16, in the sample. In a clinical setting, samples may be prepared (e.g., by digestion) to include a glycopeptide consisting of, or essentially of amino acid sequence SEQ ID NO: 17, in the sample. In a clinical setting, samples may be prepared (e.g., by digestion) to include a glycopeptide consisting of, or essentially of amino acid sequence SEQ ID NO: 18, in the sample. In a clinical setting, samples may be prepared (e.g., by digestion) to include a glycopeptide consisting of, or essentially of amino acid sequence SEQ ID NO: 19, in the sample. In a clinical setting, samples may be prepared (e.g., by digestion) to include a glycopeptide consisting of, or essentially of amino acid sequence SEQ ID NO: 20, in the sample. In a clinical setting, samples may be prepared (e.g., by digestion) to include a glycopeptide consisting of, or essentially of amino acid sequence SEQ ID NO: 21, in the sample. In a clinical setting, samples may be prepared (e.g., by digestion) to include a glycopeptide consisting of, or essentially of amino acid sequence SEQ ID NO: 22, in the sample. In a clinical setting, samples may be prepared (e.g., by digestion) to include a glycopeptide consisting of, or essentially of amino acid sequence SEQ ID NO: 26, in the sample. In a clinical setting, samples may be prepared (e.g., by digestion) to include a glycopeptide consisting of, or essentially of amino acid sequence SEQ ID NO: 27, in the sample. In a clinical setting, samples may be prepared (e.g., by digestion) to include a glycopeptide consisting of, or essentially of amino acid sequence SEQ ID NO: 28, in the sample. In a clinical setting, samples may be prepared (e.g., by digestion) to include a glycopeptide consisting of, or essentially of amino acid sequence SEQ ID NO: 30, in the sample. In a clinical setting, samples may be prepared (e.g., by digestion) to include a glycopeptide consisting of, or essentially of amino acid sequence SEQ ID NO: 31, in the sample. In a clinical setting, samples may be prepared (e.g., by digestion) to include a glycopeptide consisting of, or essentially of amino acid sequence SEQ ID NO: 34, in the sample. In a clinical setting, samples may be prepared (e.g., by digestion) to include a glycopeptide consisting of, or essentially of amino acid sequence SEQ ID NO: 35, in the sample. In a clinical setting, samples may be prepared (e.g., by digestion) to include a glycopeptide consisting of, or essentially of amino acid sequence SEQ ID NO: 36, in the sample. In a clinical setting, samples may be prepared (e.g., by digestion) to include a glycopeptide consisting of, or essentially of amino acid sequence SEQ ID NO: 37, in the sample. In a clinical setting, samples may be prepared (e.g., by digestion) to include a glycopeptide consisting of, or essentially of amino acid sequence SEQ ID NO: 38, in the sample. In some examples, as described below, the presence, absolute amount, and/or relative amount of a glycopeptide is determined by analyzing the MS results. In some examples, the MS results are analyzed using machine learning.

In a clinical setting, samples may be prepared (e.g., by digestion) to include one or more glycopeptides consisting essentially of an amino acid sequence selected from the group consisting of SEQ ID NOs: 5, 8-11, 13-14, 16-22, 26-28, 30-31, and 34-38. In a clinical setting, samples may be prepared (e.g., by digestion) to include one or more glycopeptides consisting essentially of an amino acid sequence selected from the group consisting of SEQ ID NOs: 3, 7, 9, 28, 29, 32, and 33. In a clinical setting, samples may be prepared (e.g., by digestion) to include one or more glycopeptides consisting essentially of an amino acid sequence selected from the group consisting of SEQ ID NOs: 1-4, 6-7, 12, 15, 23-25, 28, 29, and 32.

In some examples, the amount of a glycan or glycopeptide may be assessed by comparing the amount of one or more glycopeptides consisting of an amino acid sequence selected from the group consisting of SEQ ID NOs: 5, 8-11, 13-14, 16-22, 26-28, 30-31, and 34-38 to the concentration of another biomarker. In some examples, the amount of a glycan or glycopeptide may be assessed by comparing the amount of one or more glycopeptides consisting of an amino acid sequence selected from the group consisting of SEQ ID NOs: 3, 7, 9, 28, 29, 32, and 33 to the concentration of another biomarker. In some examples, the amount of a glycan or glycopeptide may be assessed by comparing the amount of one or more glycopeptides consisting of an amino acid sequence selected from the group consisting of SEQ ID NOs: 1-4, 6-7, 12, 15, 23-25, 28, 29, and 32 to the concentration of another biomarker.

In some examples, the amount of a glycan or glycopeptide may be assessed by comparing the amount of one or more glycopeptides consisting essentially of an amino acid sequence selected from the group consisting of SEQ ID NOs: 5, 8-11, 13-14, 16-22, 26-28, 30-31, and 34-38 to the concentration of another biomarker. In some examples, the amount of a glycan or glycopeptide may be assessed by comparing the amount of one or more glycopeptides consisting essentially of an amino acid sequence selected from the group consisting of SEQ ID NOs: 3, 7, 9, 28, 29, 32, and 33 to the concentration of another biomarker. In some examples, the amount of a glycan or glycopeptide may be assessed by comparing the amount of one or more glycopeptides consisting essentially of an amino acid sequence selected from the group consisting of SEQ ID NOs: 1-4, 6-7, 12, 15, 23-25, 28, 29, and 32 to the concentration of another biomarker.

In some examples, the amount of a glycan or glycopeptide may be assessed by comparing the amount of one or more glycopeptides consisting of an amino acid sequence selected from the group consisting of SEQ ID NOs: 5, 8-11, 13-14, 16-22, 26-28, 30-31, and 34-38 to the amount of one or more glycopeptides consisting of an amino acid sequence selected from the group consisting of SEQ ID NOs: 5, 8-11, 13-14, 16-22, 26-28, 30-31, and 34-38. In some examples, the amount of a glycan or glycopeptide may be assessed by comparing the amount of one or more glycopeptides consisting of an amino acid sequence selected from the group consisting of SEQ ID NOs: 3, 7, 9, 28, 29, 32, and 33 to the amount of one or more glycopeptides consisting of an amino acid sequence selected from the group consisting of SEQ ID NOs: 3, 7, 9, 28, 29, 32, and 33. In some examples, the amount of a glycan or glycopeptide may be assessed by comparing the amount of one or more glycopeptides consisting of an amino acid sequence selected from the group consisting of SEQ ID NOs: 1-4, 6-7, 12, 15, 23-25, 28, 29, and 32 to the amount of one or more glycopeptides consisting of an amino acid sequence selected from the group consisting of SEQ ID NOs: 1-4, 6-7, 12, 15, 23-25, 28, 29, and 32.

In some examples, the amount of a glycan or glycopeptide may be assessed by comparing the amount of one or more glycopeptides consisting essentially of an amino acid sequence selected from the group consisting of SEQ ID NOs: 5, 8-11, 13-14, 16-22, 26-28, 30-31, and 34-38 to the amount of one or more glycopeptides consisting essentially of an amino acid sequence selected from the group consisting of SEQ ID NOs: 5, 8-11, 13-14, 16-22, 26-28, 30-31, and 34-38. In some examples, the amount of a glycan or glycopeptide may be assessed by comparing the amount of one or more glycopeptides consisting essentially of an amino acid sequence selected from the group consisting of SEQ ID NOs: 3, 7, 9, 28, 29, 32, and 33 to the amount of one or more glycopeptides consisting essentially of an amino acid sequence selected from the group consisting of SEQ ID NOs: 3, 7, 9, 28, 29, 32, and 33. In some examples, the amount of a glycan or glycopeptide may be assessed by comparing the amount of one or more glycopeptides consisting essentially of an amino acid sequence selected from the group consisting of SEQ ID NOs: 1-4, 6-7, 12, 15, 23-25, 28, 29, 32 to the amount of one or more glycopeptides consisting essentially of an amino acid sequence selected from the group consisting of SEQ ID NOs: 1-4, 6-7, 12, 15, 23-25, 28, 29, 32.

In some examples, including any of the foregoing, the kit may include software for computing the normalization of a glycopeptide MRM transition signal.

In some examples, including any of the foregoing, the kit may include software for quantifying the amount of a glycopeptide consisting of, or consisting essentially of, an amino acid sequence selected from the group consisting of SEQ ID NOs: 5, 8-11, 13-14, 16-22, 26-28, 30-31, and 34-38. In some examples, including any of the foregoing, the kit may include software for quantifying the amount of a glycopeptide consisting of, or consisting essentially of, an amino acid sequence selected from the group consisting of SEQ ID NOs: 3, 7, 9, 28, 29, 32, and 33. In some examples, including any of the foregoing, the kit may include software for quantifying the amount of a glycopeptide consisting of, or consisting essentially of, an amino acid sequence selected from the group consisting of SEQ ID NOs: 1-4, 6-7, 12, 15, 23-25, 28, 29, and 32.

In some examples, including any of the foregoing, the kit may include software for quantifying the relative amount of a glycopeptide consisting of, or consisting essentially of, an amino acid sequence selected from the group consisting of SEQ ID NOs: 5, 8-11, 13-14, 16-22, 26-28, 30-31, and 34-38. In some examples, including any of the foregoing, the kit may include software for quantifying the relative amount of a glycopeptide consisting of, or consisting essentially of, an amino acid sequence selected from the group consisting of SEQ ID NOs: 3, 7, 9, 28, 29, 32, and 33. In some examples, including any of the foregoing, the kit may include software for quantifying the relative amount of a glycopeptide consisting of, or consisting essentially of, an amino acid sequence selected from the group consisting of SEQ ID NOs: 1-4, 6-7, 12, 15, 23-25, 28, 29, 32.

In some examples, including any of the foregoing, a trained model is stored on a server which is accessed by a clinician performing a method, set forth herein. In some examples, the clinician inputs the quantification of the MRM transition signals from a patient's sample into a trained model which are stored on a server. In some examples, the server is accessed by the internet, wireless communication, or other digital or telecommunication methods.

In some examples, including any of the foregoing, a trained model is stored on a server which is accessed by a clinician performing a method, set forth herein. In some examples, the clinician inputs the quantification of the glycopeptide or glycopeptides consisting of, or consisting essentially of, an amino acid sequence selected from the group consisting of SEQ ID NOs: 5, 8-11, 13-14, 16-22, 26-28, 30-31, and 34-38 from a patient's sample into a trained model which are stored on a server. In some examples, the clinician inputs the quantification of the glycopeptide or glycopeptides consisting of, or consisting essentially of, an amino acid sequence selected from the group consisting of SEQ ID NOs: 3, 7, 9, 28, 29, 32, and 33 from a patient's sample into a trained model which are stored on a server. In some examples, the clinician inputs the quantification of the glycopeptide or glycopeptides consisting of, or consisting essentially of, an amino acid sequence selected from the group consisting of SEQ ID NOs: 1-4, 6-7, 12, 15, 23-25, 28, 29, 32 from a patient's sample into a trained model which are stored on a server.

In some examples, the server is accessed by the internet, wireless communication, or other digital or telecommunication methods.

### VIII. EXAMPLES

Chemicals and Reagents. Glycoprotein standards purified from human serum/plasma were purchased from Sigma-Aldrich (St. Louis, MO). Sequencing grade trypsin was purchased from Promega (Madison, WI). Dithiothreitol (DTT) and iodoacetamide (IAA) were purchased from Sigma-Aldrich (St. Louis, MO). Human serum was purchased from Sigma-Aldrich (St. Louis, MO).

Sample Preparation. Serum samples and glycoprotein standards were reduced, alkylated and then digested with trypsin in a water bath at 37° C. for 18 hours.

LC-MS/MS Analysis. For quantitative analysis, tryptic digested serum samples were injected into an high performance liquid chromatography (HPLC) system coupled to triple quadrupole (QqQ) mass spectrometer. The separation was conducted on a reverse phase column. Solvents A and B used in the binary gradient were composed of mixtures of water, acetonitrile and formic acid. Typical positive ionization source parameters were utilized after source tuning with vendor supplied standards. The following ranges were evaluated: source spray voltage between 3-5 kV, temperature 250-350° C., and nitrogen sheath gas flow rate 20-40 psi. The scan mode of instrument used was dMRM.

For the glycoproteomic analysis, enriched serum glycopeptides were analyzed with a Q Exactive™ Hybrid Quadrupole-Orbitrap™ Mass spectrometer or an Agilent 6495B Triple Quadrupole LC/MS.

MRM Mass Spectroscopy settings, sample preparation, and reagents are set forth in Li, et al., Site-Specific Glycosylation Quantification of 50 serum Glycoproteins Enhanced by Predictive Glycopeptidomics for Improved Disease Biomarker Discovery, Anal. Chem. 2019, 91, 5433-5445; DOI: 10.1021/acs.analchem.9b00776, the entire contents of which are herein incorporated by reference in its entirety for all purposes.

### Example 1—Identifying Glycopeptide Biomarkers

This Example refers to FIG. 15 illustrated in International PCT Patent Application No. PCT/US2020/0162861, filed Jan. 31, 2020, which is herein incorporated by reference in its entirety for all purposes.

As shown in FIG. 15, in step 1, samples from patients having colorectal cancer or advanced adenoma and samples from patients not having colorectal cancer or advanced adenoma were provided. In step 2, the samples were digested using protease enzymes to form glycopeptide fragments. In step 3, the glycopeptide fragments were introduced into a tandem LC-MS/MS instrument to analyze the retention time and MRM-MS transition signals associated with the aforementioned samples. In step 4, glycopeptides and glycan biomarkers were identified. Machine learning algorithms selected MRM-MS transition signals from a series of MS spectra and associated those signals with the calculated mass of certain glycopeptide fragments. An automated detection method of boundaries for mass spectrometry peaks was used, the method as disclosed in U.S. patent Ser. No. 16/833,324, as filed on Mar. 27, 2020, is herein incorporated by reference in its entirety for all purposes.

In step 5, the glycopeptides identified in samples from patients having colorectal cancer or advanced adenoma were compared using machine learning algorithms, including lasso regression, with the glycopeptides identified in samples from patients not having colorectal cancer or advanced adenoma. This comparison included a comparison of the types, absolute amounts, and relative amounts of glycopeptides. From this comparison, normalization of peptides, and relative abundance of glycopeptides was calculated.

### Example 2—Identifying Glycopeptide Biomarkers

This Example refers to FIG. 16 illustrated in International PCT Patent Application No. PCT/US2020/0162861, filed Jan. 31, 2020, which is herein incorporated by reference in its entirety for all purposes.

As shown in FIG. 16, in step 1, samples from patients are provided. In step 2, the samples were digested using protease enzymes to form glycopeptide fragments. In step 3, the glycopeptide fragments were introduced into a tandem LC-MS/MS instrument to analyze the retention time and MRM-MS transition signals associated with the sample. In step 4, the glycopeptides were identified using machine learning algorithms which select MRM-MS transition signals and associate those signals with the calculated mass of certain glycopeptide fragments. In step 5, the data is normalized. In step 6, machine learning is used to analyzed the normalized data to identify biomarkers indicative of a patient having colorectal cancer or advanced adenoma.

TABLE 1
Transition Numbers for Glycopeptides from Glycopeptide Groups.

| Transition | | |
|---|---|---|
| Number | Compound Name | Compound. Group |
| 1 | A1AT- | GP001-P01009\|Alpha-1- |
| | GP001_107_5411 | antitrypsin\|A1AT |
| 2 | A1AT- | GP001-P01009\|Alpha-1- |
| | GP001_271_5402 | antitrypsin\|A1AT |
| 3 | A1AT- | GP001-P01009\|Alpha-1- |
| | GP001_271_6503 | antitrypsin\|A1AT |
| 4 | A1BG- | GP002-P04217\|Alpha-1B- |
| | GP002_179_5421/5402 | glycoprotein\|A1BG |
| 5 | A2MG- | GP004-P01023\|Alpha-2- |
| | GP004_1424_5402 | macroglobulin\|A2MG |
| 6 | A2MG- | GP004-P01023\|Alpha-2- |
| | GP004_1424_5412 | macroglobulin\|A2MG |
| 7 | A2MG- | GP004-P01023\|Alpha-2- |
| | GP004_55_5402 | macroglobulin\|A2MG |
| 8 | A2MG- | GP004-P01023\|Alpha-2- |
| | GP004_869_5401 | macroglobulin\|A2MG |
| 9 | A2MG- | GP004-P01023\|Alpha-2- |
| | GP004_869_6301 | macroglobulin\|A2MG |
| 10 | AACT- | GP005-P01011\|Alpha-1- |
| | GP005_271_7603 | antichymotrypsin\|AACT |
| 11 | AGP1- | GP007-P02763\|Alpha-1-acid |
| | GP007_103_9804 | glycoprotein 1\|AGP1 |
| 12 | AGP1- | GP007-P02763\|Alpha-1-acid |
| | GP007_33_6501 | glycoprotein 1\|AGP1 |
| 13 | AGP1- | GP007-P02763\|Alpha-1-acid |
| | GP007_93_6502 | glycoprotein 1\|AGP1 |
| 14 | AGP1- | GP007-P02763\|Alpha-1-acid |
| | GP007_93_7611 | glycoprotein 1\|AGP1 |
| 15 | AGP2- | GP008-P19652\|Alpha-1-acid |
| | GP008_103_6503 | glycoprotein 2\|AGP2 |
| 16 | APOC3- | GP012-P02656\|Apolipoprotein |
| | GP012_74Aoff_1102 | C-III\|APOC3 |
| 17 | APOD- | GP014-P05090\|Apolipoprotein |
| | GP014_98_5402/5421 | D\|APOD |
| 18 | APOD- | GP014-P05090\|Apolipoprotein |
| | GP014_98_5410 | D\|APOD |
| 19 | APOD- | GP014-P05090\|Apolipoprotein |
| | GP014_98_6510 | D\|APOD |
| 20 | APOD- | GP014-P05090\|Apolipoprotein |
| | GP014_98_6530 | D\|APOD |
| 21 | APOD- | GP014-P05090\|Apolipoprotein |
| | GP014_98_9800 | D\|APOD |
| 22 | CAN3- | GP022-P20807\|Calpain-3\|CAN3 |
| | GP022_366_6513 | |
| 23 | CERU- | GP023- |
| | GP023_138_5412 | P00450\|Ceruloplasmin\|CERU |
| 24 | CERU- | GP023- |
| | GP023_138_5421/5402 | P00450\|Ceruloplasmin\|CERU |
| 25 | FETUA- | GP036-P02765\|Alpha-2-HS- |
| | GP036_176_5401 | glycoprotein\|FETUA |
| 26 | FETUA- | GP036-P02765\|Alpha-2-HS- |
| | GP036_176_6513 | glycoprotein\|FETUA |
| 27 | HPT-GP044_207_5401 | GP044-P00738\|Haptoglobin\|HPT |
| 28 | HPT- | GP044-P00738\|Haptoglobin\|HPT |
| | GP044_241_5402/5421 | |
| 29 | HPT-GP044_241_5511 | GP044-P00738\|Haptoglobin\|HPT |
| 30 | HPT-GP044_241_6511 | GP044-P00738\|Haptoglobin\|HPT |
| 31 | HPT-GP044_241_7511 | GP044-P00738\|Haptoglobin\|HPT |
| 32 | IgM-GP053_46_4310 | GP053-P01871\|Immunoglobulin |
| | | heavy chain constant μ\|IgM |
| 33 | KLKB1- | GP056-P03952\|Plasma |
| | GP056_494_6503 | Kallikrein\|KLKB1 |
| 34 | PON1- | GP060-P27169\|Serum |
| | GP060_324_5420 | paraoxonase/arylesterase 1\|PON1 |
| 35 | PON1- | GP060-P27169\|Serum |
| | GP060_324_6501 | paraoxonase/arylesterase 1\|PON1 |
| 36 | PON1- | GP060-P27169\|Serum |
| | GP060_324_6502 | paraoxonase/arylesterase 1\|PON1 |
| 37 | UN13A- | GP066-Q9UPW8\|Protein unc- |
| | GP066_1005_5431 | 13HomologA\|UN13A |
| 38 | UN13A- | GP066-Q9UPW8\|Protein unc- |
| | GP066_1005_7420 | 13HomologA\|UN13A |

TABLE 2
Transition Numbers with Precursor Ion and Product Ion (m/z)

| Transition | | |
|---|---|---|
| number | Precursor ion | Product ion |
| 1 | 1151.6 | 366.1 |
| 2 | 991.2 | 366.1 |
| 3 | 1155 | 274.1 |
| 4 | 1209.5 | 366.1 |
| 5 | 1093.1 | 366.1 |
| 6 | 1129.6 | 366.1 |
| 7 | 1151.6 | 366.1 |
| 8 | 1066.7 | 366.1 |
| 9 | 1322.3 | 366.1 |
| 10 | 1245.8 | 366.1 |
| 11 | 1256.8 | 366.1 |
| 12 | 1215 | 366.1 |
| 13 | 1122.5 | 366.1 |
| 14 | 1177.2 | 366.1 |
| 15 | 1208.6 | 366.1 |
| 16 | 1005.1 | 274.1 |
| 17 | 1115.7 | 366.1 |
| 18 | 1341.6 | 366.1 |
| 19 | 1098 | 366.1 |
| 20 | 1171 | 366.1 |
| 21 | 1335.3 | 366.1 |
| 22 | 1236.2 | 366.1 |
| 23 | 1061.9 | 366.1 |
| 24 | 1367.2 | 366.1 |
| 25 | 1070.4 | 366.1 |
| 26 | 1343.8 | 366.1 |
| 27 | 1124.8 | 366.1 |
| 28 | 1001 | 366.1 |
| 29 | 1015.5 | 366.1 |
| 30 | 1055.7 | 366.1 |
| 31 | 1096.2 | 366.1 |
| 32 | 896.7 | 204.1 |
| 33 | 1277.8 | 366.1 |
| 34 | 1057.7 | 366.1 |
| 35 | 1149.3 | 366.1 |
| 36 | 1221.5 | 366.1 |
| 37 | 1227.5 | 366.1 |
| 38 | 1199.2 | 366.1 |

MS1 and MS2 resolution was 1 unit.

TABLE 3
Transition Numbers with Retention Time, ΔRetention
Time, Fragmentor and Collision Energy

| | Ret | | | |
|---|---|---|---|---|
| Transition | Time | | | Collision |
| Number | (min) | Delta Ret Time | Fragmentor | Energy |
| 1 | 42.5 | 3 | 380 | 28 |
| 2 | 29.8 | 3 | 380 | 24 |
| 3 | 30.2 | 3 | 380 | 30 |
| 4 | 24 | 2 | 380 | 36 |
| 5 | 43.7 | 3 | 380 | 22 |
| 6 | 43.7 | 3 | 380 | 23 |
| 7 | 41.1 | 3 | 380 | 23 |
| 8 | 26 | 2 | 380 | 22 |
| 9 | 26.2 | 2 | 380 | 24 |
| 10 | 19.7 | 1.2 | 380 | 35 |
| 11 | 4 | 1 | 380 | 31 |
| 12 | 27.9 | 2 | 380 | 30 |
| 13 | 17.1 | 1.2 | 380 | 28 |
| 14 | 17 | 1.2 | 380 | 30 |
| 15 | 3 | 1.2 | 380 | 30 |
| 16 | 30 | 2 | 380 | 24 |
| 17 | 21.8 | 1.2 | 380 | 34 |
| 18 | 21.8 | 1.2 | 380 | 35 |
| 19 | 21.5 | 1.2 | 380 | 34 |
| 20 | 22.2 | 1.2 | 380 | 35 |
| 21 | 21.8 | 1.2 | 380 | 39 |
| 22 | 24.3 | 2 | 380 | 37 |
| 23 | 13 | 2 | 380 | 33 |
| 24 | 13.2 | 2 | 380 | 40 |
| 25 | 21.3 | 1.2 | 380 | 26 |
| 26 | 22 | 1.2 | 380 | 34 |
| 27 | 10.6 | 1.4 | 380 | 30 |
| 28 | 20.7 | 1.2 | 380 | 24 |
| 29 | 21.6 | 1.2 | 380 | 25 |
| 30 | 20.6 | 1.2 | 380 | 30 |
| 31 | 21.4 | 1.2 | 380 | 30 |
| 32 | 3.9 | 1.2 | 380 | 25 |
| 33 | 20.8 | 1.2 | 380 | 38 |
| 34 | 25.3 | 2 | 380 | 33 |
| 35 | 25.2 | 2 | 380 | 35 |
| 36 | 24.9 | 2 | 380 | 37 |
| 37 | 24.3 | 2 | 380 | 37 |
| 38 | 25.9 | 2 | 380 | 36 |

Cell accelerator voltage was 5.

TABLE 4
Glycan Residue Compound Numbers, Molecular Mass, and Glycan
Fragment mass-to-charge (m/z) (+2) & (m/z) (+3) ratios

| Composition | mass | m/z (+2) | m/z (+3) |
|---|---|---|---|
| 3200 | 910.327 | 456.1708 | 304.449633 |
| 3210 | 1056.386 | 529.2003 | 353.135967 |
| 3300 | 1113.407 | 557.7108 | 372.142967 |
| 3310 | 1259.465 | 630.7398 | 420.828967 |
| 3320 | 1405.523 | 703.7688 | 469.514967 |
| 3400 | 1316.487 | 659.2508 | 439.8363 |
| 3410 | 1462.544 | 732.2793 | 488.521967 |
| 3420 | 1608.602 | 805.3083 | 537.207967 |
| 3500 | 1519.566 | 760.7903 | 507.5293 |
| 3510 | 1665.624 | 833.8193 | 556.2153 |
| 3520 | 1811.682 | 906.8483 | 604.9013 |
| 3600 | 1722.645 | 862.3298 | 575.2223 |
| 3610 | 1868.703 | 935.3588 | 623.9083 |
| 3620 | 2014.761 | 1008.3878 | 672.5943 |
| 3630 | 2160.89 | 1081.4523 | 721.303967 |
| 3700 | 1925.724642 | 963.869621 | 642.915514 |
| 3710 | 2071.782551 | 1036.898576 | 691.601484 |
| 3720 | 2217.84046 | 1109.92753 | 740.287453 |
| 3730 | 2363.898369 | 1182.956485 | 788.973423 |
| 3740 | 2509.956277 | 1255.985439 | 837.659392 |
| 4200 | 1072.380603 | 537.1976015 | 358.467501 |
| 4210 | 1218.438512 | 610.226556 | 407.153471 |
| 4300 | 1275.459976 | 638.737288 | 426.160625 |
| 4301 | 1566.555392 | 784.284996 | 523.192431 |
| 4310 | 1421.517884 | 711.766242 | 474.846595 |
| 4311 | 1712.613301 | 857.3139505 | 571.8784 |
| 4320 | 1567.575793 | 784.7951965 | 523.532564 |
| 4400 | 1478.539348 | 740.276974 | 493.853749 |
| 4401 | 1769.634765 | 885.8246825 | 590.885555 |
| 4410 | 1624.597257 | 813.3059285 | 542.539719 |
| 4411 | 1915.692673 | 958.8536365 | 639.571524 |
| 4420 | 1770.655166 | 886.334883 | 591.225689 |
| 4421 | 2061.750582 | 1031.882591 | 688.257494 |
| 4430 | 1916.713074 | 959.363837 | 639.911658 |
| 4431 | 2207.808491 | 1104.911546 | 736.943464 |
| 4500 | 1681.618721 | 841.8166605 | 561.546874 |
| 4501 | | 1.0073 | 1.0073 |
| 4510 | 1972.714137 | 987.3643685 | 658.578679 |
| 4511 | 2118.772046 | 1060.393323 | 707.264649 |
| 4520 | 1973.734538 | 987.874569 | 658.918813 |
| 4521 | 2264.829955 | 1133.422278 | 755.950618 |
| 4530 | 2119.792447 | 1060.903524 | 707.604782 |
| 4531 | 2410.887864 | 1206.451232 | 804.636588 |
| 4540 | 2265.850356 | 1133.932478 | 756.290752 |
| 4541 | 2556.945772 | 1279.480186 | 853.322557 |
| 4600 | 1884.698093 | 943.3563465 | 629.239998 |
| 4601 | 2175.79351 | 1088.904055 | 726.271803 |
| 4610 | 2030.756002 | 1016.385301 | 677.925967 |
| 4611 | 2321.851418 | 1161.933009 | 774.957773 |
| 4620 | 2176.813911 | 1089.414256 | 726.611937 |
| 4621 | 2467.909327 | 1234.961964 | 823.643742 |
| 4630 | 2322.87182 | 1162.44321 | 775.297907 |
| 4631 | 2613.967236 | 1307.990918 | 872.329712 |
| 4641 | 2760.025145 | 1381.019873 | 921.015682 |
| 4650 | 2614.987637 | 1308.501119 | 872.669846 |
| 4700 | 2087.777466 | 1044.896033 | 696.933122 |
| 4701 | 2378.872882 | 1190.443741 | 793.964927 |
| 4710 | 2233.835374 | 1117.924987 | 745.619091 |
| 4711 | 2524.930791 | 1263.472696 | 842.650897 |
| 4720 | 2379.893283 | 1190.953942 | 794.305061 |
| 4730 | 2525.951192 | 1263.982896 | 842.991031 |
| 5200 | 1234.433426 | 618.224013 | 412.485109 |
| 5210 | 1380.491335 | 691.2529675 | 461.171078 |
| 5300 | 1437.512799 | 719.7636995 | 480.178233 |
| 5301 | 1728.608215 | 865.3114075 | 577.210038 |
| 5310 | 1583.570708 | 792.792654 | 528.864203 |
| 5311 | 1874.666124 | 938.340362 | 625.896008 |
| 5320 | 1729.628617 | 865.8216085 | 577.550172 |
| 5400 | 1640.592171 | 821.3033855 | 547.871357 |
| 5401 | 1931.687588 | 966.851094 | 644.903163 |
| 5402 | 2222.783005 | 1112.398803 | 741.934968 |
| 5410 | 1786.65008 | 894.33234 | 596.557327 |
| 5411 | 2077.745497 | 1039.880049 | 693.589132 |
| 5412 | 2368.840913 | 1185.427757 | 790.620938 |
| 5420 | 1932.707989 | 967.3612945 | 645.243296 |
| 5421 | 2223.803406 | 1112.909003 | 742.275102 |
| 5430 | 2078.765898 | 1040.390249 | 693.929266 |
| 5431 | 2369.861314 | 1185.937957 | 790.961071 |
| 5432 | 2660.956731 | 1331.485666 | 887.992877 |
| 5500 | 1843.671544 | 922.843072 | 615.564481 |
| 5501 | 2134.766961 | 1068.390781 | 712.596287 |
| 5502 | 2425.862377 | 1213.938489 | 809.628092 |
| 5510 | 1989.729453 | 995.8720265 | 664.250451 |
| 5511 | 2280.824869 | 1141.419735 | 761.282256 |
| 5512 | 2571.920286 | 1286.967443 | 858.314062 |
| 5520 | 2135.787362 | 1068.900981 | 712.936421 |
| 5521 | 2426.882778 | 1214.448689 | 809.968226 |
| 5522 | 2717.978195 | 1359.996398 | 907.000032 |
| 5530 | 2281.84527 | 1141.929935 | 761.62239 |
| 5531 | 2572.940687 | 1287.477644 | 858.654196 |
| 5541 | 2718.998596 | 1360.506598 | 907.340165 |
| 5600 | 2046.750917 | 1024.382759 | 683.257606 |
| 5601 | 2337.846333 | 1169.930467 | 780.289411 |
| 5602 | 2628.94175 | 1315.478175 | 877.321217 |
| 5610 | 2192.808825 | 1097.411713 | 731.943575 |
| 5611 | 2483.904242 | 1242.959421 | 828.975381 |
| 5612 | 2774.999658 | 1388.507129 | 926.007186 |
| 5620 | 2338.866734 | 1170.440667 | 780.629545 |
| 5621 | 2629.962151 | 1315.988376 | 877.66135 |
| 5631 | 2776.020059 | 1389.01733 | 926.34732 |
| 5650 | 2777.040461 | 1389.527531 | 926.687454 |
| 5700 | 2249.830289 | 1125.922445 | 750.95073 |
| 5701 | 2540.925706 | 1271.470153 | 847.982535 |
| 5702 | 2832.021122 | 1417.017861 | 945.014341 |
| 5710 | 2395.888198 | 1198.951399 | 799.636699 |
| 5711 | 2686.983614 | 1344.499107 | 896.668505 |
| 5712 | 2978.079031 | 1490.046816 | 993.70031 |
| 5720 | 2541.946107 | 1271.980354 | 848.322669 |
| 5721 | 2833.041523 | 1417.528062 | 945.354474 |
| 5730 | 2688.004016 | 1345.009308 | 897.008639 |
| 5731 | 2979.099432 | 1490.557016 | 994.040444 |
| 6200 | 1396.48625 | 699.250425 | 466.502717 |
| 6210 | 1542.544159 | 772.2793795 | 515.188686 |
| 6300 | 1599.565622 | 800.790111 | 534.195841 |
| 6301 | 1890.661039 | 946.3378195 | 631.227646 |
| 6310 | 1745.623531 | 873.8190655 | 582.88181 |
| 6311 | 2036.718948 | 1019.366774 | 679.913616 |
| 6320 | 1891.68144 | 946.84802 | 631.56778 |
| 6400 | 1802.644995 | 902.3297975 | 601.888965 |
| 6401 | 2093.740411 | 1047.877506 | 698.92077 |
| 6402 | 2384.835828 | 1193.425214 | 795.952576 |
| 6410 | 1948.702904 | 975.358752 | 650.574935 |
| 6411 | 2239.79832 | 1120.90646 | 747.60674 |
| 6412 | 2530.893737 | 1266.454169 | 844.638546 |
| 6420 | 2094.760813 | 1048.387707 | 699.260904 |
| 6421 | 2385.856229 | 1193.935415 | 796.29271 |
| 6432 | 2823.009554 | 1412.512077 | 942.010485 |
| 6500 | 2005.724367 | 1003.869484 | 669.582089 |
| 6501 | 2296.819784 | 1149.417192 | 766.613895 |
| 6502 | 2587.9152 | 1294.9649 | 863.6457 |
| 6503 | 2879.010617 | 1440.512609 | 960.677506 |
| 6510 | 2151.782276 | 1076.898438 | 718.268059 |
| 6511 | 2442.877693 | 1222.446147 | 815.299864 |
| 6512 | 2733.973109 | 1367.993855 | 912.33167 |
| 6513 | 3025.068526 | 1513.541563 | 1009.36348 |
| 6520 | 2297.840185 | 1149.927393 | 766.954028 |
| 6521 | 2588.935602 | 1295.475101 | 863.985834 |
| 6522 | 2880.031018 | 1441.022809 | 961.017639 |
| 6530 | 2443.898094 | 1222.956347 | 815.639998 |
| 6531 | 2734.99351 | 1368.504055 | 912.671803 |
| 6532 | 3026.088927 | 1514.051764 | 1009.70361 |
| 6540 | 2589.956003 | 1295.985302 | 864.325968 |
| 6541 | 2881.051419 | 1441.53301 | 961.357773 |
| 6600 | 2208.80374 | 1105.40917 | 737.275213 |
| 6601 | 2499.899157 | 1250.956879 | 834.307019 |
| 6602 | 2790.994573 | 1396.504587 | 931.338824 |
| 6603 | 3082.08999 | 1542.052295 | 1028.37063 |
| 6610 | 2354.861649 | 1178.438125 | 785.961183 |
| 6611 | 2645.957065 | 1323.985833 | 882.992988 |
| 6612 | 2937.052482 | 1469.533541 | 980.024794 |
| 6613 | 3228.147898 | 1615.081249 | 1077.0566 |
| 6620 | 2500.919558 | 1251.467079 | 834.647153 |
| 6621 | 2792.014974 | 1397.014787 | 931.678958 |
| 6622 | 3083.110391 | 1542.562496 | 1028.71076 |
| 6623 | 3374.205807 | 1688.110204 | 1125.74257 |
| 6630 | 2646.977466 | 1324.496033 | 883.333122 |
| 6631 | 2938.072883 | 1470.043742 | 980.364928 |
| 6632 | 3229.168299 | 1615.59145 | 1077.39673 |
| 6640 | 2793.035375 | 1397.524988 | 932.019092 |
| 6641 | 3084.130792 | 1543.072696 | 1029.0509 |
| 6642 | 3375.226208 | 1688.620404 | 1126.0827 |
| 6652 | 3521.284117 | 1761.649359 | 1174.76867 |
| 6700 | 2411.883113 | 1206.948857 | 804.968338 |
| 6701 | 2702.978529 | 1352.496565 | 902.000143 |
| 6703 | 3285.169362 | 1643.591981 | 1096.06375 |
| 6710 | 2557.941021 | 1279.977811 | 853.654307 |
| 6711 | 2849.036438 | 1425.525519 | 950.686113 |
| 6711 | 2849.036438 | 1425.525519 | 950.686113 |
| 6712 | 3140.131854 | 1571.073227 | 1047.71792 |
| 6713 | 3431.227271 | 1716.620936 | 1144.74972 |
| 6713 | 3431.227271 | 1716.620936 | 1144.74972 |
| 6720 | 2703.99893 | 1353.006765 | 902.340277 |
| 6721 | 2995.094347 | 1498.554474 | 999.372082 |
| 6721 | 2995.094347 | 1498.554474 | 999.372082 |
| 6730 | 2850.056839 | 1426.03572 | 951.026246 |
| 6731 | 3141.152255 | 1571.583428 | 1048.05805 |
| 6740 | 2996.114748 | 1499.064674 | 999.712216 |
| 7200 | 1558.539073 | 780.2768365 | 520.520324 |
| 7210 | 1704.596982 | 853.305791 | 569.206294 |
| 7400 | 1964.697818 | 983.356209 | 655.906573 |
| 7401 | 2255.793235 | 1128.903918 | 752.938378 |
| 7410 | 2110.755727 | 1056.385164 | 704.592542 |
| 7411 | 2401.851144 | 1201.932872 | 801.624348 |
| 7412 | 2692.94656 | 1347.48058 | 898.656153 |
| 7420 | 2256.813636 | 1129.414118 | 753.278512 |
| 7421 | 2547.909052 | 1274.961826 | 850.310317 |
| 7430 | 2402.871545 | 1202.443073 | 801.964482 |
| 7431 | 2693.966961 | 1347.990781 | 898.996287 |
| 7432 | 2985.062378 | 1493.538489 | 996.028093 |
| 7500 | 2167.777191 | 1084.895896 | 723.599697 |
| 7501 | 2458.872607 | 1230.443604 | 820.631502 |
| 7510 | 2313.8351 | 1157.92485 | 772.285667 |
| 7511 | 2604.930516 | 1303.472558 | 869.317472 |
| 7512 | 2896.025933 | 1449.020267 | 966.349278 |
| 7600 | 2370.856563 | 1186.435582 | 791.292821 |
| 7601 | 2661.95198 | 1331.98329 | 888.324627 |
| 7602 | 2953.047396 | 1477.530998 | 985.356432 |
| 7603 | 3244.142813 | 1623.078707 | 1082.38824 |
| 7604 | 3535.23823 | 1768.626415 | 1179.42004 |
| 7610 | 2516.914472 | 1259.464536 | 839.978791 |
| 7611 | 2808.009889 | 1405.012245 | 937.010596 |
| 7612 | 3099.105305 | 1550.559953 | 1034.0424 |
| 7613 | 3390.200722 | 1696.107661 | 1131.07421 |
| 7614 | 3681.296138 | 1841.655369 | 1228.10601 |
| 7620 | 2662.972381 | 1332.493491 | 888.66476 |
| 7621 | 2954.067798 | 1478.041199 | 985.696566 |
| 7622 | 3245.163214 | 1623.588907 | 1082.72837 |
| 7623 | 3536.258631 | 1769.136616 | 1179.76018 |
| 7632 | 3391.221123 | 1696.617862 | 1131.41434 |
| 7640 | 2955.088199 | 1478.5514 | 986.0367 |
| 7700 | 2573.935936 | 1287.975268 | 858.985945 |
| 7701 | 2865.031352 | 1433.522976 | 956.017751 |
| 7702 | 3156.126769 | 1579.070685 | 1053.04956 |
| 7703 | 3447.222186 | 1724.618393 | 1150.08136 |
| 7710 | 2719.993845 | 1361.004223 | 907.671915 |
| 7711 | 3011.089261 | 1506.551931 | 1004.70372 |
| 7712 | 3302.184678 | 1652.099639 | 1101.73553 |
| 7713 | 3593.280094 | 1797.647347 | 1198.76733 |
| 7714 | 3884.375511 | 1943.195056 | 1295.79914 |
| 7720 | 2866.051754 | 1434.033177 | 956.357885 |
| 7721 | 3157.14717 | 1579.580885 | 1053.38969 |
| 7722 | 3448.242587 | 1725.128594 | 1150.4215 |
| 7730 | 3012.109662 | 1507.062131 | 1005.04385 |
| 7731 | 3303.205079 | 1652.60984 | 1102.07566 |
| 7732 | 3594.300495 | 1798.157548 | 1199.10747 |
| 7740 | 3158.167571 | 1580.091086 | 1053.72982 |
| 7741 | 3449.262988 | 1725.638794 | 1150.76163 |
| 7751 | 3595.320897 | 1798.667749 | 1199.4476 |
| 8200 | 1720.591897 | 861.3032485 | 574.537932 |
| 9200 | 1882.64472 | 942.32966 | 628.55554 |
| 9210 | 2028.702629 | 1015.358615 | 677.24151 |
| 10200 | 2044.697544 | 1023.356072 | 682.573148 |
| 11200 | 2206.750367 | 1104.382484 | 736.590756 |
| 12200 | 2368.80319 | 1185.408895 | 790.608363 |

TABLE 5
Glycan Residue Compound Numbers,
Molecular Mass, and Classification

| Compound | Glycan Mass | Glycan Composition | Class |
|---|---|---|---|
| 3200 | 910.328 | GlcNAc₂Man₃ | HM |
| 3200 | | | |
| 3210 | 1056.386 | GlcNAc₂Man₃Fuc₁ | HM-F |
| 3210 | | | |
| 3300 | 1113.407 | Hex₃HexNAc₃ | C |
| 3300 | | | |
| 3310 | 1259.465 | Hex₃HexNAc₃Fuc₁ | C-F |
| 3310 | | | |
| 3320 | 1405.523 | Hex₃HexNAc₃Fuc₂ | C-F |
| 3400 | 1316.487 | Hex₃HexNAc₄ | C |
| 3410 | 1462.544 | Hex₃HexNAc₄Fuc₁ | C-F |
| 3410 | | | |
| 3420 | 1608.602 | Hex₃HexNAc₄Fuc₂ | C-F |
| 3500 | 1519.566 | Hex₃HexNAc₅ | C |
| 3510 | 1665.624 | Hex₃HexNAc₅Fuc₁ | C-F |
| 3520 | 1811.682 | Hex₃HexNAc₅Fuc₂ | C-F |
| 3600 | 1722.645 | Hex₃HexNAc₆ | C |
| 3610 | 1868.703 | Hex₃HexNAc₆Fuc₁ | C-F |
| 3620 | 2014.761 | Hex₃HexNAc₆Fuc₂ | C-F |
| 3630 | 2160.819 | Hex₃HexNAc₆Fuc₃ | C-F |
| 3700 | 1925.725 | Hex₃HexNAc₇ | C |
| 3710 | 2071.783 | Hex₃HexNAc₇Fuc₁ | C-F |
| 3720 | 2217.841 | Hex₃HexNAc₇Fuc₂ | C-F |
| 3720 | 2217.841 | Hex₃HexNAc₇Fuc₂ | C-F |
| 3730 | 2363.898 | Hex₃HexNAc₇Fuc₃ | C-F |
| 3740 | 2509.956 | Hex₃HexNAc₇Fuc₄ | C-F |
| 4200 | 1072.381 | GlcNAc₂Man₄ | HM |
| 4200 | | | |
| 4210 | 1218.438 | GlcNAc₂Man₄Fuc₁ | HM-F |
| 4210 | | | |
| 4300 | 1275.460 | Hex₄HexNAc₃ | C/H |
| 4300 | | | |
| 4301 | 1566.555 | Hex₄HexNAc₃Neu5Ac₁ | C-S |
| 4301 | 1566.555 | Hex₄HexNAc₃Neu₅Ac₁ | C-S |
| 4301 | | | |
| 4310 | 1421.518 | Hex₄HexNAc₃Fuc₁ | C/H-F |
| 4310 | 1566.555 | Hex₄HexNAc₃Neu₅Ac₁ | C-S |
| 4310 | | | |
| 4311 | 1712.613 | Hex₄HexNAc₃Fuc₁Neu₅Ac₁ | C-FS |
| 4311 | | | |
| 4320 | | | |
| 4400 | 1478.539 | Hex₄HexNAc₄ | C/H |
| 4400 | | | |
| 4401 | 1769.635 | Hex₄HexNAc₄Neu5Ac₁ | C-S |
| 4410 | 1624.597 | Hex₄HexNAc₄Fuc₁ | C/H-F |
| 4410 | | | |
| 4411 | 1915.693 | Hex₄HexNAc₄Fuc₁Neu₅Ac₁ | C-FS |
| 4411 | | | |
| 4420 | 1770.655 | Hex₄HexNAc₄Fuc₂ | C/H-F |
| 4420 | | | |
| 4421 | 2061.751 | Hex₄HexNAc₄Fuc₂Neu5Ac₁ | C-FS |
| 4430 | 1916.713 | Hex₄HexNAc₄Fuc₃ | C/H-F |
| 4431 | 2207.808 | Hex₄HexNAc₄Fuc₃Neu5Ac₁ | C-FS |
| 4431 | 2207.808 | Hex₄HexNAc₄Fuc₃Neu5Ac₁ | C-FS |
| 4531 | 2410.888 | Hex₄HexNAc₅Fuc₃Neu5Ac₁ | C-FS |
| 4541 | 2556.946 | Hex₄HexNAc₅Fuc₄Neu5Ac₁ | C-FS |
| 4600 | 1884.698 | Hex₄HexNAc₆ | C |
| 4601 | 2175.794 | Hex₄HexNAc₆Neu5Ac₁ | C-S |
| 4610 | 2030.756 | Hex₄HexNAc₆Fuc₁ | C-F |
| 4611 | 2321.851 | Hex₄HexNAc₆Fuc₁Neu5Ac₁ | C-FS |
| 4620 | 2176.814 | Hex₄HexNAc₆Fuc₂ | C-F |
| 4621 | 2467.909 | Hex₄HexNAc₆Fuc₂Neu5Ac₁ | C-FS |
| 4630 | 2322.872 | Hex₄HexNAc₆Fuc₃ | C-F |
| 4641 | 2760.025 | Hex₄HexNAc₆Fuc₄Neu5Ac₁ | C-FS |
| 4650 | 2614.988 | Hex₄HexNAc₆Fuc₅ | C-F |
| 4700 | 2087.778 | Hex₄HexNAc₇ | C |
| 4701 | 2378.873 | Hex₄HexNAc₇Neu5Ac₁ | C-S |
| 4710 | 2233.835 | Hex₄HexNAc₇Fuc₁ | C-F |
| 4711 | 2524.931 | Hex₄HexNAc₇Fuc₁Neu5Ac₁ | C-FS |
| 4720 | 2379.893 | Hex₄HexNAc₇Fuc₂ | C-F |
| 4730 | 2525.951 | Hex₄HexNAc₇Fuc₃ | C-F |
| 5200 | | | |
| 5200 | | | |
| 5210 | 1380.491 | GlcNAc₂Man₅Fuc₁ | HM-F |
| 5300 | 1437.513 | Hex₅HexNAc₃ | H |
| 5300 | | | |
| 5301 | 1728.608 | Hex₅HexNAc₃Neu5Ac₁ | H-S |
| 5301 | | | |
| 5310 | 1583.571 | Hex₅HexNAc₃Fuc₁ | H-F |
| 5310 | | | |
| 5311 | 1874.666 | Hex₅HexNAc₃Fuc₁Neu5Ac₁ | H-FS |
| 5311 | | | |
| 5320 | 1729.629 | Hex₅HexNAc₃Fuc₂ | H-F |
| 5320 | | | |
| 5400 | | | |
| 5401 | | | |
| 5401 | | | |
| 5402 | | | |
| 5410 | | | |
| 5411 | | Hex₅HexNAc₄Fuc₁Neu5Ac₁ | C-FS |
| 5411 | | | |
| 5412 | | | |
| 5420 | | | |
| 5421 | | | |
| 5430 | | | |
| 5431 | 2369.861 | Hex₅HexNAc₄Fuc₃Neu5Ac₁ | C/H-FS |
| 5432 | 2660.957 | Hex₅HexNAc₄Fuc₃Neu5Ac₂ | C-FS |
| 5432 | 2660.957 | Hex₅HexNAc₄Fuc₃Neu5Ac₂ | C-FS |
| 5531 | 2572.941 | Hex₅HexNAc₅Fuc₃Neu5Ac₁ | C/H-FS |
| 5541 | 2718.999 | Hex₅HexNAc₅Fuc₄Neu5Ac₁ | C-FS |
| 5631 | 2776.020 | Hex₅HexNAc₆Fuc₃Neu5Ac₁ | C-FS |
| 5650 | 2777.040 | Hex₅HexNAc₆Fuc₅ | C-F |
| 5700 | 2249.830 | Hex₅HexNAc₇ | C |
| 5701 | 2540.926 | Hex₅HexNAc₇Neu5Ac₁ | C-S |
| 5702 | 2832.021 | Hex₅HexNAc₇Neu5Ac₂ | C-S |
| 5710 | 2395.888 | Hex₅HexNAc₇Fuc₁ | C-F |
| 5711 | 2686.984 | Hex₅HexNAc₇Fuc₁Neu5Ac₁ | C-FS |
| 5712 | 2978.079 | Hex₅HexNAc₇Fuc₁Neu5Ac₂ | C-FS |
| 5720 | 2541.946 | Hex₅HexNAc₇Fuc₂ | C-F |
| 5721 | 2833.042 | Hex₅HexNAc₇Fuc₂Neu5Ac₁ | C-FS |
| 5730 | 2688.004 | Hex₅HexNAc₇Fuc₃ | C-F |
| 5730 | 2688.004 | Hex₅HexNAc₇Fuc₃ | C-F |
| 5731 | 2979.099 | Hex₅HexNAc₇Fuc₃Neu5Ac₁ | C-FS |
| 6200 | | | |
| 6200 | | | |
| 6210 | 1542.544 | GlcNAc₂Man₆Fuc₁ | HM-F |
| 6300 | 1599.566 | Hex₆HexNAc₃ | H |
| 6300 | | | |
| 6301 | 1890.661 | Hex₆HexNAc₃Neu5Ac₁ | H-S |
| 6301 | | | |
| 6310 | 1745.623 | Hex₆HexNAc₃Fuc₁ | H-F |
| 6310 | | | |
| 6311 | 2036.719 | Hex₆HexNAc₃Fuc₁Neu5Ac₁ | H-FS |
| 6311 | 2036.719 | Hex₆HexNAc₃Fuc₁Neu5Ac₁ | H-FS |
| 6311 | | | |
| 6320 | 1891.681 | Hex₆HexNAc₃Fuc₂ | H-F |
| 6400 | 1802.645 | Hex₆HexNAc₄ | H |
| 6401 | 2093.740 | Hex₆HexNAc₄Neu5Ac₁ | H-S |
| 6401 | | | |
| 6402 | 2384.836 | Hex₆HexNAc₄Neu5Ac₂ | H-S |
| 6410 | 1948.703 | Hex₆HexNAc₄Fuc₁ | H-F |
| 6410 | | | |
| 6411 | 2239.798 | Hex₆HexNAc₄Fuc₁Neu5Ac₁ | H-FS |
| 6421 | 2385.856 | Hex₆HexNAc₄Fuc₂Neu5Ac₁ | H-FS |
| 6432 | 2823.009 | Hex₆HexNAc₄Fuc₃Neu5Ac₂ | H-FS |
| 6500 | 2005.724 | Hex₆HexNAc₅ | C/H |
| 6500 | | | |
| 6501 | 2296.820 | Hex₆HexNAc₅Neu5Ac₁ | C/H-S |
| 6501 | | | |
| 6502 | 2587.915 | Hex₆HexNAc₅Neu5Ac₂ | C/H-S |
| 6503 | 2879.011 | Hex₆HexNAc₅Neu5Ac₃ | C-S |
| 6510 | 2151.782 | Hex₆HexNAc₅Fuc₁ | C/H-F |
| 6510 | | | |
| 6511 | 2442.878 | Hex₆HexNAc₅Fuc₁Neu5Ac₁ | C/H-FS |
| 6511 | 2442.878 | Hex₆HexNAc₅Fuc₁Neu5Ac₁ | C/H-FS |
| 6511 | | | |
| 6512 | 2733.973 | Hex₆HexNAc₅Fuc₁Neu5Ac₂ | C/H-FS |
| 6513 | 3025.068 | Hex₆HexNAc₅Fuc₁Neu5Ac₃ | C-FS |
| 6520 | | | |
| 6521 | 2588.936 | Hex₆HexNAc₅Fuc₂Neu5Ac₁ | C/H-FS |
| 6522 | 2880.031 | Hex₆HexNAc₅Fuc₂Neu5Ac₂ | C/H-FS |
| 6530 | 2443.898 | Hex₆HexNAc₅Fuc₃ | C/H-F |
| 6530 | 2879.011 | Hex₆HexNAc₅Neu5Ac₃ | C-S |
| 6531 | 2734.993 | Hex₆HexNAc₅Fuc₃Neu5Ac₁ | C/H-FS |
| 6532 | 3026.089 | Hex₆HexNAc₅Fuc₃Neu5Ac₂ | C/H-FS |
| 6603 | 3082.090 | Hex₆HexNAc₆Neu5Ac₃ | C-S |
| 6623 | 3374.206 | Hex₆HexNAc₆Fuc₂Neu5Ac₃ | C-FS |
| 6630 | 3082.090 | Hex₆HexNAc₆Neu5Ac₃ | C-S |
| 6631 | 2938.073 | Hex₆HexNAc₆Fuc₃Neu5Ac₁ | C-FS |
| 6632 | 3229.168 | Hex₆HexNAc₆Fuc₃Neu5Ac₂ | C-FS |
| 6641 | 3084.131 | Hex₆HexNAc₆Fuc₄Neu5Ac₁ | C-FS |
| 6642 | 3375.226 | Hex₆HexNAc₆Fuc₄Neu5Ac₂ | C-FS |
| 6652 | 3521.284 | Hex₆HexNAc₆Fuc₅Neu5Ac₂ | C-FS |
| 6713 | 3431.227 | Hex₆HexNAc₇Fuc₁Neu5Ac₃ | C-FS |
| 6731 | 3141.152 | Hex₆HexNAc₇Fuc₃Neu5Ac₁ | C-FS |
| 6740 | 2996.115 | Hex₆HexNAc₇Fuc₄ | C-F |
| 7200 | 1558.539 | GlcNAc₂Man₇ | HM |
| 7200 | | | |
| 7200 | | | |
| 7210 | 1704.597 | GlcNAc₂Man₇Fuc₁ | HM-F |
| 7400 | 1964.698 | Hex₇HexNAc₄ | H |
| 7400 | | | |
| 7401 | 2255.793 | Hex₇HexNAc₄Neu5Ac₁ | H-S |
| 7410 | 2110.756 | Hex₇HexNAc₄Fuc₁ | H-F |
| 7411 | 2401.851 | Hex₇HexNAc₄Fuc₁Neu5Ac₁ | H-FS |
| 7412 | 2692.946 | Hex₇HexNAc₄Fuc₁Neu5Ac₂ | H-FS |
| 7420 | 2256.814 | Hex₇HexNAc₄Fuc₂ | H-F |
| 7421 | 2547.909 | Hex₇HexNAc₄Fuc₂Neu5Ac₁ | H-FS |
| 7430 | 2402.871 | Hex₇HexNAc₄Fuc₃ | H-F |
| 7431 | 2693.967 | Hex₇HexNAc₄Fuc₃Neu5Ac₁ | H-FS |
| 7432 | 2985.062 | Hex₇HexNAc₄Fuc₃Neu5Ac₂ | H-FS |
| 7500 | 2167.777 | Hex₇HexNAc₅ | H |
| 7500 | 2167.777 | Hex₇HexNAc₅ | H |
| 7511 | 2604.930 | Hex₇HexNAc₅Fuc₁Neu5Ac₁ | H-FS |
| 7512 | 2896.026 | Hex₇HexNAc₅Fuc₁Neu5Ac₂ | H-FS |
| 7601 | 2661.952 | Hex₇HexNAc₆Neu5Ac₁ | C-S |
| 7602 | 2953.047 | Hex₇HexNAc₆Neu5Ac₂ | C-S |
| 7610 | 2516.914 | Hex₇HexNAc₆Fuc₁ | C-F |
| 7610 | | | |
| 7611 | 2808.010 | Hex₇HexNAc₆Fuc₁Neu5Ac₁ | C-FS |
| 7611 | | | |
| 7612 | 3099.105 | Hex₇HexNAc₆Fuc₁Neu5Ac₂ | C-FS |
| 7613 | 3390.201 | Hex₇HexNAc₆Fuc₁Neu5Ac₃ | C-FS |
| 7620 | 2662.972 | Hex₇HexNAc₆Fuc₂ | C-F |
| 7621 | 2954.068 | Hex₇HexNAc₆Fuc₂Neu5Ac₁ | C-FS |
| 7640 | 2955.088 | Hex₇HexNAc₆Fuc₄ | C-F |
| 7713 | 3593.280 | Hex₇HexNAc₇Fuc₁Neu5Ac₃ | C-FS |
| 7731 | 3303.205 | Hex₇HexNAc₇Fuc₃Neu5Ac₁ | C-FS |
| 7740 | 3158.168 | Hex₇HexNAc₇Fuc₄ | C-F |
| 7741 | 3449.263 | Hex₇HexNAc₇Fuc₄Neu5Ac₁ | C-FS |
| 8200 | 1720.592 | GlcNAc₂Man₉ | HM |
| 8200 | | GlcNAc₂Man₉ | |
| 8200 | | | |
| 9200 | 1882.645 | GlcNAc₂Man₉ | HM |
| 9200 | | GlcNAc₂Man₉ | |
| 9200 | | | |
| 9210 | 2028.702 | GlcNAc₂Man₉Fuc₁ | HM-F |
| 9210 | 2028.702 | GlcNAc₂Man₉Fuc₁ | HM-F |
| 10200 | 2044.697 | GlcNAc₂Man₁₀ | HM |
| 10200 | | | |
| 11200 | | | |

### Example 3—Glycoproteomic Trained Model Test

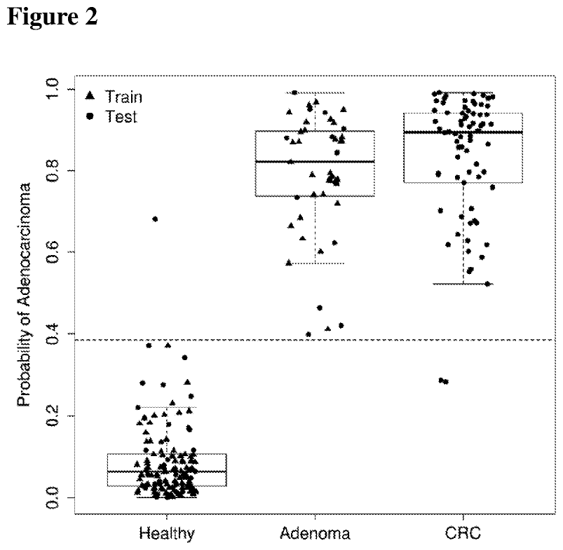

This Example refers to FIG. 1 and FIG. 2.

Markers were identified by association with diagnosed advanced adenomas (AA) or colorectal cancer (CRC). Forty-seven advanced adenoma (AA) patients and 74 colorectal cancer (CRC) patients were analyzed across all four stages of disease. Additionally, 121 age-and-sex-matched healthy controls through the InterVenn platform were analyzed. The resulting glycopeptide abundances were normalized to the levels of pooled human serum run throughout the batch, as well as non-glycosylated peptides from the same protein.

Three sets of glycopeptides were identified.

The first set included those that individually differentiated CRC from AA (FDR<0.05). These also differentiated either an individual CRC vs healthy individual (FDR<0.05, in the same direction as CRC versus AA); or an individual AA vs healthy individual (FDR<0.05, in the same direction as CRC versus AA). Table 6 below includes scores (CRC.FC) wherein high scores on this model may indicated a need to perform a colonoscopy.

The second set included those utilized in multivariable LASSO models built from CRC and Healthy samples (Model 1). Model 1 used SEQ ID NOs: 3, 7, 9, 28, 29, 32, and 33 to create an analytical model. FIG. 1 shows the results of model 1. Training set data is shown with triangles, while patient samples are shown in circles. The model is able to identify patients with CRC versus those who are healthy. Model 1 still also predictive of advanced adenomas even though advanced adenoma data was not used to build the model. Model 1 used a probability threshold for classification of 0.318.

The third set included those utilized in multivariable LASSO models built from AA vs Healthy samples (Model 2). Model 2 used SEQ ID NOs: 1-4, 6-7, 12, 15, 23-25, 28, 29, 32 to create an analysis model. FIG. 2 shows the results of model 2. Training set data is shown with triangles, while patient samples are shown in circles. The model is able to identify patients with AA versus those who are healthy. Model 2 also predictive of CRC even though CRC data was not used to build the model. Model 2 used a probability threshold for classification of 0.385.

Multivariable modeling was performed by splitting the 242 sample set into 70% training and 30% test sets, balanced on cancer stage, age, and sex. Ten-fold cross-validation was repeated five times in the training set to identify optimal LASSO hyperparameters, and models based on those parameters were built utilizing the entire training data set. Model performance was assessed blindly in the test set.

TABLE 6
Analysis of markers used in models 1 and 2.

| Transition | | | | | | CRC.P- |
|---|---|---|---|---|---|---|
| Number | Compound Name | individual.diff | model1 | model2 | CRC.FC | value |
| 1 | A1AT- | no | no | yes | 0.885 | 7.59E−02 |
| | GP001_107_5411 | | | | | |
| 2 | A1AT- | no | no | yes | 1.006 | 7.83E−01 |
| | GP001_271_5402 | | | | | |
| 3 | A1AT- | no | yes | yes | 0.875 | 3.04E−01 |
| | GP001_271_6503 | | | | | |
| 4 | A1BG- | no | no | yes | 1.030 | 1.37E−01 |
| | GP002_179_5421/5402 | | | | | |
| 5 | A2MG- | yes | no | no | 1.155 | 4.95E−03 |
| | GP004_1424_5402 | | | | | |
| 6 | A2MG- | no | no | yes | 1.068 | 4.01E−01 |
| | GP004_1424_5412 | | | | | |
| 7 | A2MG- | no | yes | yes | 1.093 | 9.38E−03 |
| | GP004_55_5402 | | | | | |
| 8 | A2MG- | yes | no | no | 1.100 | 2.30E−03 |
| | GP004_869_5401 | | | | | |
| 9 | A2MG- | yes | yes | no | 1.122 | 3.75E−03 |
| | GP004_869_6301 | | | | | |
| 10 | AACT- | yes | no | no | 0.776 | 1.77E−03 |
| | GP005_271_7603 | | | | | |
| 11 | AGP1- | yes | no | no | 0.753 | 2.52E−03 |
| | GP007_103_9804 | | | | | |
| 12 | AGP1- | no | no | yes | 0.932 | 3.87E−01 |
| | GP007_33_6501 | | | | | |
| 13 | AGP1- | yes | no | no | 0.831 | 5.25E−03 |
| | GP007_93_6502 | | | | | |
| 14 | AGP1- | yes | no | no | 0.665 | 1.06E−03 |
| | GP007_93_7611 | | | | | |
| 15 | AGP2- | no | no | yes | 1.206 | 1.01E−01 |
| | GP008_103_6503 | | | | | |
| 16 | APOC3- | yes | no | no | 1.301 | 4.08E−04 |
| | GP012_74Aoff_1102 | | | | | |
| 17 | APOD- | yes | no | no | 1.532 | 1.41E−04 |
| | GP014_98_5402/5421 | | | | | |
| 18 | APOD- | yes | no | no | 1.773 | 4.68E−06 |
| | GP014_98_5410 | | | | | |
| 19 | APOD- | yes | no | no | 1.347 | 1.48E−03 |
| | GP014_98_6510 | | | | | |
| 20 | APOD- | yes | no | no | 1.404 | 1.70E−03 |
| | GP014_98_6530 | | | | | |
| 21 | APOD- | yes | no | no | 1.734 | 4.57E−06 |
| | GP014_98_9800 | | | | | |
| 22 | CAN3- | yes | no | no | 1.461 | 1.15E−03 |
| | GP022_366_6513 | | | | | |
| 23 | CERU- | no | no | yes | 1.070 | 2.74E−01 |
| | GP023_138_5412 | | | | | |
| 24 | CERU- | no | no | yes | 1.007 | 8.93E−01 |
| | GP023_138_5421/5402 | | | | | |
| 25 | FETUA- | no | no | yes | 1.254 | 3.63E−02 |
| | GP036_176_5401 | | | | | |
| 26 | FETUA- | yes | no | no | 1.365 | 9.88E−05 |
| | GP036_176_6513 | | | | | |
| 27 | HPT-GP044_207_5401 | yes | no | no | 0.705 | 4.21E−04 |
| 28 | HPT- | yes | yes | yes | 1.091 | 2.64E−03 |
| | GP044_241_5402/5421 | | | | | |
| 29 | HPT-GP044_241_5511 | no | yes | yes | 1.012 | 7.24E−01 |
| 30 | HPT-GP044_241_6511 | yes | no | no | 0.600 | 2.96E−06 |
| 31 | HPT-GP044_241_7511 | yes | no | no | 0.607 | 1.36E−04 |
| 32 | IgM-GP053_46_4310 | no | yes | yes | 0.976 | 6.71E−01 |
| 33 | KLKB1- | no | yes | no | 1.237 | 1.91E−02 |
| | GP056_494_6503 | | | | | |
| 34 | PON1- | yes | no | no | 1.626 | 7.83E−05 |
| | GP060_324_5420 | | | | | |
| 35 | PON1- | yes | no | no | 1.751 | 2.19E−05 |
| | GP060_324_6501 | | | | | |
| 36 | PON1- | yes | no | no | 1.816 | 1.35E−05 |
| | GP060_324_6502 | | | | | |
| 37 | UN13A- | yes | no | no | 1.649 | 4.57E−05 |
| | GP066_1005_5431 | | | | | |
| 38 | UN13A- | yes | no | no | 1.427 | 1.28E−03 |
| | GP066_1005_7420 | | | | | |

The CRC.FC (full change) is the average multiplicative difference between the CRC and healthy patients groups for an individual marker. As an example, for an individual marker, a CRC.FC of 2 means that the marker is twice as likely to be expressed in CRC when compared to an healthy patients. As a further example, if the value is 0.5, then the expression of the marker is actually half as much when compared to healthy patients.

The CRC.P-value is the statistical P-value for the CRC.FC and measures the significance of CRC.FC.

The individual. diff is an assignment of whether the individual marker can differentiate CRC versus AA or CRC versus healthy cells and is based on whether the CRC.P-value was deemed significant, specifically whether there was an observed difference between groups. A “yes” response indicates that the marker is capable of distinguishing CRC from AA or CRC from healthy cells. The transition numbers 5, 8-11, 13-14, 16-22, 26-28, 30-31, and 34-38 can be used to distinguish CRC from AA or CRC from healthy cells. Each individual transition number 5, 8-11, 13-14, 16-22, 26-28, 30-31, and 34-38 can be used individually to distinguish CRC from AA or CRC from healthy cells. One or more transition numbers can be combined to distinguish with greater probability CRC from AA or CRC from healthy cells. The transition numbers 1-38 of any Table above correspond to the amino acid sequence set forth in Table 10.

The models will be compared to standard tests to determine CRC and AA. In some cases these standard tests include DNA samples taken from patient stool samples. The methods and models in this application will be shown to have superior predictive performance. Further, the methods and models of this application will not have to rely solely on stool samples for diagnosis purposes.

### Example 4: Area Under the Curve Analysis of Model 1 and Model 2

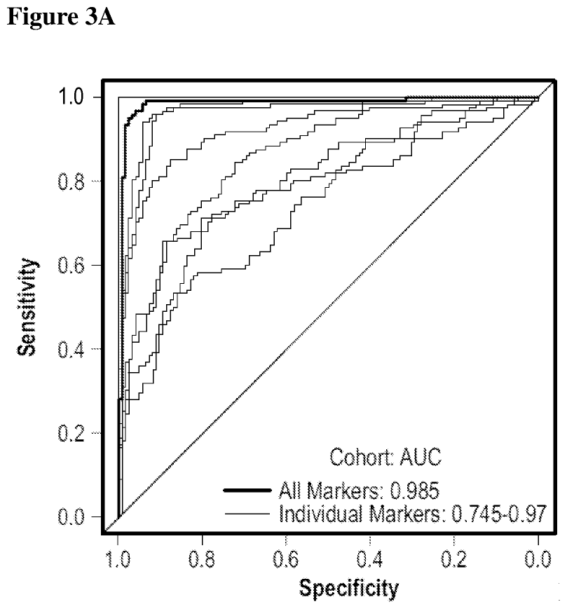

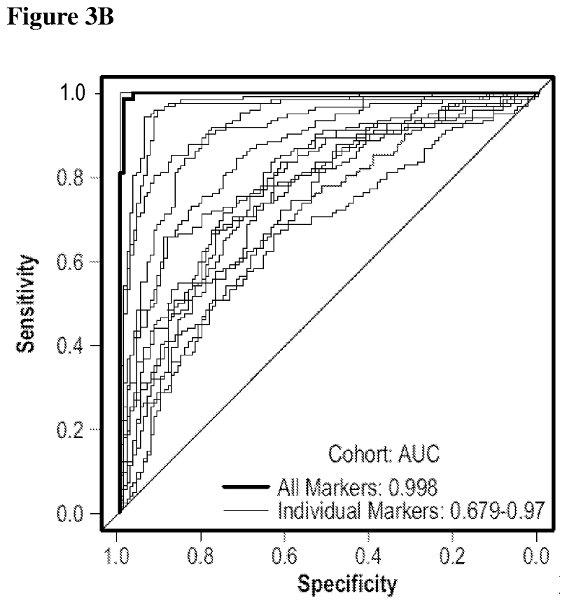

Models 1 and 2 were analyzed using an AUC analysis for specific biomarkers and total biomarkers as shown in FIG. 3A and FIG. 3B. The higher the AUC, the better the model is at predicting whether a biomarker identifies a disease state. Diagnostic accuracy refers to the number of correct test results divided by the number of patients tested. Sensitivity (positive in disease) refers to the proportion of subjects who have the target condition and give positive test results. Specificity is the proportion of subjects without the target condition and give negative test results.

TABLE 7
AUC analysis of individual markers used in model 1.

| Marker | AUC |
|---|---|
| A1AT.GP001_271_6503 | 0.927 |
| A2MG.GP004_55_5402 | 0.858 |
| A2MG.GP004_869_6301 | 0.778 |
| HPT.GP044_241_5402.5421 | 0.955 |
| HPT.GP044_241_5511 | 0.969 |
| IGM.GP053_46_4310 | 0.803 |
| KLKB1.GP056_494_6503 | 0.745 |

TABLE 8
AUC analysis of individual markers used in model 2.

| Marker | AUC |
|---|---|
| A1AT.GP001_107_5411 | 0.909 |
| A1AT.GP001_271_5402 | 0.751 |
| A1AT.GP001_271_6503 | 0.927 |
| A1BG.GP002_179_5421.5402 | 0.722 |
| A2MG.GP004_1424_5412 | 0.771 |
| A2MG.GP004_55_5402 | 0.858 |
| AGP1.GP007_33_6501 | 0.679 |
| AGP2.GP008_103_6503 | 0.733 |
| CERU.GP023_138_5412 | 0.730 |
| CERU.GP023_138_5421.5402 | 0.793 |
| FETUA.GP036_176_5401 | 0.774 |
| HPT.GP044_241_5402.5421 | 0.955 |
| HPT.GP044_241_5511 | 0.969 |
| IGM.GP053_46_4310 | 0.803 |

Model 1 comprises the amino acid sequence set forth in SEQ ID NOs: 3, 7, 9, 28, 29, 32, and 33. Model 1 described herein uses one or more glycopeptide to distinguish an individual having colorectal cancer (CRC) from a healthy individual with excellent predictive results. For Model 1, the accuracy was measured at 0.962, the sensitivity was measured at 0.971, and the specificity was measured at 0.944. The high values for Model 1 AUC, accuracy, sensitivity and specificity indicate that Model 1 provide excellent predictive results.

Model 2 described herein uses one or more glycopeptide comprises the amino acid sequence set forth in SEQ ID NOs: 1-4, 6-7, 12, 15, 23-25, 28, 29, 32. Model 2 described herein uses one or more glycopeptide to distinguish an individual having advanced adenoma (AA) from a healthy individual with excellent predictive results. For Model 2, the accuracy was measured at 0.976, the sensitivity was measured at 0.977, and the specificity was measured at 0.972. The high values for Model 2 AUC, accuracy, sensitivity and specificity indicate that Model 2 provide excellent predictive results.

The high values for the AUC, accuracy, sensitivity and specificity indicate that the models provide excellent predictive results.

TABLE 9
Glycoproteins associated with healthy control and disease samples

| SEQ | Protein | | | |
|---|---|---|---|---|
| ID | Abbrev | | UniProt | |
| NO: | iation | Protein Name | ID | Protein Sequence |
| 39 | A1AT | Alpha-1- | P01009 | MPSSVSWGILLLAGLCCLVPVSLAEDPQGDAAQ |
| | | antitrypsin | | KTDTSHHDQDHPTFNKITPNLAEFAFSLYRQLAH |
| | | | | QSNSTNIFFSPVSIATAFAMLSLGTKADTHDEILE |
| | | | | GLNFNLTEIPEAQIHEGFQELLRTLNQPDSQLQLT |
| | | | | TGNGLFLSEGLKLVDKFLEDVKKLYHSEAFTVN |
| | | | | FGDTEEAKKQINDYVEKGTQGKIVDLVKELDRD |
| | | | | TVFALVNYIFFKGKWERPFEVKDTEEEDFHVDQ |
| | | | | VTTVKVPMMKRLGMFNIQHCKKLSSWVLLMKY |
| | | | | LGNATAIFFLPDEGKLQHLENELTHDIITKFLENE |
| | | | | DRRSASLHLPKLSITGTYDLKSVLGQLGITKVFSN |
| | | | | GADLSGVTEEAPLKLSKAVHKAVLTIDEKGTEA |
| | | | | AGAMFLEAIPMSIPPEVKFNKPFVFLMIEQNTKSP |
| | | | | LFMGKVVNPTQK |
| 40 | A1BG | Alpha-1B- | P04217 | MSMLVVFLLLWGVTWGPVTEAAIFYETQPSLW |
| | | glycoprotein | | AESESLLKPLANVTLTCQAHLETPDFQLFKNGVA |
| | | | | QEPVHLDSPAIKHQFLLTGDTQGRYRCRSGLSTG |
| | | | | WTQLSKLLELTGPKSLPAPWLSMAPVSWITPGLK |
| | | | | TTAVCRGVLRGVTFLLRREGDHEFLEVPEAQED |
| | | | | VEATFPVHQPGNYSCSYRTDGEGALSEPSATVTI |
| | | | | EELAAPPPPVLMHHGESSQVLHPGNKVTLTCVA |
| | | | | PLSGVDFQLRRGEKELLVPRSSTSPDRIFFHLNAV |
| | | | | ALGDGGHYTCRYRLHDNQNGWSGDSAPVELILS |
| | | | | DETLPAPEFSPEPESGRALRLRCLAPLEGARFALV |
| | | | | REDRGGRRVHRFQSPAGTEALFELHNISVADSAN |
| | | | | YSCVYVDLKPPFGGSAPSERLELHVDGPPPRPQL |
| | | | | RATWSGAVLAGRDAVLRCEGPIPDVTFELLREG |
| | | | | ETKAVKTVRTPGAAANLELIFVGPQHAGNYRCR |
| | | | | YRSWVPHTFESELSDPVELLVAES |
| 41 | A2MG | Alpha-2- | P01023 | MGKNKLLHPSLVLLLLVLLPTDASVSGKPQYMV |
| | | macroglobulin | | LVPSLLHTETTEKGCVLLSYLNETVTVSASLESV |
| | | | | RGNRSLFTDLEAENDVLHCVAFAVPKSSSNEEV |
| | | | | MFLTVQVKGPTQEFKKRTTVMVKNEDSLVFVQT |
| | | | | DKSIYKPGQTVKFRVVSMDENFHPLNELIPLVYI |
| | | | | QDPKGNRIAQWQSFQLEGGLKQFSFPLSSEPFQG |
| | | | | SYKVVVQKKSGGRTEHPFTVEEFVLPKFEVQVT |
| | | | | VPKIITILEEEMNVSVCGLYTYGKPVPGHVTVSIC |
| | | | | RKYSDASDCHGEDSQAFCEKFSGQLNSHGCFYQ |
| | | | | QVKTKVFQLKRKEYEMKLHTEAQIQEEGTVVEL |
| | | | | TGRQSSEITRTITKLSFVKVDSHFRQGIPFFGQVR |
| | | | | LVDGKGVPIPNKVIFIRGNEANYYSNATTDEHGL |
| | | | | VQFSINTTNVMGTSLTVRVNYKDRSPCYGYQWV |
| | | | | SEEHEEAHHTAYLVFSPSKSFVHLEPMSHELPCG |
| | | | | HTQTVQAHYILNGGTLLGLKKLSFYYLIMAKGGI |
| | | | | VRTGTHGLLVKQEDMKGHFSISIPVKSDIAPVAR |
| | | | | LLIYAVLPTGDVIGDSAKYDVENCLANKVDLSFS |
| | | | | PSQSLPASHAHLRVTAAPQSVCALRAVDQSVLL |
| | | | | MKPDAELSASSVYNLLPEKDLTGFPGPLNDQDN |
| | | | | EDCINRHNVYINGITYTPVSSTNEKDMYSFLEDM |
| | | | | GLKAFTNSKIRKPKMCPQLQQYEMHGPEGLRVG |
| | | | | FYESDVMGRGHARLVHVEEPHTETVRKYFPETW |
| | | | | IWDLVVVNSAGVAEVGVTVPDTITEWKAGAFCL |
| | | | | SEDAGLGISSTASLRAFQPFFVELTMPYSVIRGEA |
| | | | | FTLKATVLNYLPKCIRVSVQLEASPAFLAVPVEK |
| | | | | EQAPHCICANGRQTVSWAVTPKSLGNVNFTVSA |
| | | | | EALESQELCGTEVPSVPEHGRKDTVIKPLLVEPE |
| | | | | GLEKETTFNSLLCPSGGEVSEELSLKLPPNVVEES |
| | | | | ARASVSVLGDILGSAMQNTQNLLQMPYGCGEQ |
| | | | | NMVLFAPNIYVLDYLNETQQLTPEIKSKAIGYLN |
| | | | | TGYQRQLNYKHYDGSYSTFGERYGRNQGNTWL |
| | | | | TAFVLKTFAQARAYIFIDEAHITQALIWLSQRQK |
| | | | | DNGCFRSSGSLLNNAIKGGVEDEVTLSAYITIALL |
| | | | | EIPLTVTHPVVRNALFCLESAWKTAQEGDHGSH |
| | | | | VYTKALLAYAFALAGNQDKRKEVLKSLNEEAV |
| | | | | KKDNSVHWERPQKPKAPVGHFYEPQAPSAEVE |
| | | | | MTSYVLLAYLTAQPAPTSEDLTSATNIVKWITKQ |
| | | | | QNAQGGFSSTQDTVVALHALSKYGAATFTRTGK |
| | | | | AAQVTIQSSGTFSSKFQVDNNNRLLLQQVSLPEL |
| | | | | PGEYSMKVTGEGCVYLQTSLKYNILPEKEEFPFA |
| | | | | LGVQTLPQTCDEPKAHTSFQISLSVSYTGSRSASN |
| | | | | MAIVDVKMVSGFIPLKPTVKMLERSNHVSRTEV |
| | | | | SSNHVLIYLDKVSNQTLSLFFTVLQDVPVRDLKP |
| | | | | AIVKVYDYYETDEFAIAEYNAPCSKDLGNA |
| 42 | AACT | Alpha-1- | P01011 | MERMLPLLALGLLAAGFCPAVLCHPNSPLDEEN |
| | | antichymotrypsin | | LTQENQDRGTHVDLGLASANVDFAFSLYKQLVL |
| | | | | KAPDKNVIFSPLSISTALAFLSLGAHNTTLTEILKG |
| | | | | LKFNLTETSEAEIHQSFQHLLRTLNQSSDELQLSM |
| | | | | GNAMFVKEQLSLLDRFTEDAKRLYGSEAFATDF |
| | | | | QDSAAAKKLINDYVKNGTRGKITDLIKDLDSQT |
| | | | | MMVLVNYIFFKAKWEMPFDPQDTHQSRFYLSK |
| | | | | KKWVMVPMMSLHHLTIPYFRDEELSCTVVELKY |
| | | | | TGNASALFILPDQDKMEEVEAMLLPETLKRWRD |
| | | | | SLEFREIGELYLPKFSISRDYNLNDILLQLGIEEAF |
| | | | | TSKADLSGITGARNLAVSQVVHKAVLDVFEEGT |
| | | | | EASAATAVKITLLSALVETRTIVRFNRPFLMIIVPT |
| | | | | DTQNIFFMSKVTNPKQA |
| 43 | AGP1 | Alpha-1-acid | P02763 | MALSWVLTVLSLLPLLEAQIPLCANLVPVPITNA |
| | | glycoprotein 1 | | TLDRITGKWFYIASAFRNEEYNKSVQEIQATFFYF |
| | | | | TPNKTEDTIFLREYQTRQDQCIYNTTYLNVQREN |
| | | | | GTISRYVGGQEHFAHLLILRDTKTYMLAFDVND |
| | | | | EKNWGLSVYADKPETTKEQLGEFYEALDCLRIP |
| | | | | KSDVVYTDWKKDKCEPLEKQHEKERKQEEGES |
| 44 | AGP2 | Alpha-1-acid | P19652 | MALSWVLTVLSLLPLLEAQIPLCANLVPVPITNA |
| | | glycoprotein 2 | | TLDRITGKWFYIASAFRNEEYNKSVQEIQATFFYF |
| | | | | TPNKTEDTIFLREYQTRQNQCFYNSSYLNVQREN |
| | | | | GTVSRYEGGREHVAHLLFLRDTKTLMFGSYLDD |
| | | | | EKNWGLSFYADKPETTKEQLGEFYEALDCLCIPR |
| | | | | SDVMYTDWKKDKCEPLEKQHEKERKQEEGES |
| 45 | APOC3 | Apolipoprotein | P02656 | MQPRVLLVVALLALLASARASEAEDASLLSFMQ |
| | | C-III | | GYMKHATKTAKDALSSVQESQVAQQARGWVT |
| | | | | DGFSSLKDYWSTVKDKFSEFWDLDPEVRPTSAV |
| | | | | AA |
| 46 | APOD | Apolipoprotein | P05090 | MVMLLLLLSALAGLFGAAEGQAFHLGKCPNPPV |
| | | D | | QENFDVNKYLGRWYEIEKIPTTFENGRCIQANYS |
| | | | | LMENGKIKVLNQELRADGTVNQIEGEATPVNLT |
| | | | | EPAKLEVKFSWFMPSAPYWILATDYENYALVYS |
| | | | | CTCIIQLFHVDFAWILARNPNLPPETVDSLKNILT |
| | | | | SNNIDVKKMTVTDQVNCPKLS |
| 47 | CAN3 | Calpain-3 | P20807 | MPTVISASVAPRTAAEPRSPGPVPHPAQSKATEA |
| | | | | GGGNPSGIYSAIISRNFPIIGVKEKTFEQLHKKCLE |
| | | | | KKVLYVDPEFPPDETSLFYSQKFPIQFVWKRPPEI |
| | | | | CENPRFIIDGANRTDICQGELGDCWFLAAIACLTL |
| | | | | NQHLLFRVIPHDQSFIENYAGIFHFQFWRYGEWV |
| | | | | DVVIDDCLPTYNNQLVFTKSNHRNEFWSALLEK |
| | | | | AYAKLHGSYEALKGGNTTEAMEDFTGGVAEFFE |
| | | | | IRDAPSDMYKIMKKAIERGSLMGCSIDDGTNMT |
| | | | | YGTSPSGLNMGELIARMVRNMDNSLLQDSDLDP |
| | | | | RGSDERPTRTIIPVQYETRMACGLVRGHAYSVTG |
| | | | | LDEVPFKGEKVKLVRLRNPWGQVEWNGSWSDR |
| | | | | WKDWSFVDKDEKARLQHQVTEDGEFWMSYED |
| | | | | FIYHFTKLEICNLTADALQSDKLQTWTVSVNEGR |
| | | | | WVRGCSAGGCRNFPDTFWTNPQYRLKLLEEDD |
| | | | | DPDDSEVICSFLVALMQKNRRKDRKLGASLFTIG |
| | | | | FAIYEVPKEMHGNKQHLQKDFFLYNASKARSKT |
| | | | | YINMREVSQRFRLPPSEYVIVPSTYEPHQEGEFIL |
| | | | | RVFSEKRNLSEEVENTISVDRPVKKKKTKPIIFVS |
| | | | | DRANSNKELGVDQESEEGKGKTSPDKQKQSPQP |
| | | | | QPGSSDQESEEQQQFRNIFKQIAGDDMEICADEL |
| | | | | KKVLNTVVNKHKDLKTHGFTLESCRSMIALMDT |
| | | | | DGSGKLNLQEFHHLWNKIKAWQKIFKHYDTDQS |
| | | | | GTINSYEMRNAVNDAGFHLNNQLYDIITMRYAD |
| | | | | KHMNIDFDSFICCFVRLEGMFRAFHAFDKDGDGI |
| | | | | IKLNVLEWLQLTMYA |
| 48 | CERU | Ceruloplasmin | P00450 | MKILILGIFLFLCSTPAWAKEKHYYIGIIETTWDY |
| | | | | ASDHGEKKLISVDTEHSNIYLQNGPDRIGRLYKK |
| | | | | ALYLQYTDETFRTTIEKPVWLGFLGPIIKAETGDK |
| | | | | VYVHLKNLASRPYTFHSHGITYYKEHEGAIYPDN |
| | | | | TTDFQRADDKVYPGEQYTYMLLATEEQSPGEGD |
| | | | | GNCVTRIYHSHIDAPKDIASGLIGPLIICKKDSLDK |
| | | | | EKEKHIDREFVVMFSVVDENFSWYLEDNIKTYCS |
| | | | | EPEKVDKDNEDFQESNRMYSVNGYTFGSLPGLS |
| | | | | MCAEDRVKWYLFGMGNEVDVHAAFFHGQALT |
| | | | | NKNYRIDTINLFPATLFDAYMVAQNPGEWMLSC |
| | | | | QNLNHLKAGLQAFFQVQECNKSSSKDNIRGKHV |
| | | | | RHYYIAAEEIIWNYAPSGIDIFTKENLTAPGSDSA |
| | | | | VFFEQGTTRIGGSYKKLVYREYTDASFTNRKERG |
| | | | | PEEEHLGILGPVIWAEVGDTIRVTFHNKGAYPLSI |
| | | | | EPIGVRFNKNNEGTYYSPNYNPQSRSVPPSASHV |
| | | | | APTETFTYEWTVPKEVGPTNADPVCLAKMYYSA |
| | | | | VDPTKDIFTGLIGPMKICKKGSLHANGRQKDVD |
| | | | | KEFYLFPTVFDENESLLLEDNIRMFTTAPDQVDK |
| | | | | EDEDFQESNKMHSMNGFMYGNQPGLTMCKGDS |
| | | | | VVWYLFSAGNEADVHGIYFSGNTYLWRGERRD |
| | | | | TANLFPQTSLTLHMWPDTEGTFNVECLTTDHYT |
| | | | | GGMKQKYTVNQCRRQSEDSTFYLGERTYYIAAV |
| | | | | EVEWDYSPQREWEKELHHLQEQNVSNAFLDKG |
| | | | | EFYIGSKYKKVVYRQYTDSTFRVPVERKAEEEHL |
| | | | | GILGPQLHADVGDKVKIIFKNMATRPYSIHAHGV |
| | | | | QTESSTVTPTLPGETLTYVWKIPERSGAGTEDSA |
| | | | | CIPWAYYSTVDQVKDLYSGLIGPLIVCRRPYLKV |
| | | | | FNPRRKLEFALLFLVFDENESWYLDDNIKTYSDH |
| | | | | PEKVNKDDEEFIESNKMHAINGRMFGNLQGLTM |
| | | | | HVGDEVNWYLMGMGNEIDLHTVHFHGHSFQYK |
| | | | | HRGVYSSDVFDIFPGTYQTLEMFPRTPGIWLLHC |
| | | | | HVTDHIHAGMETTYTVLQNEDTKSG |
| 49 | FETUA | Alpha-2-HS- | P02765 | MKSLVLLLCLAQLWGCHSAPHGPGLIYRQPNCD |
| | | glycoprotein | | DPETEEAALVAIDYINQNLPWGYKHTLNQIDEVK |
| | | | | VWPQQPSGELFEIEIDTLETTCHVLDPTPVARCSV |
| | | | | RQLKEHAVEGDCDFQLLKLDGKFSVVYAKCDSS |
| | | | | PDSAEDVRKVCQDCPLLAPLNDTRVVHAAKAAL |
| | | | | AAFNAQNNGSNFQLEEISRAQLVPLPPSTYVEFT |
| | | | | VSGTDCVAKEATEAAKCNLLAEKQYGFCKATLS |
| | | | | EKLGGAEVAVTCMVFQTQPVSSQPQPEGANEAV |
| | | | | PTPVVDPDAPPSPPLGAPGLPPAGSPPDSHVLLAA |
| | | | | PPGHQLHRAHYDLRHTFMGVVSLGSPSGEVSHP |
| | | | | RKTRTVVQPSVGAAAGPVVPPCPGRIRHFKV |
| 50 | HPT | Haptoglobin | P00738 | MSALGAVIALLLWGQLFAVDSGNDVTDIADDGC |
| | | | | PKPPEIAHGYVEHSVRYQCKNYYKLRTEGDGVY |
| | | | | TLNDKKQWINKAVGDKLPECEADDGCPKPPEIA |
| | | | | HGYVEHSVRYQCKNYYKLRTEGDGVYTLNNEK |
| | | | | QWINKAVGDKLPECEAVCGKPKNPANPVQRILG |
| | | | | GHLDAKGSFPWQAKMVSHHNLTTGATLINEQW |
| | | | | LLTTAKNLFLNHSENATAKDIAPTLTLYVGKKQL |
| | | | | VEIEKVVLHPNYSQVDIGLIKLKQKVSVNERVMP |
| | | | | ICLPSKDYAEVGRVGYVSGWGRNANFKFTDHLK |
| | | | | YVMLPVADQDQCIRHYEGSTVPEKKTPKSPVGV |
| | | | | QPILNEHTFCAGMSKYQEDTCYGDAGSAFAVHD |
| | | | | LEEDTWYATGILSFDKSCAVAEYGVYVKVTSIQ |
| | | | | DWVQKTIAEN |
| 51 | IgM | Immunoglobulin | P01871 | GSASAPTLFPLVSCENSPSDTSSVAVGCLAQDFLP |
| | | heavy chain | | DSITFSWKYKNNSDISSTRGFPSVLRGGKYAATS |
| | | constant μ | | QVLLPSKDVMQGTDEHVVCKVQHPNGNKEKNV |
| | | | | PLPVIAELPPKVSVFVPPRDGFFGNPRKSKLICQA |
| | | | | TGFSPRQIQVSWLREGKQVGSGVTTDQVQAEAK |
| | | | | ESGPTTYKVTSTLTIKESDWLGQSMFTCRVDHRG |
| | | | | LTFQQNASSMCVPDQDTAIRVFAIPPSFASIFLTK |
| | | | | STKLTCLVTDLTTYDSVTISWTRQNGEAVKTHT |
| | | | | NISESHPNATFSAVGEASICEDDWNSGERFTCTV |
| | | | | THTDLPSPLKQTISRPKGVALHRPDVYLLPPARE |
| | | | | QLNLRESATITCLVTGFSPADVFVQWMQRGQPL |
| | | | | SPEKYVTSAPMPEPQAPGRYFAHSILTVSEEEWN |
| | | | | TGETYTCVVAHEALPNRVTERTVDKSTGKPTLY |
| | | | | NVSLVMSDTAGTCY |
| 52 | KLKB1 | Plasma | P03952 | MILFKQATYFISLFATVSCGCLTQLYENAFFRGG |
| | | Kallikrein | | DVASMYTPNAQYCQMRCTFHPRCLLFSFLPASSI |
| | | | | NDMEKRFGCFLKDSVTGTLPKVHRTGAVSGHSL |
| | | | | KQCGHQISACHRDIYKGVDMRGVNFNVSKVSSV |
| | | | | EECQKRCTNNIRCQFFSYATQTFHKAEYRNNCLL |
| | | | | KYSPGGTPTAIKVLSNVESGFSLKPCALSEIGCHM |
| | | | | NIFQHLAFSDVDVARVLTPDAFVCRTICTYHPNC |
| | | | | LFFTFYTNVWKIESQRNVCLLKTSESGTPSSSTPQ |
| | | | | ENTISGYSLLTCKRTLPEPCHSKIYPGVDFGGEEL |
| | | | | NVTFVKGVNVCQETCTKMIRCQFFTYSLLPEDC |
| | | | | KEEKCKCFLRLSMDGSPTRIAYGTQGSSGYSLRL |
| | | | | CNTGDNSVCTTKTSTRIVGGTNSSWGEWPWQVS |
| | | | | LQVKLTAQRHLCGGSLIGHQWVLTAAHCFDGLP |
| | | | | LQDVWRIYSGILNLSDITKDTPFSQIKEIIIHQNYK |
| | | | | VSEGNHDIALIKLQAPLNYTEFQKPICLPSKGDTS |
| | | | | TIYTNCWVTGWGFSKEKGEIQNILQKVNIPLVTN |
| | | | | EECQKRYQDYKITQRMVCAGYKEGGKDACKGD |
| | | | | SGGPLVCKHNGMWRLVGITSWGEGCARREQPG |
| | | | | VYTKVAEYMDWILEKTQSSDGKAQMQSPA |
| 53 | P0N1 | Serum | P27169 | MAKLIALTLLGMGLALFRNHQSSYQTRLNALRE |
| | | paraoxonase/ | | VQPVELPNCNLVKGIETGSEDLEILPNGLAFISSG |
| | | arylesterase 1 | | LKYPGIKSFNPNSPGKILLMDLNEEDPTVLELGIT |
| | | | | GSKFDVSSFNPHGISTFTDEDNAMYLLVVNHPDA |
| | | | | KSTVELFKFQEEEKSLLHLKTIRHKLLPNLNDIVA |
| | | | | VGPEHFYGTNDHYFLDPYLQSWEMYLGLAWSY |
| | | | | VVYYSPSEVRVVAEGFDFANGINISPDGKYVYIA |
| | | | | ELLAHKIHVYEKHANWTLTPLKSLDFNTLVDNIS |
| | | | | VDPETGDLWVGCHPNGMKIFFYDSENPPASEVL |
| | | | | RIQNILTEEPKVTQVYAENGTVLQGSTVASVYKG |
| | | | | KLLIGTVFHKALYCEL |
| 54 | UN13A | Protein unc- | Q9UPW8 | MSLLCVGVKKAKFDGAQEKFNTYVTLKVQNVK |
| | | 13HomologA | | STTIAVRGSQPSWEQDFMFEINRLDLGLTVEVWN |
| | | | | KGLIWDTMVGTVWIPLRTIRQSNEEGPGEWLTL |
| | | | | DSQVIMADSEICGTKDPTFHRILLDTRFELPLDIPE |
| | | | | EEARYWAKKLEQLNAMRDQDEYSFQDEQDKPL |
| | | | | PVPSNQCCNWNYFGWGEQHNDDPDSAVDDRDS |
| | | | | DYRSETSNSIPPPYYTTSQPNASVHQYSVRPPPLG |
| | | | | SRESYSDSMHSYEEFSEPQALSPTGSSRYASSGEL |
| | | | | SQGSSQLSEDFDPDEHSLQGSDMEDERDRDSYHS |
| | | | | CHSSVSYHKDSPRWDQDEEELEEDLEDFLEEEEL |
| | | | | PEDEEELEEEEEEVPDDLGSYAQREDVAVAEPKD |
| | | | | FKRISLPPAAPGKEDKAPVAPTEAPDMAKVAPKP |
| | | | | ATPDKVPAAEQIPEAEPPKDEESFRPREDEEGQE |
| | | | | GQDSMSRAKANWLRAFNKVRMQLQEARGEGE |
| | | | | MSKSLWFKGGPGGGLIIIDSMPDIRKRKPIPLVSD |
| | | | | LAMSLVQSRKAGITSALASSTLNNEELKNHVYK |
| | | | | KTLQALIYPISCTTPHNFEVWTATTPTYCYECEGL |
| | | | | LWGIARQGMRCTECGVKCHEKCQDLLNADCLQ |
| | | | | RAAEKSSKHGAEDRTQNIIMVLKDRMKIRERNK |
| | | | | PEIFELIQEIFAVTKTAHTQQMKAVKQSVLDGTS |
| | | | | KWSAKISITVVCAQGLQAKDKTGSSDPYVTVQV |
| | | | | GKTKKRTKTIYGNLNPVWEENFHFECHNSSDRIK |
| | | | | VRVWDEDDDIKSRVKQRFKRESDDFLGQTIIEVR |
| | | | | TLSGEMDVWYNLDKRTDKSAVSGAIRLHISVEIK |
| | | | | GEEKVAPYHVQYTCLHENLFHFVTDVQNNGVV |
| | | | | KIPDAKGDDAWKVYYDETAQEIVDEFAMRYGV |
| | | | | ESIYQAMTHFACLSSKYMCPGVPAVMSTLLANI |
| | | | | NAYYAHTTASTNVSASDRFAASNFGKERFVKLL |
| | | | | DQLHNSLRIDLSMYRNNFPASSPERLQDLKSTVD |
| | | | | LLTSITFFRMKVQELQSPPRASQVVKDCVKACLN |
| | | | | STYEYIFNNCHELYSREYQTDPAKKGEVLPEEQG |
| | | | | PSIKNLDFWSKLITLIVSIIEEDKNSYTPCLNQFPQ |
| | | | | ELNVGKISAEVMWNLFAQDMKYAMEEHDKHRL |
| | | | | CKSADYMNLHFKVKWLYNEYVTELPAFKDRVP |
| | | | | EYPAWFEPFVIQWLDENEEVSRDFLHGALERDK |
| | | | | KDGFQQTSEHALFSCSVVDVFSQLNQSFEIIKKLE |
| | | | | CPDPQIVGHYMRRFAKTISNVLLQYADIISKDFAS |
| | | | | YCSKEKEKVPCILMNNTQQLRVQLEKMFEAMG |
| | | | | GKELDAEASDILKELQVKLNNVLDELSRVFATSF |
| | | | | QPHIEECVKQMGDILSQVKGTGNVPASACSSVA |
| | | | | QDADNVLQPIMDLLDSNLTLFAKICEKTVLKRVL |
| | | | | KELWKLVMNTMEKTIVLPPLTDQTMIGNLLRKH |
| | | | | GKGLEKGRVKLPSHSDGTQMIFNAAKELGQLSK |
| | | | | LKDHMVREEAKSLTPKQCAVVELALDTIKQYFH |
| | | | | AGGVGLKKTFLEKSPDLQSLRYALSLYTQATDL |
| | | | | LIKTFVQTQSAQGLGVEDPVGEVSVHVELFTHPG |
| | | | | TGEHKVTVKVVAANDLKWQTSGIFRPFIEVNIIG |
| | | | | PQLSDKKRKFATKSKNNSWAPKYNESFQFTLSA |
| | | | | DAGPECYELQVCVKDYCFAREDRTVGLAVLQLR |
| | | | | ELAQRGSAACWLPLGRRIHMDDTGLTVLRILSQ |
| | | | | RSNDEVAKEFVKLKSDTRSAEEGGAAPAP |

TABLE 10
Details of glycopeptides with different abundances in healthy control and disease
samples

| | | | Glycan | Glycan | |
|---|---|---|---|---|---|
| | | | Linking | Linking | |
| | | | Site Pos. | Site Pos. | Glycan |
| SEQ ID | Peptide Structure | | in Peptide | in rotein | Structure |
| NO: | (PS) NAME | Peptide Sequence | Sequence | Sequence | GL NO |
| 1 | A1AT-GP001_107_5411 | ADTHDEILEGLN | 14 | 107 | 5411 |
| | | FNLTEIPEAQIHE | | | |
| | | GFQELLR | | | |
| 2 | A1AT-GP001_271_5402 | YLGNATAIFFLP | 4 | 271 | 5402 |
| | | DEGK | | | |
| 3 | A1AT-GP001_271_6503 | YLGNATAIFFLP | 4 | 271 | 6503 |
| | | DEGK | | | |
| 4 | A1BG-GP002_179_5402 | EGDHEFLEVPEA | 27 | 179 | 5402 |
| | | QEDVEATFPVHQ | | | |
| | | PGNYSCSYR | | | |
| 5 | A2MG-GP004_1424_5402 | VSNQTLSLFFTV | 3 | 1424 | 5402 |
| | | LQDVPVR | | | |
| 6 | A2MG-GP004_1424_5412 | VSNQTLSLFFTV | 3 | 1424 | 5412 |
| | | LQDVPVR | | | |
| 7 | A2MG-GP004_55_5402 | GCVLLSYLNETV | 9 | 55 | 5402 |
| | | TVSASLESVR | | | |
| 8 | A2MG-GP004_869_5401 | SLGNVNFTVSAE | 6 | 869 | 5401 |
| | | ALESQELCGTEV | | | |
| | | PSVPEHGR | | | |
| 9 | A2MG-GP004_869_6301 | SLGNVNFTVSAE | 6 | 869 | 6301 |
| | | ALESQELCGTEV | | | |
| | | PSVPEHGR | | | |
| 10 | AACT-GP005_271_7603 | YTGNASALFILP | 4 | 271 | 7603 |
| | | DQDK | | | |
| 11 | AGP1-GP007_103_9804 | ENGTISR | 2 | 103 | 9804 |
| 12 | AGP1-GP007_33_6501 | QIPLCANLVPVPI | 15 | 33 | 6501 |
| | | TNATLDQITGK | | | |
| 13 | AGP1-GP007_93_6502 | QDQCIYNTTYLN | 7 | 93 | 6502 |
| | | VQR | | | |
| 14 | AGP1-GP007_93_7611 | QDQCIYNTTYLN | 7 | 93 | 7611 |
| | | VQR | | | |
| 15 | AGP2-GP008_103_6503 | ENGTVSR | 2 | 103 | 6503 |
| 16 | APOC3- | FSEFWDLDPEVR | 14 | 74 | 1102 |
| | GP012_74Aoff_1102 | PTSAVA | | | |
| 17 | APOD-GP014_98_5402 | ADGTVNQIEGEA | 16 | 98 | 5402 |
| | | TPVNLTEPAK | | | |
| 18 | APOD-GP014_98_5410 | ADGTVNQIEGEA | 16 | 98 | 5410 |
| | | TPVNLTEPAK | | | |
| 19 | APOD-GP014_98_6510 | ADGTVNQIEGEA | 16 | 98 | 6510 |
| | | TPVNLTEPAK | | | |
| 20 | APOD-GP014_98_6530 | ADGTVNQIEGEA | 16 | 98 | 6530 |
| | | TPVNLTEPAK | | | |
| 21 | APOD-GP014_98_9800 | ADGTVNQIEGEA | 16 | 98 | 9800 |
| | | TPVNLTEPAK | | | |
| 22 | CAN3-GP022_366_6513 | NPWGQVEWNGS | 9 | 366 | 6513 |
| | | WSDR | | | |
| 23 | CERU-GP023_138_5412 | EHEGAIYPDNTT | 10 | 138 | 5412 |
| | | DFQR | | | |
| 24 | CERU-GP023_138_5402 | EHEGAIYPDNTT | 10 | 138 | 5402 |
| | | DFQR | | | |
| 25 | FETUA-GP036_176_5401 | AALAAFNAQNN | 11 | 176 | 5401 |
| | | GSNFQLEEISR | | | |
| 26 | FETUA-GP036_176_6513 | AALAAFNAQNN | 11 | 176 | 6513 |
| | | GSNFQLEEISR | | | |
| 27 | HPT-GP044_207_5401 | NLFLNHSENATA | 5 | 207 | 5401 |
| | | K | | | |
| 28 | HPT-GP044_241_5402 | VVLHPNYSQVDI | 6 | 241 | 5402 |
| | | GLIK | | | |
| 29 | HPT-GP044_241_5511 | VVLHPNYSQVDI | 6 | 241 | 5511 |
| | | GLIK | | | |
| 30 | HPT-GP044_241_6511 | VVLHPNYSQVDI | 6 | 241 | 6511 |
| | | GLIK | | | |
| 31 | HPT-GP044_241_7511 | VVLHPNYSQVDI | 6 | 241 | 7511 |
| | | GLIK | | | |
| 32 | IgM-GP053_46_4310 | YKNNSDISSTR | 3 | 46 | 4310 |
| 33 | KLKB1-GP056_494_6503 | LQAPLNYTEFQK | 6 | 494 | 6503 |
| | | PICL | | | |
| 34 | PON1-GP060_324_5420 | VTQVYAENGTV | 8 | 324 | 5420 |
| | | LQGSTVASVYK | | | |
| 35 | PON1-GP060_324_6501 | VTQVYAENGTV | 8 | 324 | 6501 |
| | | LQGSTVASVYK | | | |
| 36 | PON1-GP060_324_6502 | VTQVYAENGTV | 8 | 324 | 6502 |
| | | LQGSTVASVYK | | | |
| 37 | UN13A-GP066_1005_5431 | ACLNSTYEYIFN | 4 | 1005 | 5431 |
| | | NCHELYSR | | | |
| 38 | UN13A-GP066_1005_7420 | ACLNSTYEYIFN | 4 | 1005 | 7420 |
| | | NCHELYSR | | | |

TABLE 11A
Glycan structure GL NO, symbol structure, and composition of detected glycan
moieties for O-linked glycans

| Glycan Structure GL | | |
|---|---|---|
| NO. | Symbol Structure | Composition |
| 1102 | 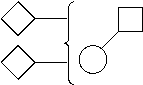 | Hex(1)HexNAc(1)Fuc(0)NeuAc(2) |

TABLE 11B
Glycan structure GL NO, symbol structure, and composition of detected glycan moieties for N-linked glycans

| Glycan Structure GL | | |
|---|---|---|
| NO. | Symbol Structure | Composition |
| 4310 | 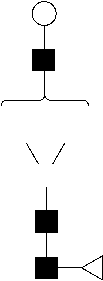 | Hex(4)HexNAc(3)Fuc(1)NeuAc(0) |
| 5401 | 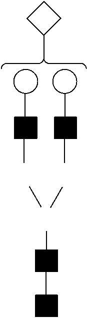 | Hex(5)HexNAc(4)Fuc(0)NeuAc(1) |
| 5402 | 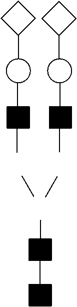 | Hex(5)HexNAc(4)Fuc(0)NeuAc(2) |
| 5410 | 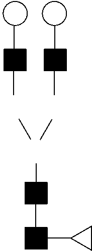 | Hex(5)HexNAc(4)Fuc(1)NeuAc(0) |
| 5411 | 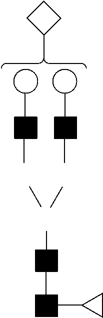 | Hex(5)HexNAc(4)Fuc(1)NeuAc(1) |
| 5412 | 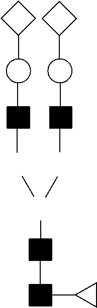 | Hex(5)HexNAc(4)Fuc(1)NeuAc(2) |
| 5420 | 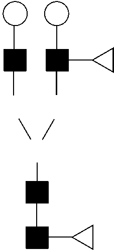 | Hex(5)HexNAc(4)Fuc(2)NeuAc(0) |
| 5421 | 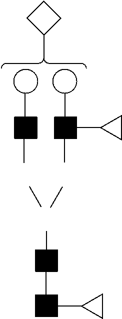 | Hex(5)HexNAc(4)Fuc(2)NeuAc(1) |
| 5431 | 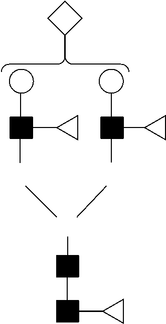 | Hex(5)HexNAc(4)Fuc(3)NeuAc(1) |
| 5511 | 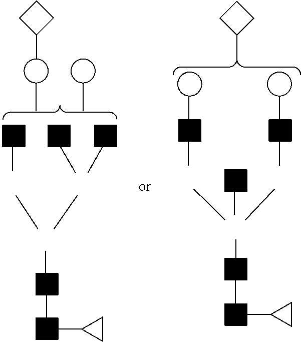 | Hex(5)HexNAc(5)Fuc(1)NeuAc(1) |
| 6301 | 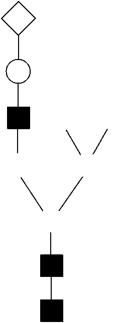 | Hex(6)HexNAc(3)Fuc(0)NeuAc(1) |
| 6501 | 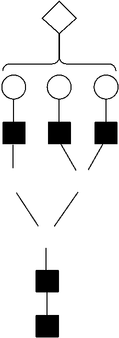 | Hex(6)HexNAc(5)Fuc(0)NeuAc(1) |
| 6502 | 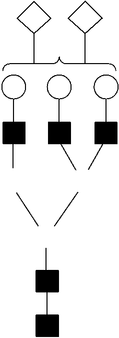 | Hex(6)HexNAc(5)Fuc(0)NeuAc(2) |
| 6503 | 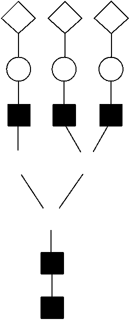 | Hex(6)HexNAc(5)Fuc(0)NeuAc(3) |
| 6510 | 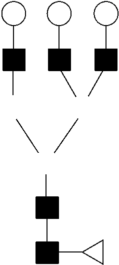 | Hex(6)HexNAc(5)Fuc(1)NeuAc(0) |
| 6511 | 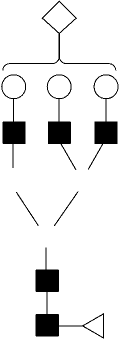 | Hex(6)HexNAc(5)Fuc(1)NeuAc(1) |
| 6513 | 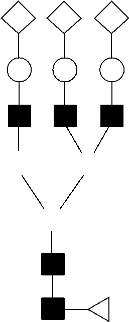 | Hex(6)HexNAc(5)Fuc(1)NeuAc(3) |
| 6530 | 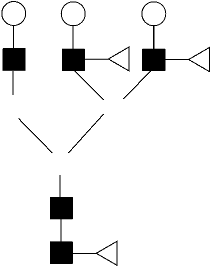 | Hex(6)HexNAc(5)Fuc(3)NeuAc(0) |
| 7420 | 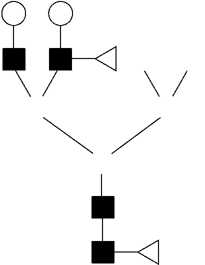 | Hex(7)HexNAc(4)Fuc(2)NeuAc(0) |
| 7511 | 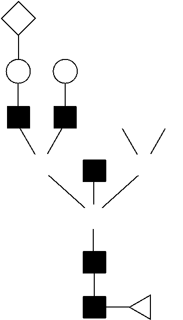 | Hex(7)HexNAc(5)Fuc(1)NeuAc(1) |
| 7603 | 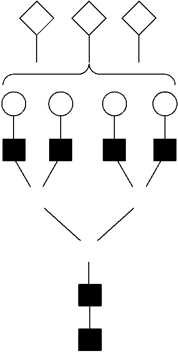 | Hex(7)HexNAc(6)Fuc(0)NeuAc(3) |
| 7611 | 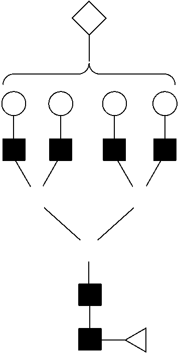 | Hex(7)HexNAc(6)Fuc(1)NeuAc(1) |
| 9800 | n/a | Hex(9)HexNAc(8)Fuc(0)NeuAc(0) |
| 9804 | 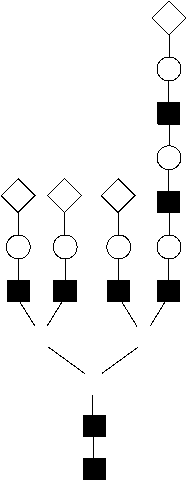 | Hex(9)HexNAc(8)Fuc(0)NeuAc(4) |

Legend for Tables 11A and 11B

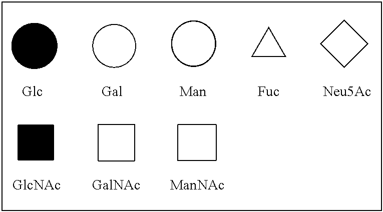

Table 11A and Table 11B illustrate the symbol structure and composition of detected glycan moieties that correspond to glycopeptides of Table 10 based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. It should be noted that the Glycan Structure GL NO 1102 is an O-linked glycan that is in Table 11A and that N-linked glycans are in Table 11B. For reference, N-linked glycans have a glycan attached to the amino acid asparagine and O-linked glycans have a glycan attached to either a serine or a threonine.

The identity of the various monosaccharides is illustrated by the Legend section located at the end of Table 11B. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

### IX. Exemplary Methods

In some aspects, provided herein is a method of classifying a biological sample obtained from a subject with respect to a plurality of states associated with colorectal cancer (CRC) or advanced adenoma (AA), the method comprising receiving peptide structure data corresponding to a set of proteins in the biological sample. In some embodiments, the peptide structure data corresponds to a set of glycoproteins in the biological sample. In some embodiments, the peptide structure data corresponding to a set of glycoproteins in a biological sample obtained from a subject, wherein the peptide structure data comprises at least one peptide structure from Table 10. In some embodiments, the method further comprises inputting quantification data identified from the peptide structure data for a set of peptide structures into a machine-learning model trained to identify a disease indicator based on the quantification data, wherein the set of peptide structures comprises at least one peptide structure identified from a plurality of peptide structures in Table 10. In some embodiments, the method further comprises identifying, by the machine-learning model, the disease indicator. In some embodiments, the method further comprises classifying the biological sample with respect to a plurality of states associated with CRC or AA based upon the identified disease indicator. In some embodiments, the method comprises classifying the sample as having CRC or not having CRC based upon the disease indicator. In some embodiments, the method comprises classifying the sample as having AA or not having AA based upon the disease indicator. In some embodiments, the presence, absence, and/or amount of one or more peptides and/or glycopeptide is determined by MRM-MS.

In some aspects, provided herein is a method of detecting the presence of colorectal cancer (CRC) or advanced adenoma (AA) in a subject, the method comprising receiving peptide structure data corresponding to a set of proteins in a biological sample obtained from a subject, wherein the peptide structure data comprises at least one peptide structure from Table 10. In some embodiments, the peptide structure data corresponds to a set of glycoproteins in the biological sample. In some embodiments, the method further comprises inputting quantification data identified from the peptide structure data for a set of peptide structures into a machine-learning model trained to identify a disease indicator based on the quantification data. In some embodiments, the method further comprises detecting the presence of CRC or AA in response to a determination that the identified disease indicator falls within a selected range associated with CRC or AA. In some embodiments, the presence, absence, and/or amount of one or more peptides and/or glycopeptide is determined by MRM-MS.

In some embodiments, the set of proteins comprises one or more glycoproteins wherein the glycoprotein comprises at least one glycoprotein from Table 9. In some embodiments, the one or more glycoproteins comprise the amino acid sequence of SEQ ID NOs: 39-54. In some embodiments, the at least one peptide structure comprises a glycopeptide wherein the peptide structure comprises at least one glycopeptide from Table 10. In some embodiments, the one or more glycopeptide comprise the amino acid sequence of SEQ ID NOs: 1-38. In some embodiments, the method comprises classifying a biological sample with respect to a plurality of states associated with CRC or AA based upon one or more glycopeptides provided in Table 10. In some embodiments, the plurality of states comprises at least one of a CRC state, an AA state, or a healthy state. In some embodiments, the plurality of states comprises at least two of a CRC state, an AA state, and a healthy state. In some embodiments, the plurality of states comprises each of a CRC state, an AA state, and a healthy state.

In some embodiments, the machine-learning model comprises a logistic regression model. In some embodiments, the machine-learning model comprises a regularized regression model. In some embodiments, the regularized regression model comprises a least absolute shrinkage and selection operator (LASSO) regression model.

In some embodiments, the quantification data for a peptide structure of the set of peptide structures comprises at least one of an abundance, a relative abundance, a normalized abundance, or a differential abundance. In some embodiments, the quantification data for a peptide structure of the set of peptide structures comprises at least one of a relative quantity, an adjusted quantity, a normalized quantity, a relative concentration, an adjusted concentration, or a normalized concentration. In some embodiments, the quantification data is generated using a liquid chromatography-mass spectrometry (LC-MS) system. In some embodiments, the peptide structure data is generated using multiple reaction monitoring mass spectrometry (MRM-MS). For example, a first data set of MRM transition signals indicative of a sample from an individual with CRC or AA and a second data set of MRM transition signals indicative of a control sample are collected. Comparison of the first data set with the second data set enable the calculation of the relative abundance, the normalized abundance, or the differential abundance of the glycopeptide associated with the sample from an individual with CRC or AA and the control sample.

In some embodiments, the machine-learning model was trained utilizing a portion of the quantification data corresponding to a set of peptide structures that is a subset of the panel of peptide structures to determine which state of the plurality of states the biological sample from the subject corresponds. In some embodiments, the set of peptide structures comprises the amino acid sequence of SEQ ID NOs: 1-38. In some embodiments, the subset of peptide structures comprises one or more amino acid sequence of SEQ ID NOs: 5, 8-11, 13-14, 16-22, 26-28, 30-31, and 34-38. In some embodiments, the subset of peptide structures comprises one or more amino acid sequence of SEQ ID NOs: 3, 7, 9, 28, 29, 32, and 33. In some embodiments, the subset of peptide structures comprises one or more amino acid sequence of SEQ ID NOs: 1-4, 6-7, 12, 15, 23-25, 28, 29, and 32. In some embodiments, the method further comprises performing a differential expression analysis using the quantification data for the plurality of subjects. In some embodiments, the CRC full change (CRC.FC) is used, wherein the CRC.FC is the average multiplicative difference between the CRC and healthy patients groups for an individual marker. In some embodiments, the CRC.FC is equal to 2, meaning that the transition is twice as likely to be expressed in CRC when compared to an healthy patients. In some embodiments, the CRC.FC is equal to 0.5, meaning that the transition is half as likely to be expressed in CRC when compared to an healthy patients. In some embodiments, the CRC.FC is a differential expression analysis of a peptide transition from a first biological sample from an individual with CRC or AA and a second control sample from an individual not having CRC or AA. In some embodiments, the differential expression analysis is determining an expression fold-change of a peptide transition from a first biological sample from an individual with CRC or AA and a second control sample from an individual not having CRC or AA. In some embodiments, the differential expression analysis is determining an abundance fold-change of a peptide transition from a first biological sample from an individual with CRC or AA and a second control sample from an individual not having CRC or AA.

In some embodiments, the biological sample comprises at least one of blood, serum, plasma, or stool. In some embodiments, the biological sample comprises a blood sample. In some embodiments, the biological sample comprises a whole blood sample. In some embodiments, the biological sample comprises a serum sample. In some embodiments, the biological sample comprises a plasma sample. In some embodiments, the biological sample comprises a stool sample.

In some aspects, provided herein is a method of treating colorectal cancer (CRC) or advanced adenoma (AA) in a subject comprising receiving peptide structure data corresponding to a set of proteins in a biological sample obtained from a subject, wherein the peptide structure data comprises at least one peptide structure from Table 10. In some embodiments, the at least one peptide structure identified from a plurality of peptide structures comprise the amino acid of SEQ ID NOs: 1-38. In some embodiments, the method further comprises inputting quantification data for the at least one peptide structure into a machine-learning model trained to generate disease indicator for CRC or AA based on the quantification data. In some embodiments, the method further comprises identifying, by the machine-learning model, the disease indicator. In some embodiments, the method further comprises selecting at least one of a plurality of treatment regimens described herein to treat CRC or AA based upon the disease indicator. In some embodiments, the set of proteins comprises one or more glycoproteins. In some embodiments, the one or more glycoproteins comprises the amino acid sequences of SEQ ID NOs: 39-54.

In some aspects, provided herein is a method of treating colorectal cancer (CRC) or advanced adenoma (AA) in a subject comprising receiving peptide structure data corresponding to a set of proteins in the biological sample. In some embodiments, the method further comprises inputting quantification data identified from the peptide structure data for a set of peptide structures into a machine-learning model trained to identify a disease indicator based on the quantification data, wherein the peptide structure data comprises at least one peptide structure identified from a plurality of peptide structures in Table 10. In some embodiments, the at least one peptide structure identified from a plurality of peptide structures comprise the amino acid of SEQ ID NOs: 1-38. In some embodiments, the method further comprises identifying, by the machine-learning model, the disease indicator. In some embodiments, the method further comprises determining a classification for CRC or AA based upon the identified disease indicator. In some embodiments, the method further comprises selecting at least one of a plurality of treatment regimens described herein to treat CRC or AA based upon the classification. In some embodiments, the set of proteins comprises one or more glycoproteins. In some embodiments, the one or more glycoproteins comprises the amino acid sequences of SEQ ID NOs: 39-54.

In some embodiment, the method further comprises administering a selected treatment regimen to the subject. In some embodiments, the treatment regimen for an individual having colorectal cancer (CRC) or advanced adenoma (AA) or an individual suspected of having CRC or AA is selected from a surgery, an antimetabolite, a chemotherapeutic therapy, a topoisomerase inhibitor, an alkylating agent, a targeted therapeutic agent, an immune-therapeutic, an immunotherapy, an antibody, a T-cell related therapy, a radiotherapy, or a combination thereof.

In some aspects, provided herein is a method of diagnosing an individual with colorectal cancer (CRC) or advanced adenoma (AA), comprising detecting the presence or amount of at least one peptide structure structures from Table 10. In some embodiments, the method further comprises inputting a quantification of the detected at least one peptide structure into a machine-learning model trained to generate a class label. In some embodiments, the method further comprises determining if the class label is above or below a threshold for a classification; identifying a diagnostic classification for the individual based on whether the class label is above or below a threshold for the classification. In some embodiments, the method further comprises and diagnosing the individual as having CRC or AA based on the diagnostic classification.

In some embodiments, the quantification data is generated using a liquid chromatography-mass spectrometry (LC-MS) system. In some embodiments, the peptide structure data is generated using multiple reaction monitoring mass spectrometry (MRM-MS). In some embodiments, the amount of at least one peptide structure is none, or below a detection limit. In some embodiments, the at least one peptide structure is a glycopeptide from Table 10. In some embodiments, the glycopeptide comprise the amino acid of SEQ ID NOs: 1-38.

In some embodiments, the CRC is one of early-stage. In some embodiments, the CRC is one of late-stage CRC. In some embodiments, the CRC is one of stage I CRC, stage II CRC, stage III CRC, or stage IV CRC. In some embodiments, the CRC is one of severe CRC.

In some embodiments, the at least one peptide structure comprises one or more peptide structures identified in Table 10. In some embodiments, the at least one peptide structure comprises two or more peptide structures identified in Table 10. In some embodiments, the at least one peptide structure comprises three or more peptide structures identified in Table 10. In some embodiments, the at least one peptide structure comprises four or more peptide structures identified in Table 10. In some embodiments, the at least one peptide structure comprises five or more peptide structures identified in Table 10. In some embodiments, the at least one peptide structure comprises 10 or more peptide structures identified in Table 10. In some embodiments, the at least one peptide structure comprises 15 or more peptide structures identified in Table 10. In some embodiments, the at least one peptide structure comprises 20 or more peptide structures identified in Table 10. In some embodiments, the at least one peptide structure comprises 25 or more peptide structures identified in Table 10. In some embodiments, the at least one peptide structure comprises 30 or more peptide structures identified in Table 10. In some embodiments, the at least one peptide structure comprises 35 or more peptide structures identified in Table 10. In some embodiments, the at least one peptide structure comprises at least one peptide comprising the sequence set forth in any one of SEQ ID NOs: 1-38. In some embodiments, the at least one peptide structure comprises at least one peptide comprising the sequence set forth in any one of SEQ ID NOs: 5, 8-11, 13-14, 16-22, 26-28, 30-31, and 34-38. In some embodiments, the at least one peptide structure comprises at least one peptide comprising the sequence set forth in any one of SEQ ID NOs: 3, 7, 9, 28, 29, 32, and 33. In some embodiments, the at least one peptide structure comprises at least one peptide comprising the sequence set forth in any one of SEQ ID NOs: 1-4, 6-7, 12, 15, 23-25, 28, 29, and 32.

In some embodiments, the subject has one or more risk factors or clinical indicators of colorectal cancer (CRC). In some embodiments, the subject has one or more risk factors associated with CRC. In some embodiments, the risk factor for CRC is selected from the group consisting of age, irritable bowel disease, type 2 diabetes, a family history of CRC, a genetic syndrome (e.g., Lynch syndrome), obesity, smoking, alcohol consumption, dietary choices, and limited physical activity. In some embodiments, the clinical indicator of CRC is selected from the group consisting of changes in bowel habits, bloody stool, diarrhea, constipation, persistent abdominal pain, persistent abdominal cramps, and unexplained weight loss. In some embodiments, the individual is determined have a healthy state, wherein a healthy state comprises the absence of CRC or AA. In some embodiments, the method further comprises generating a report that includes a diagnosis based on the corresponding state detected for the subject.

In some aspects, provided herein is a method of training a model to diagnose a subject with one of a plurality of states associated with colorectal cancer (CRC) or advanced adenoma (AA), the method comprising receiving quantification data for a panel of peptide structures for a plurality of subjects diagnosed with the plurality of states associated with CRC or AA. In some embodiments, the method further comprises training a machine-learning model to determine a state of the plurality of states a biological sample from the subject based on the quantification data.

In some embodiments, the training the machine-learning model to determine the state of the plurality of states comprises training the machine-learning model to generate a class label for the state of the plurality of states. In some embodiments, the plurality of states comprises at least one of a CRC state, an AA state, or a healthy state. In some embodiments, the plurality of states comprises at least two of a CRC state, an AA state, or a healthy state. In some embodiments, the plurality of states comprises each of a CRC state, an AA state, or a healthy state. In some embodiments, the machine-learning model comprises a logistic regression model. In some embodiments, the machine-learning model comprises a regularized regression model. In some embodiments, the regularized regression model comprises a least absolute shrinkage and selection operator (LASSO) regression model.

In some embodiments, the at least one peptide structure comprises at least one, at least two, at least three, at least five, at least 10, at least 15, at least 20, at least 25, at least 30, or at least 35 different peptides comprising the sequence set forth in any one of SEQ ID NOs: 1-38. In some embodiments, the at least one peptide structure comprises at least one peptide comprising the sequence set forth in any one of SEQ ID NOs: 1-38. In some embodiments, the at least one peptide structure comprises at least two different peptides comprising the sequence set forth in any one of SEQ ID NOs: 1-38. In some embodiments, the at least one peptide structure comprises at least three different peptides comprising the sequence set forth in any one of SEQ ID NOs: 1-38. In some embodiments, the at least one peptide structure comprises at least four different peptides comprising the sequence set forth in any one of SEQ ID NOs: 1-38. In some embodiments, the at least one peptide structure comprises at least five different peptides comprising the sequence set forth in any one of SEQ ID NOs: 1-38. In some embodiments, the at least one peptide structure comprises at least 10 different peptides comprising the sequence set forth in any one of SEQ ID NOs: 1-38. In some embodiments, the at least one peptide structure comprises at least 20 different peptides comprising the sequence set forth in any one of SEQ ID NOs: 1-38. In some embodiments, the at least one peptide structure comprises at least 30 different peptides comprising the sequence set forth in any one of SEQ ID NOs: 1-38.

In some embodiments, the at least one peptide structure comprises at least one, at least two, at least three, at least four, or at least five different peptides comprising the sequence set forth in any one of SEQ ID NOs: 3, 7, 9, 28, 29, 32, and 33. In some embodiments, the at least one peptide structure comprises at least one peptide comprising the sequence set forth in any one of SEQ ID NOs: 3, 7, 9, 28, 29, 32, and 33. In some embodiments, the at least one peptide structure comprises at least two different peptides comprising the sequence set forth in any one of SEQ ID NOs: 3, 7, 9, 28, 29, 32, and 33. In some embodiments, the at least one peptide structure comprises at least three different peptides comprising the sequence set forth in any one of SEQ ID NOs: 3, 7, 9, 28, 29, 32, and 33. In some embodiments, the at least one peptide structure comprises at least four different peptides comprising the sequence set forth in any one of SEQ ID NOs: 3, 7, 9, 28, 29, 32, and 33. In some embodiments, the at least one peptide structure comprises at least five different peptides comprising the sequence set forth in any one of SEQ ID NOs: 3, 7, 9, 28, 29, 32, and 33. In some embodiments, the at least one peptide structure comprises at least six different peptides comprising the sequence set forth in any one of SEQ ID NOs: 3, 7, 9, 28, 29, 32, and 33. In some embodiments, the at least one peptide structure comprises seven different peptides comprising the sequence set forth in any one of SEQ ID NOs: 3, 7, 9, 28, 29, 32, and 33.

In some embodiments, the at least one peptide structure comprises at least one, at least two, at least three, at least five, at least 10 different peptides comprising the sequence set forth in any one of SEQ ID NOs: 1-4, 6-7, 12, 15, 23-25, 28, 29, and 32. In some embodiments, the at least one peptide structure comprises at least one peptide comprising the sequence set forth in any one of SEQ ID NOs: 1-4, 6-7, 12, 15, 23-25, 28, 29, and 32. In some embodiments, the at least one peptide structure comprises at least two different peptides comprising the sequence set forth in any one of SEQ ID NOs: 1-4, 6-7, 12, 15, 23-25, 28, 29, and 32. In some embodiments, the at least one peptide structure comprises at least three different peptides comprising the sequence set forth in any one of SEQ ID NOs: 1-4, 6-7, 12, 15, 23-25, 28, 29, and 32. In some embodiments, the at least one peptide structure comprises at least five different peptides comprising the sequence set forth in any one of SEQ ID NOs: 1-4, 6-7, 12, 15, 23-25, 28, 29, and 32. In some embodiments, the at least one peptide structure comprises at least 10 different peptides comprising the sequence set forth in any one of SEQ ID NOs: 1-4, 6-7, 12, 15, 23-25, 28, 29, and 32.

In some embodiments, the at least one peptide structure comprises at least one, at least two, at least three, at least five, at least 10, at least 15, or at least 20 different peptides comprising the sequence set forth in any one of SEQ ID NOs: 5, 8-11, 13-14, 16-22, 26-28, 30-31, and 34-38. In some embodiments, the at least one peptide structure comprises at least one peptide comprising the sequence set forth in any one of SEQ ID NOs: 5, 8-11, 13-14, 16-22, 26-28, 30-31, and 34-38. In some embodiments, the at least one peptide structure comprises at least two different peptides comprising the sequence set forth in any one of SEQ ID NOs: 5, 8-11, 13-14, 16-22, 26-28, 30-31, and 34-38. In some embodiments, the at least one peptide structure comprises at least three different peptides comprising the sequence set forth in any one of SEQ ID NOs: 5, 8-11, 13-14, 16-22, 26-28, 30-31, and 34-38. In some embodiments, the at least one peptide structure comprises at least five different peptides comprising the sequence set forth in any one of SEQ ID NOs: 5, 8-11, 13-14, 16-22, 26-28, 30-31, and 34-38. In some embodiments, the at least one peptide structure comprises at least 10 different peptides comprising the sequence set forth in any one of SEQ ID NOs: 5, 8-11, 13-14, 16-22, 26-28, 30-31, and 34-38. In some embodiments, the at least one peptide structure comprises at least 20 different peptides comprising the sequence set forth in any one of SEQ ID NOs: 5, 8-11, 13-14, 16-22, 26-28, 30-31, and 34-38.

In some embodiments, the at least one peptide structure comprises a peptide sequence and a glycan structure, wherein the glycan structure is attached to a linking site position in the peptide sequence in accordance with Table 10. In some embodiments, the glycan structure of the peptide sequence corresponds to a glycan structure GL number in accordance with Table 10, wherein the glycan structure comprises a symbol structure in accordance with the glycan structure GL number according to Table 10, Table 11A, and Table 11B. In some embodiments, the glycan structure of the peptide sequence corresponds to a glycan structure GL number in accordance with Table 10, wherein the glycan structure comprises a composition in accordance with the glycan structure GL number, Table 10, Table 11A, and Table 11B. In some embodiments, a rightmost N-acetylgalactosamine of the glycan structure in Table 11A is attached to a linking site position in the peptide sequence in accordance with Table 10, and wherein a bottommost N-acetylglucosamine of the glycan structure in Table 11B is attached to a linking site position in the peptide sequence in accordance with Table 10.

In some embodiments, provided herein is a composition comprising one or more peptide structures from Table 10. In some embodiments, the at least one peptide structure comprises a peptide sequence and a glycan structure, wherein the glycan structure is attached to a linking site position in the peptide sequence in accordance with Table 10. In some embodiments, the glycan structure of the peptide sequence corresponds to a glycan structure GL number in accordance with Table 10, wherein the glycan structure comprises a symbol structure in accordance with the glycan structure GL number according to Table 10, Table 11A, and Table 11B. In some embodiments, the glycan structure of the peptide sequence corresponds to a glycan structure GL number in accordance with Table 10, wherein the glycan structure comprises a composition in accordance with the glycan structure GL number, Table 10, Table 11A, and Table 11B. In some embodiments, a rightmost N-acetylgalactosamine (GalNAc) of the glycan structure in Table 11A is attached to a linking site position in the peptide sequence in accordance with Table 10. In some embodiments, a bottommost N-acetylglucosamine (GlcNAc) of the glycan structure in Table 11B is attached to a linking site position in the peptide sequence in accordance with Table 10.

In certain aspects, provided herein is a method of classifying a biological sample obtained from a subject with respect to a plurality of states associated with colorectal cancer (CRC) or advanced adenoma (AA), the method comprising: receiving mass spectrometry (MS) quantification data obtained from the biological sample, wherein the quantification data comprises a quantification level associated with each of one or more peptides derived from one or more proteins of Table 9; inputting the MS quantification data into a machine-learning model, wherein the machine-learning model is trained on one or more training MS quantification data sets comprising quantification data from training samples characterized as having CRC, having AA, or not having CRC or AA, wherein, for each training sample, the associated training MS quantification data comprises a quantification level associated with each of one or more peptides derived from one or more proteins of Table 9; and classifying the biological sample with respect to the plurality of states associated with CRC or AA.

In some embodiments, the method is performed on a system comprising one or more processors. In some embodiments, the biological sample is classified via the method as having a CRC. In some embodiments, the biological sample is classified via the method as having an AA. In some embodiments, the biological sample is classified as not having a CRC or an AA. In some embodiments, at least one of the training samples characterized as not having CRC or AA is obtained from a healthy subject, such as a subject not suffering from any gastrointestinal or colon-associated conditions or diseases.

In certain aspects, the MS quantification data comprises information in addition to a quantity that is useful to the methods described herein, e.g., information relevant to the identity of a quantified compound or an attribute thereof, such as chromatography retention time.

In some embodiments, the MS quantification data comprises peptide sequence information. In some embodiments, the MS quantification data comprises post-translational modification information, including the amino acid site of a post-translation modification. In some embodiments, the post-translation modification information comprises glycan information, including glycan structure(s) and/or amino acid site of attachment information. In some embodiments, the MS quantification data comprises the quantification level associated with one or more peptides derived from each protein of Model 1 or Model 2 of Table 9. In some embodiments, the MS quantification data comprises the quantification level associated with one or more peptides of Table 10. In some embodiments, the peptide of Table 10 is a glycopeptide. In some embodiments, the MS quantification data comprises a quantification level associated with at least one peptide derived from each protein of Model 1 or Model 2 of Table 9. In some embodiments, the training MS quantification data comprises the quantification level associated with one or more peptides derived from each protein of Model 1 or Model 2 of Table 9. In some embodiments, the training MS quantification data comprises the quantification level associated with one or more peptides of Table 10. In some embodiments, the peptide of Table 10 is a glycopeptide. In some embodiments, the quantification level of a peptide reflects an absolute amount of the peptide or a relative amount of the peptide, such as based on various MS quantification techniques described herein. In some embodiments, the quantification level of a peptide reflects an absence of the peptide. In some embodiments, the MS quantification data and/or the training MS quantification data are obtained, in whole or in part, from an automated peak detection technique, including, e.g., automated AUC determination, such as described in U.S. Patent Application Publication No. 2020/0372973, which is incorporated herein by reference in its entirety and for all purposes.

In some embodiments, the MS quantification data is obtained from analysis of the biological sample, or a derivative thereof, using a MS technique. In some embodiments, the MS technique is a targeted MS technique, such as an MS technique designed to interrogate a sample, such as a biological sample, for the presence or absence (including amount thereof) of one or more peptides derived from one or more proteins of Table 9. In some embodiments, an MS technique designed to interrogate a sample, such as a biological sample, for the presence or absence (including amount thereof) of one or more peptides derived from each protein of Model 1 and/or Model 2 of Table 9. In some embodiments, the MS technique is an MRM technique. In some embodiments, the MRM technique is configured based on one or more of transitions 1-38, including sets of transitions such as (a) 3, 7, 9, 28, 29, 32, and/or (b) 1-4, 6-7, 12, 15, 23-25, 28, 29, 32. In some embodiments, the MRM technique is a dynamic MRM technique that designs mass spectrometry data acquisition in view of chromatography retention times.

In certain aspects, provided herein is a method of determining a glycopeptide profile of a biological sample obtained from a subject, wherein the glycopeptide profile is based on a quantification level associated with one or more peptides derived from one or more proteins of Table 9; the method comprising: subjecting the biological sample, or a derivative thereof, to a mass spectrometry (MS) technique configured to assess the one or more peptides derived from one or more proteins of Table 9 to obtain MS information; determining the quantification level associated with the one or more peptides derived from one or more proteins of Table 9 based on the MS information; and determining the glycopeptide profile based on the quantification level associated with the one or more peptides derived from one or more proteins of Table 9.

In certain aspects, provided herein is a method of performing a mass spectrometry analysis, the method comprising subjecting a biological sample, or a derivative thereof, to a mass spectrometry (MS) technique configured to assess one or more peptides derived from one or more proteins of Table 9. In some embodiments, the MS technique is a targeted MS technique, such as described herein. In some embodiments, the targeted MS technique is an MRM technique, such as described herein.

In certain aspects, provided herein is a method of treating colorectal cancer (CRC) or advanced adenoma (AA) in a subject, the method comprising: classifying the subject with respect to a plurality of states associated with CRC or AA; and administering to the subject a treatment regimen based on the classification.

In certain aspects, provided herein is a system comprising one or more processors, and memory storing one or more programs, the one or more programs configured to be executed by the one or more processors, the one or more programs including instructions for the methods provided herein, such as a method of classifying a biological sample obtained from a subject with respect to a plurality of states associated with colorectal cancer (CRC) or advanced adenoma (AA).

### X. EMBODIMENTS

1. A method of detecting one or more multiple-reaction-monitoring (MRM) transitions, comprising obtaining, or having obtained, a biological sample from a patient, wherein the biological sample comprises one or more glycoproteins, glycans, or glycopeptides; digesting and/or fragmenting a glycoprotein in the sample; and detecting an MRM transition selected from the group consisting of transitions 1-38.

2. The method of embodiment 1, wherein the fragmenting a glycopeptide in the sample occurs after introducing the sample, or a portion thereof, into the mass spectrometer.

3. The method of any one of embodiments 1-2, wherein the fragmenting a glycoprotein or glycopeptide in the sample produces a peptide or glycopeptide consisting essentially of an amino acid sequence selected from the group consisting of SEQ ID NOs: 1-38, and combinations thereof.

4. The method of any one of embodiments 1-3, wherein the fragmenting a glycoprotein or glycopeptide in the sample produces a peptide or glycopeptide consisting essentially of an amino acid sequence selected from the group consisting of SEQ ID NOs: 5, 8-11, 13-14, 16-22, 26-28, 30-31, and 34-38, and combinations thereof.

5. The method of any one of embodiments 1-3, wherein the fragmenting a glycopeptide in the sample produces a peptide or glycopeptide consisting essentially of an amino acid sequence selected from the group consisting of SEQ ID NOs: 3, 7, 9, 28, 29, 32, and 33, and combinations thereof.

6. The method of any one of embodiments 1-3, wherein the fragmenting a glycopeptide in the sample produces a peptide or glycopeptide consisting essentially of an amino acid sequence selected from the group consisting of SEQ ID NOs: 1-4, 6-7, 12, 15, 23-25, 28, 29, and 32, and combinations thereof.

7. The method of any one of embodiments 1-6, wherein the MRM transition is selected from the transitions, or any combinations thereof, in any one of Tables 1-3.

8. The method of any one of embodiments 1-7, wherein detecting a MRM transition selected from the group consisting of transitions 1-38 comprises detecting a MRM transition using a triple quadrupole (QQQ) mass spectrometer or a quadrupole time-of-flight (qTOF) mass spectrometer.

9. The method of any one of embodiments 1-8, wherein the one or more glycopeptides comprises a peptide or glycopeptide consisting essentially of an amino acid sequence selected from the group consisting of SEQ ID NOs: 1-38, and combinations thereof.

10. The method of any one of embodiments 1-9, comprising detecting one or more MRM transitions indicative of one or more glycans selected from the group consisting of glycan 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof.

11. The method of embodiment 10, further comprising quantifying a first glycan and quantifying a second glycan; and further comprising comparing the quantification of the first glycan with the quantification of the second glycan.

12. The method of embodiment 10 or 11, further comprising associating the detected glycan with a peptide residue site, whence the glycan was bonded.

13. The method of any one of embodiments 1-12, comprising normalizing the amount of glycopeptide based on the amount of a peptide or glycopeptide consisting essentially of an amino acid having a SEQ ID. No: 1-38.

14. A method for identifying a classification for a sample, the method comprising quantifying by mass spectrometry (MS) one or more glycopeptides in a sample wherein the glycopeptides each, individually in each instance, consist essentially of an amino acid sequence selected from the group consisting of SEQ ID NOs: 1-38, and combinations thereof; and inputting the quantification into a trained model to generate an output probability; determining if the output probability is above or below a threshold for a classification; and identifying a classification for the sample based on whether the output probability is above or below a threshold for a classification.

15. The method of embodiment 14, wherein the sample is a biological sample from a patient or individual having a disease or condition.

16. The method of embodiment 15, wherein the patient has colorectal cancer or an adenoma, including advanced adenoma.

17. The method of any one of embodiments 14-16, wherein the MS is MRM-MS with a QQQ and/or qTOF mass spectrometer.

18. The method of embodiment any one of embodiments 14-17 wherein the trained model was trained using a machine learning algorithm selected from the group consisting of a deep learning algorithm, a neural network algorithm, an artificial neural network algorithm, a supervised machine learning algorithm, a linear discriminant analysis algorithm, a combined discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof.

19. The method of embodiment any one of embodiments 14-18, wherein the classification is a disease classification or a disease severity classification.

20. The method of embodiment 19, wherein the classification is identified with greater than 80% confidence, greater than 85% confidence, greater than 90% confidence, greater than 95% confidence, greater than 99% confidence, or greater than 99.9999% confidence.

21. The method of embodiment of any one of embodiments 14-20, further comprising quantifying by MS one or several glycopeptide(s) in a sample at a first time point; quantifying by MS one or several glycopeptide(s) in a sample at a second time point; and comparing the quantification at the first time point with the quantification at the second time point.

22. The method of embodiment 21, further comprising quantifying by MS one or several glycopeptide in a sample at a third time point; quantifying by MS one or several glycopeptide in a sample at a fourth time point; and comparing the quantification at the fourth time point with the quantification at the third time point.

23. The method of any one of embodiments 14-22, further comprising monitoring the health status of a patient.

24. The method of embodiment 23, wherein monitoring the health status of a patient comprises monitoring the onset and progression of disease in a patient with risk factors such as genetic mutations, as well as detecting cancer recurrence.

25. The method of any one of embodiments 14-24, further comprising quantifying by MS an amino acid sequence selected from the group consisting of SEQ ID NOs: 1-38.

26. The method of any one of embodiments 14-25, further comprising quantifying by MS an amino acid sequence selected from the group consisting of SEQ ID NOs: 5, 8-11, 13-14, 16-22, 26-28, 30-31, and 34-38, and combinations thereof.

27. The method of any one of embodiments 14-25, further comprising quantifying by MS an amino acid sequence selected from the group consisting of SEQ ID NOs: 3, 7, 9, 28, 29, 32, and 33, and combinations thereof.

28. The method of any one of embodiments 14-25, further comprising quantifying by MS an amino acid sequence selected from the group consisting of SEQ ID NOs: 1-4, 6-7, 12, 15, 23-25, 28, 29, and 32, and combinations thereof.

29. The method of any one of embodiments 14-25, further comprising quantifying by MS one or more glycans selected from the group consisting of glycans 3200, 3210, 3300, 3310, 3320, 3400, 3410, 3420, 3500, 3510, 3520, 3600, 3610, 3620, 3630, 3700, 3710, 3720, 3730, 3740, 4200, 4210, 4300, 4301, 4310, 4311, 4320, 4400, 4401, 4410, 4411, 4420, 4421, 4430, 4431, 4500, 4501, 4510, 4511, 4520, 4521, 4530, 4531, 4540, 4541, 4600, 4601, 4610, 4611, 4620, 4621, 4630, 4631, 4641, 4650, 4700, 4701, 4710, 4711, 4720, 4730, 5200, 5210, 5300, 5301, 5310, 5311, 5320, 5400, 5401, 5402, 5410, 5411, 5412, 5420, 5421, 5430, 5431, 5432, 5500, 5501, 5502, 5510, 5511, 5512, 5520, 5521, 5522, 5530, 5531, 5541, 5600, 5601, 5602, 5610, 5611, 5612, 5620, 5621, 5631, 5650, 5700, 5701, 5702, 5710, 5711, 5712, 5720, 5721, 5730, 5731, 6200, 6210, 6300, 6301, 6310, 6311, 6320, 6400, 6401, 6402, 6410, 6411, 6412, 6420, 6421, 6432, 6500, 6501, 6502, 6503, 6510, 6511, 6512, 6513, 6520, 6521, 6522, 6530, 6531, 6532, 6540, 6541, 6600, 6601, 6602, 6603, 6610, 6611, 6612, 6613, 6620, 6621, 6622, 6623, 6630, 6631, 6632, 6640, 6641, 6642, 6652, 6700, 6701, 6711, 6721, 6703, 6713, 6710, 6711, 6712, 6713, 6720, 6721, 6730, 6731, 6740, 7200, 7210, 7400, 7401, 7410, 7411, 7412, 7420, 7421, 7430, 7431, 7432, 7500, 7501, 7510, 7511, 7512, 7600, 7601, 7602, 7603, 7604, 7610, 7611, 7612, 7613, 7614, 7620, 7621, 7622, 7623, 7632, 7640, 7700, 7701, 7702, 7703, 7710, 7711, 7712, 7713, 7714, 7720, 7721, 7722, 7730, 7731, 7732, 7740, 7741, 7751, 8200, 9200, 9210, 10200, 11200, 12200, and combinations thereof.

30. The method of any one of embodiments 14-29, further comprising diagnosing a patient with a disease or condition based on the classification.

31. The method of embodiment 30, further comprising diagnosing the patient as having colorectal cancer or adenoma, including advanced adenoma based on the classification.

32. The method of any one of embodiments 14-31, comprising diagnosing the patient as having adenoma, including advanced adenoma, and treating the patient with resection.

33. The method of any one of embodiments 14-31, comprising diagnosing the patient as having colorectal cancer, and treating the patient with a therapeutically effective amount of a therapeutic agent selected from the group consisting of a therapeutic, an adjuvant, a neo-adjuvant, a chemo-embolization, a hyperthermic intraperitoneal, and combinations thereof.

34. The method of any one of embodiments 14-31, comprising diagnosing the patient as having colorectal cancer, and treating the patient with a therapeutically effective amount of an alkylating agent, an antimetabolite, a topoisomerase inhibitor, a cytotoxic agent, and combinations thereof.

35. The method of any one of embodiments 14-31, comprising diagnosing the patient as having colorectal cancer, and treating the patient with a therapeutically effective amount of a targeted therapeutic agent.

36. The method of any one of embodiments 14-31, comprising diagnosing the patient as having colorectal cancer, and treating the patient with a therapeutically effective amount of an immune-therapeutic.

37. The method of embodiment 36, wherein the immune-therapeutic is selected from the group consisting of immune checkpoint inhibitors.

38. The method of embodiment 37, wherein the checkpoint inhibitors are selected from the group consisting of PD-1-, PD-L1-, CTLA-4-inhibitors, and combinations thereof.

39. The method of any one of embodiments 14-31, comprising diagnosing the patient as having colorectal cancer, and treating the patient with a therapeutically effective amount of T-cell-related therapies,

40. The method of embodiment 39, wherein the T-cell-related therapies are selected from the group consisting of CAR-T-approaches, TCR-approaches, and combinations thereof.

41. The method of any one of embodiments 14-31, comprising diagnosing the patient as having colorectal cancer, and treating the patient with a therapeutically effective amount of a cancer vaccine.

42. The method of any one of embodiments 14-31, comprising diagnosing the patient as having colorectal cancer, and treating the patient with a therapeutically effective amount of radiotherapy.

43. The method of embodiment 42, wherein the radiotherapy is selected from the group consisting of external beam-radiotherapy and internal-radiotherapy, chemoradiation, brachytherapy, and combinations thereof.

44. The method of any one of embodiments 15-43, comprising obtaining, or having obtained, a biological sample from a patient, wherein the biological sample comprises one or more glycoproteins or glycopeptides; digesting and/or fragmenting one or more glycoproteins or glycopeptides in the sample; detecting and quantifying at least one or more multiple-reaction-monitoring (MRM) transition(s) selected from the group consisting of transitions 1-38.

45. The method of embodiment 44, further comprising using a machine learning algorithm to train a model using the MRM transitions as inputs.

46. A method for classifying a biological sample, comprising obtaining, or having obtained, a biological sample from a patient, wherein the biological sample comprises one or more glycoproteins or glycopeptides; digesting and/or fragmenting one or more glycoproteins or glycopeptides in the sample; detecting and quantifying at least one or more multiple-reaction-monitoring (MRM) transition associated with at least one or more glycopeptides consisting essentially of an amino acid sequence selected from the group consisting of SEQ ID NOs: 1-38, and combinations thereof; and inputting the quantification into a trained model to generate an output probability; determining if the output probability is above or below a threshold for a classification; and classifying the biological sample based on whether the output probability is above or below a threshold for a classification.

47. The method of embodiment 46, comprising detecting and quantifying at least one or more multiple-reaction-monitoring (MRM) transition associated with at least one or more glycopeptides consisting essentially of an amino acid sequence selected from the group consisting of SEQ ID NOs: 5, 8-11, 13-14, 16-22, 26-28, 30-31, and 34-38, and combinations thereof.

48. The method of embodiment 46, comprising detecting and quantifying at least one or more multiple-reaction-monitoring (MRM) transition associated with at least one or more glycopeptides consisting essentially of an amino acid sequence selected from the group consisting of SEQ ID NOs: 3, 7, 9, 28, 29, 32, and 33, and combinations thereof.

49. The method of embodiment 46, comprising detecting and quantifying at least one or more multiple-reaction-monitoring (MRM) transition associated with at least one or more glycopeptides consisting essentially of an amino acid sequence selected from the group consisting of SEQ ID NOs: 1-4, 6-7, 12, 15, 23-25, 28, 29, and 32, and combinations thereof, and combinations thereof.

50. The method of embodiment 46, comprising training a machine learning algorithm using the MRM transitions as inputs.

51. A method for treating a patient having colorectal cancer or adenoma, including advanced adenoma; the method comprising obtaining, or having obtained, a biological sample from the patient; digesting and/or fragmenting, or having digested or having fragmented, one or more glycoproteins or glycopeptides in the sample; and detecting and quantifying one or more multiple-reaction-monitoring (MRM) transitions selected from the group consisting of transitions 1-38; inputting the quantification into a trained model to generate an output probability; determining if the output probability is above or below a threshold for a classification; and classifying the patient based on whether the output probability is above or below a threshold for a classification, wherein the classification is selected from the group consisting of: (A) a patient in need of resection; (B) a patient in need of a therapeutic agent; (C) a patient in need of an alkylating agent; (D) a patient in need of a targeted therapeutic agent; (E) a patient in need of immune-therapeutic; (F) a patient in need of immune checkpoint inhibitors; (G) a patient in need of T-cell-related therapies; (H) a patient in need of a cancer vaccine; (I) a patient in need of radiotherapy; (J) a patient in need of a colonoscopy; (K) or a combination thereof; performing, or having performed, a resection if classification A or K is determined; performing, or having performed, a radiotherapy if classification I or K is determined; performing, or having performed, a colonoscopy if classification J or K is determined; or administering a therapeutically effective amount of a therapeutic agent to the patient; wherein the therapeutic agent is selected from a therapeutic agent if classification B or K is determined; or wherein the therapeutic agent is selected from alkylating agent if classification C or K is determined; or wherein the therapeutic agent is selected from a targeted therapeutic agent if classification D or K is determined; wherein the therapeutic agent is selected from an immune-therapeutic agent if classification E or K is determined; wherein the therapeutic agent is selected from an immune checkpoint inhibitor if classification F or K is determined; wherein the therapeutic agent is selected from a T-cell-related therapy if classification G or K is determined; and wherein the therapeutic agent is selected from a cancer vaccine if classification H or K is determined.

52. The method of embodiment 51, comprising conducting multiple-reaction-monitoring mass spectrometry (MRM-MS) on the biological sample.

53. The method of embodiment 51 or 52, comprising quantifying one or more glycopeptides consisting essentially of an amino acid sequence selected from the group consisting of SEQ ID NOs: 1-38 and combinations thereof.

54. The method of any one of embodiments 51-53, comprising quantifying one or more glycopeptides consisting essentially of an amino acid sequence selected from the group consisting of SEQ ID NOs: 5, 8-11, 13-14, 16-22, 26-28, 30-31, and 34-38, and combinations thereof.

55. The method of any one of embodiments 51-53, comprising quantifying one or more glycopeptides consisting essentially of an amino acid sequence selected from the group consisting of SEQ ID NOs: 3, 7, 9, 28, 29, 32, and 33, and combinations thereof.

56. The method of any one of embodiments 51-53, comprising quantifying one or more glycopeptides consisting essentially of an amino acid sequence selected from the group consisting of SEQ ID NOs: 1-4, 6-7, 12, 15, 23-25, 28, 29, and 32, and combinations thereof.

57. The method of any one of embodiments 51-56, comprising inputting the quantification of the amount of a glycopeptide consisting essentially of an amino acid sequence selected from the group consisting of SEQ ID NOs: 1-38 into a trained model or using the quantification to train a model.

58. The method of embodiment 57, wherein the machine learning algorithm is selected from the group consisting of a deep learning algorithm, a neural network algorithm, an artificial neural network algorithm, a supervised machine learning algorithm, a linear discriminant analysis algorithm, a combined discriminant analysis algorithm, a quadratic discriminant analysis algorithm, a support vector machine algorithm, a linear basis function kernel support vector algorithm, a radial basis function kernel support vector algorithm, a random forest algorithm, a genetic algorithm, a nearest neighbor algorithm, k-nearest neighbors, a naive Bayes classifier algorithm, a logistic regression algorithm, or a combination thereof.

59. The method of any one of embodiments 51-58, wherein detecting and quantifying one or more multiple-reaction-monitoring (MRM) transitions selected from the group consisting of transitions 1-38 comprises selecting peaks and/or quantifying detected glycopeptide fragments with a machine learning algorithm.

60. A method for training a machine learning algorithm, comprising providing a first data set of MRM transition signals indicative of a sample comprising one or more glycopeptides, each glycopeptide, individually, consisting essentially of an amino acid sequence selected from the group consisting of SEQ ID NOs: 1-38; providing a second data set of MRM transition signals indicative of a control sample; and comparing the first data set with the second data set using a machine learning algorithm.

61. The method of embodiment 60, wherein the sample comprising a glycopeptide consisting essentially of an amino acid sequence selected from the group consisting of SEQ ID NOs: 1-38 is a sample from a patient having colorectal cancer or adenoma, including advanced adenoma.

62. The method of embodiment 61, wherein the sample comprising a glycopeptide consisting essentially of an amino acid sequence selected from the group consisting of SEQ ID NOs: 5, 8-11, 13-14, 16-22, 26-28, 30-31, and 34-38, and combinations thereof is a sample from a patient having colorectal cancer or adenoma, including advanced adenoma.

63. The method of embodiment 61, wherein the sample comprising a glycopeptide consisting essentially of an amino acid sequence selected from the group consisting of SEQ ID NOs: 3, 7, 9, 28, 29, 32, and 33, and combinations thereof is a sample from a patient having colorectal cancer or adenoma, including advanced adenoma.

64. The method of embodiment 61, wherein the sample comprising a glycopeptide consisting essentially of an amino acid sequence selected from the group consisting of SEQ ID NOs: 1-4, 6-7, 12, 15, 23-25, 28, 29, and 32, and combinations thereof is a sample from a patient having colorectal cancer or adenoma, including advanced adenoma.

65. The method of any one of embodiments 60-64, wherein the control sample is a sample from a patient not having colorectal cancer or adenoma, including advanced adenoma.

66. The method of any one of embodiments 60-65, wherein the sample comprising a glycopeptide consisting essentially of an amino acid sequence selected from the group consisting of SEQ ID NOs: 1-38 is a pooled sample from one or more patients having colorectal cancer or adenoma, including advanced adenoma.

67. The method of any one of embodiments 60-66, wherein the control sample is a pooled sample from one or more patients not having colorectal cancer or adenoma, including advanced adenoma.

68. A method for diagnosing a patient having colorectal cancer or adenoma, including advanced adenoma; the method comprising obtaining, or having obtained, a biological sample from the patient; performing mass spectrometry of the biological sample using MRM-MS with a QQQ and/or qTOF spectrometer to detect and quantify one or more glycopeptides consisting essentially of an amino acid sequence selected from the group consisting of SEQ ID NOs: 1-38; or to detect one or more MRM transitions selected from transitions 1-38; inputting the quantification of the detected glycopeptides or the MRM transitions into a trained model to generate an output probability; determining if the output probability is above or below a threshold for a classification; and identifying a diagnostic classification for the patient based on whether the output probability is above or below a threshold for a classification; and diagnosing the patient as having colorectal cancer or adenoma, including advanced adenoma based on the diagnostic classification.

69. The method of embodiment 68, wherein the analyzing the detected glycopeptides comprises using a machine learning algorithm.

70. The method of embodiment 68, comprising training a machine learning algorithm using the MRM transitions as inputs.

71. The method of embodiment 68, comprising performing mass spectrometry of the biological sample using MRM-MS with a QQQ and/or qTOF spectrometer to detect and quantify one or more glycopeptides consisting essentially of an amino acid sequence selected from the group consisting of SEQ ID NOs: 5, 8-11, 13-14, 16-22, 26-28, 30-31, and 34-38, and combinations thereof.

72. The method of embodiment 68, comprising performing mass spectrometry of the biological sample using MRM-MS with a QQQ and/or qTOF spectrometer to detect and quantify one or more glycopeptides consisting essentially of an amino acid sequence selected from the group consisting of SEQ ID NOs: 3, 7, 9, 28, 29, 32, and 33, and combinations thereof.

73. The method of embodiment 68, comprising performing mass spectrometry of the biological sample using MRM-MS with a QQQ and/or qTOF spectrometer to detect and quantify one or more glycopeptides consisting essentially of an amino acid sequence selected from the group consisting of SEQ ID NOs: 1-4, 6-7, 12, 15, 23-25, 28, 29, and 32, and combinations thereof.

74. A glycopeptide consisting of an amino acid sequence selected from the group consisting of SEQ ID NOs: 1-38, and combinations thereof.

75. A glycopeptide consisting of an amino acid sequence selected from the group consisting of SEQ ID NOs: 5, 8-11, 13-14, 16-22, 26-28, 30-31, and 34-38, and combinations thereof.

76. A glycopeptide consisting of an amino acid sequence selected from the group consisting of SEQ ID NOs: 3, 7, 9, 28, 29, 32, and 33, and combinations thereof.

77. A glycopeptide consisting of an amino acid sequence selected from the group consisting of SEQ ID NOs: 1-4, 6-7, 12, 15, 23-25, 28, 29, and 32, and combinations thereof.

78. A glycopeptide consisting essentially an amino acid sequence selected from the group consisting essentially of SEQ ID NOs: 1-38, and combinations thereof.

79. A glycopeptide consisting essentially an amino acid sequence selected from the group consisting essentially of SEQ ID NOs: 5, 8-11, 13-14, 16-22, 26-28, 30-31, and 34-38, and combinations thereof.

80. A glycopeptide consisting essentially an amino acid sequence selected from the group consisting essentially of SEQ ID NOs: 3, 7, 9, 28, 29, 32, and 33, and combinations thereof.

81. A glycopeptide consisting essentially an amino acid sequence selected from the group consisting essentially of SEQ ID NOs: 1-4, 6-7, 12, 15, 23-25, 28, 29, and 32, and combinations thereof.

82. A kit comprising one or more glycopeptide standard(s), a buffer, and one or more glycopeptides consisting essentially of an amino acid sequence selected from the group consisting of SEQ ID NOs: 1-38.

83. A kit comprising one or more glycopeptide standard(s), a buffer, and one or more glycopeptides consisting essentially of an amino acid sequence selected from the group consisting of SEQ ID NOs: 5, 8-11, 13-14, 16-22, 26-28, 30-31, and 34-38, and combinations thereof.

84. A kit comprising one or more glycopeptide standard(s), a buffer, and one or more glycopeptides consisting essentially of an amino acid sequence selected from the group consisting of SEQ ID NOs: 3, 7, 9, 28, 29, 32, and 33, and combinations thereof.

85. A kit comprising one or more glycopeptide standard(s), a buffer, and one or more glycopeptides consisting essentially of an amino acid sequence selected from the group consisting of SEQ ID NOs: 1-4, 6-7, 12, 15, 23-25, 28, 29, and 32, and combinations thereof.

86. A computer-implemented method of training a neural network for detecting one or more MRM transition(s), comprising collecting a set of mass spectrometry spectra of one or more glycopeptides consisting essentially of an amino acid sequence selected from the group consisting of SEQ ID NOs: 1-38; annotating the spectra including identifying at least one of a start, stop, maximum, or combination thereof, of a peak in a spectrum or spectra to create an annotated set of mass spectrometry spectra; creating a first training set comprising the collected set of mass spectrometry spectra, the annotated set of mass spectrometry spectra, and a second set of mass spectrometry spectra of one or more glycopeptides consisting essentially of an amino acid sequence selected from the group consisting of SEQ ID NOs: 1-38; training the neural network in a first stage using the first training set; creating a second training set for a second stage of training comprising the first training set and mass spectrometry spectra that are incorrectly detected as comprising one or more glycopeptides consisting essentially of an amino acid sequence selected from the group consisting of SEQ ID NOs: 1-38 after the first stage of training; and training the neural network in a second stage using the second training set.

87. The method of embodiment 86, wherein the one or more glycopeptides are each individually in each instance selected from glycopeptides consisting essentially of an amino acid sequence selected from the group consisting of SEQ ID NOs: 5, 8-11, 13-14, 16-22, 26-2830-31, and 34-38, and combinations thereof.

88. The method of embodiment 86, wherein the one or more glycopeptides are each individual in each instance glycopeptides consisting essentially of an amino acid sequence selected from the group consisting of SEQ ID NOs: 3, 7, 9, 28, 29, 32, and 33, and combinations thereof.

89. The method of embodiment 86, wherein the one or more glycopeptides are each individual in each instance glycopeptides consisting essentially of an amino acid sequence selected from the group consisting of SEQ ID NOs: 1-4, 6-7, 12, 15, 23-25, 28, 29, and 32, and combinations thereof.

90. The method of embodiment 46, comprising using the MRM transitions as inputs to train a model.

1.-80. (canceled)

81. A method of classifying a biological sample with respect to a plurality of states associated with colorectal cancer (CRC) or advanced adenoma (AA), the method comprising:
receiving peptide structure data corresponding to a set of proteins in the biological sample, wherein the peptide structure data comprises at least one peptide structure from Table 10;
inputting quantification data identified from the peptide structure data into a machine-learning model trained to identify a disease indicator based on the quantification data;
identifying, by the machine-learning model, the disease indicator; and
classifying the biological sample with respect to the plurality of states associated with the CRC or the AA based upon the identified disease indicator.

82. The method of claim 81, wherein the set of proteins comprises one or more glycoproteins, and
wherein the at least one peptide structure comprises a glycopeptide.

83. The method of claim 81, wherein the machine-learning model comprises a least absolute shrinkage and selection operator (LASSO) regression model; and
wherein the quantification data is generated using a liquid chromatography-mass spectrometry (LC-MS) system or reaction monitoring mass spectrometry (MRM-MS).

84. The method of claim 81, wherein the quantification data for a peptide structure of the set of peptide structures comprises at least one of an abundance, a relative abundance, a normalized abundance, a differential abundance, a relative quantity, an adjusted quantity, a normalized quantity, a relative concentration, an adjusted concentration, or a normalized concentration.

85. The method of claim 81, further comprising:
receiving biological samples from a plurality of subjects; and
performing a differential expression analysis using the quantification data for the plurality of subjects.

86. The method of claim 81, further comprising:
selecting at least one of a plurality of treatment regimens to treat the CRC or the AA based upon the classification; and
administering the at least one of the plurality of treatment regimens to treat the CRC or the AA based upon the classification.

87. A method of diagnosing an individual with colorectal cancer (CRC) or advanced adenoma (AA), comprising:
detecting a presence or amount of at least one peptide structure from a plurality of peptide structures from Table 10;
inputting a quantification of the detected at least one peptide structure into a machine-learning model trained to generate a class label;
determining if the class label is above or below a threshold for a classification;
identifying a diagnostic classification for the individual based on whether the class label is above or below the threshold for the classification; and
diagnosing the individual as having the CRC or the AA based on the diagnostic classification.

88. The method of claim 87, wherein the quantification is generated using a liquid chromatography-mass spectrometry (LC-MS) system, and the at least one peptide structure is generated using multiple reaction monitoring mass spectrometry (MRM-MS).

89. The method of claim 87, wherein the CRC is one of stage I CRC, stage II CRC, stage III CRC, or stage IV CRC; and
wherein the individual is determined have a healthy state, in response to an absence of the CRC or the AA.

90. The method of claim 87, further comprising, training the machine-learning model based on:
receiving quantification data for a panel of peptide structures for a plurality of subjects diagnosed with a plurality of states associated with the CRC or the AA, wherein the plurality of states comprises at least one of a CRC state, an AA state, or a healthy state; and
training the machine-learning model to determine a state of the plurality of states based on a biological sample from the subject based on the quantification data.

91. The method of claim 90, wherein training of the machine-learning model to determine the state of the plurality of states comprises training the machine-learning model to generate a class label for the state of the plurality of states.

92. The method of claim 87, wherein the at least one peptide structure comprises a peptide sequence and a glycan structure, and wherein the glycan structure of the peptide sequence corresponds to a glycan structure GL number in accordance with the Table 10, wherein the glycan structure comprises a symbol structure in accordance with the glycan structure GL number according to the Table 10, Table 11A, and Table 11B.

93. The method of claim 92,
wherein a rightmost N-acetylgalactosamine of the glycan structure in Table 11A is attached to a linking site position in the peptide sequence in accordance with Table 10, and
wherein a bottommost N-acetylglucosamine of the glycan structure in Table 11B is attached to a linking site position in the peptide sequence in accordance with Table 10.

94. A composition comprising one or more peptide structures from Table 10.

95. A method of classifying a biological sample obtained from a subject with respect to a plurality of states associated with colorectal cancer (CRC) or advanced adenoma (AA), the method comprising:
receiving mass spectrometry (MS) quantification data obtained from the biological sample,
wherein the quantification data comprises a quantification level associated with each of one or more peptides derived from one or more proteins of Table 9;
inputting the MS quantification data into a machine-learning model,
wherein the machine-learning model is trained on one or more training MS quantification data sets comprising quantification data from training samples characterized as having the CRC, having the AA, or not having the CRC or the AA,
wherein, for each training sample, the associated training MS quantification data comprises a quantification level associated with each of one or more peptides derived from the one or more proteins of the Table 9; and
classifying the biological sample with respect to the plurality of states associated with the CRC or the AA.

96. The method of claim 95, wherein the biological sample is classified as having the CRC, or the AA, or not having the CRC and the AA.

97. The method of claim 95, wherein the MS quantification data comprises one or more of peptide sequence information, post-translational modification information comprising glycan information, the quantification level associated with one or more peptides derived from each protein of Model 1 or Model 2 of Table 9, the quantification level associated with one or more peptides of Table 10, and a quantification level associated with at least one peptide derived from each protein of Model 1 or Model 2 of Table 9.

98. The method of claim 95, wherein the training MS quantification data comprises one or more of the quantification level associated with one or more peptides derived from each protein of Model 1 or Model 2 of Table 9 and the quantification level associated with one or more peptides of Table 10.

99. A method of determining a glycopeptide profile of a biological sample obtained from a subject,
wherein the glycopeptide profile is based on a quantification level associated with one or more peptides derived from one or more proteins of Table 9;
the method comprising:
subjecting the biological sample, or a derivative thereof, to a mass spectrometry (MS) technique configured to assess the one or more peptides derived from one or more proteins of Table 9 to obtain MS information;
determining the quantification level associated with the one or more peptides derived from one or more proteins of Table 9 based on the MS information; and
determining the glycopeptide profile based on the quantification level associated with the one or more peptides derived from one or more proteins of Table 9.

100. The method of claim 99, wherein the one or more peptides comprise a sequence set forth in SEQ ID NO:5 and/or SEQ ID NO:6.


SEQUENCE LISTING (54 sequences)

SEQ ID NO: 1 - protein, 32 aa, synthetic construct
ADTHDEILEGLNFNLTEIPEAQIHEGFQELLR

SEQ ID NO: 2 - protein, 16 aa, synthetic construct
YLGNATAIFFLPDEGK

SEQ ID NO: 3 - protein, 16 aa, synthetic construct
YLGNATAIFFLPDEGK

SEQ ID NO: 4 - protein, 33 aa, synthetic construct
EGDHEFLEVPEAQEDVEATFPVHQPGNYSCSYR

SEQ ID NO: 5 - protein, 19 aa, synthetic construct
VSNQTLSLFFTVLQDVPVR

SEQ ID NO: 6 - protein, 19 aa, synthetic construct
VSNQTLSLFFTVLQDVPVR

SEQ ID NO: 7 - protein, 22 aa, synthetic construct
GCVLLSYLNETVTVSASLESVR

SEQ ID NO: 8 - protein, 32 aa, synthetic construct
SLGNVNFTVSAEALESQELCGTEVPSVPEHGR

SEQ ID NO: 9 - protein, 32 aa, synthetic construct; identical to SEQ ID NO: 8

SEQ ID NO: 10 - protein, 16 aa, synthetic construct
YTGNASALFILPDQDK

SEQ ID NO: 11 - protein, 7 aa, synthetic construct
ENGTISR

SEQ ID NO: 12 - protein, 24 aa, synthetic construct
QIPLCANLVPVPITNATLDQITGK

SEQ ID NO: 13 - protein, 15 aa, synthetic construct
QDQCIYNTTYLNVQR

SEQ ID NO: 14 - protein, 15 aa, synthetic construct
QDQCIYNTTYLNVQR

SEQ ID NO: 15 - protein, 7 aa, synthetic construct
ENGTVSR

SEQ ID NO: 16 - protein, 18 aa, synthetic construct
FSEFWDLDPEVRPTSAVA

SEQ ID NO: 17 - protein, 22 aa, synthetic construct
ADGTVNQIEGEATPVNLTEPAK

SEQ ID NO: 18 - protein, 22 aa, synthetic construct
ADGTVNQIEGEATPVNLTEPAK

SEQ ID NO: 19 - protein, 22 aa, synthetic construct
ADGTVNQIEGEATPVNLTEPAK

SEQ ID NO: 20 - protein, 22 aa, synthetic construct
ADGTVNQIEGEATPVNLTEPAK

SEQ ID NO: 21 - protein, 22 aa, synthetic construct
ADGTVNQIEGEATPVNLTEPAK

SEQ ID NO: 22 - protein, 15 aa, synthetic construct
NPWGQVEWNGSWSDR

SEQ ID NO: 23 - protein, 16 aa, synthetic construct
EHEGAIYPDNTTDFQR

SEQ ID NO: 24 - protein, 16 aa, synthetic construct
EHEGAIYPDNTTDFQR

SEQ ID NO: 25 - protein, 22 aa, synthetic construct
AALAAFNAQNNGSNFQLEEISR

SEQ ID NO: 26 - protein, 22 aa, synthetic construct
AALAAFNAQNNGSNFQLEEISR

SEQ ID NO: 27 - protein, 13 aa, synthetic construct
NLFLNHSENATAK

SEQ ID NO: 28 - protein, 16 aa, synthetic construct
VVLHPNYSQVDIGLIK

SEQ ID NO: 29 - protein, 16 aa, synthetic construct
VVLHPNYSQVDIGLIK

SEQ ID NO: 30 - protein, 16 aa, synthetic construct
VVLHPNYSQVDIGLIK

SEQ ID NO: 31 - protein, 16 aa, synthetic construct
VVLHPNYSQVDIGLIK

SEQ ID NO: 32 - protein, 11 aa, synthetic construct
YKNNSDISSTR

SEQ ID NO: 33 - protein, 16 aa, synthetic construct
LQAPLNYTEFQKPICL

SEQ ID NO: 34 - protein, 22 aa, synthetic construct
VTQVYAENGTVLQGSTVASVYK

SEQ ID NO: 35 - protein, 22 aa, synthetic construct
VTQVYAENGTVLQGSTVASVYK

SEQ ID NO: 36 - protein, 22 aa, synthetic construct
VTQVYAENGTVLQGSTVASVYK

SEQ ID NO: 37 - protein, 20 aa, synthetic construct
ACLNSTYEYIFNNCHELYSR

SEQ ID NO: 38 - protein, 20 aa, synthetic construct
ACLNSTYEYIFNNCHELYSR

SEQ ID NO: 39 - protein, 418 aa, synthetic construct
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 40 - protein, 495 aa, synthetic construct
MSMLVVFLLLWGVTWGPVTEAAIFYETQPSLWAESESLLKPLANVTLTCQAHLETPDFQLFKNGVAQEPVHLDSPAIKHQFLLTGDTQGRYRCRSGLSTGWTQLSKLLELTGPKSLPAPWLSMAPVSWITPGLKTTAVCRGVLRGVTFLLRREGDHEFLEVPEAQEDVEATFPVHQPGNYSCSYRTDGEGALSEPSATVTIEELAAPPPPVLMHHGESSQVLHPGNKVTLTCVAPLSGVDFQLRRGEKELLVPRSSTSPDRIFFHLNAVALGDGGHYTCRYRLHDNQNGWSGDSAPVELILSDETLPAPEFSPEPESGRALRLRCLAPLEGARFALVREDRGGRRVHRFQSPAGTEALFELHNISVADSANYSCVYVDLKPPFGGSAPSERLELHVDGPPPRPQLRATWSGAVLAGRDAVLRCEGPIPDVTFELLREGETKAVKTVRTPGAAANLELIFVGPQHAGNYRCRYRSWVPHTFESELSDPVELLVAES

SEQ ID NO: 41 - protein, 1474 aa, synthetic construct
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 42 - protein, 423 aa, synthetic construct
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 43 - protein, 201 aa, synthetic construct
MALSWVLTVLSLLPLLEAQIPLCANLVPVPITNATLDRITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 44 - protein, 201 aa, synthetic construct
MALSWVLTVLSLLPLLEAQIPLCANLVPVPITNATLDRITGKWFYIASAFRNEEYNKSVQEIQATFFYFTPNKTEDTIFLREYQTRQNQCFYNSSYLNVQRENGTVSRYEGGREHVAHLLFLRDTKTLMFGSYLDDEKNWGLSFYADKPETTKEQLGEFYEALDCLCIPRSDVMYTDWKKDKCEPLEKQHEKERKQEEGES

SEQ ID NO: 45 - protein, 99 aa, synthetic construct
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 46 - protein, 189 aa, synthetic construct
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 47 - protein, 821 aa, synthetic construct
MPTVISASVAPRTAAEPRSPGPVPHPAQSKATEAGGGNPSGIYSAIISRNFPIIGVKEKTFEQLHKKCLEKKVLYVDPEFPPDETSLFYSQKFPIQFVWKRPPEICENPRFIIDGANRTDICQGELGDCWFLAAIACLTLNQHLLFRVIPHDQSFIENYAGIFHFQFWRYGEWVDVVIDDCLPTYNNQLVFTKSNHRNEFWSALLEKAYAKLHGSYEALKGGNTTEAMEDFTGGVAEFFEIRDAPSDMYKIMKKAIERGSLMGCSIDDGTNMTYGTSPSGLNMGELIARMVRNMDNSLLQDSDLDPRGSDERPTRTIIPVQYETRMACGLVRGHAYSVTGLDEVPFKGEKVKLVRLRNPWGQVEWNGSWSDRWKDWSFVDKDEKARLQHQVTEDGEFWMSYEDFIYHFTKLEICNLTADALQSDKLQTWTVSVNEGRWVRGCSAGGCRNFPDTFWTNPQYRLKLLEEDDDPDDSEVICSFLVALMQKNRRKDRKLGASLFTIGFAIYEVPKEMHGNKQHLQKDFFLYNASKARSKTYINMREVSQRFRLPPSEYVIVPSTYEPHQEGEFILRVFSEKRNLSEEVENTISVDRPVKKKKTKPIIFVSDRANSNKELGVDQESEEGKGKTSPDKQKQSPQPQPGSSDQESEEQQQFRNIFKQIAGDDMEICADELKKVLNTVVNKHKDLKTHGFTLESCRSMIALMDTDGSGKLNLQEFHHLWNKIKAWQKIFKHYDTDQSGTINSYEMRNAVNDAGFHLNNQLYDIITMRYADKHMNIDFDSFICCFVRLEGMFRAFHAFDKDGDGIIKLNVLEWLQLTMYA

SEQ ID NO: 48 - protein, 1065 aa, synthetic construct
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYKHRGVYSSDVFDIFPGTYQTLEMFPRTPGIWLLHCHVTDHIHAGMETTYTVLQNEDTKSG

SEQ ID NO: 49 - protein, 367 aa, synthetic construct
MKSLVLLLCLAQLWGCHSAPHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 50 - protein, 406 aa, synthetic construct
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 51 - protein, 453 aa, synthetic construct
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 52 - protein, 638 aa, synthetic construct
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 53 - protein, 355 aa, synthetic construct [sequence omitted]

SEQ ID NO: 54 - protein, 1703 aa, synthetic construct
MSLLCVGVKKAKFDGAQEKFNTYVTLKVQNVKSTTIAVRGSQPSWEQDFMFEINRLDLGLTVEVWNKGLIWDTMVGTVWIPLRTIRQSNEEGPGEWLTLDSQVIMADSEICGTKDPTFHRILLDTRFELPLDIPEEEARYWAKKLEQLNAMRDQDEYSFQDEQDKPLPVPSNQCCNWNYFGWGEQHNDDPDSAVDDRDSDYRSETSNSIPPPYYTTSQPNASVHQYSVRPPPLGSRESYSDSMHSYEEFSEPQALSPTGSSRYASSGELSQGSSQLSEDFDPDEHSLQGSDMEDERDRDSYHSCHSSVSYHKDSPRWDQDEEELEEDLEDFLEEEELPEDEEELEEEEEEVPDDLGSYAQREDVAVAEPKDFKRISLPPAAPGKEDKAPVAPTEAPDMAKVAPKPATPDKVPAAEQIPEAEPPKDEESFRPREDEEGQEGQDSMSRAKANWLRAFNKVRMQLQEARGEGEMSKSLWFKGGPGGGLIIIDSMPDIRKRKPIPLVSDLAMSLVQSRKAGITSALASSTLNNEELKNHVYKKTLQALIYPISCTTPHNFEVWTATTPTYCYECEGLLWGIARQGMRCTECGVKCHEKCQDLLNADCLQRAAEKSSKHGAEDRTQNIIMVLKDRMKIRERNKPEIFELIQEIFAVTKTAHTQQMKAVKQSVLDGTSKWSAKISITVVCAQGLQAKDKTGSSDPYVTVQVGKTKKRTKTIYGNLNPVWEENFHFECHNSSDRIKVRVWDEDDDIKSRVKQRFKRESDDFLGQTIIEVRTLSGEMDVWYNLDKRTDKSAVSGAIRLHISVEIKGEEKVAPYHVQYTCLHENLFHFVTDVQNNGVVKIPDAKGDDAWKVYYDETAQEIVDEFAMRYGVESIYQAMTHFACLSSKYMCPGVPAVMSTLLANINAYYAHTTASTNVSASDRFAASNFGKERFVKLLDQLHNSLRIDLSMYRNNFPASSPERLQDLKSTVDLLTSITFFRMKVQELQSPPRASQVVKDCVKACLNSTYEYIFNNCHELYSREYQTDPAKKGEVLPEEQGPSIKNLDFWSKLITLIVSIIEEDKNSYTPCLNQFPQELNVGKISAEVMWNLFAQDMKYAMEEHDKHRLCKSADYMNLHFKVKWLYNEYVTELPAFKDRVPEYPAWFEPFVIQWLDENEEVSRDFLHGALERDKKDGFQQTSEHALFSCSVVDVFSQLNQSFEIIKKLECPDPQIVGHYMRRFAKTISNVLLQYADIISKDFASYCSKEKEKVPCILMNNTQQLRVQLEKMFEAMGGKELDAEASDILKELQVKLNNVLDELSRVFATSFQPHIEECVKQMGDILSQVKGTGNVPASACSSVAQDADNVLQPIMDLLDSNLTLFAKICEKTVLKRVLKELWKLVMNTMEKTIVLPPLTDQTMIGNLLRKHGKGLEKGRVKLPSHSDGTQMIFNAAKELGQLSKLKDHMVREEAKSLTPKQCAVVELALDTIKQYFHAGGVGLKKTFLEKSPDLQSLRYALSLYTQATDLLIKTFVQTQSAQGLGVEDPVGEVSVHVELFTHPGTGEHKVTVKVVAANDLKWQTSGIFRPFIEVNIIGPQLSDKKRKFATKSKNNSWAPKYNESFQFTLSADAGPECYELQVCVKDYCFAREDRTVGLAVLQLRELAQRGSAACWLPLGRRIHMDDTGLTVLRILSQRSNDEVAKEFVKLKSDTRSAEEGGAAPAP